(19) United States
(12) Patent Application Publication
(10) Pub. No.: US 2022/0062371 A1
(43) Pub. Date: Mar. 3, 2022

(54) LIGAND-DRUG-CONJUGATES AS SUBSTRATES FOR SELECTIVE CLEAVAGE BY THE EXOPEPTIDASE ACTIVITY OF CATHEPSIN B

(71) Applicant: DEBIOPHARM RESEARCH & MANUFACTURING S.A., Martigny (CH)

(72) Inventors: Manfred MUTTER, Grenzach-Wyhlen (DE); Nathalie BELLOCQ, Fully (CH); Daniel BIASSE, Avully (CH); Alain RAZANAME, Martigny (CH); Léo MARX, Martigny (CH); Christophe CHARDONNENS, La Tour-de-Trême (CH); Patrick GARROUSTE, Saxon (CH)

(21) Appl. No.: 16/764,343

(22) PCT Filed: Nov. 14, 2018
(86) PCT No.: PCT/EP2018/081269

§ 371 Date: May 14, 2020

(30) Foreign Application Priority Data
Nov. 14, 2017 (EP) 17201589.3

(51) Int. Cl. A61K 38/07; A61K 47/54; A61K 38/12; A61K 31/537; A61K 31/704; A61K 31/4745; A61K 31/337; A61K 31/475; A61K 31/519; A61K 31/5517; A61K 47/64; A61K 47/65

(52) U.S. Cl. CPC A61K 38/07; A61K 47/542; A61K 38/12; A61K 31/537; A61K 31/704; A61K 47/65; A61K 31/337; A61K 31/475; A61K 31/519; A61K 31/5517; A61K 47/64; A61K 31/4745

(57) ABSTRACT

The present invention relates to ligand-drug-conjugates for the treatment of disease. In particular, the present invention relates to ligand-drug-conjugates comprising a linker system, which is selectively recognized and cleaved by the exopeptidase (i.e. carboxydipeptidase) activity of Cathepsin B, resulting in improved intracellular delivery of a drug to a target cell. The present invention also relates to ligand-drug-conjugates for the intracellular delivery of cytotoxic agents in tumor cells.

### STATEMENT REGARDING SEQUENCE LISTING

The Sequence Listing associated with this application is provided in text format in lieu of a paper copy, and is hereby incorporated by reference into the specification. The name of the text file containing the Sequence Listing is 250112_401USPC_SequenceListing.txt. The text file is 110 KB, was created on Jul. 26, 2021, and is being submitted electronically via EFS-Web.

### DESCRIPTION

The present invention relates to ligand-drug-conjugates (LDCs) for the treatment of disease. In particular, the present invention relates to ligand-drug-conjugates comprising a linker system, which is selectively recognized and cleaved by the exopeptidase (i.e. carboxydipeptidase) activity of Cathepsin B, resulting in improved delivery of a drug to a target cell. The present invention also relates to ligand-drug-conjugates comprising a linker system, which allows the release of multiple drugs, resulting in improved efficacy. In some aspects, the present invention relates to ligand-drug-conjugates, which achieve high drug loading (e.g. high drug-antibody-ratio) thus resulting in significantly improved efficacy. In some aspects, the present invention also relates to ligand-drug-conjugates for the intracellular delivery of cytotoxic drugs to tumor or cancer cells.

### BACKGROUND OF THE INVENTION

Recently, a great deal of interest has surrounded the use of enzyme-triggered drug release systems such as antibody-drug-conjugates (ADCs) for the targeted delivery of cytotoxic agents to tumor cells. Antibody-drug-conjugates generally consist of three components: an antibody (e.g. a monoclonal antibody) that targets an antigen highly expressed on tumor cells, a cytotoxic agent (sometimes called “toxin” or “payload”), and a linker system which can release the cytotoxic agent (payload) from the antibody upon internalization into cancer cells. Ideally, antibody-drug-conjugates should retain the favorable pharmacokinetic and functional properties of antibodies, remain intact and nontoxic in systemic circulation (blood), and become active at the target site with drug released in sufficient amount to kill the target cell. Thus, one of the biggest challenges in the development of antibody-drug-conjugates represents the design of linker systems for the conjugation of antibody and drug, which are nontoxic and stable in systemic circulation, but which are nevertheless capable of releasing the drug inside the target cell in sufficient amount and at a satisfactory rate.

A large number of linker systems have been developed for the specific intracellular release of cytotoxic drugs. There are two main families of linkers: cleavable and non-cleavable. Cleavable linkers usually utilize an inherent property of the target cell, e.g. a tumor cell, for selectively releasing the drug (e.g. a cytotoxic agent) from the conjugate, namely (1) protease-sensitivity (enzyme-triggered release systems), (2) pH-sensitivity, or (3) glutathione-sensitivity. Non-cleavable linkers usually rely on the complete degradation of the antibody after internalization of the conjugate in the target cell. An example of antibody-drug-conjugate using a non-cleavable linker is the humanized anti-HER2 (anti-ErbB2) antibody-maytansine conjugate trastuzumab-emtansine (T-DM1 or Kadcyla®, LoRusso et al. Clin. Cancer Res. 2011, 17, 6437-6447).

Peptide linkers have also been proposed as they combine good stability in the systemic circulation with rapid intracellular drug release by specific enzymes. In particular, peptide linkers comprising a valine-citrulline (Val-Cit) dipeptide as substrate for intracellular cleavage by Cathepsin B (Cat B) have been described (Lu et al. Int. J. Mol. Sci. 2016, 17, 561-582; Jain et al. Pharm. Res. 2015, 32(11), 3526-3540; Dubowchik et al. Bioconj. Chem. 2002, 13, 855-859). Cat B is a lysosomal cysteine protease implicated in a number of physiological processes, which differs from other cysteine proteases in that it possesses endopeptidase activity and also exopeptidase activity, meaning that it can remove dipeptide units from the C-termini of proteins and peptides (Turk et al. Biochim. Biophys. Acta 2012, 1824(1), 68-88). Therefore, the exopeptidase activity of Cat B is a carboxydipeptidase activity.

Typically, enzymatic cleavage of a conjugate (e.g. by Cat B) releases the antibody and a linker-drug conjugate at the target site. The linker must, in turn, allow rapid release of the drug from the linker-drug conjugate. Thus, “self-immolative” spacers between linker and drug have been proposed for enhancing drug release rate after enzymatic cleavage. Self-immolative spacers can usually release a drug, e.g. a cytotoxic drug, by elimination- or cyclization-based mechanisms.

An example of a linker system comprising a self-immolative spacer is the para-amino benzyloxycarbonyl (PABC) linker as used e.g. in the bremtuximab-vedotin conjugate Adcetris® (Younes et al. N. Engl. J. Med. 2010, 363, 1812-1821; Jain et al. Pharm. Res. 2015, 32(11), 3526-3540). The PABC linker system as used in antibody-drug-conjugates utilizes a protease-sensitive Val-Cit-PABC dipeptide linker, which can be recognized and cleaved by Cathepsin B. A maleimidocaproyl moiety is typically used for attaching the linker unit to the antibody and serves as a spacer between drug and antibody for avoiding steric conflicts in substrate recognition by Cathepsin B. After enzymatic cleavage of the citrulline-PABC amide bond, the resulting PABC-substituted drug e.g. monomethyl auristatin E (MMAE) spontaneously undergoes a 1,6-elimination that releases the free drug (e.g. MMAE) as the product.

However, the efficacy of the PABC linker system for delivering a drug to a target cell may be limited due to the slow intracellular drug release and the limited stability of the Val-Cit-PABC moiety in plasma (Dorywalska et al. Mol. Cancer Ther. 2016, 15(5), 958-970).

Furthermore, in vivo studies indicate that the pharmacokinetic (PK) properties (e.g. distribution, hepatic clearance) of Val-Cit-PABC-based conjugates depends on the number of drug molecules attached to the antibody moiety (Strop et al. Chem. & Biol. 2013, 20, 161-167). Thus an important parameter of antibody-drug-conjugates is the drug-antibody-ratio (DAR) (or drug load(ing)) referring to the average number of drug molecule(s) attached to one antibody moiety. The DAR value not only affects efficacy, but also the PK properties and toxicity of the conjugates. In particular, a high DAR value (i.e. high drug loading) has been associated with decreased PK properties and/or higher toxicity due to aggregation of the conjugate molecules and/or premature cleavage.

To overcome these problems, it has been proposed to employ hydrophilic linker systems containing negatively charged sulfonate groups, polyethylene glycol groups or pyrophosphate diester groups in order to reduce conjugate aggregation. Likewise, WO 2015/123679 A1 discloses hydrophilic antibody-drug-conjugates based on the combination of a hydrophilic linker with a hydrophilic drug such as an auristatin chemically modified with a hydrophilic amino acid, e.g. Thr. The hydrophilic conjugates of WO 2015/123679 A1 are said to exhibit good PK properties in an in vivo model. However, the efficacy of the hydrophilic linker systems e.g. as disclosed in WO 2015/123679 A1 may be limited due to unspecific enzymatic cleavage, slow intracellular drug release and/or increased lysosomal trapping.

Furthermore, the achievement of high DAR values (high drug loading) is often limited by increased tendency for aggregation of ADCs, steric factors (e.g. originating from multiple attachment sites) or lack of systemic stability.

There is thus a need for novel compounds comprising a linker system, which is stable in the systemic circulation and can rapidly release and deliver a drug to a target cell.

It is hence an object of the present invention to provide compounds comprising a linker system that is stable in the systemic circulation, and allows the rapid release and delivery of a drug to a target cell in a traceless manner. It is a further object of the present invention to provide pharmaceutical compositions comprising such compounds.

It is yet another object of the present invention to provide compounds comprising a linker system that is stable in the systemic circulation and, at the same time, is capable of releasing multiple drug molecules (e.g. multiple payloads), wherein the individual drug molecules may be the same or different.

A further object of the present invention is to provide compounds or compositions that can be used in methods of treating or preventing a cancer, an autoimmune disease and/or an infectious disease.

### SUMMARY OF THE PRESENT INVENTION

The present invention provides a new cleavable linker system which can be used in ligand-drug-conjugates. The linker system is characterized by a C-terminal dipeptide unit carrying a drug or a vector group on a side chain thereof. The C-terminal dipeptide unit acts as highly specific substrate for the exopeptidase (i.e. carboxydipeptidase) activity of Cathepsin B, resulting in improved intracellular cleavage and drug release. The linker system is stable and enables the release of multiple drug molecules (e.g. multiple payloads), wherein the individual drug molecules may be the same or different, resulting in improved efficacy. The linker system also enables to achieve a high drug loading (e.g. high DAR) thus resulting in significantly improved efficacy.

The present invention thus relates to a compound represented by the general formula (I) or (I′):

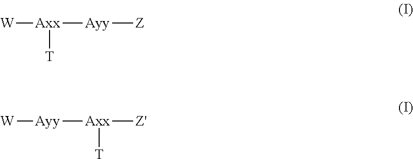

wherein, in formulae (I) and (I′),
W represents from a moiety represented by the following formula (III):

W₁-Dxx-Dyy---- (III)
wherein
W₁ represents a moiety derived from a drug that differs from a native drug only by virtue of the covalent attachment to Dxx as shown in formula (III), if the drug is an auristatin analog, the auristatin analog is auristatin Phe (AF), auristatin Cit (ACit), auristatin Arg (AArg), auristatin Lys (ALys), auristatin Orn (AOrn), auristatin 2,3-diamino-propionic acid (ADab) or auristatin 2,4-diamino-butyric acid (ADap), preferably AF; or
W₁ represents a moiety derived from a drug with the proviso that W₁ is not an auristatin analog;
Dxx represents a single covalent bond or an amino acid having a hydrophobic side chain, preferably an amino acid selected from Phe, Val, Tyr, homo-Phe and Ala, preferably Phe or Val, wherein the single covalent bond or amino acid having a hydrophobic side chain is optionally attached to moiety W₁ via a divalent moiety selected from maleimides, triazoles, hydrazones, carbonyl-containing groups, and derivatives thereof, preferably via a divalent maleimide derivative;
Dyy represents a single covalent bond, Phe or an amino acid having a basic side chain, preferably an amino acid selected from Arg, Lys, Citrulline (Cit), Ornithine (Orn), 2,3-diamino-propionic acid (Dap), 2,4-diamino-butyric acid (Dab), more preferably Arg or Cit;
with the proviso that if Dxx is an amino acid having a hydrophobic side chain, Dyy is Phe or an amino acid having a basic side chain, and if Dxx is a single covalent bond, Dyy is a single covalent bond, Phe or an amino acid with a basic side chain, preferably Arg or Cit;
and the broken line indicates covalent attachment to the N-terminus of Axx in formula (I) or the N-terminus of Ayy in formula (I′);
or W represents a peptide moiety represented by formula (Ia), (Ia′) or (Ib):

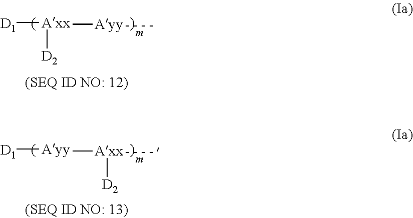

wherein, in formulae (Ia) and (Ia′),
A′yy represents an amino acid selected from Phe, Ala, Trp, Tyr, phenylglycine (Phg), Met, Val, His, Lys, Arg, Cit, 2-amino-butyric acid (Abu), Orn, with the proviso that A′yy in formula (Ia′) is not an amino acid in the (D) configuration;
D₁ represents a moiety derived from a drug;
m is an integer of 1 to 10;
if m=1, then A′xx represents a trifunctional amino acid such as an amino dicarboxylic acid or a diamino carboxylic acid with the proviso that A′xx in formula (Ia) is not an amino acid in the (D) configuration, D₂ represents a moiety derived from a drug, optionally a moiety derived from the same drug as D₁;
if m is more than 1, then each D₂ is independently selected from a hydrogen atom and moieties derived from a drug, wherein multiple moieties D₂ can be the same or different with the proviso that at least one D₂ is not a hydrogen atom, if D₂ is a hydrogen atom then A′xx represents an amino acid with the proviso that A′xx in formula (Ia) is not an amino acid in the (D) configuration, if D₂ is a moiety derived from a drug, then A′xx represents a trifunctional amino with the proviso that A′xx in formula (Ia) is not an amino acid in the (D) configuration;
and the broken line indicates covalent attachment to the N-terminus of Axx or Ayy;

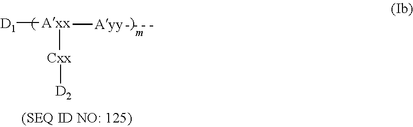

wherein, in formula (Ib),
A′yy represents an amino acid selected from Phe, Ala, Trp, Tyr, Phg, Met, Val, His, Lys, Arg, Cit, Abu, Orn;
D₁ represents a moiety derived from a drug;
m is an integer of 1 to 10;
if m=1, then A′xx represents a trifunctional amino acid selected from Glu, α-amino adipic acid (Aaa), Dap, Dab, Ser, Thr, homo-serine (homo-Ser), homo-threonine (homo-Thr) and amino-malonic acid (Ama) with the proviso that A′xx is not an amino acid in the (D) configuration; D₂ represents a moiety derived from a drug, optionally a moiety derived from the same drug as D₁, Cxx represents a single covalent bond unless A′xx is Ama, if A′xx is Ama, Cxx represents (L)- or (D)-Pro, or an N-methyl amino acid such as sarcosine (Sar), the N-terminus of Cxx binds to a carboxyl end of Ama and the C-terminus of Cxx binds to a moiety D₂;
if m is more than 1, then each D₂ is independently selected from a hydrogen atom and moieties derived from a drug, wherein multiple moieties D₂ can be the same or different with the proviso that at least one D₂ is not a hydrogen atom, if D₂ is a hydrogen atom then A′xx represents an amino acid with the proviso that A′xx is not in the (D) configuration and Cxx represents a single covalent bond, if D₂ is a moiety derived from a drug then A′xx represents an amino acid selected from Glu, Aaa, Dap, Dab, Ser, Thr, homo-Ser, Homo-Thr and Ama with the proviso that A′xx is not an amino acid in the (D) configuration, Cxx represents a single covalent bond unless A′xx is Ama, if A′xx is Ama, Cxx represents (L)- or (D)-Pro, or an N-methyl amino acid such as Sar wherein the N-terminus of Cxx binds to a carboxyl end of Ama and the C-terminus of Cxx binds to moiety D₂;
and the broken line indicates covalent attachment to the N-terminus of Axx or Ayy;
Axx represents a trifunctional amino acid such as an amino-dicarboxylic acid or a diamino-carboxylic acid; with the proviso that Axx in formula (I) is not an amino acid in the (D) configuration;
Ayy represents an amino acid selected from Phe, Ala, Trp, Tyr, Phenylglycine (Phg), Met, Val, His, Lys, Arg, Cit, 2-amino-butyric acid (Abu), Orn, Ser, Thr, Leu and IIe; or Ayy in formula (I) represents an amino acid selected from homo-tyrosine (homo-Tyr), homo-phenylalanine (homo-Phe), beta-phenylalanine (beta-Phe) and beta-homo-phenylalanine (beta-homo-Phe), Tyr(OR₁) and homo-Tyr(OR₁) wherein R₁ is —(CH₂CH₂O)ₙ₁—R₂, wherein R₂ is a hydrogen atom or a methyl group and n1 is an integer of 2 to 24; with the proviso that Ayy in formula (I′) is not an amino acid in the (D) configuration;
T is a moiety being represented by the following formula (Ia₁):

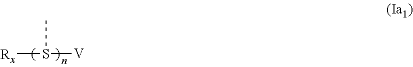

wherein, in formula (Ia₁),
S represents a group containing one or more atoms selected from carbon, nitrogen, oxygen, and sulfur;
V represents a moiety derived from a vector group capable of interacting with a target cell;
n is an integer of 1 to 10;
Rₓ is an atom or group which is optionally present to saturate a free valency of S, if present;
and the broken line indicates covalent attachment to the side chain of Axx; if n is more than 1, each broken line represents a covalent bond to an individual, separate group of formula (I) or formula (I′), wherein multiple groups of formula (I) or formula (I′) can be the same or different; if n is more than 1, each S can be the same or different;
Z represents a group covalently bonded to the C-terminus of Ayy or Axx selected from —OH, —N(H)(R), wherein R represents a hydrogen atom, an alkyl group, a cycloalkyl group or an aromatic group; and a labeling agent such as a coumarin derivative.

The present invention further relates to a compound represented by the general formula (II), (II′) or (IIa):

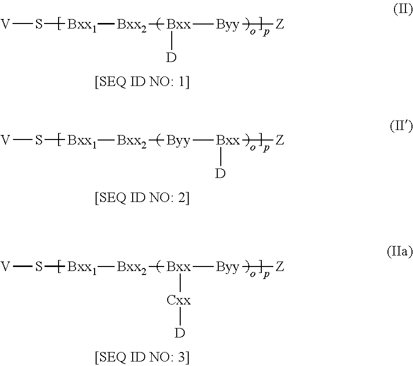

wherein,
D represents a moiety derived from a drug; if o*p>1 one or more D's may be hydrogen or a solubilizing group such as —(CH₂CH₂O)ₙ₁—R₂ wherein R₂ is a hydrogen atom or a methyl group and n1 is an integer of 2 to 24, with the proviso that at least one D represents a moiety derived from a drug;
Bxx in formulae (II) and (II′) represents Phe, a trifunctional amino acid such as an amino-dicarboxylic acid or a diamino-carboxylic acid, preferably selected from Glu, Asp, Aaa, Lys, Dap, Dab, Ser, Thr, homo-Ser and homo-Thr; with the proviso that Bxx in formula (II) is not an amino acid in the (D) configuration;
Bxx in formula (IIa) represents a carboxylic amino acid (i.e. an amino acid having a carboxylic acid group on its side chain) such as Ama, Glu, Aaa, Apa or a trifunctional amino acid selected from Dap, Dab, Ser, Thr, Lys, Orn, homoLys, homoSer and homoThr; with the proviso that Bxx is not an amino acid in the (D) configuration; Cxx represents a single covalent bond unless Bxx is Ama, if Bxx is Ama, Cxx represents (L)- or (D)-Pro, or an N-methyl amino acid such as Sar, the N-terminus of Cxx binds to a carboxyl end of Ama and the C-terminus of Cxx binds to moiety D;
in those instances where Bxx in formulae (II), (II′) and (IIa) carries a hydrogen as D group, Bxx may also be any other amino acid, with the proviso that Bxx in formulae (II) and (IIa) is not an amino acid in the (D) configuration;
Byy represents an amino acid selected from Phe, Ala, Trp, Tyr, Phg, Val, His, Lys, Abu, Met, Cit, Orn, Ser, Thr, Leu, IIe, Arg and Tyr(OR₁) wherein R₁ is —(CH₂CH₂O)ₙ₁—R₂, wherein R₂ is a hydrogen atom or a methyl group and n1 is an integer of 2 to 24; or Byy in formulae (II) and (IIa) represents an amino acid selected from homo-Tyr, homo-Tyr(OR₁), homo-Phe, beta-Phe and beta-homo-Phe; with the proviso that Byy in formula (II′) is not an amino acid in the (D) configuration and with the proviso that if o*p>1, only the C-terminal Byy in formulae (II) and (IIa) may represent an amino acid selected from homo-Phe, beta-Phe and beta-homo-Phe;
Bxx1 represents a single covalent bond or an amino acid having a hydrophobic side chain;
Bxx2 represents an amino acid having a hydrophobic or basic side chain; S and V are as defined in formula (I) or (I′);
Z is covalently bonded to the C-terminus of Byy and represents a group selected from —OH; —N(H)(R), wherein R represents a hydrogen atom, an alkyl group, a cycloalkyl group or an aromatic group; and a labelling agent; and
o and p each independently is an integer of 1 to 10; if p is more than 1, Bxx₁ is not an amino acid in the (D) configuration; if p is more than 1 and/or o is more than 1, then each D can be independently selected from moieties derived from a drug.

The present invention also relates to a compound as hereinbefore described or composition thereof for use in a method of treating or preventing a cancer, an autoimmune disease and/or an infectious disease.

The present invention in particular includes the following embodiments (“Items”):
1. A compound represented by the general formula (I) or (I′):

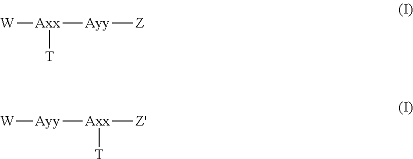

wherein, in formulae (I) and (I′),
W represents a moiety represented by the following formula (III):

W₁-Dxx-Dyy---- (III)
wherein
W₁ represents a moiety derived from a drug that differs from a native drug only by virtue of the covalent attachment to Dxx as shown in formula (III), if the drug is an auristatin analog, the auristatin is auristatin Phe (AF), auristatin Cit (ACit), auristatin Arg (AArg), auristatin Lys (ALys), auristatin Orn (AOrn), auristatin Dab (ADab) or auristatin Dap (ADap), preferably AF;
Dxx represents a single covalent bond or an amino acid having a hydrophobic side chain, preferably an amino acid selected from Phe, Val, Tyr, homo-Phe and Ala, preferably Phe or Val, wherein the single covalent bond or amino acid having a hydrophobic side chain is optionally attached to moiety W₁ via a divalent moiety selected from maleimides, triazoles, hydrazones, carbonyl-containing groups, and derivatives thereof, preferably via a divalent maleimide derivative;
Dyy represents a single covalent bond, Phe or an amino acid having a basic side chain, preferably an amino acid selected from Arg, Lys, Citrulline (Cit), Ornithine (Orn), 2,3-diamino-propionic acid (Dap), 2,4-diamino-butyric acid (Dab), more preferably Arg or Cit;
with the proviso that if Dxx is an amino acid having a hydrophobic side chain, Dyy is Phe or an amino acid having a basic side chain, and if Dxx is a single covalent bond, Dyy is a single covalent bond, Phe or an amino acid with a basic side chain, preferably Arg or Cit;
and the broken line indicates covalent attachment to the N-terminus of Axx in formula (I) or the N-terminus of Ayy in formula (I′);
Axx represents a trifunctional amino acid such as an amino-dicarboxylic acid or a diamino-carboxylic acid; with the proviso that Axx in formula (I) is not an amino acid in the (D) configuration;
Ayy represents an amino acid selected from Phe, Ala, Trp, Tyr, Phenylglycine (Phg), Met, Val, His, Lys, Arg, Cit, 2-amino-butyric acid (Abu), Orn, Ser, Thr, Leu and IIe; or Ayy in formula (I) represents an amino acid selected from homo-tyrosine (homo-Tyr), homo-phenylalanine (homo-Phe), beta-phenylalanine (beta-Phe) and beta-homo-phenylalanine (beta-homo-Phe), Tyr(OR₁) and homo-Tyr(OR₁) wherein R₁ is —(CH₂CH₂O)ₙ₁—R₂, wherein R₂ is a hydrogen atom or a methyl group and n1 is an integer of 2 to 24; with the proviso that Ayy in formula (I′) is not an amino acid in the (D) configuration;
T is a moiety being represented by the following formula (Ia₁):

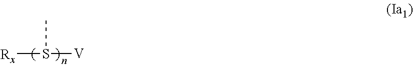

wherein, in formula (Ia₁),
S represents a group containing one or more atoms selected from carbon, nitrogen, oxygen, and sulfur;
V represents a moiety derived from a vector group capable of interacting with a target cell;
n is an integer of 1 to 10;
Rₓ is an atom or group which is optionally present to saturate a free valency of S, if present;
and the broken line indicates covalent attachment to the side chain of Axx; if n is more than 1, each broken line represents a covalent bond to an individual, separate group of formula (I) or formula (I′), wherein multiple groups of formula (I) or formula (I′) can be the same or different; if n is more than 1, each S can be the same or different;
Z represents a group covalently bonded to the C-terminus of Ayy or Axx selected from —OH; —N(H)(R), wherein R represents a hydrogen atom, an alkyl group, a cycloalkyl group or an aromatic group; and a labeling agent such as a coumarin derivative.
2. A compound represented by the general formula (I) or (I′):

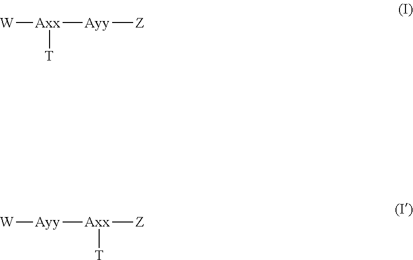

wherein, in formulae (I) and (I′),
W represents a moiety represented by the following formula (III):

W₁-Dxx-Dyy---- (III)
wherein
W₁ represents a moiety derived from a drug with the proviso that W₁ is not an auristatin analog;
Dxx represents a single covalent bond or an amino acid having a hydrophobic side chain, preferably an amino acid selected from Phe, Val, Tyr, homo-Phe and Ala, more preferably Phe or Val, wherein the single covalent bond or amino acid having a hydrophobic side chain is optionally attached to moiety W₁ via a divalent moiety selected from maleimides, triazoles, hydrazones, carbonyl-containing groups, and derivatives thereof, preferably via a divalent maleimide derivative;
Dyy represents a single covalent bond, Phe or an amino acid having a basic side chain, preferably an amino acid selected from Arg, Lys, Cit, Orn, Dap, and Dab, more preferably Arg or Cit;
with the proviso that if Dxx is an amino acid having a hydrophobic side chain, Dyy is Phe or an amino acid having a basic side chain, and if Dxx is a single covalent bond, Dyy is a single covalent bond, Phe or an amino acid having a basic side chain, preferably Arg or Cit;
and the broken line indicates covalent attachment to the N-terminus of Axx in formula (I), or the N-terminus of Ayy in formula (I′);
Axx represents a trifunctional amino acid such as an amino-dicarboxylic acid or a diamino-carboxylic acid; with the proviso that Axx in formula (I) is not an amino acid in the (D) configuration;
Ayy represents an amino acid selected from Phe, Ala, Trp, Tyr, Phg, Met, Val, His, Lys, Arg, Cit, Abu, Orn, Ser, Thr, Leu and IIe; or Ayy in formula (I) represents an amino acid selected from homo-Tyr, homo-Phe, beta-Phe and beta-homo-Phe), Tyr(OR₁) and homo-Tyr(OR₁) wherein R₁ is —(CH₂CH₂O)ₙ₁—R₂, wherein R₂ is a hydrogen atom or a methyl group and n1 is an integer of 2 to 24; with the proviso that Ayy in formula (I′) is not an amino acid in the (D) configuration;
T is a moiety being represented by the following formula (Ia₁):
   wherein, in formula

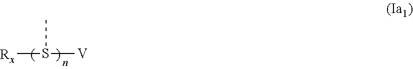

S represents a group containing one or more atoms selected from carbon, nitrogen, oxygen, and sulfur;
V represents a moiety derived from a vector group capable of interacting with a target cell;
n is an integer of 1 to 10;
Rₓ is an atom or group which is optionally present to saturate a free valency of S, if present;
and the broken line indicates covalent attachment to the side chain of Axx; if n is more than 1, each broken line represents a covalent bond to an individual, separate group of formula (I) or formula (I′), wherein multiple groups of formula (I) or formula (I′) can be the same or different; if n is more than 1, each S can be the same or different;
Z represents a group covalently bonded to the C-terminus of Ayy or Axx selected from —OH; —N(H)(R), wherein R represents a hydrogen atom, an alkyl group, a cycloalkyl group or an aromatic group; and a labeling agent such as a coumarin derivative.
3. A compound represented by the general formula (I) or (I′):

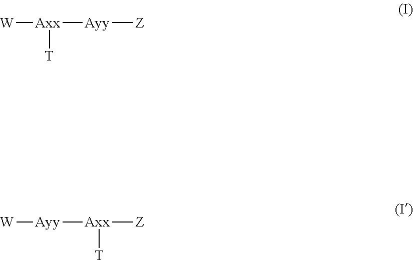

wherein, in formulae (I) and (I′),
W represents a peptide moiety represented by formula (Ia), (Ia′) or (Ib):

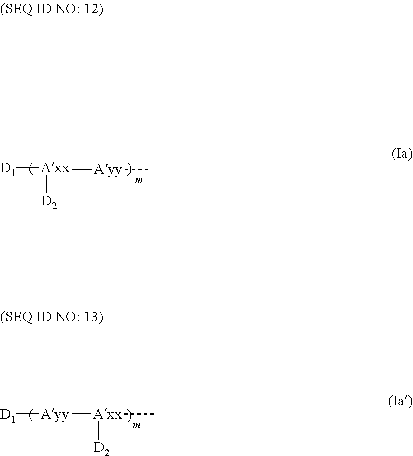

wherein, in formulae (Ia) and (Ia′),
A′yy represents an amino acid selected from Phe, Ala, Trp, Tyr, Phg, Met, Val, His, Lys, Arg, Cit, Abu, Orn, with the proviso that A′yy in formula (Ia′) is not an amino acid in the (D) configuration;
D₁ represents a moiety derived from a drug;
m is an integer of 1 to 10;
if m=1, then A′xx represents a trifunctional amino acid such as an amino dicarboxylic acid or a diamino carboxylic acid with the proviso that A′xx in formula (Ia) is not an amino acid in the (D) configuration, D₂ represents a moiety derived from a drug, optionally a moiety derived from the same drug as D₁;
if m is more than 1, then each D₂ is independently selected from a hydrogen atom and moieties derived from a drug, wherein multiple moieties D₂ can be the same or different with the proviso that at least one D₂ is not a hydrogen atom, if D₂ is a hydrogen atom then A′xx represents an amino acid with the proviso that A′xx in formula (Ia) is not an amino acid in the (D) configuration, if D₂ is a moiety derived from a drug, then A′xx represents a trifunctional amino with the proviso that A′xx in formula (Ia) is not an amino acid in the (D) configuration;
and the broken line indicates covalent attachment to the N-terminus of Axx or Ayy;

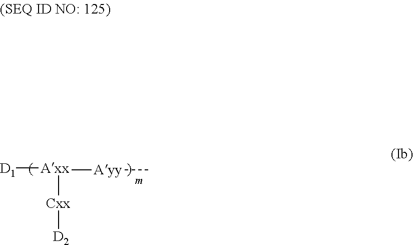

wherein, in formula (Ib),
A′yy represents an amino acid selected from Phe, Ala, Trp, Tyr, Phg, Met, Val, His, Lys, Arg, Cit, Abu, Orn;
D₁ represents a moiety derived from a drug;
m is an integer of 1 to 10;
if m=1, then A′xx represents a trifunctional amino acid selected from Glu, α-amino adipic acid (Aaa), Dap, Dab, Ser, Thr, homo-serine (homo-Ser), homo-threonine (homo-Thr) and amino-malonic acid (Ama) with the proviso that A′xx is not an amino acid in the (D) configuration; D₂ represents a moiety derived from a drug, optionally a moiety derived from the same drug as D₁, Cxx represents a single covalent bond unless A′xx is Ama, if A′xx is Ama, Cxx represents (L)- or (D)-Pro, or an N-methyl amino acid such as sarcosine (Sar), the N-terminus of Cxx binds to a carboxyl end of Ama and the C-terminus of Cxx binds to a moiety D₂;
if m is more than 1, then each D₂ is independently selected from a hydrogen atom and moieties derived from a drug, wherein multiple moieties D₂ can be the same or different with the proviso that at least one D₂ is not a hydrogen atom, if D₂ is a hydrogen atom then A′xx represents an amino acid with the proviso that A′xx is not in the (D) configuration and Cxx represents a single covalent bond, if D₂ is a moiety derived from a drug then A′xx represents an amino acid selected from Glu, Aaa, Dap, Dab, Ser, Thr, homo-Ser, Homo-Thr and Ama with the proviso that A′xx is not an amino acid in the (D) configuration, Cxx represents a single covalent bond unless A′xx is Ama, if A′xx is Ama, Cxx represents (L)- or (D)-Pro, or an N-methyl amino acid such as Sar wherein the N-terminus of Cxx binds to a carboxyl end of Ama and the C-terminus of Cxx binds to moiety D₂;
and the broken line indicates covalent attachment to the N-terminus of Axx or Ayy;
Axx represents a trifunctional amino acid such as an amino-dicarboxylic acid or a diamino-carboxylic acid; with the proviso that Axx in formula (I) is not an amino acid in the (D) configuration;
Ayy represents an amino acid selected from Phe, Ala, Trp, Tyr, Phg, Met, Val, His, Lys, Arg, Cit, Abu, Orn, Ser, Thr, Leu and IIe; or Ayy in formula (I) represents an amino acid selected from homo-Tyr, homo-Phe, beta-Phe and beta-homo-Phe, Tyr(OR₁) and homo-Tyr(OR₁) wherein R₁ is —(CH₂CH₂O)ₙ₁—R₂, wherein R₂ is a hydrogen atom or a methyl group and n1 is an integer of 2 to 24; with the proviso that Ayy in formula (I′) is not an amino acid in the (D) configuration;
T is a moiety being represented by the following formula (Ia₁):

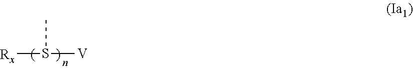

wherein, in formula (Ia₁),
S represents a group containing one or more atoms selected from carbon, nitrogen, oxygen, and sulfur;
V represents a moiety derived from a vector group capable of interacting with a target cell;
n is an integer of 1 to 10;
Rₓ is an atom or group which is optionally present to saturate a free valency of S, if present;
and the broken line indicates covalent attachment to the side chain of Axx; if n is more than 1, each broken line represents a covalent bond to an individual, separate group of formula (I) or formula (I′), wherein multiple groups of formula (I) or formula (I′) can be the same or different; if n is more than 1, each S can be the same or different;
Z represents a group covalently bonded to the C-terminus of Ayy or Axx selected from —OH; —N(H)(R), wherein R represents a hydrogen atom, an alkyl group, a cycloalkyl group or an aromatic group; and a labeling agent such as a coumarin derivative.
4. The compound of any of items 1 to 3, wherein at least one of Axx and Ayy is defined as follows:
   Axx represents an amino acid selected from Glu, 2-amino-pimelic acid (Apa), Aaa, Dap, Dab, Lys, Orn, Ser, Ama, and homo-lysine (homo-Lys), preferably an amino acid selected from Dap, Dab, Lys, Orn and homo-Lys;
   Ayy in formula (I) represents an amino acid selected from Phe, homo-Phe, Ala, Trp, Phg, Leu, Val, Tyr, homo-Tyr, Tyr(OR₁) and homo-Tyr(OR₁) wherein R₁ is —(CH₂CH₂O)ₙ₁—R₂, wherein R₂ is a hydrogen atom or a methyl group and n1 is an integer of 2 to 24, preferably Phe, homo-Phe, Tyr, homo-Tyr, Tyr(OR₁) or homo-Tyr(OR₁), more preferably Phe or Tyr;
   Ayy in formula (I′) represents an amino acid selected from Phe, homo-Phe, Ala, Trp, Phg, Leu, Val, Tyr and Ser, preferably Phe, home-Phe or Ser, more preferably Phe or Ser.
5. The compound of item 3 or 4, wherein at least one of A′xx, A′yy and m is defined as follows:
   A′xx in formulae (Ia) and (Ia′) represents an amino acid selected from Dap, Dap, Lys, Orn and homo-Lys, preferably Lys;
   A′yy in formulae (Ia), (Ia′) and (Ib) represents an amino acid selected from Phe, Ala, Trp, Phg and Tyr, preferably Phe or Tyr;
   m is an integer of 1 to 4.
6. A compound represented by one of the following general formulae (II), (II′) and (IIa):

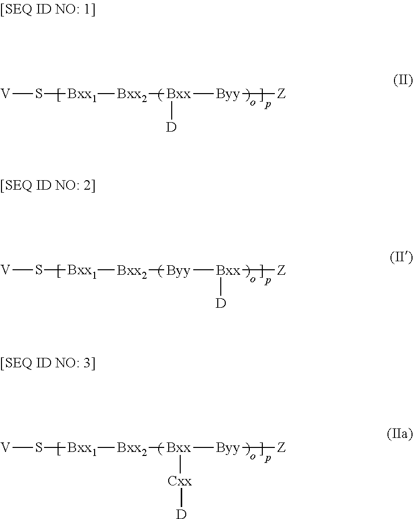

wherein,
D represents a moiety derived from a drug; if o*p>1 one or more D's may be hydrogen or a solubilizing group such as —(CH₂CH₂O)ₙ₁— R₂, wherein R₂ is a hydrogen atom or a methyl group and n1 is an integer of 2 to 24, with the proviso that at least one D represents a moiety derived from a drug;
Bxx in formulae (II) and (II′) represents a trifunctional amino acid such as an amino-dicarboxylic acid or a diamino-carboxylic acid; with the proviso that Bxx in formula (II) is not an amino acid in the (D) configuration;
Bxx in formula (IIa) represents a carboxylic amino acid such as Ama, Glu, Aaa, Apa or a trifunctional amino acid selected from Dap, Dab, Ser, Thr, Lys, Orn, homoLys, homoSer and homoThr; with the proviso that Bxx is not an amino acid in the (D) configuration; Cxx represents a single covalent bond unless Bxx is Ama; if Bxx is Ama, Cxx represents (L)- or (D)-Pro, or an N-methyl amino acid such as Sar, the N-terminus of Cxx binds to a carboxyl end of Ama and the C-terminus of Cxx binds to moiety D;
in those instances where Bxx in formulae (II), (II′) and (IIa) carries a hydrogen as D group, Bxx may be any other amino acid, with the proviso that Bxx in formulae (II) and (IIa) is not an amino acid in the (D) configuration;
Byy represents an amino acid selected from Phe, homo-Phe, Ala, Trp, Tyr, Phg, Val, His, Lys, Abu, Met, Cit, Orn, Ser, Thr, Leu, IIe, Arg and Tyr(OR₁) wherein R₁ is —(CH₂CH₂O)ₙ₁— R₂, wherein R₂ is a hydrogen atom or a methyl group and n1 is an integer of 2 to 24; or Byy in formulae (II) and (IIa) represents an amino acid selected from homo-Tyr, homo-Tyr(OR₁), homo-Phe, beta-Phe and beta-homo-Phe; with the proviso that Byy in formula (II′) is not an amino acid in the (D) configuration and with the proviso that if o*p>1, only the C-terminal Byy in formulae (II) and (IIa) may represent an amino acid selected from beta-Phe and beta-homo-Phe;
Bxx₁ represents a single covalent bond or an amino acid having a hydrophobic or basic side chain;
Bxx₂ represents an amino acid having a hydrophobic or basic side chain;
S and V are as defined in item 1;
Z is covalently bonded to the C-terminus of Byy in formulae (II) and (IIa) and the C-terminus of Bxx in formula (II′), and represents a group selected from —OH; —N(H)(R), R being as defined in item 1; and a labelling agent; and
o and p each independently is an integer of 1 to 10, if p is more than 1, Bxx₁ is not an amino acid in the (D) configuration; if p is more than 1 and/or if o is more than 1, then each D can be independently selected from moieties derived from a drug.
7. The compound of item 6, wherein at least one of Bxx₁, Bxx₂, Bxx, Byy, o and p is defined as follows:
   Bxx₁ represents a single covalent bond or an amino acid selected from Phe, homo-Phe, Phg, Val, Ser, Tyr, Ala, Leu, IIe; preferably an amino acid selected from Phe, homo-Phe, Tyr and Val, more preferably Phe, homo-Phe or Tyr;
   Bxx₂ represents an amino acid selected from Arg, Lys, Cit, Val, Leu, Ser, Ala, Gly, His, Gln, Phg and Phe; preferably an amino acid selected from Arg, Lys, Cit and Phe, more preferably Arg or Cit;
   Bxx in formulae (II) and (II′) represents an amino acid selected from Dap, Dab, Lys, Orn, Ser, Glu, Ama, Thr, Tyr, Aaa, homo-Ser and homo-Thr; preferably Lys or Dab, more preferably Lys;
   Byy represents Cit, Phe, homo-Phe, Ser, Trp, Tyr or Tyr(OR₁) wherein R₁ is —(CH₂CH₂O)ₙ₁—R₂, wherein R₂ is a hydrogen atom or a methyl group and n1 is an integer of 2 to 24, preferably Phe, Tyr or Tyr(OR₁); if o*p>1, Byy represents preferably Tyr or Tyr(OR₁); and
   o and p each independently is an integer of 1 to 4.
8. The compound of any of items 1 to 7, wherein in formulae (Ia₁), (II), (II′) and (IIa),
   S represents a divalent group selected from a divalent alkylene group, a divalent alkenylene group, a divalent alkynylene group, and a divalent polyalkylene oxide;
   preferably a divalent group having formula —(CH₂)_{q}-Azz₅-, or —(OCH₂CH₂)_{q}-Azz₅-; wherein q is an integer of 1 to 50; and Azz₅ is absent, or represents a solubilizing group preferably selected from an amino acid such as Arg or (D)-Arg and a divalent group containing an ammonium group, a sulfate group, a sulfonate group or a pyrophosphate diester group.
9. The compound of any of items 1 to 8, wherein in formulae (Ia1), (II), (II′) and (IIa),
   S represents a divalent group having formula —(CH₂)_{q}-Azz₅-Y—, or a divalent group having formula —(OCH₂CH₂)_{q}-Azz₅-Y—;
   wherein Y represents a divalent moiety covalently bonded to the C-terminus of Azz₅ and to moiety V; if Azz₅ is absent, Y is covalently bonded to the alkyl group or polyethylene oxide group and to moiety V; Y is derived from a compound selected from maleimides, triazoles, especially 1,2,3-triazole, hydrazones, carbonyl-containing groups, and derivatives thereof, preferably from maleimides and derivatives thereof; q is an integer of 1 to 50; and Azz₅ is as defined in item 8.
10. The compound of item 1, 2 or 3, wherein the compound of formula (I) and formula (Ia) is selected from W₁-Arg-Lys(T)-Phe-Z, W₁-Arg-Lys(T)-homoPhe-Z, W₁—Cit-Lys(T)-Phe-Z, W₁—Cit-Lys(T)-Tyr-Z, W₁—Cit-Lys(T)-homoTyr-Z, W₁-Lys(T)-Phe-Z, W₁-Lys(T)-Tyr-Z, W₁-Lys(T)-homoTyr-Z, W₁-Mal-Phe-Cit-Lys(T)-Phe-Z (SEQ ID NO: 4), W₁-Mal-Phe-Cit-Lys(T)-Tyr-Z (SEQ ID NO: 5), W₁-Mal-Phe-Cit-Lys(T)-homoTyr-Z (SEQ ID NO: 6), W₁-Mal-Phe-Lys-Lys(T)-Phe-Z (SEQ ID NO: 7), W₁-Mal-homoPhe-Arg-Lys(T)-Phe-Z (SEQ ID NO: 8), W₁-Mal-homoPhe-Cit-Lys(T)-Tyr-Z (SEQ ID NO: 9), W₁-Mal-homoPhe-Cit-Lys(T)-Tyr(OR₁)—Z (SEQ ID NO: 10) with R₁—(CH₂CH₂O)n1-R₂, wherein R₂ is a hydrogen atom or a methyl group and n1 is an integer of 2 to 24 e.g. 12, W₁-Mal-Cit-Lys(T)-Tyr-Z, W₁-Mal-Cit-Lys(T)-homoTyr-Z, W₁-Mal-Arg-Lys(T)-homoTyr-Z, W₁-Cit-(Lys(D₂)-Phe)m-Lys(T)-Phe-Z (SEQ ID NO: 14), W₁—Cit-(Lys(D₂)-Phe)ₘ-Lys(T)-homoTyr-Z (SEQ ID NO: 15), W₁-Cit-(Lys(D₂)-Phe)ₘ-Lys(T)-Tyr(OR₁)—Z (SEQ ID NO: 16) with R₁—(CH₂CH₂O)ₙ₁—R₂, wherein R₂ is a hydrogen atom or a methyl group and n1 is an integer of 2 to 24 e.g. 12, W₁-(Lys(D₂)-Phe)ₘ-Lys(T)-Phe-Z (SEQ ID NO: 17), W₁-Phe-(Phe-Lys(D₂))ₘ-Lys(T)-Tyr-Z (SEQ ID NO: 18), W₁-(Phe-Lys(D₂))ₘ-Lys(T)-Tyr-Z (SEQ ID NO: 19), W₁-Phe-(Phe-Lys(D₂))ₘ-Lys(T)-homoTyr-Z (SEQ ID NO: 20) and W₁-Arg-(Phe-Lys(D₂))ₘ-Lys(T)-Tyr(OR₁)—Z (SEQ ID NO: 21);
   and the compound of formula (I′) is selected from W₁-Arg-Phe-Lys(T)-Z, W₁-Arg-Ser-Lys(T)-Z, W₁—Cit-Phe-Lys(T)-Z, W₁—Cit-Ser-Lys(T)-Z, W₁—Cit-homoPhe-Lys(T)-Z, W₁-Phe-Lys(T)-Z, W₁—Ser-Lys(T)-Z, W₁-Mal-Phe-Cit-Phe-Lys(T)-Z (SEQ ID NO: 22), W₁-Mal-homoPhe-Cit-Phe-Lys(T)-Z (SEQ ID NO: 23), W₁-Mal-Phe-Arg-Phe-Lys(T)-Z (SEQ ID NO: 24), W₁-Mal-Cit-Phe-Lys(T)-Z, W₁-Mal-Phe-Ser-Lys(T)-Z, W₁-Mal-Ala-Phe-Lys(T)-Z, W₁-Mal-Cit-Ser-Lys(T)-Z and W₁-Mal-Arg-homoPhe-Lys(T)-Z.

wherein W₁, T, Z, D₂ and m have the same meanings as specified in item 1, 2 or 3; and Z is preferably —OH.
11. The compound of item 6 which is selected from from V—S-Phe-Arg-Phe-Lys(D)-Ser-Lys(D)-Z (SEQ ID NO: 30), V—S-Phe-Arg-(Phe-Lys(D))ₒ-Z (SEQ ID NO: 31), V—S-Phe-Arg-(Ser-Lys(D))ₒ-Z (SEQ ID NO: 32), V—S-Phe-Arg-(Tyr(OR₁)-Lys(D))ₒ—Z (SEQ ID NO: 33), V—S-Phe-Arg-(Phe-Lys(D))ₒ-Phe-Tyr(OR₁)—Z (SEQ ID NO: 34); preferably V—S-Phe-Arg-Phe-Lys(D)-Ser-Lys(D)-Z (SEQ ID NO: 30), V—S-Phe-Arg-(Phe-Lys(D))ₒ—Z (SEQ ID NO: 31) or V—S-Phe-Arg-(Ser-Lys(D))ₒ—Z (SEQ ID NO: 32); more preferably V—S-Phe-Arg-(Phe-Lys(D))ₒ—Z (SEQ ID NO: 31);

wherein V, S, D, Z and o have the same meanings as specified in item 6; and Z is preferably —OH.
12. The compound of any of items 1 to 11, wherein each moiety derived from a drug is independently selected from:
   (i) antineoplastic agents including alkylating agents, alkaloids such as taxanes and maytansinoids, anti-metabolites, endocrine therapies, kinase inhibitors;
   (ii) immunomodulatory agents such as immunostimulants and immunosuppressants;
   (iii) anti-infectious disease agents including antibacterial drugs, antimitotic drugs, antimycobacterial drugs and antiviral drugs;
   radioisotopes and/or pharmaceutically acceptable salts thereof.
13. The compound of any of items 1 to 12, wherein each moiety derived from a drug is independently derived from amanitin, duocarmycin, auristatin, maytansine, tubulysin, calicheamicin, camptothecin, SN-38, taxol, daunomycin, vinblastine, doxorubicin, methotrexate, pyrrolobenzodiazepine, or radioisotopes and/or pharmaceutically acceptable salts thereof.
14. The compound of any of items 3, 4, 8, 9, 10, 12 and 13, wherein each moiety D₁ is independently represented by the following formula (III):

W₁-Dxx-Dyy---- (III)
wherein
W₁ represents a moiety derived from amanitin, duocarmycin, auristatin, maytansine, tubulysin, calicheamicin, camptothecin, SN-38, taxol, daunomycin, vinblastine, doxorubicin, methotrexate, pyrrolobenzodiazepine, or radioisotopes and/or pharmaceutically acceptable salts thereof;
Dxx represents a single covalent bond or an amino acid having a hydrophobic side chain, preferably an amino acid selected from Phe, homo-Phe, Val and Ala, wherein the single covalent bond or amino acid having a hydrophobic side chain is optionally attached to moiety W₁ via a divalent moiety selected from maleimides, triazoles, hydrazones, carbonyl-containing groups, and derivatives thereof, preferably via a divalent maleimide derivative;
Dyy represents a single covalent bond, Phe or an amino acid having a basic side chain, preferably an amino acid selected from Arg, Lys, Cit, Orn, Dap, and Dab, more preferably Arg or Cit;
with the proviso that if Dxx is an amino acid having a hydrophobic side chain, Dyy is Phe or an amino acid having a basic side chain, and if Dxx is a single covalent bond, Dyy is a single covalent bond, Phe or an amino acid with a basic side chain, preferably Arg or Cit;
and the broken line indicates covalent attachment to the N-terminus of Axx in formula (I), the N-terminus of Ayy in formula (I′), the N-terminus of A′xx in formulae (Ia) and (Ib), or the N-terminus A′yy in formula (Ia′).
15. The compound of any of items 3 to 14, wherein each moiety D₂ and D is independently represented by the following formula (IIIa):

W₂-Exx---- (IIIa)
wherein
W₂ represents a moiety derived from amanitin, duocarmycin, auristatin, maytansine, tubulysin, calicheamicin, camptothecin, SN-38, taxol, daunomycin, vinblastine, doxorubicin, methotrexate, pyrrolobenzodiazepine, or radioisotopes and/or pharmaceutically acceptable salts thereof;
Exx represents a single covalent bond or a divalent moiety selected from maleimides, triazoles, hydrazones, carbonyl-containing groups, amino acids, dipeptide moieties and derivatives thereof, preferably a divalent maleimide derivative;
and the broken line indicates covalent attachment to the side chain of A′xx in formulae (Ia) and (Ia′), the side chain of A′xx or C-terminus of Cxx if present in formula (Ib), the side chain of Bxx in formulae (II) and (II′), the side chain of Bxx or C-terminus of Cxx if present in formula (IIa).
16. The compound of any of items 1 to 15, wherein V represents a moiety derived from a vector group selected from antibodies, antibody fragments, proteins, peptides and non-peptidic molecules;
   preferably an antibody or an antibody fragment such as a single chain antibody, a monoclonal antibody, a single chain monoclonal antibody, a monoclonal antibody fragment, a chimeric antibody, a chimeric antibody fragment, a domain antibody or fragment thereof, a cytokine, a hormone, a growth factor, a colony stimulating factor, a neurotransmitter or a nutrient-transport molecule.
17. The compound of any of items 1 to 16, wherein
   V represents a moiety derived from a vector group capable of interacting with a target cell, wherein the target cell is selected from tumor cells, virus infected cells, microorganism infected cells, parasite infected cells, cells involved in autoimmune diseases, activated cells, myeloid cells, lymphoid cells, melanocytes and infectious agents including bacteria, viruses, mycobacteria, fungi,
   preferably the target cell is selected from lymphoma cells, myeloma cells, renal cancer cells, breast cancer cells, prostate cancer cells, ovarian cancer cells, colorectal cancer cells, gastric cancer cells, squamous cancer cells, small-cell lung cancer cells, testicular cancer cells, and any cells growing and dividing at an unregulated and quickened pace to cause cancers.
18. Composition comprising a therapeutically effective amount of the compound of any of items 1 to 17 or a pharmaceutically acceptable salt thereof, and one or more components selected from a carrier, a diluent and other excipients.
19. The compound or composition of any of items 1 to 18 for use in a method of treating or preventing a cancer, an autoimmune disease and/or an infectious disease.
20. The compound or composition for use of items 19, wherein in the method of treating or preventing a cancer, an autoimmune disease and/or an infectious disease, the compound or composition is administered concurrently with, before or after one or more other therapeutic agents or therapies such as chemotherapeutic agents, radiation therapy, immunotherapy agents, autoimmune disorder agents, anti-infectious agents or other compounds of formula (I)/(I′) or (II)/(II′)/(IIa).
21. Method for treating or preventing a cancer, an autoimmune disease and/or an infectious disease, wherein a therapeutically effective amount of the compound or composition of any of items 1 to 18 is administered to a patient in need thereof.

In some other aspects, the present invention includes the following embodiments (“Items”):
1. A compound represented by the general formula (I) or (I′):

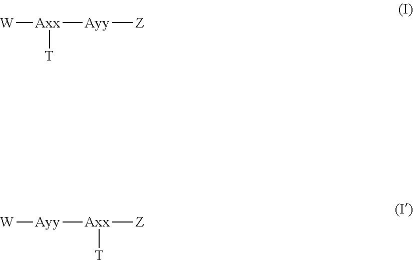

wherein, in formulae (I) and (I′),
W represents a moiety D₁ derived from a drug; or a peptide moiety represented
by formula (Ia), (Ia′) or (Ib):

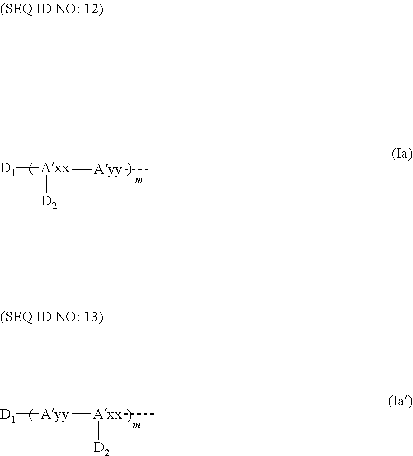

wherein, in formulae (Ia) and (Ia′),
A′yy represents an amino acid selected from Phe, Ala, Trp, Tyr, Phenylglycine (Phg), Met, Val, His, Lys, Arg, Citrulline (Cit), 2-amino-butyric acid (Abu), Ornithine (Orn), with the proviso that A′yy in formula (Ia′) is not an amino acid in the (D) configuration;
D₁ represents a moiety derived from a drug;
m is an integer of 1 to 10;
if m=1, then A′xx represents a trifunctional amino acid such as an amino dicarboxylic acid or a diamino carboxylic acid with the proviso that A′xx in formula (Ia) is not an amino acid in the (D) configuration, D₂ represents a moiety derived from a drug, optionally a moiety derived from the same drug as D₁;
if m is more than 1, then each D₂ is independently selected from a hydrogen atom and moieties derived from a drug, wherein multiple moieties D₂ can be the same or different with the proviso that at least one D₂ is not a hydrogen atom, if D₂ is a hydrogen atom then A′xx represents an amino acid with the proviso that A′xx in formula (Ia) is not an amino acid in the (D) configuration, if D₂ is a moiety derived from a drug, then A′xx represents a trifunctional amino with the proviso that A′xx in formula (Ia) is not an amino acid in the (D) configuration;
and the broken line indicates covalent attachment to the N-terminus of Axx or Ayy;

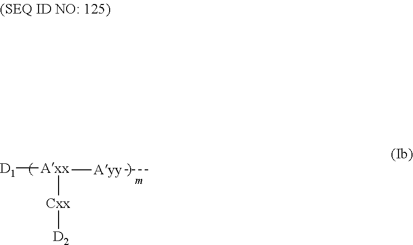

wherein, in formula (Ib),
A′yy represents an amino acid selected from Phe, Ala, Trp, Tyr, Phg, Met, Val, His, Lys, Arg, Cit, Abu, Orn;
D₁ represents a moiety derived from a drug;
m is an integer of 1 to 10;
if m=1, then A′xx represents a trifunctional amino acid selected from Glu, α-amino adipic acid (Aaa), 2,3-diamino-propionic acid (Dap), 2,4-diamino-butyric acid (Dab), Ser, Thr, homoserine (homoSer), homothreonine (homoThr) and amino-malonic acid (Ama) with the proviso that A′xx is not an amino acid in the (D) configuration; D₂ represents a moiety derived from a drug, optionally a moiety derived from the same drug as D₁, Cxx represents a single covalent bond unless A′xx is Ama, if A′xx is Ama, Cxx represents (L)- or (D)-Pro, or an N-methyl amino acid such as sarcosine (Sar), the N-terminus of Cxx binds to a carboxyl end of Ama and the C-terminus of Cxx binds to a moiety D₂;
if m is more than 1, then each D₂ is independently selected from a hydrogen atom and moieties derived from a drug, wherein multiple moieties D₂ can be the same or different with the proviso that at least one D₂ is not a hydrogen atom, if D₂ is a hydrogen atom then A′xx represents an amino acid with the proviso that A′xx is not in the (D) configuration and Cxx represents a single covalent bond, if D₂ is a moiety derived from a drug then A′xx represents an amino acid selected from Glu, Aaa, Dap, Dab, Ser, Thr, homoSer, HomoThr and Ama with the proviso that A′xx is not an amino acid in the (D) configuration, Cxx represents a single covalent bond unless A′xx is Ama, if A′xx is Ama, Cxx represents (L)- or (D)-Pro, or an N-methyl amino acid such as Sar, the N-terminus of Cxx binds to a carboxyl end of Ama and the C-terminus of Cxx binds to moiety D₂;
and the broken line indicates covalent attachment to the N-terminus of Axx or Ayy;
Axx represents a trifunctional amino acid such as an amino-dicarboxylic acid or a diamino-carboxylic acid; with the proviso that Axx in formula (I) is not an amino acid in the (D) configuration;
Ayy represents an amino acid selected from Phe, Ala, Trp, Tyr, Phg, Met, Val, His, Lys, Arg, Cit, Abu, Orn, Ser, Thr, Leu and IIe; or Ayy in formula (I) represents an amino acid selected from homo-Phe, beta-Phe and beta-homo-Phe; with the proviso that Ayy in formula (I′) is not an amino acid in the (D) configuration;
T is a moiety being represented by the following formula (Ia₁):

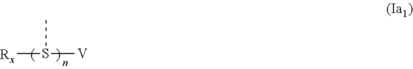

wherein, in formula (Ia₁),
S represents a group containing one or more atoms selected from carbon, nitrogen, oxygen, and sulfur;
V represents a moiety derived from a vector group capable of interacting with a target cell;
n is an integer of 1 to 10;
Rₓ is an atom or group which is optionally present to saturate a free valency of S, if present;
and the broken line indicates covalent attachment to the side chain of Axx; if n is more than 1, each broken line represents a covalent bond to an individual, separate group of formula (I) or formula (I′), wherein multiple groups of formula (I) or formula (I′) can be the same or different; if n is more than 1, each S can be the same or different;
Z represents a group covalently bonded to the C-terminus of Ayy or Axx selected from —OH; —N(H)(R), wherein R represents a hydrogen atom, an alkyl group, a cycloalkyl group or an aromatic group; and a labeling agent such as a coumarin derivative.
2. The compound of item 1, wherein at least one of Axx, Ayy, A′xx, A′yy and m is defined as follows:
   Axx represents an amino acid selected from Glu, 2-amino-pimelic acid (Apa), Aaa, Dap, Dab, Lys, Orn, Ser, Ama, and homolysine (homoLys), preferably an amino acid selected from Dap, Dab, Lys, Orn and homoLys;
   Ayy represents an amino acid selected from Phe, Ala, Trp, Phg and Tyr, preferably Phe, Phg or Trp, more preferably Phe or Phg;
   A′xx in formula (Ia) represents an amino acid selected from Dap, Dap, Lys, Orn and homoLys;
   A′yy in formulae (Ia) or (Ib) represents an amino acid selected from Phe, Ala, Trp, Phg and Tyr, preferably Phe, Phg or Trp, more preferably Phe or Phg;
   m is an integer of 1 to 4.
3. A compound represented by one of the following general formulae (II), (II′) and (IIa):

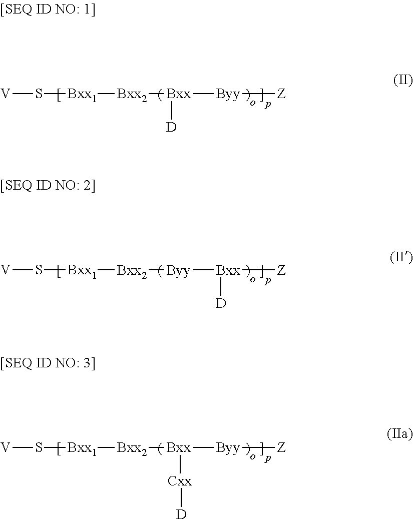

wherein
D represents a moiety derived from a drug; if o*p>1 one or more D's may be hydrogen with the proviso that at least one D represents a moiety derived from a drug;
Bxx in formulae (II) and (II′) represents a trifunctional amino acid such as an amino-dicarboxylic acid or a diamino-carboxylic acid; with the proviso that Bxx in formula (II) is not an amino acid in the (D) configuration;
Bxx in formulae (IIa) represents a carboxylic amino acid such as Ama, Glu, Aaa, Apa or a trifunctional amino acid selected from Dap, Dab, Ser, Thr, Lys, Orn, homoLys, homoSer and homoThr; with the proviso that Bxx is not an amino acid in the (D) configuration; Cxx represents a single covalent bond unless Bxx is Ama; if Bxx is Ama, Cxx represents (L)- or (D)-Pro, or an N-methyl amino acid such as Sar, the N-terminus of Cxx binds to a carboxyl end of Ama and the C-terminus of Cxx binds to moiety D;
in those instances where Bxx in formulae (II), (II′) and (IIa) carries a hydrogen as D group, Bxx may also be any other amino acid, with the proviso that Bxx in formulae (II) and (IIa) is not an amino acid in the (D) configuration;
Byy represents an amino acid selected from Phe, Ala, Trp, Tyr, Phg, Val, His, Lys, Abu, Met, Cit, Orn, Ser, Thr, Leu, IIe and Arg; or Byy in formulae (II) and (IIa) represents an amino acid selected from homo-Phe, beta-Phe and beta-homo-Phe; with the proviso that Byy in formula (II′) is not an amino acid in the (D) configuration and with the proviso that if o*p>1, only the C-terminal Byy in formulae (II) and (IIa) may represent an amino acid selected from homo-Phe, beta-Phe and beta-homo-Phe;
Bxx₁ represents a single covalent bond or an amino acid having a hydrophobic or basic side chain;
Bxx₂ represents an amino acid having a hydrophobic or basic side chain;
S and V are as defined in item 1;
Z is covalently bonded to the C-terminus of Byy in formulae (II) and (IIa) and the C-terminus of Bxx in formula (II′), and represents a group selected from —OH; —N(H)(R), R being as defined in item 1; and a labelling agent; and
o and p each independently is an integer of 1 to 10, if p is more than 1, Bxx₁ is not an amino acid in the (D) configuration; if p is more than 1 and/or if o is more than 1, then each D can be independently selected from moieties derived from a drug.
4. The compound of item 3, wherein at least one of Bxx₁, Bxx₂, Bxx, Byy, o and p is defined as follows:
   Bxx₁ represents a single covalent bond or an amino acid selected from Phe, Phg, Val, Ser, Tyr, Ala, Leu, IIe; preferably an amino acid selected from Phe, Phg, Tyr and Val, more preferably Phe, Phg or Tyr;
   Bxx₂ represents an amino acid selected from Arg, Lys, Cit, Val, Leu, Ser, Ala, Gly, His, Gln, Phg and Phe; preferably an amino acid selected from Arg, Lys, Cit and Phe;
   Bxx in formulae (II) and (II′) represents an amino acid selected from Dap, Dab, Lys, Orn, Ser, Glu, Ama, Thr, Aaa, homoSer and homoThr; preferably Lys or Dab;
   Byy represents Phe, Phg or Trp, preferably Phe or Phg; and
   o and p each independently is an integer of 1 to 4.
5. The compound of any of items 1 to 4, wherein in formulae (Ia1), (II), (II′) and (IIa),
   S represents a divalent group selected from a divalent alkylene group, a divalent alkenylene group, a divalent alkynylene group, and a divalent polyalkylene oxide;
   preferably a divalent group having formula —(CH₂)_{q}-Azz₅-, or —(OCH₂CH₂)_{q}-Azz₅-; wherein q is an integer of 1 to 50; and Azz₅ is absent, or represents a solubilizing group preferably selected from an amino acid such as Arg and a divalent group containing an ammonium group or a sulfate group.
6. The compound of any of items 1 to 5, wherein in formulae (Ia1), (II), (II′) and (IIa),
   S represents a divalent group having formula —(CH₂)_{q}-Azz₅-Y—, or a divalent group having formula —(OCH₂CH₂)_{q}-Azz₅-Y—;
   wherein Y represents a divalent moiety covalently bonded to the C-terminus of Azz₅ and to moiety V; if Azz₅ is absent, Y is covalently bonded to the alkyl group or polyethylene oxide group and to moiety V; Y is derived from a compound selected from maleimides, triazoles, especially 1,2,3-triazole, hydrazones, carbonyl-containing groups, and derivatives thereof, preferably from maleimides and derivatives thereof; q is an integer of 1 to 50; and Azz₅ is as defined in item 5.
7. The compound of item 1, which is selected from:

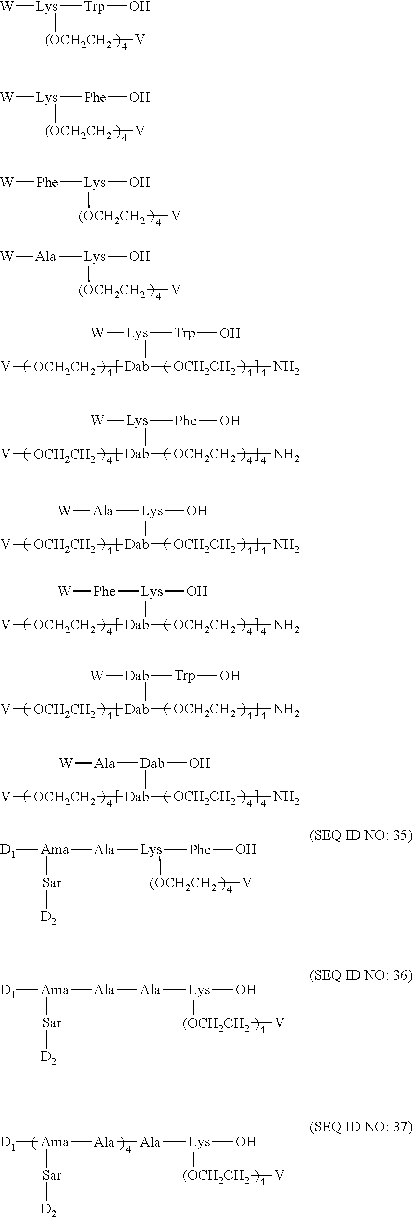

wherein W, V, D₁ and D₂ have the same meanings as specified in item 1.
8. The compound of item 3, which is selected from:

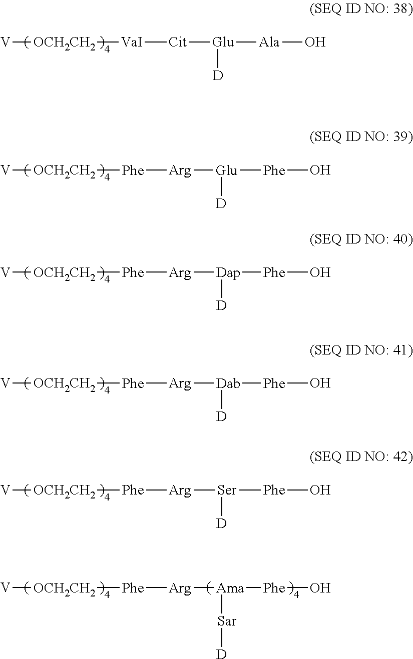

wherein V and D have the same meanings as specified in item 4.
9. The compound of any of items 1 to 8, wherein each moiety derived from a drug is independently selected from:
   (i) antineoplastic agents including alkylating agents, alkaloids such as taxanes and maytansinoids, anti-metabolites, endocrine therapies, kinase inhibitors;
   (ii) immunomodulatory agents such as immunostimulants and immunosuppressants;
   (iii) anti-infectious disease agents including antibacterial drugs, antimitotic drugs, antimycobacterial drugs and antiviral drugs;
   radioisotopes and/or pharmaceutically acceptable salts thereof.
10. The compound of any of items 1 to 9, wherein each moiety derived from a drug is independently derived from duocarmycin, auristatin, maytansine, tubulysin, calicheamicin, camptothecin, SN-38, taxol, daunomycin, vinblastine, doxorubicin, methotrexate, pyrrolobenzodiazepine, or radioisotopes and/or pharmaceutically acceptable salts thereof.
11. The compound of any of items 1, 2, 5, 6, 7, 9 and 10, wherein each moiety D₁ is independently represented by the following formula (III):

W₁-Dxx-Dyy---- (III)
wherein
W₁ represents a moiety derived from duocarmycin, auristatin, maytansine, tubulysin, calicheamicin, camptothecin, SN-38, taxol, daunomycin, vinblastine, doxorubicin, methotrexate, pyrrolobenzodiazepine, or radioisotopes and/or pharmaceutically acceptable salts thereof;
Dxx represents a single covalent bond, an amino acid having a hydrophobic side chain, preferably an amino acid selected from Phe, Val and Ala, or an amino acid having a hydrophobic side chain that is attached to moiety W₁ via a divalent moiety selected from maleimides, triazoles, hydrazones, carbonyl-containing groups, and derivatives thereof, preferably via a divalent maleimide derivative;
Dyy represents a single covalent bond or an amino acid having a basic side chain, preferably an amino acid selected from Arg, Lys, Phe, Cit, Orn, Dap, and Dab, more preferably Arg or Cit;
and the broken line indicates covalent attachment to the N-terminus of Axx in formula (I), the N-terminus of Ayy in formula (I′), the N-terminus of A′xx in formulae (Ia) and (Ib), or the N-terminus A′yy in formula (Ia′).
12. The compound of any of items 1 to 11, wherein each moiety D₂ and D is independently represented by the following formula (IIIa):

W₂-Exx---- (IIIa)
wherein
W₂ represents a moiety derived from duocarmycin, auristatin, maytansine, tubulysin, calicheamicin, camptothecin, SN-38, taxol, daunomycin, vinblastine, doxorubicin, methotrexate, pyrrolobenzodiazepine, or radioisotopes and/or pharmaceutically acceptable salts thereof;
Exx represents a single covalent bond or a divalent moiety selected from maleimides, triazoles, hydrazones, carbonyl-containing groups, amino acids, dipeptide moieties and derivatives thereof, preferably a maleimide derivative;
and the broken line indicates covalent attachment to the side chain of A′xx in formulae (Ia) and (Ia′), the side chain of A′xx or C-terminus of Cxx if present in formula (Ib), the side chain of Bxx in formulae (II) and (II′), the side chain of Bxx or C-terminus of Cxx if present in formula (IIa).
13. The compound of any of items 1 to 12, wherein V represents a moiety derived from a vector group selected from antibodies, antibody fragments, proteins, peptides and non-peptidic molecules;
   preferably an antibody or an antibody fragment such as a single chain antibody, a monoclonal antibody, a single chain monoclonal antibody, a monoclonal antibody fragment, a chimeric antibody, a chimeric antibody fragment, a domain antibody or fragment thereof, a cytokine, a hormone, a growth factor, a colony stimulating factor, a neurotransmitter or a nutrient-transport molecule.
14. The compound of any of items 1 to 13, wherein
   V represents a moiety derived from a vector group capable of interacting with a target cell, wherein the target cell is selected from tumor cells, virus infected cells, microorganism infected cells, parasite infected cells, cells involved in autoimmune diseases, activated cells, myeloid cells, lymphoid cells, melanocytes, and infectious agents including bacteria, viruses, mycobacteria, fungi,
   preferably the target cell is selected from lymphoma cells, myeloma cells, renal cancer cells, breast cancer cells, prostate cancer cells, ovarian cancer cells, colorectal cancer cells, gastric cancer cells, squamous cancer cells, small-cell lung cancer cells, testicular cancer cells, and any cells growing and dividing at an unregulated and quickened pace to cause cancers.
15. Composition comprising a therapeutically effective amount of the compound of any of items 1 to 14 or a pharmaceutically acceptable salt thereof, and one or more components selected from a carrier, a diluent and other excipients.
16. The compound or composition of any of items 1 to 15 for use in a method of treating or preventing a cancer, an autoimmune disease and/or an infectious disease.
17. The compound or composition for use of item 16, wherein in the method of treating or preventing a cancer, an autoimmune disease and/or an infectious disease, the compound or composition is administered concurrently with, before or after one or more other therapeutic agents or therapies such as chemotherapeutic agents, radiation therapy, immunotherapy agents, autoimmune disorder agents, anti-infectious agents or other compounds of formula (I)/(I′) or (II)/(II′)/(IIa).
18. Method for treating or preventing a cancer, an autoimmune disease and/or an infectious disease, wherein a therapeutically effective amount of the compound or composition of any of items 1 to 15 is administered to a patient in need thereof.

### FIGURES

FIG. 1—(a) Exo-Cat B-induced drug release mechanism from a compound of formula (I), wherein W represents a moiety represented by formula (III) or a peptide moiety represented by formula (Ia), (Ia′) or (Ib), and T is a moiety of formula (Ia₁) with n=1. Intracellular exo-Cat B cleavage at the N-terminus of dipeptide Axx-Ayy releases the free drug in the target cell. According to this embodiment, enzymatic cleavage and drug release occur simultaneously. (b) Exo-Cat B-induced dug release mechanism from a compound of formula (I), wherein W represents a moiety represented by formula (III), and T is a moiety of formula (Ia₁) with n=1. Intracellular exo-Cat B cleavage at the N-terminus of the dipeptide Axx-Ayy releases the modified drug W₁-Dxx-Dyy, wherein W₁ is drug derived from a native drug only by virtue of the covalent attachment to Dxx, in the target cell.

FIG. 2—Exo-Cat B-induced drug release mechanism from a compound of formula (I′), wherein W represents a moiety represented by formula (III) or a peptide moiety represented by formula (Ia), (Ia′) or (Ib), and T is a moiety of formula (Ia₁) with n=1. Intracellular exo-Cat B cleavage at the N-terminus of dipeptide Ayy-Axx releases the free drug in the target cell. According to this embodiment, enzymatic cleavage and drug release occur simultaneously.

FIG. 3—Exo-Cat B-induced drug release mechanism from a compound of formula (I) or formula (I′), wherein W represents a moiety represented by formula (III) or a peptide moiety represented by formula (Ia), (Ia′) or (Ib) and T is a moiety of formula (Ia₁) with n=4. Intracellular exo-Cat B cleavage at the N-terminus of each C-terminal dipeptide Axx-Ayy of formula (I) or each C-terminal dipeptide Ayy-Axx of formula (I′) releases the free drug(s) in the target cell. According to this embodiment, multiple enzymatic cleavage and drug(s) release occur simultaneously.

FIG. 4—Exo-Cat B-induced drug release mechanism from a compound of formula (I) wherein W represents a peptide moiety having formula (Ia) with m=1 and T is a moiety of formula (Ia₁) with n=1. Intracellular exo-Cat B cleavage at the N-terminus of Axx-Ayy and A′xx-A′yy releases moiety D₁ and A′xx(D₂)-A′yy. According to this embodiment, enzymatic cleavage and release of D₁ and A′xx(D₂)-A′yy occur simultaneously. According to one further embodiment of the present invention, A′xx(D₂)-A′yy can undergo intramolecular aminolysis (i.e. 5- or 6-membered ring formation), enzyme- or acid-catalyzed hydrolysis, or diketopiperazine (DKP) formation to release D₂.

FIG. 5—Exo-Cat B-induced drug release mechanism from a compound of formula (I) wherein W represents a peptide moiety having formula (Ia) with m≥1 and T represents a moiety of formula (Ia₁ with n=1. Intracellular exo-Cat B cleavage at the N-terminus of Axx-Ayy and sequential exo-Cat B cleavage of each A′xx-A′yy unit releases moiety D₁ and m A′xx(D₂)-A′yy moieties. According to one embodiment of the present invention, one or more moieties A′xx(D₂)-A′yy can undergo intramolecular aminolysis or hydrolysis to release D₂.

FIG. 6—Exo-Cat B-induced drug release mechanism from a compound of formula (I′) wherein W represents a peptide moiety having formula (Ia) with m=1 and T is a moiety of formula (Ia₁ with n=1. Intracellular exo-Cat B cleavage at the N-terminus of Ayy-Axx and A′xx-A′yy releases D₁ and A′xx(D₂)-A′yy. According to one embodiment of the present invention, moiety A′xx(D₂)-A′yy can undergo intramolecular aminolysis or hydrolysis to release D₂.

FIG. 7—Exo-Cat B-induced drug release mechanism from a compound of formula (I′) wherein W represents a peptide moiety having formula (Ia) with m>1 and T represents a moiety of formula (Ia₁ with n=1. Intracellular exo-Cat B cleavage at the N-terminus of Ayy-Axx and sequential exo-Cat B cleavage of each A′xx-A′yy releases D₁ and m A′xx(D₂)-A′yy moieties. According to one embodiment of the present invention, one or more moieties A′xx(D₂)-A′yy can undergo intramolecular aminolysis or hydrolysis to release D₂.

FIG. 8—Exo-Cat B-induced drug release mechanism from a compound of formula (II). Intracellular exo-Cat B cleavage at the N-terminus of each peptide Bxx-Byy releases V—S-Bxx₁-Bxx₂ and o moieties Bxx(D)-Byy-OH, which can undergo intramolecular aminolysis or hydrolysis to release o moieties D.

FIG. 9—Exo-Cat B-induced drug release mechanism from a compound of formula (I′) wherein D represents a moiety derived from maytansine (DMR), and T represents a moiety of formula (Ia₁ with n=1, in which S is —(OCH₂CH₂)_{q} with q=4 (PEG4). Dyy in FIG. 9 represents an amino acid selected from Arg, Lys, Cit and Phe. According to this embodiment, Cat B-induced enzymatic cleavage at the N-terminus of Axx releases the drug in the target cell.

FIG. 10—Schematic drawing illustrating the use of LDCs in diagnostics. Cat B-induced cleavage at the C-terminus of the 7-amino-4-methylcoumarin (AMC) fluorescent probe allows monitoring drug release in the target cell.

FIGS. 11-17—Schematic drawings showing the synthetic preparation of compounds 1-7 (compounds of formula (I) or (I′)).

FIG. 11: preparation of compound 1, i.e. AF-Arg-Lys(PEG₄-Mal-Cys-Ac)-Phe-OH (compound of formula (I) with n=1, W=AF-Arg, W₁=AF, Dxx=single bond, Dyy=Arg, T=PEG₄-Mal-Cys-Ac, Axx=Lys, Ayy=Phe and Z═OH).

FIG. 12: preparation of compound 2, i.e. AF-Arg-Lys(PEG₄-Mal)-Phe-OH (compound of formula (I) with n=1, W=AF-Arg, W₁=AF, Dxx=single bond, Dyy=Arg, Axx=Lys, Ayy=Phe and Z═OH; “Mal” represents a maleimide derivative for vector attachment).

FIG. 13: preparation of compound 3, i.e. AF-Arg-Phe-Lys(PEG₄-Mal-Cys-Ac)—OH (compound of formula (I′) with n=1, W=AF-Arg, W₁=AF, Dxx=single bond, Dyy=Arg, T=PEG₄-Mal-Cys-Ac, Axx=Lys, Ayy=Phe and Z═OH).

FIG. 14: preparation of compound 4, i.e. AF-Arg-Phe-Lys(PEG₄-Mal)-OH (compound of formula (I′) with n=1, W=AF-Arg, W₁=AF, Dxx=single bond, Dyy=Arg, Axx=Lys, Ayy=Phe and Z═OH; “Mal” represents a maleimide derivative for vector attachment).

FIG. 15: preparation of compound 5, i.e. DM1-Mal-Phe-Lys-Lys(PEG₄-Mal-Cys-Ac)-Phe-OH (SEQ ID NO: 44) (compound of formula (I) with n=1, W₁=DM1, Dxx=Mal-Phe, Dyy=Lys, T=PEG₄-Mal-Cys-Ac, Axx=Lys, Ayy=Phe and Z═OH).

FIG. 16: preparation of compound 6, i.e. DM1-Mal-Phe-Cit-Lys(PEG₄-Mal-Cys-Ac)-Phe-OH (SEQ ID NO: 45) (compound of formula (I) with n=1, W₁=DM1, Dxx=Mal-Phe, Dyy=Cit, T=PEG₄-Mal-Cys-Ac, Axx=Lys, Ayy=Phe and Z═OH).

FIG. 17: preparation of compound 7, i.e. DM1-Mal-Phe-Cit-Phe-Lys(PEG₄-Mal-Cys-Ac)—OH (SEQ ID NO: 46) (compound of formula (I′) with n=1, W₁=DM1, Dxx=Mal-Phe, Dyy=Cit, T=PEG₄-Mal-Cys-Ac, Axx=Lys, Ayy=Phe and Z═OH).

FIGS. 18-28—Schematic drawings showing the synthetic preparation of compounds 8-18 (compounds of formula (II)).

FIG. 18: preparation of compound 8, i.e. Arg-PEG₄-Phe-Arg-Glu(Sar-OCPT)-Phe-OH (compound of formula (IIa) with o=1 and p=1, D=CPT, V=Arg, S=PEG₄, Bxx=Glu, Byy=Phe, Bxx₁=Phe, Bxx₂=Arg, Cxx=Sar and Z═OH).

FIG. 19: preparation of compound 9, i.e. Arg-PEG₄-Phe-Arg-Dap(CO-CPT)-Phe-OH (SEQ ID NO: 48) (compound of formula (II) with o=1 and p=1, D=CPT, V=Arg, S=PEG₄, Bxx=Dap, Byy=Phe, Bxx₁=Phe, Bxx₂=Arg and Z═OH).

FIG. 20: preparation of compound 10, i.e. Arg-PEG₄-Phe-Arg-Dab(CO-CPT)-Phe-OH (SEQ ID NO: 49) (compound of formula (II) with o=1 and p=1, D=CPT, V=Arg, S=PEG₄, Bxx=Dab, Byy=Phe, Bxx₁=Phe, Bxx₂=Arg and Z═OH).

FIG. 21: preparation of compound 11, i.e. Arg-PEG₄-Phe-Arg-Ser(CO-CPT)-Phe-OH (SEQ ID NO: 50) (compound of formula (II) with o=1 and p=1, D=CPT, V=Arg, S=PEG₄, Bxx=Ser, Byy=Phe, Bxx₁=Phe, Bxx₂=Arg and Z═OH).

FIG. 22: preparation of compound 12, i.e. Ac-Cys-Mal-PEG₄-Phe-Lys-Lys(Mal-DM1)-Phe-OH (SEQ ID NO: 51) (compound of formula (II) with o=1 and p=1, D=DM1-Mal, V═Ac-Cys-Mal, S=PEG₄, Bxx=Lys, Byy=Phe, Bxx₁=Phe, Bxx₂=Lys and Z═OH).

FIG. 23: preparation of compound 13, i.e. Ac-Cys-Mal-PEG₄-Phe-Lys-Lys(AF)-Phe-OH (SEQ ID NO: 52) (compound of formula (II) with o=1 and p=1, D=AF, V═Ac-Cys-Mal, S=PEG₄, Bxx=Lys, Byy=Phe, Bxx₁=Phe, Bxx₂=Lys and Z═OH).

FIG. 24: preparation of compound 14, i.e. Mal-PEG₄-Phe-Lys-Lys(AF)-Phe-OH (SEQ ID NO: 53) (compound of formula (II) with o=1 and p=1, D=AF, S=Mal-PEG₄, Bxx=Lys, Byy=Phe, Bxx₁=Phe, Bxx₂=Lys and Z═OH; “Mal” represents a maleimide derivative for vector attachment).

FIG. 25: preparation of compound 15, i.e. Arg-PEG₄-Phe-Arg-Glu(Sar-OCPT)-Arg-OH (compound of formula (IIa) with o=1 and p=1, D=CPT, V=Arg, S=PEG₄, Bxx=Glu, Byy=Arg, Bxx₁=Phe, Bxx₂=Arg, Cxx=Sar and Z═OH).

FIG. 26: preparation of reference compound 16, i.e. Arg-PEG₄-Phe-Arg-Glu(Sar-OCPT)-Arg-Phe-Arg-OH.

FIG. 27: preparation of compound 17, i.e. Arg-PEG₄-Phe-Arg-[Glu(Sar-OCPT)-Arg]₂-OH (compound of formula (IIa) with o=2 and p=1, D=CPT, V=Arg, S=PEG₄, Bxx=Glu, Byy=Arg, Bxx₁=Phe, Bxx₂=Arg, Cxx=Sar and Z═OH).

FIG. 28: preparation of compound 18, i.e. Arg-PEG₄-[Phe-Arg-Glu(Sar-OCPT)-Arg]2-OH (compound of formula (IIa) with o=1 and p=2, D=CPT, V=Arg, S=PEG₄, Bxx=Glu, Byy=Arg, Bxx₁=Phe, Bxx₂=Arg, Cxx=Sar and Z═OH).

FIG. 29—Exo-Cat B-induced drug release study from compound 1 i.e. AF-Arg-Lys(PEG₄-Mal-Cys-Ac)-Phe-OH (formula (I)). Cleavage of compound 1 quantitatively released pharmacologically active moiety AF-Arg (t_{1/2}=1.5 min).

FIG. 30—Exo-Cat B-induced drug release study from compound 3, i.e. AF-Arg-Phe-Lys(PEG₄-Mal-Cys-Ac)—OH (formula (I′)). Cleavage of compound 3 quantitatively released pharmacologically active moiety AF-Arg (t_{1/2}=1.4 min).

FIG. 31—Exo-Cat B-induced drug release study from compound 5, i.e. DM1-Mal-Phe-Lys-Lys(PEG₄-Mal-Cys-Ac)-Phe-OH (SEQ ID NO: 44) (formula (I)). Cleavage of compound 5 quantitatively released pharmacologically active moiety DM1-Mal-Phe-Lys (t_{1/2}=0.59 min).

FIG. 32—Exo-Cat B-induced drug release study from compound 12, i.e. Ac-Cys-Mal-PEG₄-Phe-Lys-Lys(Mal-DM1)-Phe-OH (SEQ ID NO: 51) (formula (II)). Cleavage of compound 12 released pharmacologically active moiety Lys(Mal-DM1)-Phe (t_{1/2}=1.53 min).

FIG. 33—Exo-Cat B-induced drug release study from compound 17, i.e. Arg-PEG₄-Phe-Arg-[Glu(Sar-CPT)-Phe]₂-OH (formula (II)). C-terminal dipeptide cleavage of compound 17 results in the temporary formation of intermediate Arg-PEG₄-Phe-Arg-(Glu(Sar-CPT)-Phe-OH (compound 8) which, in turn, is cleaved by Cat B to release a second Glu(Sar-CPT)-Phe-OH, which by ester hydrolysis releases camptothecin (CPT).

FIG. 34—Exo-Cat B-induced drug release study from compound 18, i.e. Arg-PEG₄-[Phe-Arg-Glu(Sar-CPT)-Arg]₂-OH (formula (II)). Sequential cleavage of compound 18 results in the temporary formation of intermediates Arg-PEG₄-Phe-Arg-(Glu(Sar-CPT)-Arg-Phe-Arg-OH (compound 16) and Arg-PEG₄-Phe-Arg-(Glu(Sar-CPT)-Arg-OH (compound 15), which in turn is cleaved by Cat B to release a second Glu(Sar-CPT)-Arg-OH. Subsequently, native CPT is released via ester hydrolysis.

FIG. 35—Cytotoxicity activity study of compounds AF and AF-Arg in ErbB2-expressing SK-BR-3 and SK-OV-3 cell lines at incubation time 120 h (example 9). Upper trace: SK-OV-3 survival ratio (%) after 120 h incubation with decreasing concentrations of AF or AF-Arg (log-dilution) (two runs; IC₅₀(AFᵣᵤₙ₁)=100.2 nM, IC₅₀(AFᵣᵤₙ₂)=145.2 nM; IC₅₀ (AF-Argᵣᵤₙ₁)=146.7 nM, IC₅₀ (AF-Argrun₂)=204.7 nM). Lower trace: SK-BR-3 survival ratio (%) after 120 h incubation with decreasing concentrations of AF or AF-Arg (log-dilution)(two runs; IC₅₀ (AFᵣᵤₙ₁)=18.82 nM, IC₅₀(AFᵣᵤₙ₂)=21.75 nM; IC₅₀ (AF-Argᵣᵤₙ₁)=20.9 nM, IC₅₀ (AF-Argrun₂)=24.92 nM).

FIGS. 36-41—Schematic drawings showing the synthetic preparation of compounds 19-24 (compounds of formula (I)/(I′)).

FIG. 36: preparation of compound 19, i.e. AF-Cit-Lys(PEG₄-Mal-Cys-Ac)-Phe-OH (compound of formula (I)/(III) with W₁=AF, Dxx=single bond, Dyy=Cit, T=PEG₄-Mal-Cys-Ac, Axx=Lys, Ayy=Phe and Z═OH).

FIG. 37: preparation of compound 20, i.e. ACit-Lys(PEG₄-Mal-Cys-Ac)-Phe-OH (compound of formula (I)/(111) with W₁=ACit, Dxx=Dyy=single bond, T=PEG₄-Mal-Cys-Ac, Axx=Lys, Ayy=Phe and Z═OH).

FIG. 38: preparation of compound 21, i.e. ACit-Phe-Lys(PEG₄-Mal-Cys-Ac)—OH (compound of formula (I′)/(III) with W₁=ACit, Dxx=single bond, Dyy=Phe, T=PEG₄-Mal-Cys-Ac, Axx=Lys, Ayy=Phe and Z═OH).

FIG. 39: preparation of compound 22, i.e. DM1-Mcc-Phe-Cit-Lys(PEG₅-Ma-Cys-Ac)-Tyr-OH (SEQ ID NO: 58) (compound of formula (I)/(III) with W₁=DM1, Dxx=Mcc-Phe, Dyy=Cit, T=PEG₅-Ma-Cys-Ac, Axx=Lys, Ayy=Tyr and Z═OH).

FIG. 40: preparation of compound 23, i.e. DM1-Mcc-Cit-Lys(PEG₅-Ma-Cys-Ac)-Tyr-OH (compound of formula (I)/(III) with W₁=DM1, Dxx=single bond, Dyy=Cit, T=PEG₅-Ma-Cys-Ac, Axx=Lys, Ayy=Tyr and Z═OH).

FIG. 41: preparation of compound 24, i.e. DM1-Mcc-Phe-Lys(PEG₅-Ma-Cys-Ac)-Tyr-OH (compound of formula (I)/(III) with W₁=DM1, Dxx=Mcc-single bond, Dyy=Phe, T=PEG₅-Ma-Cys-Ac, Axx=Lys, Ayy=Tyr and Z═OH).

FIGS. 42-45—Schematic drawings showing the synthetic preparation of compounds 25-28 (compounds of formula (II)/(II′)).

FIG. 42: preparation of compound 25, i.e. Ac-Cys-Ma-PEG₅-Phe-Cit-Lys(Mcc-DM1)-Cit-OH (SEQ ID NO: 59) (compound of formula (II) with o=1 and p=1, D=DM1-Mcc, V═Ac-Cys-Ma, S=PEG₅, Bxx=Lys, Byy=Cit, Bxx₁=Phe, Bxx₂=Cit and Z═OH).

FIG. 43: preparation of compound 26, i.e. Ma-PEG₅-Phe-Cit-Lys(Mcc-DM1)-Cit-OH (SEQ ID NO: 60) (compound of formula (II) with o=1 and p=1, D=DM1-Mcc, S=PEG₅, Bxx=Lys, Byy=Cit, Bxx₁=Phe, Bxx₂=Cit and Z═OH).

FIG. 44: preparation of compound 27, i.e. Ac-Cys-Ma-PEG₅-Phe-Cit-Lys(Mcc-DM1)-Tyr-OH (SEQ ID NO: 61) (compound of formula (II) with o=1 and p=1, D=DM1-Mcc, V═Ac-Cys-Ma, S=PEG₅, Bxx=Lys, Byy=Tyr, Bxx₁=Phe, Bxx₂=Cit and Z═OH).

FIG. 45: preparation of compound 28, i.e. Ac-Cys-Mal-PEG₄-Phe-Lys-Lys(Mal-DM1)-Phe-Phe-Lys-OH (SEQ ID NO: 62) (intermediate for compound 29).

FIGS. 46-47—Schematic drawings showing the synthetic preparation of compounds 29-30 (compounds of formula (II) for multiple drug release).

FIG. 46: preparation of compound 29, i.e. Ac-Cys-Mal-PEG₄-[Phe-Lys-Lys(Mal-DM1)-Phe]2-OH (SEQ ID NO: 63) (compound of formula (II) with o=1 and p=2, D=Mal-DM1, V═Ac-Cys, S=PEG₄, Bxx=Lys, Byy=Phe, Bxx₁=Phe, Bxx₂=Lys and Z═OH).

FIG. 47: preparation of compound 30, i.e. Ac-Cys-Mal-PEG₄-Phe-Arg-Lys(Mal-DM1)-Arg-Lys(AF)-Phe-OH (SEQ ID NO: 64) (compound of formula (II) with o=2 and p=1, D=Mal-DM1/AF, V═Ac-Cys, S=PEG₄, Bxx=Lys/Lys, Byy=Arg/Phe, Bxx₁=Phe, Bxx₂=Arg and Z═OH).

FIGS. 48-49—Schematic drawings showing the synthetic preparation of compounds 31-32 (compounds of formula (I) for multiple drug release).

FIG. 48: preparation of compound 31, i.e. AF-Cit-Lys(Mal-DM1)-Phe-Lys(PEG₄-Mal-Cys-Ac)-Phe-OH (SEQ ID NO: 65) (compound of formula (I) wherein W is a peptide moiety of formula (Ia) with m=1, D₁=AF-Cit, D₂=Mal-DM1, Axx/A′xx=Lys, Ayy/A′yy=Phe, T=PEG₄-Mal-Cys-Ac and Z═OH).

FIG. 49: preparation of compound 32, i.e. Ac-Cys-Mal-[PEG₅-Lys(AF-Cit-Lys(Y)-Phe-OH)]2-Gly-NH₂ (SEQ ID NO: 11) (compound of formula (I) wherein W is a moiety of formula (III) in which W₁=AF, Dxx=single bond and Dyy=Cit, Axx=Lys, Ayy=Phe and Z═OH; T is a moiety of formula (Ia₁) with V═Ac-Cys-Mal, S=PEG₅-Lys(Y), Y=triazole, Rₓ=Gly-NH₂ and n=2).

FIG. 50—Exo-Cat B-induced drug release study from compound 13, i.e. Ac-Cys-Mal-PEG₄-Phe-Lys-Lys(AF)-Phe-OH (SEQ ID NO: 52) (formula (II)). Cleavage of compound 13 released pharmacologically active moiety Lys(AF)-Phe (t_{1/2}=1.62 min).

FIG. 51—Exo-Cat B-induced drug release study from compound 29, i.e. Ac-Cys-Mal-PEG₄-[Phe-Lys-Lys(Mal-DM1)-Phe]2-OH (SEQ ID NO: 63) (formula (II)). Multiple cleavages of compound 29 released two pharmacologically active moieties Lys(Mal-DM1)-Phe.

FIG. 52—Exo-Cat B-induced drug release study from compound 30, i.e. Ac-Cys-Mal-PEG₄-Phe-Arg-Lys(Mal-DM1)-Arg-Lys(AF)-Phe-OH (SEQ ID NO: 64) (formula (II)). Multiple cleavages of compound 30 released pharmacologically active moieties Lys(Mal-DM1)-Arg and Lys(AF)-Phe.

FIG. 53—Exo-Cat B-induced drug release study from compound 31, i.e. AF-Cit-Lys(Mal-DM1)-Phe-Lys(PEG₄-Mal-Cys-Ac)-Phe-OH (SEQ ID NO: 65) (formula (I)). Multiple cleavages of compound 31 released pharmacologically active moieties AF-Cit and Lys(Mal-DM1)-Phe.

FIG. 54—Exo-Cat B-induced drug release study from compound 32, i.e. Ac-Cys-Mal-[PEG₅-Lys(AF-Cit-Lys(Y)-Phe-OH)]2-Gly-NH₂ (formula (I), Y=triazole). Multiple cleavages of compound 32 released two pharmacologically active moieties AF-Cit.

FIG. 55—Exo-Cat B-induced drug release study from antibody-drug-conjugate ADC1, i.e. AF-Arg-Lys(PEG₄-Mal-trastuzumab)-Phe-OH (formula (I)). Cleavage of ADC1 released the pharmacologically active moiety AF-Arg (t_{1/2}=4.22 min).

FIG. 56—Plasma stability study of antibody-drug-conjugate ADC1 over 24 h. The results demonstrate that ADC1 exhibits excellent stability in (human and mouse) plasma.

FIG. 57—Binding assay of antibody-drug-conjugate ADC1 and trastuzumab in ErbB2-expressing and ErbB2-negative cell lines (SK-BR-3 and MDA-MB-231, respectively). The results show that ADC1 has the same affinity and specificity for ErbB2-expressing cells than trastuzumab.

FIG. 58—Binding assay of antibody-drug-conjugate ADC3 and trastuzumab in ErbB2-expressing and ErbB2-negative cell lines (BT-474 and MDA-MB-231, respectively). The results show that ADC3 has the same affinity and specificity for ErbB2-expressing cells than trastuzumab.

FIG. 59—Cytotoxicity activity study of ADC1 in ErbB2-expressing and ErbB2-negative cell lines. FIG. 59a: ErbB2-expressing SK-OV-3 survival ratio (% viability) after 96 h incubation with increasing concentrations of ADC1 (squares), trastuzumab (stars), AF-Arg (triangles) and compound 2 (dots) (two runs; IC₅₀(ADC1ᵣᵤₙ₁)=34.4 pM, IC₅₀(ADC1ᵣᵤₙ₂)=93.9 pM). FIG. 59b: ErbB2-expressing SK-BR-3 survival ratio (% viability) after 96 h incubation with increasing concentrations of ADC1 (squares), trastuzumab (stars), AF-Arg (triangles) and compound 2 (dots) (two runs; IC₅₀ (ADC1ᵣᵤₙ₁)=14.8 pM, IC₅₀ (ADC1ᵣᵤₙ₂)=43.3 pM). FIG. 59c: ErbB2-negative MDA-Mb-231 survival ratio (% viability) after 96 h incubation with increasing concentrations of ADC1 (squares), trastuzumab (stars), AF-Arg (triangles) and compound 2 (dots) (two runs; IC₅₀ (ADC1ᵣᵤₙ₁)=0.128 nM, IC₅₀ (ADC1ᵣᵤₙ₂)=23.5 nM).

FIG. 60—Cytotoxicity activity study of ADC3 in ErbB2-expressing and ErbB2-negative cell lines. FIG. 60a: ErbB2-expressing BT-474 survival ratio (% viability) after 96 h incubation with increasing concentrations of ADC3 (triangles), DM1 (dots) and trastuzumab (plain dots), (two runs; IC₅₀(ADC3ᵣᵤₙ₁)=0.68 nM, IC₅₀(ADC3ᵣᵤₙ₂)=0.35 nM). FIG. 60b: ErbB2-negative MDA-MB-231 survival ratio (% viability) after 96 h incubation with increasing concentrations of ADC3 (triangles), DM1 (dots) and trastuzumab (plain dots), (two runs; IC₅₀(ADC3ᵣᵤₙ₁)=8.69 nM, IC₅₀(ADC3ᵣᵤₙ₂)=91.8 nM).

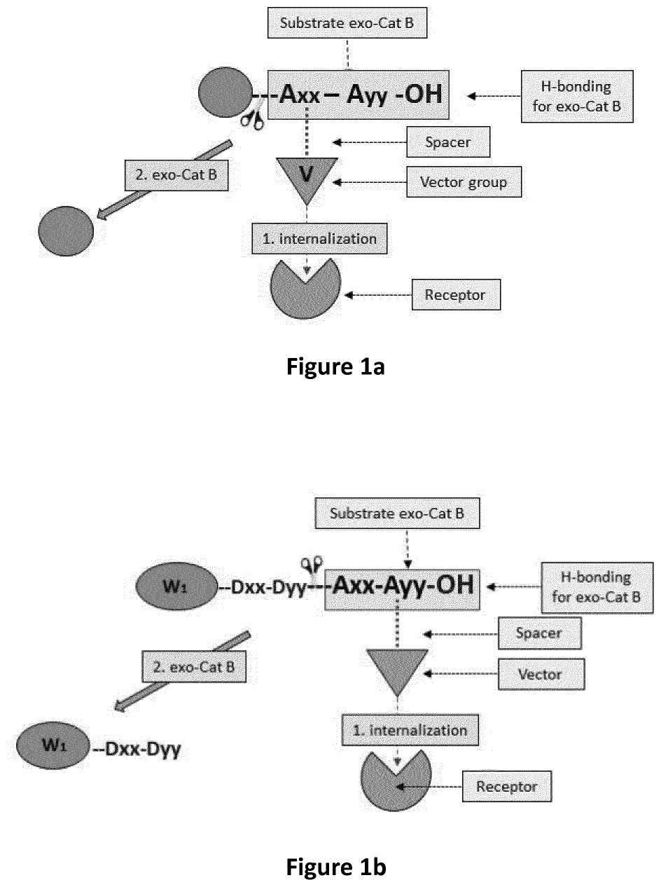

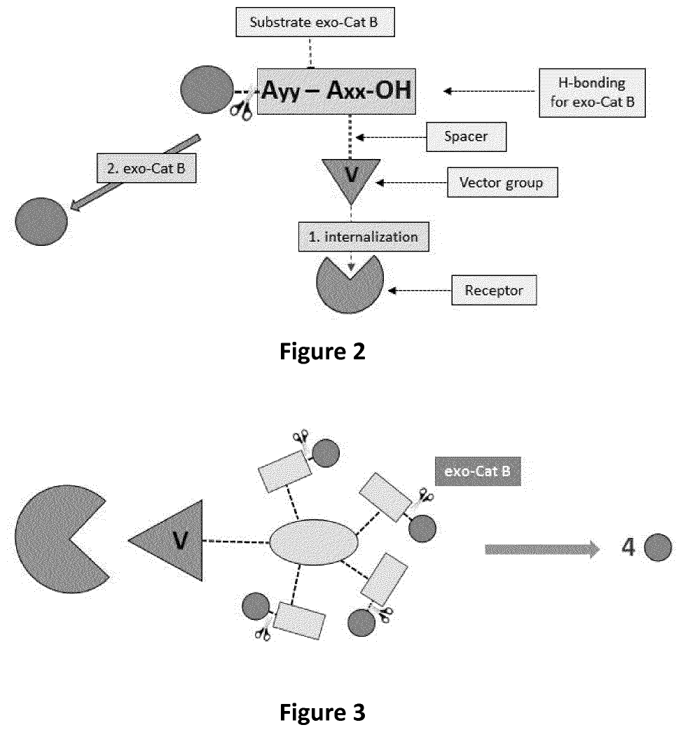

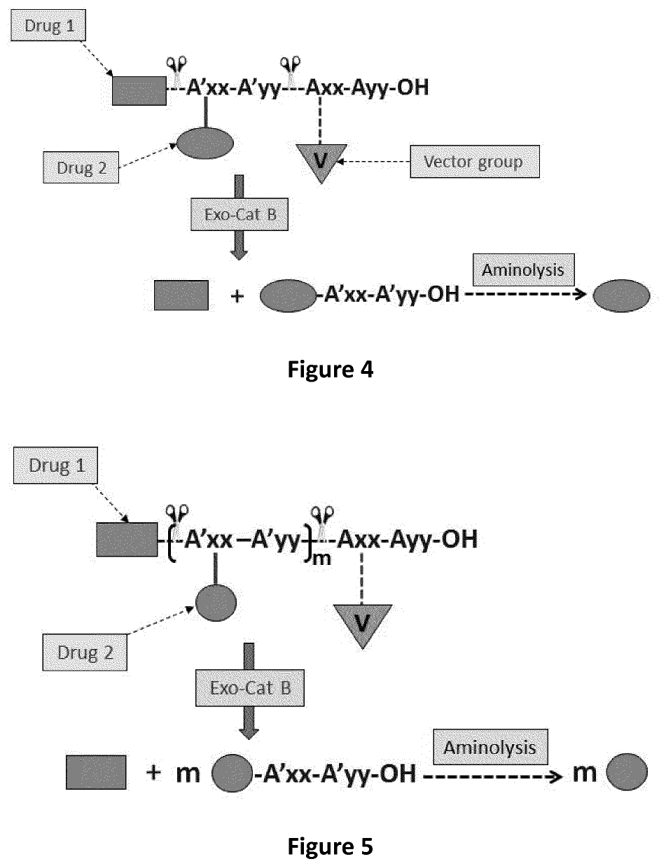

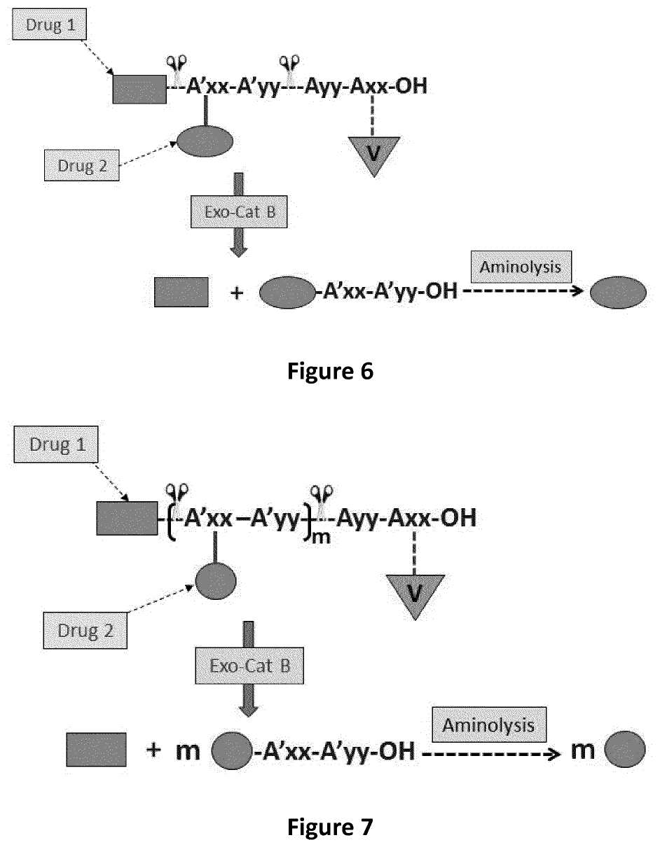

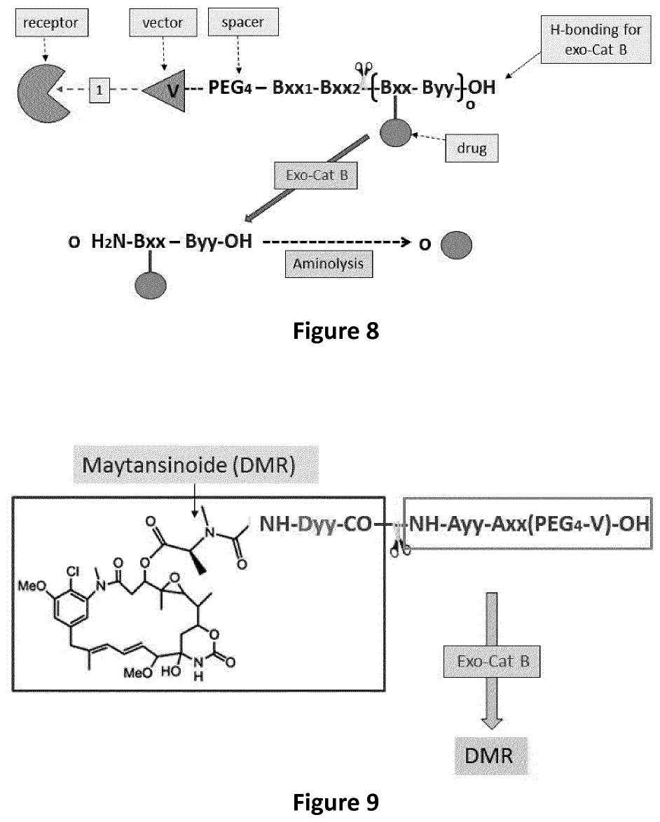

### DETAILED DESCRIPTION OF THE PRESENT INVENTION

### 1. Definitions

The term “C-terminal” as used herein refers to the C-terminal end of the amino acid chain, e.g. amino acid Ayy in dipeptide Axx-Ayy (formula (I)) or amino acid Axx in dipeptide Ayy-Axx (formula (I′)). Binding to the “C-terminus” means that a covalent bond is formed between the acid group of the amino acid residue and the binding partner. For instance, binding of group Z to the C-terminus of amino acid residue Ayy yields an ester or amide-type structural element —C(O)—X— with X being the binding partner of Z and the carbonyl group being derived from the amino acid residue Ayy.

The term “trifunctional” as used herein refers to a compound or moiety having three functional groups that can form or have formed three covalent bonds to adjacent moieties.

The term “functional group” refers to a group that is capable of bonding to another functional group by forming at least one covalent bond without need for breaking any C—C or C—H covalent bonds.

The term “amino acid” as used herein refers to a compound that contains or is derived from at least one amino group and at least one acidic group, preferably a carboxyl group. The distance between amino group and acidic group is not particularly limited. α-, β-, and γ-amino acids are suitable but α-amino acids and especially α-amino carboxylic acids are particularly preferred. This term encompasses both naturally occurring amino acids as well as synthetic amino acids that are not found in nature.

The expression “amino acid in the (D) configuration” as used herein refers to the (D)-isomer of any naturally occurring or synthetic amino acid. This applies to α-amino acids as well as to β- and γ-amino acids. The expression “amino acid in the (D) configuration” as used herein is not meant to encompass non-chiral amino acids such as glycine or other non-chiral amino acids such as aminoisobutyric acid.

Unless specified otherwise, chiral compounds and moieties may be present in the form of a pure stereoisomer or in the form of a mixture of stereoisomers, including the 50:50 racemate. In the context of the present invention, references to specific stereoisomers are to be understood as references to compounds or moieties, wherein the designated stereoisomer is present in at least 90% enantiomeric excess (ee), more preferably at least 95% ee and most preferably 100% ee.

The term “hydrophobic” is used herein to characterize compounds, groups or moieties, which lack affinity for water. For instance, the term “amino acid with hydrophobic side chain” is used to characterize amino acids with a hydrophobic or partially hydrophobic aliphatic side chain or amino acids with aromatic side chain such as Phe, Leu, IIe, Val, Tyr, Trp, Ala. Of course, any other amino acid exhibiting the same or a higher degree of hydrophobicity should also be treated as hydrophobic in the sense of the present invention. A comparison of the degree of hydrophobicity can be done by determining the n-octanol/water partition coefficient (at 25° C. and pH 7): if the ratio of concentrations in n-octanol/water for another amino acid is equal or higher than that of one or more of the amino acids Phe, Leu, IIe, Val, Tyr, Trp, Ala, such other amino acid is to be treated as a hydrophobic amino acid.

The term “amino acid with a basic side chain” is used herein to characterize natural or unnatural amino acids wherein the side chain contains one or more ionizable groups having a pKa value equal to or greater than 6. Examples of natural amino acids with a basic side chain include Arg (guanidino group, pKa=12.5), Lys (amino group, pKa=10.5), His (imidazole group, pKa=6). Examples of unnatural amino acids include citrulline (Cit), ornithine (Orn), 2,3-diamino-propionic acid (Dap), 2,4-diamino-butyric acid (Dab).

The term “solubilizing group” as used herein refers to a hydrophilic moiety, which enhances aqueous solubility of the compound to which it is bonded. Examples of solubilizing groups include ammonium groups, sulfate groups, phosphate groups, sulfonate groups and polyethylene glycol (PEG) groups, in particular groups of formula —(CH₂CH₂O)ₙ₁—H wherein n1 is 2 to 60, e.g. 2 to 24. The term “alkyl group” as used herein refers to a group having from 1 to 20 carbon atoms, preferably a methyl or an ethyl group, a cycloalkyl group having from 3 to 20 carbon atoms, preferably 5 to 8 carbon atoms, or an aromatic group having from 6 to carbon atoms, preferably 6 or 10 carbon atoms. The cycloalkyl group or the aromatic group may consist of a single ring, but it may also be formed by two or more condensed rings, e.g. a naphthyl group.

Where the present description refers to “preferred” embodiments/features, combinations of these “preferred” embodiments/features shall also be deemed as disclosed as long as this combination of “preferred” embodiments/features is technically meaningful.

Hereinafter, in the present description of the invention and the claims, the use of the terms “containing” and “comprising” is to be understood such that additional unmentioned elements may be present in addition to the mentioned elements. However, these terms should also be understood as disclosing, as a more restricted embodiment, the term “consisting of” as well, such that no additional unmentioned elements may be present, as long as this is technically meaningful. For instance, the expression “divalent carbonyl-containing group” includes as a preferred embodiment a divalent group consisting of carbonyl (—CO—). Moreover, the expression “at least one of X and Y” is to be understood broadly as disclosing one or both of X and Y, i.e. as being equivalent to the expression “at least one selected from the group of X and Y”.

The term “drug” as used herein is to be understood as a pharmacologically active substance which can inhibit or prevent the function of cells and/or kill cells. In some embodiments, the term “drug” is to be understood as being synonymous with other terms commonly used in the art such as “toxin” or “payload” used in the field of cancer therapy.

The expression “moiety derived from a drug” as used herein characterizes a moiety that contains a group, which is identical to a native drug except for the structural modifications necessary for bonding the drug to the remainder of the compound of the present invention. Depending on the functional groups available in the native drug, bonding may be effected using one of the functional groups already present in the native drug or it may be effected by incorporating a new functional or linking group. By consequence, the (native) drug can be used for bonding in unmodified or in a modified form. That is, the drug can be unmodified (in its natural form) except for the replacement of a hydrogen atom by a covalent bond, or it can be chemically modified in order to incorporate one functional group (e.g. a group selected from hydroxyl, carboxyl, amino and thiol groups) allowing covalent attachment(s) to an amino acid, e.g. amino acid Axx in formula (I) and Ayy in formula (I′), Bxx in formulae (II) and (II′), Cxx in formulae (Ib) and (IIa). The drug can also be modified by covalent attachment to a divalent group, e.g. an amino acid, a (di)peptide, or another linker or spacer such as described herein in relation to S, Sₐ, S_{b}, S₁, S₂, S₃, or combination thereof, such that bonding to the remainder of the compound of the present invention is accomplished via said divalent group. Said divalent group will however remain attached to the drug after cleavage by Cathepsin B. The expression “moiety derived from a drug” as used herein is meant to encompass both meanings and may thus refer to a moiety that differs from the unmodified (native) drug only by virtue of the covalent bond needed for bonding to the remainder of the molecule, or the modified drug as specified above additionally containing for instance a linker or spacer.

The expression “maleimide derivative” (or e.g. “triazole derivative” etc.) as used herein refers to a maleimide moiety that is modified by virtue of the covalent bonds needed for bonding to other groups, for instance for bonding to a drug and to the remainder of the compound. For example, a maleimide derivative is covalently attached via a carboxylic group (e.g. 3-maleimidopropionic acid) to a N-terminal residue of the compound of formula (I)/(I′) or to the side chain of Bxx of the compound of formula (II)/(II′)/(IIa). Subsequently, a nucleophilic group (e.g. a nucleophilic group which may occur in the native drug such as the thiol group of mertansine) is reacted to the maleimide function via Michael addition.

In an analogous manner, the term “derivative” is used in connection with other moieties to characterize the presence of covalent bonds needed for bonding to the adjacent moieties.

The term “native drug” refers to a compound, for which therapeutic efficacy has been established by in vitro and/or in vivo tests. In a preferred embodiment, the native drug is a compound for which therapeutic efficacy has been established by clinical trials. Most preferably, the native drug is a drug that is already commercially available. The type of therapeutic efficacy to be established and suitable tests to be applied depend of course on the type of medical indication to be treated.

Accordingly, the drug to be used in the ligand-drug-conjugate of the present invention can be a native drug (e.g. a drug naturally containing one or more functional groups allowing covalent attachment to the conjugate), or can be a chemically modified drug (e.g. a drug according to formula (III)), a moiety A′xx(D₂)-A′yy according to formula (Ia) or a moiety A′yy-A′xx(D₂) according to formula (Ia′), a moiety Bxx(D)-Byy according to formula (II) or a moiety Byy-Bxx(D) according to formula (II′)) provided that the drug is pharmacologically active after it is released from the conjugate. Pharmacological activity in this connection means at least 20%, preferably at least 50%, more preferably at least 80% of the pharmacological activity of the native drug.

In those instances where the drug is a cytotoxic agent (e.g. DM1) that is chemically modified by covalent attachment to an amino acid or a (di)peptide, the chemically modified drug (e.g. a moiety according to formula (III)), a moiety A′xx(D₂)-A′yy according to formula (Ia), —a moiety A′yy-A′xx(D₂)according to formula (Ia′), a moiety Bxx(D)-Byy according to formula (II) or a moiety Byy-Bxx(D) according to formula (II′)) can be referred to as an “intra-payload”, provided that the chemically modified drug is pharmacologically active after its release from the conjugate.

The expression “auristatin analog” (or simply “auristatin”) refers to a class of compounds structurally related to the naturally occurring pentapeptide dolastin 10. The auristatin analogs (auristatins) as used herein satisfy the following formula:

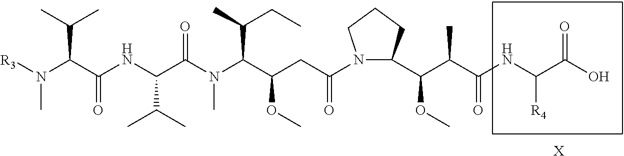

wherein R₃ represents a hydrogen atom or an alkyl group having 1 to 10 carbon atoms, preferably a hydrogen atom or a methyl group; and R₄ represents the side chain of any natural or unnatural amino acid.

In specific embodiments, the invention makes use of certain auristatin analogs. Typical examples of such auristatin analogs include monomethyl auristatin E (MMAE) and monomethyl auristatin F (MMAF). In the following, the expression “auristatin analog”, when characterizing analogs to be used in accordance with the invention, refers, in particular, to auristatin X, wherein the C-terminal amino acid X (as shown above) is selected from Phe (in which case, the auristatin analog is auristatin Phe/F (AF)), Cit (in which case, the auristatin analog is auristatin Cit (ACit)), Arg (in which case, the auristatin analog is auristatin Arg (AArg)), Lys (in which case, the auristatin analog is auristatin Lys (ALys)), Orn (in which case, the auristatin analog is auristatin Orn (AOrn)), Dab (in which case, the auristatin analog is auristatin Dab (ADab)) and Dap (in which case, the auristatin analog is auristatin Dap (ADap)). The auristatin analogs as used herein (AF, ACit, AArg, ALys, AOrn, ADab, ADap) are to be considered as native drugs, in addition to the native drugs as defined above.

In other embodiments, the auristatin analog is an analog not to be used in the context of the present invention. This would typically be an analog of the above formula wherein R₃ represents a methyl group and X is Asp, Glu, Thr, phosphoThr.

The expression “moiety derived from a vector group” as used herein indicates that the vector group can be in an unmodified or modified form. That is, the vector group can be unmodified (in its natural form) except for the replacement of a hydrogen atom by a covalent bond, or chemically modified so as to introduce one or more functional groups (e.g. a group selected from hydroxyl, carboxyl, thiol and/or amino groups) allowing covalent attachment(s) of the vector group to S (formulae (I) (I′), (II) and (II′)), S₁ (formula (Ia₂), or Azz₃ (formula (Ia₃)) provided that such modifications do not interfere to a significant degree with the interaction between vector group and target cell.

The expression “capable of interacting with a target cell” as used herein indicates that the vector group can bind to, complex with, or react with a moiety, e.g. a protein or receptor, that is exposed on the surface of a target cell. Said interaction may give rise to a targeting effect (i.e. to a local increase of the concentration of the vector-carrying compound in the vicinity of the target cell) and/or it may cause internalization of the vector-carrying compound of the present invention into the target cell.

The expression “pharmaceutically acceptable salts” as used herein refers to derivatives of the disclosed compounds wherein the parent compound is modified by making acid or base salts thereof. The pharmaceutically acceptable salts include the conventional non-toxic salts or the quaternary ammonium salts of the parent compound formed, for example, from non-toxic inorganic or organic acids. Lists of suitable salts can be found in Remington's Pharmaceutical Sciences, 17^{th} ed., Mack Publishing Company, Easton, Pa., 1985, page 1418, S. M. Berge, L. M. Bighley, and D. C. Monkhouse, “Pharmaceutical Salts,” J. Pharm. Sci. 66 (1), 1-19 (1977); P. H. Stahl and C. G. Wermuth, editors, Handbook of Pharmaceutical Salts: Properties, Selection and Use, Weinheim/Zuirich, Wiley-VCH, 2008 and in A. K. Bansal et al., Pharmaceutical Technology, 3(32), 2008. The pharmaceutical salts can be synthesized from the parent compound which contains a basic or acidic moiety by conventional chemical methods. This can be done before or after incorporating the drug moiety into the compound of the present invention.

The term “antibody” as used herein covers monoclonal antibodies, polyclonal antibodies, dimers, multimers, multispecific antibodies (e.g. bispecific antibodies), veneered antibodies, antibody fragments and small immune proteins. An antibody is a protein generated by the immune system that is capable of recognizing and binding to a specific antigen. A target antigen generally has numerous binding sites, also called epitopes, recognized by complementary-determining regions on multiple antibodies. Each antibody that specifically binds to a different epitope has a different structure. Thus, one antigen may have more than one corresponding antibody. An antibody includes a full-length immunoglobulin molecule or an immunologically active portion of a full-length immunoglobulin molecule, i.e. a molecule that contains an antigen binding site that immuno-specifically binds an antigen of a target of interest or part thereof. The antibodies can be of any type e.g. IgG, IgE, IgM, IgD, and IgA, any class e.g. IgGI, IgG2, IgG3, IgG4, IgAl and IgA2, or subclass thereof. The antibody can be human or derived from other species.

The term “antibody fragment” as used herein refers to a portion of a full length antibody, generally the antigen binding or variable region thereof. Examples of antibody fragments include Fab, Fab′, F(ab′)₂, and Fv fragments; diabodies; linear antibodies; single domain antibodies. Antibodies and their fragments can be replaced by binding molecules based on alternative non-immunoglobulin scaffolds, peptide aptamers, nucleic acid aptamers, and structured polypeptides comprising polypeptide loops subtended on a non-peptide backbone, natural receptors or domains thereof.

The term “cancer” as used herein means the physiological condition in mammals that is characterized by unregulated cell growth. A tumor comprises one or more cancer cells. Examples of cancer include carcinoma, lymphoma, blastoma, sarcoma, and leukemia or lymphoid malignancies. Further examples of cancer include squamous cell cancer (e.g. epithelial squamous cell cancer), lung cancer including small-cell lung cancer, non-small cell lung cancer, adenocarcinoma of the lung and squamous carcinoma of the lung, cancer of the peritoneum, hepatocellular cancer, gastric or stomach cancer including gastrointestinal cancer, gastrointestinal stromal tumor, pancreatic cancer, glioblastoma, cervical cancer, ovarian cancer, liver cancer, bladder cancer, hepatoma, breast cancer, colon cancer, rectal cancer, colorectal cancer, endometrial or uterine carcinoma, salivary gland carcinoma, kidney or renal cancer, prostate cancer, thyroid cancer and hepatic cancer.

Unless specified otherwise, the term “alkyl” refers to saturated hydrocarbon groups, which may be linear, branched, cyclic or any combination thereof. The linear alkyl group preferably has 1 to 20 carbon atoms, more preferably 1 to 6 carbon atoms. The branched alkyl group preferably has 3 to 20 carbon atoms, more preferably 4 to 8 carbon atoms. The cyclic alkyl group preferably has 3 to 20 carbon atoms, more preferably 5 to 8 carbon atoms.

The term “aromatic group” characterizes a moiety that contains one or more cyclic structures having a delocalized π electron system that follows the Hückel 4n+2 rule. Said one or more cyclic structures may be a monocycle such as a 6-membered ring (e.g. benzene) or a bicyclic structure such as a moiety with two condensed 6-membered rings (e.g. naphthene) or a moiety wherein two 6-membered rings are bonded to each other via a single covalent bond(e.g. biphenyl). Further aromatic groups may contain three or more condensed cycles, such as anthracene, phenanthrene, pyrene, and chrysene. Aromatic groups may also contain heterocyclic structures, including for instance 5-membered rings containing one to four nitrogen atoms (the remaining ring members being carbon or carbon and oxygen), 5-membered rings containing one oxygen or sulfur atom, 6-membered rings with one or two nitrogen, oxygen or sulfur atoms or bicyclic moieties with two condensed rings, one of which being a 5-membered heterocycle and the other one being a 6-membered carbocycle or heterocycle.

Unless specified otherwise, all valencies of the individual atoms of the compounds or moieties described herein are saturated. In particular, they are saturated by the indicated binding partners. If no binding partner or a too small number of binding partners is indicated, the remaining valencies of the respective atom are saturated by a corresponding number of hydrogen atoms.

Unless specified otherwise or dictated otherwise by the context, all connections between adjacent amino acid groups are formed by peptide (amide) bonds.

Unless the context dictates otherwise and/or alternative meanings are explicitly provided herein, all terms are intended to have meanings generally accepted in the art, as reflected by IUPAC Gold Book (status of 1 Nov. 2017), or the Dictionary of Chemistry, Oxford, 6^{th} Ed.

### 2. Overview

The present invention is based on the discovery of a C-terminal dipeptide linker that can act as highly specific substrate for the exopeptidase activity of Cathepsin B (Cat B). The C-terminal dipeptide linker of the present invention can be used in ligand-drug-conjugates (LDCs), resulting in improved cleavage and drug release from the LDCs.

Cat B is a lysosomal cysteine protease of the papain superfamily acting in intracellular protein turnover as well as in a variety of physiological and pathological processes. Extended structural and functional data are presently available, making this protease a versatile tool in the context of intracellular drug delivery.

The papain fold is composed of two domains, referred to the left (L-) and right (R-) domain. The L-domain contains three a-helices, while the R-domain forms a kind of β-barrel as described by Turk et al. (Biochim. Biophys. Acta 2012, 1824(1), 68-88). The two domain interface opens on the top, forming the active-site cleft of the enzyme. In the center of the active-site cleft are the residues Cys25 (at the N-terminus of the central helix, L-domain) and His163 (within the β-barrel residues, R-domain). These two catalytic residues form the thiolate-imidazolium ion pair, essential for the proteolytic activity of the enzyme. The substrate binds along the active-site cleft in an extended conformation as described by Turk et al. (Biochem. Soc. Symp. 2003, 70, 15-30), making alternating contacts with L- and R-domains. Most cysteine cathepsins exhibit predominantly endopeptidase activity (F, L, K, S, V), whereas Cat X and C exhibit only exopeptidase activity. In contrast, Cat B exhibits both endopeptidase and exopeptidase (i.e. carboxydipeptidase) activity. X-ray analysis reveals that exopeptidases/carboxydipeptidases such as Cat B contain additional structural features, i.e. an additional (“occluding”) loop, which modify the active site cleft and serve as rationale for substrate binding in both endopeptidase and exopeptidase activity. In particular, the occluding loop provides the structural base for the dominant exo-versus endo-Cat B activity as shown by Renko et al. (FEBS Journal 2010, 277, 4338-4345).

The Cat B-cleavable linker systems described in the prior art (e.g. the Val-Cit-PABC linker system) are mainly based on the endopeptidase activity of Cat B. On the other hand, the linker system of the present invention is specifically designed to meet the structural requirements for acting as specific substrate for the exopeptidase (carboxydipeptidase) activity of Cat B.

Therefore, the linker system can be used in LDCs as highly specific substrate for the exopeptidase (carboxydipeptidase) activity of Cat B, i.e. in the compound of formula (I) or (I′) and compound of formula (II) or (II′) described below, resulting in improved cleavage profiles (e.g. fast intracellular drug release). Moreover, the linker system enables the intracellular release of multiple drug molecules, wherein individual drug molecules may be the same or different. If the drug is a payload (i.e. a cytotoxic agent), the linker system enables the intracellular release of multiple payloads, which may be multiple drug molecules of the same drug or multiple molecules of different drugs (e.g. 2 or more different drugs/payloads). As a special feature of LDCs exhibiting high DAR-values (e.g. formula (Ia1) or (II), the—linker system can be attached/conjugated to a single site of a vector group capable of interacting with a target cell (e.g. an antibody) thereby overcoming problems of overloading and premature extracellular cleavage. Thus the linker system of the present invention provides a highly tunable technology platform, which allows achieving high drug loading (e.g. high DAR) while being stable and non-toxic in the systemic circulation.

Furthermore, it was surprisingly found that the presence of a sterically demanding moiety (T in formula (I)/(I′) or D in formula (II)/(II′)) on the side chain of residue Axx in formula (I)/(I′) or Bxx in formula (II)/(II′) has no detrimental effect on the binding affinity of the compound of the present invention to Cat B, nor on the cleavage rate of the compound by the exopeptidase activity of Cat B. Without wishing to be bound to any theory, it is believed that the sterically demanding moiety T or D is directed towards the outside of the Cat B binding groove (known as the “hydrophobic pocket” of Cat B), thus leading to superior selectivity and cleavage rate via the exopeptidase activity of Cat B.

### 3. Compound of Formulae (I) and (I′)

The present invention relates to a compound (i.e. a ligand-drug-conjugate (LDC)) represented by the general formulae (I) or (I′):

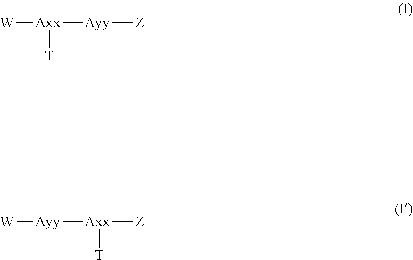

The compound of formulae (I) or (I′) contains a C-terminal dipeptide unit Axx-Ayy or Ayy-Axx, which serves as substrate for specific recognition and cleavage by the exopeptidase activity of Cat B.

Axx represents a trifunctional amino acid. Axx can be any natural or non-natural trifunctional amino acid with the proviso that Axx in formula (I) is not an amino acid in the (D) configuration. Examples of trifunctional amino acids include amino-dicarboxylic acids and diamino-carboxylic acids, such as α-amino adipic acid (Aaa), diamino propionic acid (Dap), diamino butyric acid (Dab), and amino malonic acid (Ama). Further suitable trifunctional amino acids include Glu, 2-amino pimelic acid (Apa), Lys, Orn, Ser and homo-lysine (homo-Lys).

According to one embodiment, Axx represents an amino acid selected from Glu, Apa, Aaa, Dap, Dab, Lys, Orn, Ser, Ama, and homolysine (homoLys). According to one preferred embodiment, Axx represents an amino acid selected from Dap, Dab, Lys, Orn and homoLys.

Ayy represents an amino acid selected from Phe, Ala, Trp, Tyr, Phenylglycine (Phg), Met, Val, His, Lys, Arg, Citrulline (Cit), 2-amino butyric acid (Abu), Orn, Ser, Thr, Leu and IIe; or Ayy in formula (I) represents an amino acid selected from homo-tyrosine (homo-Tyr), homo-phenylalanine (homo-Phe), beta-phenylalanine (beta-Phe), beta-homo-phenylalanine (beta-homo-Phe), Tyr(OR₁) and homo-Tyr(OR₁) wherein R₁ is a solubilizing group, preferably —(CH₂CH₂O)ₙ₁—R₂, wherein R₂ is a hydrogen atom or a methyl group and n1 is an integer of 2 to 24, e.g. an integer of 5 to 20, or 8 to 16; with the proviso that Ayy in formula (I′) is not an amino acid in the (D) configuration. Without wishing to be bound by theory, it is believed that Ayy provides the compound of the present invention with the structural features for specific recognition and cleavage by the exopeptidase activity of Cat B. As a result, the compound can release the drug at a significantly higher rate as compared to a compound cleaved by the endopeptidase activity of Cat B (e.g. in the Val-Cit-PABC system).

According to one embodiment, Ayy in formula (I) represents an amino acid selected from Phe, homo-Phe, Ala, Trp, Leu, Tyr, Phg, Met, Abu, Val, Lys, Cit, Tyr(OR₁) and homo-Tyr(OR₁), preferably an amino acid selected from Phe, homo-Phe, Ala, Trp, Leu, Val, Tyr, homo-Tyr, Tyr(OR₁) and homo-Tyr(OR₁), more preferably Phe, homo-Phe, Tyr, homo-Tyr, Tyr(OR₁) or homo-Tyr(OR₁), wherein R₁ is as specified above, in particular Phe or Tyr; and Ayy in formula (I′) represents an amino acid selected from Phe, homo-Phe, Ala, Ser, Thr, Leu, Val, Tyr, Phg, Trp, IIe and Arg, preferably an amino acid selected from Phe, homo-Phe, Ala, Trp, Phg, Leu, Val, Tyr and Ser, more preferably Phe, home-Phe or Ser, in particular Phe or Ser.

In some embodiments of the present invention, W represents a moiety represented by the following formula (III):

W₁-Dxx-Dyy---- (III)

In connection with formula (III) depicted above, Dxx represents a single covalent bond or an amino acid having a hydrophobic side chain, preferably an amino acid selected from Phe, Val and Ala. In some embodiments, Dxx may contain a further element such that the single covalent bond or amino acid having a hydrophobic side chain is optionally attached to moiety W₁ via a divalent moiety selected from maleimides, triazoles, hydrazones, carbonyl-containing groups, and derivatives thereof, preferably via a divalent maleimide derivative.

In connection with formula (III) depicted above, Dyy represents a single covalent bond, Phe or an amino acid having a basic side chain, preferably an amino acid selected from Arg, Lys, Cit, Orn, Dap, and Dab, more preferably Arg or Cit; with the proviso that if Dxx is an amino acid having a hydrophobic side chain, Dyy is Phe or an amino acid having a basic side chain, and if Dxx is a single covalent bond, Dyy is a single covalent bond, Phe or an amino acid having a basic side chain.

The broken line in formula (III) indicates covalent attachment to the N-terminus of Axx in formula (I), or the N-terminus of Ayy in formula (I′).

W₁ represents a moiety derived from a drug. In some instances, W represents a moiety derived from a drug having one or more groups selected from hydroxyl, carboxyl, amine, or thiol groups, wherein said one or more groups can optionally serve for covalent attachment to the C-terminal dipeptide unit Axx-Ayy (formula (I)) or Ayy-Axx (formula (I′)). The drug(s) suitable for use in the present invention are described in more detail below. Examples of drugs having one or more groups selected from hydroxyl, carboxyl, amine, or thiol groups include auristatins, maytansines, camptothecins and doxorubicins.

In one embodiment, W₁ represents a moiety derived from a drug that differs from a native drug (e.g. DM1) only by virtue of the covalent attachment to Dxx as shown in formula (III). If the native drug is an auristatin analog, the auristatin analog is selected from auristatin F (AF), auristatin Cit (ACit), auristatin Arg (AArg), auristatin Lys (ALys), auristatin Orn (AOrn), auristatin Dab (ADab) and auristatin Dap (ADap). Preferably, the auristatin analog is AF. In one aspect, the auristatin is not auristatin Asp (AAsp), auristatin Glu (AGlu), auristatin PhosphoThr (AphThr) or auristatin Thr (AThr).

In one further embodiment, W₁ represents a moiety derived from a drug, preferably a moiety derived from a drug that differs from a native drug only by virtue of the covalent attachment to Dxx; with the proviso that W₁ is not an auristatin analog.

According to one embodiment, W represents a peptide moiety represented by the following formula (Ia) or (Ia′):

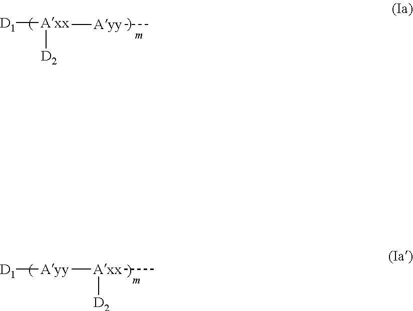

A′yy represents an amino acid selected from Phe, Ala, Trp, Tyr, Phg, Met, Val, His, Lys, Arg, Cit, Orn and Abu; with the proviso that A′yy in formula (Ia′) is not an amino acid in the (D) configuration. According to one embodiment, A′yy represents an amino acid selected from Phe, Ala, Trp, Phg and Tyr, preferably Phe or Tyr.

D₁ represents a moiety derived from a drug. Each D₂ independently represents a hydrogen atom or a moiety derived from a drug, with the proviso that at least one D₂ is not a hydrogen atom. When D₂ represents a moiety derived from a drug, D₁ and D₂ can be moieties derived from the same drug, or moieties derived from different drugs. The drug(s) suitable for use in the present invention are described in more detail below. According to one embodiment, D₁ and D₂ each independently represent a moiety derived from a drug having one or more groups selected from hydroxyl, carboxyl, amine, or thiol groups.

m is an integer of 1 to 10; and the broken line indicates covalent attachment to the N-terminus of Axx or Ayy. Accordingly, when m≥1, the compound includes one moiety D₁ and m moieties D₂, wherein multiple moieties D₂ can be the same or different. According to one preferred embodiment, m is an integer of 1 to 4.

If m=1, A′xx represents a trifunctional amino acid such as an amino-dicarboxylic acid or a diamino-carboxylic acid. A′xx can be any trifunctional natural or non-natural amino acid with the proviso that A′xx in formula (Ia) is not an amino acid in the (D) configuration. Examples of trifunctional amino acids include Glu, Apa, Aaa, Dap, Dab, Lys, Orn, Ser, Ama, and homoLys. According to one preferred embodiment, A′xx represents an amino acid selected from Dap, Dab, Lys, Orn and homoLys.

If m is more than 1, then each D₂ is independently selected from a hydrogen atom and moieties derived from a drug, wherein multiple moieties D₂ can be the same or different. If D₂ is a hydrogen atom, A′xx represents an amino acid. A′xx can be any natural or non-natural amino acid—e.g. a bifunctional or trifunctional amino acid—that collectively provides the required functionalities for attachment to amino acid A′yy and moiety D₁ or another amino acid A′xx, with the proviso that A′xx in formula (Ia) is not an amino acid in the (D) configuration. Examples of bifunctional amino acids include Gly, Ala, Abu, Cyclohexylalanine (Cha), IIe, Leu, Phe, Phg, Val. If D₂ is a moiety derived from a drug, A′xx represents a trifunctional amino acid as described above, preferably an amino acid selected from Dap, Dab, Lys, Orn and homoLys.

The peptide of formula (I)/(Ia), (I)/(Ia′), (I′)/(Ia) or (I′)/(Ia′) acts as a specific substrate for the exopeptidase activity of Cat B. That is, the compound of formula (I) or (I′) described herein can be cleaved at its N-terminus by Cat B, releasing W that is either a moiety D₁ derived from a drug or a dipeptide moiety having formula (Ia) or (Ia′). When W is a dipeptide moiety of formula (Ia) or (Ia′), it can in turn be cleaved by Cat B, thus releasing moiety D₁ and peptide (A′xx(D₂)-A′yy)/(A′yy-A′xx(D₂)). In some aspects of the present invention, moieties D₁ and peptides (A′xx(D₂)-A′yy)/(A′yy-A′xx(D₂)) exhibit pharmacological (e.g. cytotoxic) activity.

In some aspects of the present invention, the peptide (A′xx(D₂)-A′yy)/(A′yy-A′xx(D₂)) can be a “self-immolative” moiety, which can undergo intramolecular aminolysis (i.e. five- or six-membered ring formation, or diketopiperazine (DKP) formation), releasing moiety D₂ as a product. When m≥1, the peptide (A′xx(D₂)-A′yy)ₘ/(A′yy-A′xx(D₂)ₘ acts as a substrate for Cat B, which can cleave the (m-1) amide bonds between amino acids A′yy-A′xx/A′yy-A′xx, thus releasing m dipeptides (A′xx(D₂)-A′yy)/(A′yy-A′xx(D₂)). In some aspects, each dipeptide can in turn undergo intramolecular aminolysis (A′xx(D₂)-A′yy) or DKP formation (A′yy-A′xx(D₂)), releasing m moieties D₂ as product.

Therefore, when W represents a peptide having formula (Ia)/(Ia′), the linker can release two or more molecules of the same or different drugs (and thus permits accomplishing a high DAR) and the overall pharmacological activity can be enhanced. The drug release can occur according to a multi-step mechanism. For instance, W can be first released from the compound of formula (I), and then act as a substrate for Cat B releasing moiety D₁ and, eventually, m peptides (A′xx(D₂)-A′yy)/(A′yy-A′xx(D₂)), which can be pharmacologically active as such (e.g. intra-payloads) and/or undergo intramolecular aminolysis, DKP formation or hydrolysis to release m moieties D₂.

The compound of the present invention is typically stable in an extracellular environment (e.g. in plasma) in the absence of Cat B (i.e. the enzyme capable of cleaving the linker). However upon exposure to Cat B, the linker is recognized and cleaved initiating, eventually, the spontaneous self-immolative aminolysis resulting in the cleavage of the bond covalently linking the self-immolative moiety, e.g. A′xx-A′yy, to the drug, to thereby achieve release of the drug D₂ in its pharmacologically active form. Self-immolative aminolysis can occur if A′xx represents an amino acid such as Glu, Aaa, Dap, Dab, Ser, Thr, homoSer, homoThr.

T in formula (I) or (I′) represents a moiety having the following formula (Ia₁):

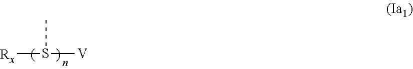

S represents a di- or multivalent group comprising 1 or more atoms selected from carbon, nitrogen, oxygen, and sulfur. S binds (links) amino acid(s) Axx (through covalent attachment to the side chain of Axx) to moiety V (described below). S can be linked to Axx and V e.g. via chemoselective ligation procedures for amide bond formation or via “click chemistry” (e.g. azide-alkyne cycloaddition). Rₓ represents an atom or group which is optionally present to saturate a free valency of S, if present.

In some embodiments, S may act as a moiety for multiple drug attachment (FIG. 3). It can be a small organic group with two or more valencies having, for instance, a molecular weight of 200 Da or less or even only 100 Da or less, but it can also be a more complex and/or larger moiety derived from functional polymers, copolymers, dendrimers or synthetic constructs including multiple reactive groups for linker-drug attachment.

In the above formula (Ia₁), n is an integer of 1 to 10, e.g. 1 to 5. When n=1, S represents a di- or trivalent group and the broken line represents covalent attachment of S to the side chain of Axx. When n>1, each S independently represents a di- or trivalent group, and each broken line represents covalent attachment to an individual group of formula (I) (to the side chain of an individual amino acid Axx), wherein each group of formula (I) can be the same or different. When n>1, the linker can release two or more molecules of the same or different drugs (and thus permits accomplishing a high DAR) and the overall pharmacological activity can be enhanced.

For instance, for n=2 and for n=3, possible structures are as follows:

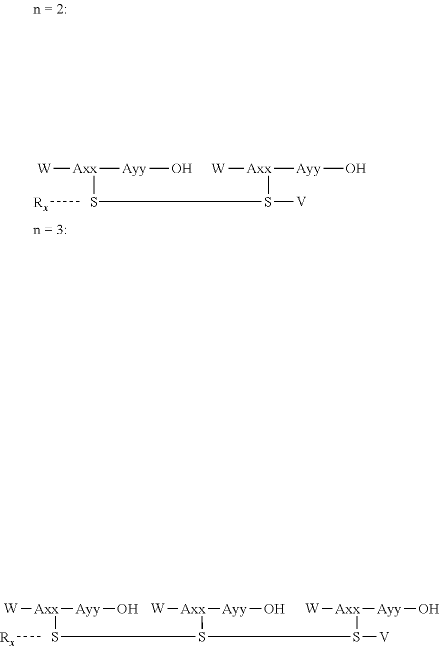

Of course, if variable groups such as W, Axx, Ayy and S are present multiple times, the individual variable groups of the same type may be the same or may differ from each other. Moreover, the position of binding of V in the above structures is not particularly limited. For instance, in the above formula for n=3, V may also be bonded to the central group S instead of the terminal group S wherein the central group S is tetravalent, as shown below:

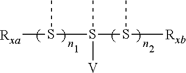

wherein n₁ and n₂ each independently is an integer of 0 to n and n₁+n₂+1=n; Rₓₐ and R_{xb} each independently represents an atom or group, which is optionally present to saturate a free valency of S, if present.

In some aspects of the present invention, the divalent or multivalent group S is selected such that it is stable to hydrolysis, meaning that typically less than 20% and preferably less than 10% of a test compound undergoes hydrolysis in phosphate-buffered saline (PBS) solution pH 7.4 at 37° C. within 24 hours, as determined by HPLC, wherein said test compound is a compound based on multivalent group S, wherein all valencies of S are saturated by hydrogen atoms.

Ideally, the compound of formula (I) or formula (II) (i.e. the LDC), containing said di- or multivalent group S, when taken as a whole, also shows such stability to hydrolysis, i.e. it is preferred that less than 20% and more preferably less than 10% of the compound of formula (I) undergoes hydrolysis in phosphate-buffered saline (PBS) solution pH 7.4 at 37° C. within 24 hours, as determined by HPLC.

S can be a polar or charged divalent or multivalent group such that water solubility of the compound of formula (I) is improved. S can also comprise an amino acid or a peptide moiety, preferably a polar or charged amino acid or peptide moiety, the peptide comprising from 2 to 10 amino acids, which can be natural or non-natural amino acids.

S can also be based on a combination of two or more of the above-mentioned multivalent groups being bonded together via covalent bonds.

Preferred S groups are (—O—CH₂CH₂-)ₙ with n being selected from 1 to 10, Dab or combinations of these two groups.

According to one further embodiment, W in formulae (I) and (I′) represents a peptide moiety having the following formula (Ib):

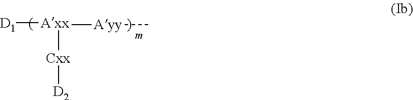

A′yy, D₁ and m in formula (Ib) are as defined in formula (Ia), and the broken line indicates covalent attachment to the N-terminus of Axx or Ayy.

If m=1, A′xx represents an amino acid selected from Glu, Aaa, Dap, Dab, Ser, Thr, homoserine (homoSer), homothreonine (homoThr) and Ama, with the proviso that A′xx is not an amino acid in the (D) configuration. D₂ represents a moiety derived from a drug, optionally the same drug as D₁. Cxx represents a single covalent bond unless A′xx is Ama. When A′xx represents Ama, an additional amino acid Cxx is present. Cxx binds to the side chain of A′xx, i.e. one of the two carboxyl ends of Ama, and it also binds to drug moiety D₂. Cxx represents (L)- or (D)-Pro or an N-methyl amino acid such sarcosine (Sar). Preferably, Cxx represents an amino acid selected from (L)- or (D)-Pro, Sar, N-methyl Val and N-methyl Leu, more preferably Sar.

If m is more than 1, then each D₂ is independently selected from a hydrogen atom and moieties derived from a drug, wherein multiple moieties D₂ can be the same or different. If D₂ is a hydrogen atom, A′xx represents an amino acid and Cxx represents a single covalent bond (even if A′xx is Ama). A′xx can be any natural or non-natural amino acid—e.g. a bifunctional or trifunctional amino acid—that collectively provides the required functionalities for attachment to amino acid A′yy and moiety D₁ or another amino acid A′xx, with the proviso that A′xx is not an amino acid in the (D) configuration. If D₂ is a moiety derived from a drug, A′xx represents an amino acid selected from Glu, Aaa, Dap, Dab, Ser, Thr, homoserine (homoSer), homothreonine (homoThr) and Ama, and Cxx represents a single covalent bond unless A′xx is Ama. When A′xx is Ama, Cxx represents an amino acid selected from (L)- or (D)-Pro, Sar, N-methyl Val and N-methyl Leu, more preferably Sar.

It is well established that peptides and proteins that possess a Pro residue at the penultimate N-terminal position undergo non-enzymatic aminolysis, resulting in DKP-formation. The mechanism of DKP formation involves nucleophilic attack of the N-terminal nitrogen on the carbonyl of the second amino acid. This intramolecular aminolysis proceeds readily and plays an important role in the biosynthetic pathway of biologically active cyclic dipeptides such as c(His-Pro), which are found throughout the central nervous system, peripheral tissues and body fluids. In the dipeptide (Ama(Cxx-D₂)-A′yy), the mechanism of DKP formation involves nucleophilic attack of the N-terminal nitrogen on the side chain of A′xx, thus releasing moiety D₂.

The peptide of formula (I)/(Ib) or (I′)/(Ib) acts as a substrate for the exopeptidase activity of Cat B, releasing a dipeptide moiety having formula (Ib), which in turn can be cleaved by Cat B to release moiety D₁ and peptide (A′xx(Cxx-D₂)-A′yy). The peptide (A′xx(Cxx-D₂)-A′yy) is a “self-immolative” moiety, which can undergo intramolecular aminolysis (i.e. five- or six-membered ring formation, or diketopiperazine (DKP) formation), releasing moiety D₂ as a product. When m≥1, the peptide (A′xx(Cxx-D₂)-A′yy)ₘ acts as a substrate for Cat B, which can cleave the (m-1) amide bonds between amino acids A′yy and A′xx, thus releasing m peptides (A′xx(Cxx-D₂)-A′yy). Each peptide (A′xx(Cxx-D₂)-A′yy) can, in turn, undergo intramolecular aminolysis, releasing m moieties D₂ as product.

Accordingly, when W represents a peptide having formula (Ib), drug release occurs according to a multi-step mechanism, for instance W can be first released from the compound of formula (I) and then act as a substrate for Cat B releasing moiety D₁ and m peptides (A′xx(Cxx-D₂)-A′yy), which finally undergo intramolecular aminolysis to release m moieties D₂. In the peptide (Ama(Cxx-D₂)-A′yy), the mechanism of DKP formation (intramolecular aminolysis) involves nucleophilic attack of the N-terminal nitrogen of Ama on the ester carbonyl of Cxx, thus releasing moiety D₂.

In the present invention, it was surprisingly found that the presence of a sterically demanding moiety (moiety T in formula (I)/(I′)) on the side chain of residue Axx in formula (I)/(I′)) has no detrimental effect on the binding affinity of the compound of the present invention to Cat B, nor on the cleavage rate of the compound by the exopeptidase mechanism of Cat B. Without wishing to be bound to any theory, it is believed that the sterically demanding moiety T is directed towards the outside of the Cat B binding groove (hydrophobic pocket), thus leading to superior cleavage rate via the exopeptidase mechanism.

V represents a moiety derived from a vector group capable of interacting with a target cell. V is described in more detail below. In some embodiments, the moiety V is covalently attached to one group S contained in the moiety T described above. In other words, the linker system of the present invention is attached to the vector group via a single attachement point. The attachment of more than one linker system at multiple sites of a vector group is not meant to be emcompassed by this embodiment. As a result, the linker system can achieve high drug loading (high DAR) and, at the same time, can overcome the problems of overloading of the vector group and premature extracellular cleavage of the conjugate (e.g. unspecific cell killing).

In some aspects, the linker system provides a novel and highly tunable technology platform allowing at least one of the following items: (1) release of one molecule of a drug (payload) into a target cell, (2) release of multiple molecules (e.g. 2 to 20 or 4 to 10) of the same drug into a target cell (high DAR), (3) release of multiple molecules (e.g. 2 to 20 or 4 to 10) of different drugs (dual-payload or multi-payload) into a target cell (high DAR). As a particularly important feature, due to a modulable solubilizing effect exerted by moiety S, high DAR-values can be achieved in keeping with favorable PK properties of the LDCs.

According to one embodiment, T represents a moiety having one of the following formulae (Ia₂) and (Ia₃):

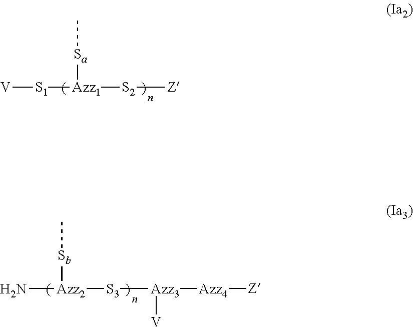

Sₐ and S_{b} each independently represents a single covalent bond or a divalent group having 1 or more atoms selected from carbon, nitrogen, oxygen and sulfur.

S₁, S₂ and S₃ each independently represents a divalent group having 1 or more atoms selected from carbon, nitrogen, oxygen and sulfur. n represents an integer from 1 to 10.

In formula (Ia₂), Azz₁ represents a trifunctional amino acid; S₁ links the N-terminus of amino acid Azz₁ to moiety V, S₂ links the C-terminus of amino acid Azz₁ to the OH group (n=1), and/or links the C-terminus of amino acid Azz₁ to the N-terminus of another amino acid Azz₁ (n>1), and Sₐ links the side chain of Azz₁ to an individual group of formula (I) or (I′); wherein if n>1, each individual group of formula (Azz₁(—Sₐ---)—S₂) can be the same or different, and each broken line represents a covalent bond to an individual group of formula (I) or (I′), wherein each group of formula (I)/(I′) can be the same or different. In some aspects of the present invention, Azz₁ is a trifunctional amino acid having a functional group enabling the chemical ligation of the group of formula (Ia₂) to an individual group of formula (I) or (I′), e.g. an azide group or an alkyne group.

In formula (Ia₃), Azz₂ and Azz₄ each independently represent an amino acid; Azz₃ represents a trifunctional amino acid such as Lys, wherein moiety V is attached to the side chain of Azz₃; S₃ links the C-terminus of amino acid Azz₂ to the N-terminus of amino acid Azz₃ (n=1), and/or links the C-terminus of amino acid Azz₂ to the N-terminus of another amino acid Azz₂ (n>1); S_{b} links the N-terminus or side chain of amino acid Azz₂ to an individual group of formula (I) or (I′). In some aspects, Azz₂ is an amino acid having a functional group enabling the chemical ligation of the group of formula (Ia₃) to an individual group of formula (I) or (I′), e.g. an azide group or an alkyne group.

If n>1, each individual group of formula Azz₁(Sₐ)—S₂ in formula (Ia₂) and of formula Azz₂(S_{b})—S₃ in formula (Ia₃) can be the same or different, and each broken line binds to an individual group of formula (I) or (I′) as specified herein, wherein each group of formula (I)/(I′) can be the same or different.

In formulae (Ia₂) and (Ia₃), Z′ represents a group covalently bonded to S₂ (formula (Ia₂)) or to the C-terminus of Azz₄ (formula (Ia₃)) selected from —OH and —N(H)(R′), wherein R′ represents a hydrogen atom, an alkyl group, a cycloalkyl group or an aromatic group.

According to one embodiment, S, Sₐ, S_{b}, S₁, S₂ and S₃ each independently represents a divalent alkylene group, a divalent alkenylene group, a divalent alkynylene group, or a divalent polyalkylene oxide group. These divalent groups preferably have a backbone chain length of 1 to 100 atoms, more preferably 2 to 50 atoms, more preferably 3 to 25.

According to one embodiment, S, Sₐ, S_{b}, S₁, S₂ and S₃ each independently represents a divalent group having formula —(CH₂)_{q}-Azz₅-, or a divalent group having the formula —(OCH₂CH₂)_{q}-Azz₅-; wherein q is an integer of 1 to 50, preferably an integer of 2 to 10; and Azz₅ is either absent, or represents a solubilizing group such as a divalent group containing as a substituent an ammonium group, a sulfate group or an amino acid. Azz₅ may for instance be an amino acid with a polar side chain, e.g. Arg.

According to one embodiment, S, Sₐ, S_{b}, S₁, S₂ and S₃ each independently represents a divalent group having formula —(CH₂)_{q}-Azz₅-Y—, or a divalent group having formula —(OCH₂CH₂)_{q}-Azz₅-Y—; wherein Y represents a divalent moiety covalently bonded to the C-terminus or the side chain of Azz₅ and to moiety V; if Azz₅ is absent, Y represents a divalent moiety covalently bonded to the alkylene or polyethylene oxide group and to moiety V; Y being a divalent moiety selected from maleimides, triazoles, hydrazones, carbonyl-containing groups, and derivatives thereof, preferably a divalent maleimide or triazole derivative; Azz₅ and q are as specified above.

For instance, when Y represents a divalent maleimide moiety (derivative), Y can be obtained by reacting a maleimido group with a nucleophilic group such as a hydroxyl, amino, or thiol group. The maleimido group to be reacted with a nucleophilic group can be introduced e.g. at the C-terminus or on the side chain of Azz₅ (and the nucleophilic group can thus be introduced in moiety V or is already present in moiety V). Accordingly, S, Sₐ, S_{b}, S₁, S₂ and S₃ each independently can be obtained from a moiety having formula —(CH₂)_{q}-Azz₅-Y′ or a moiety having formula —(OCH₂CH₂)_{q}-Azz₅-Y′, wherein Y′ represents a maleimido group, wherein q represents an integer selected from the range of from 1 to 50.

When Y represents a divalent triazole moiety, Y can be obtained by reacting an azide group with an alkyne group (i.e. “click chemistry”), the azide group or alkyne group being introduced e.g. at the C-terminus or on the side chain of Azz₅. Accordingly, S, Sₐ, S_{b}, S₁, S₂ and S₃ each independently can be obtained from a moiety having formula —(CH₂)_{q}-Azz₅-Y′ or a moiety having formula —(OCH₂CH₂)_{q}-Azz₅-Y′, wherein Y′ represents an alkyne group, or an azide group.

When Y represents a divalent hydrazone moiety, Y can be obtained by reacting a hydrazine group with an aldehyde group, the hydrazine group or aldehyde group being introduced e.g. at the C-terminus or on the side chain of Azz₅. Furthermore, when Y represents a divalent carbonyl-containing group, Y can be obtained by reacting a carboxylic acid group or derivative thereof, e.g. an acyl chloride group, with a nucleophilic group such as a hydroxyl group or an amino group.

V in formulae (Ia₁), (Ia₂) and (Ia₃) represents a moiety derived from a vector group capable of interacting with a target cell. V is described in more detail below.

Z represents a group covalently attached to the C-terminus of Ayy selected from —OH; —N(H)(R), wherein R represents a hydrogen atom, an alkyl group, a cycloalkyl group, or an aromatic group; and a labeling agent such as a coumarin derivative.

According to one embodiment, the compound of formula (I) is selected from the following compounds, wherein Z is preferably —OH: W-Glu(T)-Phe-Z, W-Glu(T)-Ala-Z, W-Glu(T)-Trp-Z, W-Glu(T)-Tyr-Z, W-Apa(T)-Phe-Z, W-Apa(T)-Ala-Z, W-Apa(T)-Trp-Z, W-Apa(T)-Tyr-Z, W-Aaa(T)-Phe-Z, W-Aaa(T)-Ala-Z, W-Aaa(T)-Trp-Z, W-Aaa(T)-Tyr-Z, W-Dap(T)-Phe-Z, W-Dap(T)-Ala-Z, W-Dap(T)-Trp-Z, W-Dap(T)-Tyr-Z, W-Dab(T)-Phe-Z, W-Dab(T)-Ala-Z, W-Dab(T)-Trp-Z, W-Dab(T)-Tyr-Z, W-Lys(T)-Phe-Z, W-Lys(T)-homoPhe-Z, W-Lys(T)-Ala-Z, W-Lys(T)-Trp-Z, W-Lys(T)-Tyr-Z, W-Lys(T)-homoTyr-Z, W-Lys(T)-homoTyr(OR₁)—Z wherein R₁—(CH₂CH₂O)ₙ₁—H and n₁ is an integer of 2 to 24, W-Orn(T)-Phe-Z, W—Orn(T)-Ala-Z, W—Orn(T)-Trp-Z, W—Orn(T)-Tyr-Z, W—Ser(T)-Phe-Z, W—Ser(T)-Ala-Z, W—Ser(T)-Trp-Z, W—Ser(T)-Tyr-Z, W-homoLys(T)-Phe-Z, W-homoLys(T)-Ala-Z, W-homoLys(T)-Trp-Z, W-homoLys(T)-Tyr-Z.

According to one embodiment, the compound of formula (I) (wherein W is a moiety of formula (III)) is selected from the following compounds, wherein Z is preferably —OH: W₁-Arg-Lys(T)-Phe-Z, W₁-Arg-Lys(T)-homoPhe-Z, W₁—Cit-Lys(T)-Phe-Z, W₁—Cit-Lys(T)-Tyr-Z, W₁—Cit-Lys(T)-homoTyr-Z, W₁-Lys(T)-Phe-Z, W₁-Lys(T)-Tyr-Z, W₁-Lys(T)-homoTyr-Z, W₁-Mal-Phe-Cit-Lys(T)-Phe-Z (SEQ ID NO: 4), W₁-Mal-Phe-Cit-Lys(T)-Tyr-Z (SEQ ID NO: 5), W₁-Mal-Phe-Cit-Lys(T)-homoTyr-Z (SEQ ID NO: 6), W₁-Mal-Phe-Lys-Lys(T)-Phe-Z (SEQ ID NO: 7), W₁-Mal-homoPhe-Arg-Lys(T)-Phe-Z (SEQ ID NO: 8), W₁-Mal-homoPhe-Cit-Lys(T)-Tyr-Z (SEQ ID NO: 9), W₁-Mal-homoPhe-Cit-Lys(T)-Tyr(OR₁)—Z (SEQ ID NO: 10) with R₁—(CH₂CH₂O)ₙ₁—H and n₁ is an integer of 2 to 24 e.g. 12, W₁-Mal-Cit-Lys(T)-Tyr-Z, W₁-Mal-Cit-Lys(T)-homoTyr-Z, W₁-Mal-Arg-Lys(T)-homoTyr-Z; preferably W₁-Arg-Lys(T)-Phe-Z, W₁—Cit-Lys(T)-Tyr-Z, W₁-Lys(T)-Phe-Z, W₁-Lys(T)-Tyr-Z, W₁-Mal-Phe-Cit-Lys(T)-Phe-Z (SEQ ID NO: 4), W₁-Mal-Phe-Cit-Lys(T)-Tyr-Z (SEQ ID NO: 5), W₁-Mal-Cit-Lys(T)-Tyr-Z or W₁-Arg-Lys(T)-Phe-Z; more preferably W₁-Lys(T)-Tyr-Z, W₁-Mal-Phe-Cit-Lys(T)-Phe-Z (SEQ ID NO: 4) or W₁-Mal-Cit-Lys(T)-Tyr-Z.

According to one preferred embodiment, the compound of formula (I) (wherein W is a moiety of formula (III)) is selected from the following compounds, wherein Z is preferably —OH: APhe-Arg-Lys(T)-Phe-Z (SEQ ID NO: 66), APhe-Arg-Lys(T)-homoPhe-Z (SEQ ID NO: 67), APhe-Cit-Lys(T)-Phe-Z (SEQ ID NO: 68), APhe-Cit-Lys(T)-Tyr-Z (SEQ ID NO: 69), APhe-Cit-Lys(T)-homoTyr-Z (SEQ ID NO: 70), ACit-Lys(T)-Phe-Z, ACit-Lys(T)-Tyr-Z, ACit-Lys(T)-homoTyr-Z, DM1-Mal-Phe-Cit-Lys(T)-Phe-Z (SEQ ID NO: 71), DM1-Mal-Phe-Cit-Lys(T)-Tyr-Z (SEQ ID NO: 72), DM1-Mal-Phe-Cit-Lys(T)-homoTyr-Z (SEQ ID NO: 73), DM1-Mal-Phe-Lys-Lys(T)-Phe-Z (SEQ ID NO: 74), DM1-Mal-homoPhe-Arg-Lys(T)-Phe-Z (SEQ ID NO: 75), DM1-Mal-homoPhe-Cit-Lys(T)-Tyr-Z (SEQ ID NO: 76), DM1-Mal-homoPhe-Cit-Lys(T)-Tyr(OR₁)—Z (SEQ ID NO: 77) with R₁—(CH₂CH₂O)ₙ₁—H and n₁ is an integer of 2 to 24 e.g. 12, DM1-Mal-Cit-Lys(T)-Tyr-Z; DM1-Mal-Cit-Lys(T)-homoTyr-Z; DM1-Mal-Arg-Lys(T)-homoTyr-Z; preferably APhe-Arg-Lys(T)-Phe-Z (SEQ ID NO: 66), APhe-Cit-Lys(T)-Tyr-Z (SEQ ID NO: 69), DM1-Mal-Phe-Cit-Lys(T)-Phe-Z (SEQ ID NO: 71), DM1-Mal-Phe-Cit-Lys(T)-Tyr-Z (SEQ ID NO: 72), DM1-Mal-Cit-Lys(T)-Tyr-Z or APhe-Arg-Lys(T)-Phe-Z (SEQ ID NO: 66); more preferably DM1-Mal-Phe-Cit-Lys(T)-Phe-Z (SEQ ID NO: 71) or DM1-Mal-Cit-Lys(T)-Tyr-Z.

In one embodiment, the compound of formula (I) contains a moiety W represented by formula (III), in which W₁ represents a moiety derived from a drug that is not an auristatin analog (e.g. AF). This compound is preferably selected from the following compounds, wherein Z is preferably —OH: DM1-Mal-Phe-Cit-Lys(T)-Phe-Z (SEQ ID NO: 71), DM1-Mal-Phe-Cit-Lys(T)-Tyr-Z (SEQ ID NO: 72), DM1-Mal-Phe-Cit-Lys(T)-homoTyr-Z (SEQ ID NO: 73), DM1-Mal-Phe-Lys-Lys(T)-Phe-Z (SEQ ID NO: 74), DM1-Mal-homoPhe-Arg-Lys(T)-Phe-Z (SEQ ID NO: 75), DM1-Mal-homoPhe-Cit-Lys(T)-Tyr-Z (SEQ ID NO: 76), DM1-Mal-homoPhe-Cit-Lys(T)-Tyr(OR₁)—Z (SEQ ID NO: 77) with R₁—(CH₂CH₂O)ₙ₁—H and n₁ is an integer of 2 to 24 e.g. 12; DM1-Mal-Cit-Lys(T)-Tyr-Z; DM1-Mal-Cit-Lys(T)-homoTyr-Z and DM1-Mal-Arg-Lys(T)-homoTyr-Z; more preferably DM1-Mal-Phe-Cit-Lys(T)-Phe-Z (SEQ ID NO: 71), DM1-Mal-Phe-Cit-Lys(T)-Tyr-Z (SEQ ID NO: 72) or DM1-Mal-Cit-Lys(T)-Tyr-Z.

According to one embodiment, the compound of formula (I) (wherein W is a moiety of formula (III)) is selected from the following compounds, wherein Z is preferably —OH: W₁—Cit-(Lys(D₂)-Phe)ₘ-Lys(T)-Phe-Z (SEQ ID NO: 14), W₁-Cit-(Lys(D₂)-Phe)ₘ-Lys(T)-homoTyr-Z (SEQ ID NO: 15), W₁-Cit-(Lys(D₂)-Phe)ₘ-Lys(T)-Tyr(OR₁)—Z (SEQ ID NO: 16) with R₁—(CH₂CH₂O)ₙ₁—H and n₁ is an integer of 2 to 24 e.g. 12, W₁-(Lys(D₂)-Phe)ₘ-Lys(T)-Phe-Z (SEQ ID NO: 17), W₁-Phe-(Phe-Lys(D₂))ₘ-Lys(T)-Tyr-Z (SEQ ID NO: 18), W₁-(Phe-Lys(D₂))ₘ-Lys(T)-Tyr-Z (SEQ ID NO: 19), W₁-Phe-(Phe-Lys(D₂))ₘ-Lys(T)-homoTyr-Z (SEQ ID NO: 20), W₁-Arg-(Phe-Lys(D₂))ₘ-Lys(T)-Tyr(OR₁)—Z (SEQ ID NO: 21); preferably from AF-Cit-(Lys(Mal-DM1)-Phe)ₘ-Lys(T)-Phe-Z, AF-Cit-(Lys(Mal-DM1)-Phe)ₘ-Lys(T)-homoTyr-Z, AF-Cit-(Lys(Mal-DM1)-Phe)ₘ-Lys(T)-Tyr(OR₁)—Z with R₁—(CH₂CH₂O)ₙ₁—H and n₁ is an integer of 2 to 24 e.g. 12, AF-Phe-(Phe-Lys(Mal-DM1))ₘ-Lys(T)-Tyr-Z, AF-Arg-(Phe-Lys(Mal-DM1))ₘ-Lys(T)-Tyr(OR₁)—Z; wherein m is preferably an integer of 1 to 8, e.g. 1 to 4.

According to one embodiment, the compound of formula (I′) is selected from the following compounds, wherein Z is preferably —OH: W-Phe-Glu(T)-Z, W-Ala-Glu(T)-Z, W-Trp-Glu(T)-Z, W-Tyr-Glu(T)-Z, W-Phe-Apa(T)-Z, W-Ala-Apa(T)-Z, W-Trp-Apa(T)-Z, W-Tyr-Apa(T)-Z, W-Phe-Aaa(T)-Z, W-Ala-Aaa(T)-Z, W-Trp-Aaa(T)-Z, W-Tyr-Aaa(T)-Z, W-Phe-Dap(T)-Z, W-Ala-Dap(T)-Z, W-Trp-Dap(T)-Z, W-Tyr-Dap(T)-Z, W-Phe-Dab(T)-Z, W-Ala-Dab(T)-Z, W-Trp-Dab(T)-Z, W-Tyr-Dab(T)-Z, W-Phe-Lys(T)-Z, W-Ala-Lys(T)-Z, W-Trp-Lys(T)-Z, W-Tyr-Lys(T)-Z, W-Phe-Orn(T)-Z, W-Ala-Orn(T)-Z, W-Trp-Orn(T)-Z, W-Tyr-Orn(T)-Z, W-Phe-Ser(T)-Z, W-Ala-Ser(T)-Z, W-Trp-Ser(T)-Z, W-Tyr-Ser(T)-Z, W-Phe-Ama(T)-Z, W-Ala-Ama(T)-Z, W-Trp-Ama(T)-Z, W-Tyr-Ama(T)-Z, W-Phe-homoLys(T)-Z, W-Ala-homoLys(T)-Z, W-Trp-homoLys(T)-Z, W-Tyr-homoLys(T)-Z.

According to one embodiment, the compound of formula (I′) (wherein W is a moiety of formula (III)) is selected from W₁-Arg-Phe-Lys(T)-Z, W₁-Arg-Ser-Lys(T)-Z, W₁—Cit-Phe-Lys(T)-Z, W₁—Cit-Ser-Lys(T)-Z, W₁—Cit-homoPhe-Lys(T)-Z, W₁-Phe-Lys(T)-Z, W₁—Ser-Lys(T)-Z, W₁-Mal-Phe-Cit-Phe-Lys(T)-Z (SEQ ID NO: 22), W₁-Mal-homoPhe-Cit-Phe-Lys(T)-Z (SEQ ID NO: 23), W₁-Mal-Phe-Arg-Phe-Lys(T)-Z (SEQ ID NO: 24), W₁-Mal-Cit-Phe-Lys(T)-Z, W₁-Mal-Phe-Ser-Lys(T)-Z, W₁-Mal-Ala-Phe-Lys(T)-Z, W₁-Mal-Cit-Ser-Lys(T)-Z, W₁-Mal-Arg-homoPhe-Lys(T)-Z; preferably W₁-Arg-Phe-Lys(T)-Z, W₁—Cit-Phe-Lys(T)-Z, W₁-Phe-Lys(T)-Z, W₁-Mal-Phe-Cit-Phe-Lys(T)-Z (SEQ ID NO: 22), W₁-Mal-Phe-Arg-Phe-Lys(T)-Z (SEQ ID NO: 24), W₁-Mal-Cit-Phe-Lys(T)-Z, W₁-Mal-Phe-Ser-Lys(T)-Z, W₁-Mal-Ala-Phe-Lys(T)-Z; more preferably W₁-Cit-Phe-Lys(T)-Z, W₁-Phe-Lys(T)-Z, W₁-Mal-Phe-Cit-Phe-Lys(T)-Z (SEQ ID NO: 22) or W₁-Mal-Phe-Ser-Lys(T)-Z.

According to one preferred embodiment, the compound of formula (I′) (wherein W is a moiety of formula (III)) is selected from the following compounds, wherein Z is preferably —OH: APhe-Arg-Phe-Lys(T)-Z (SEQ ID NO: 29), APhe-Arg-Ser-Lys(T)-Z (SEQ ID NO: 28), APhe-Cit-Phe-Lys(T)-Z (SEQ ID NO: 81), APhe-Cit-Ser-Lys(T)-Z (SEQ ID NO: 82) APhe-Cit-homoPhe-Lys(T)-Z (SEQ ID NO: 83), ACit-Phe-Lys(T)-Z, ACit-Ser-Lys(T)-Z, DM1-Mal-Phe-Cit-Phe-Lys(T)-Z (SEQ ID NO: 84), DM1-Mal-homoPhe-Cit-Phe-Lys(T)-Z (SEQ ID NO: 85), DM1-Mal-Phe-Arg-Phe-Lys(T)-Z (SEQ ID NO: 86), DM1-Mal-Cit-Phe-Lys(T)-Z, DM1-Mal-Phe-Ser-Lys(T)-Z, DM1-Mal-Ala-Phe-Lys(T)-Z, DM1-Mal-Cit-Ser-Lys(T)-Z, DM1-Mal-Arg-homoPhe-Lys(T)-Z; more preferably APhe-Arg-Phe-Lys(T)-Z (SEQ ID NO: 29), APhe-Cit-Phe-Lys(T)-Z (SEQ ID NO: 81), DM1-Mal-Phe-Cit-Phe-Lys(T)-Z (SEQ ID NO: 84), DM1-Mal-Phe-Arg-Phe-Lys(T)-Z (SEQ ID NO: 86), DM1-Mal-Cit-Phe-Lys(T)-Z, DM1-Mal-Phe-Ser-Lys(T)-Z, DM1-Mal-Ala-Phe-Lys(T)-Z; even more preferably APhe-Cit-Phe-Lys(T)-Z (SEQ ID NO: 81), DM1-Mal-Phe-Cit-Phe-Lys(T)-Z (SEQ ID NO: 84) or DM1-Mal-Phe-Ser-Lys(T)-Z.

In one embodiment, the compound of formula (I′) contains a moiety W represented by formula (III), in which W₁ represents a moiety derived from a drug that is not an auristatin analog (e.g. AF). This compound is preferably selected from the following compounds, wherein Z is preferably —OH: DM1-Mal-Phe-Cit-Phe-Lys(T)-Z (SEQ ID NO: 84), DM1-Mal-homoPhe-Cit-Phe-Lys(T)-Z (SEQ ID NO: 85), DM1-Mal-Phe-Arg-Phe-Lys(T)-Z (SEQ ID NO: 86), DM1-Mal-Cit-Phe-Lys(T)-Z, DM1-Mal-Phe-Ser-Lys(T)-Z, DM1-Mal-Ala-Phe-Lys(T)-Z, DM1-Mal-Cit-Ser-Lys(T)-Z, DM1-Mal-Arg-homoPhe-Lys(T)-Z; more preferably DM1-Mal-Phe-Cit-Phe-Lys(T)-Z (SEQ ID NO: 84), DM1-Mal-Phe-Arg-Phe-Lys(T)-Z (SEQ ID NO: 86), DM1-Mal-Cit-Phe-Lys(T)-Z, DM1-Mal-Phe-Ser-Lys(T)-Z or DM1-Mal-Ala-Phe-Lys(T)-Z; even more preferably DM1-Mal-Phe-Cit-Phe-Lys(T)-Z (SEQ ID NO: 84) or DM1-Mal-Phe-Ser-Lys(T)-Z.

The compound of formula (I) or (I′) can be selected from:

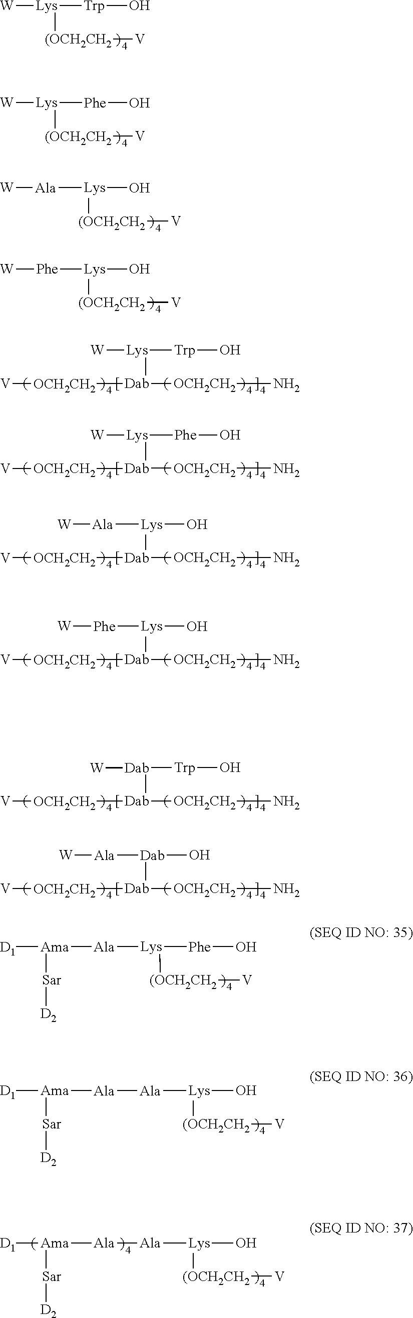

In the above compounds, the variable groups W, W₁, V, D₁ and D₂ have the same meanings as described above and below. Preferably, in the compounds of formulae (I) and (I′) exemplified above, W₁, D₁, and D₂ each independently represent a moiety derived from a drug, and especially a moiety derived from Auristatin F (AF), Auristatin X (AX; “AX” refers to analogs of Auristatin wherein X represents the C-terminal amino acid of the auristatin peptide chain), Campthotecin (CPT). Moreover, in the compounds shown above, if an ethylene oxide group (i.e. a group of formula (OCH₂CH₂)) binds the N-terminus of an amino acid, an additional carboxyl group (CO) may be present (not shown in the above compounds) such that an amide bond is present between the ethylene oxide group and the N-terminus of the amino acid.

In one embodiment of the present invention, the compound of formula (I) or formula (I′) is selected from:

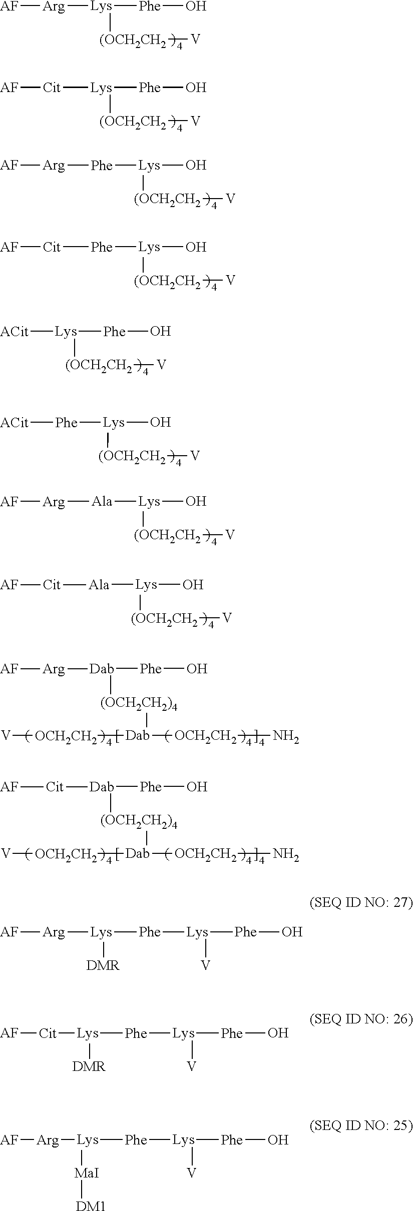

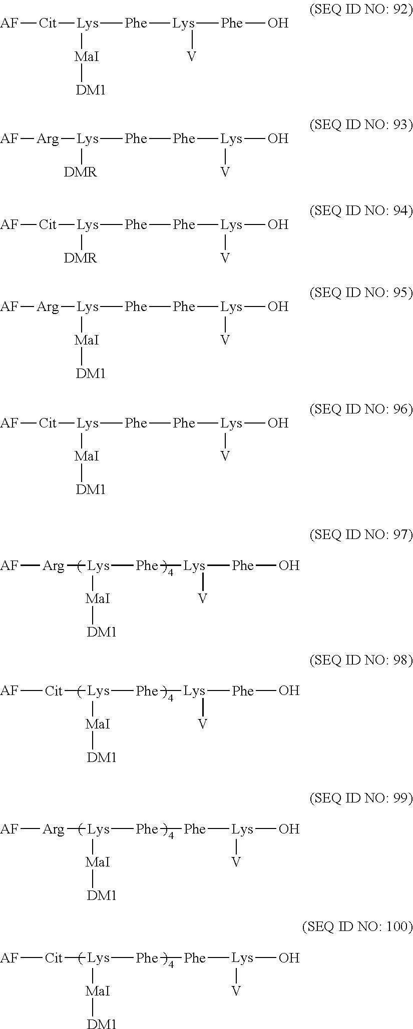

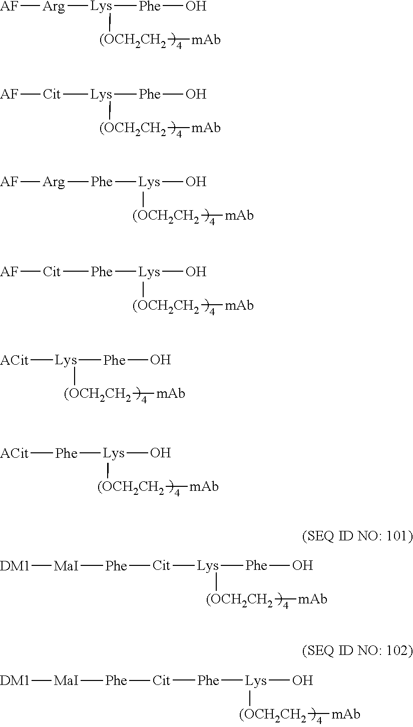

wherein in the above compounds DMR and DM1 represent maytansinoid drugs (e.g. mertansine) and mAb represents a monoclonal antibody vector capable of interacting with a target cell (described below).

### 4. Compound of Formula (II) and (II′)

The present invention also relates to a compound (i.e. a LDC) represented by the general formula (II) or (II′):

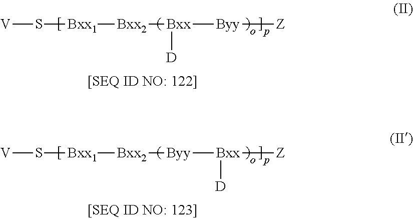

The compound of formula (II) or (II′) contains a C-terminal dipeptide unit Bxx-Byy or Byy-Bxx, which serves as substrate for recognition and cleavage by Cat B (through the exopeptidase activity of Cat B).

The term “C-terminal” as used herein refers to the C-terminal end (C-terminus) of the amino acid chain, e.g. amino acid Byy in dipeptide Bxx-Byy, and means that no drug or vector group is attached to the C-terminus of Byy. Yet, if o>1 and/or p>1, the C-terminus of Byy can bind to the N-terminus of another Bxx-Byy dipeptide unit or Bxx₁-Bxx₂ dipeptide unit as described in more detail below.

D represents a moiety derived from a drug. If p>1 and/or o>1, it is possible that up to (o*p)-1 groups D are absent, i.e. that the respective D groups represent a hydrogen atom or a solubilizing group such as as —(CH₂CH₂O)ₙ₁—H wherein n₁ is an integer of 2 to 24. According to one embodiment, D represents a moiety derived from a drug having one or more groups selected from hydroxyl, carboxyl, amino, or thiol groups. The drug(s) suitable for use in the present invention are described in more detail below. Examples of suitable drugs include auristatins, maytansines, camptothecins and doxorubicins.

Bxx represents a trifunctional amino acid such as an amino-dicarboxylic acid or a diamino-carboxylic acid. Bxx can be any natural or non-natural amino acid that provides the required three functionalities for attachment to the adjacent groups such as amino acids Bxx₂ and/or Byy and moiety D in formula (II); with the proviso that Bxx in formula (II) is not an amino acid in the (D) configuration. Examples of trifunctional amino acids include amino-dicarboxylic acids and diamino-carboxylic acids, such as Aaa, Dap, Dab, and Ama. Further suitable trifunctional amino acids include Glu, Apa, Lys, Orn, Ser and homoLys. In those instances were Bxx carries a hydrogen as D group, Bxx may also be any other amino acid, with the proviso that Bxx in formula (II) is not an amino acid in the (D) configuration.

According to one embodiment, Bxx represents an amino acid selected from Glu, Apa, Aaa, Dap, Dab, Lys, Orn, Ser, Thr, Ama, homoSer, homoThr and homoLys. According to one preferred embodiment, Bxx represents an amino acid selected from Dap, Dab, Lys, Orn and homoLys, preferably Lys or Dab, more preferably Lys.

Byy represents an amino acid selected from Phe, Ala, Trp, Tyr, Phg, Val, His, Lys, Abu, Met, Cit, Orn, Ser, Thr, Leu, IIe, Arg and Tyr(OR₁) wherein R₁ is —(CH₂CH₂O)ₙ₁—R₂, wherein R₂ is a hydrogen atom or a methyl group and n₁ is an integer of 2 to 24; or Byy in formula (II) represents an amino acid selected from homo-Tyr, homo-Phe, beta-Phe and beta-homo-Phe; with the proviso that Byy in formula (II′) is not an amino acid in the (D) configuration and with the proviso that if o*p>1, only the C-terminal Byy in formula (II) may represent an amino acid selected from homo-Phe, beta-Phe and beta-homo-Phe. Preferably, Byy in formulae (II) and (II′) represents Cit, Phe, Phg, Ser, Trp, Tyr or Tyr(OR₁) wherein R₁ is —(CH₂CH₂O)ₙ₁— R₂, wherein R₂ is a hydrogen atom or a methyl group and n₁ is an integer of 2 to 24, more preferably Phe, Tyr or Tyr(OR₁); if o*p>1, Byy represents preferably Tyr or Tyr(OR₁). Without wishing to be bound by theory, it is believed that Byy provides the compound of formula (II) or (II′) with the structural requirements for recognition and cleavage by Cat B.

Bxx₁ is either absent (represents a single covalent bond), or represents an amino acid (i.e. a natural or unnatural amino acid) having a hydrophobic or basic side chain; with the proviso that if p is more than 1, Bxx₁ is not an amino acid in the (D) configuration. Examples of natural amino acids having a hydrophobic or basic side chain include Phe, Tyr, Val, Ala, IIe, Leu, Ser, His, Met. Examples of unnatural amino acids having a hydrophobic side chain include Phenylglycine (Phg), cyclohexyl Ala (Cha), 2-amino isobutyric acid (Aib), butyl Gly (Tle), norleucine (NIe), norvaline (Nva).

According to one embodiment, Bxx₁ represents an amino acid selected from Phe, homo-Phe, Phg, Val, Ser, Leu, Tyr, Ala, IIe; preferably an amino acid selected from Phe, homo-Phe, Tyr and Val, more preferably Phe, homo-Phe or Tyr.

Bxx₂ represents an amino acid (i.e. a natural or unnatural amino acid) having a hydrophobic or basic side chain. According to one embodiment, Bxx₂ represents an amino acid selected from Arg, Lys, Cit, Val, Leu, Ser, Ala, Gly, His, Gln, Phg and Phe. According to one preferred embodiment, Bxx₂ represents an amino acid selected from Arg, Lys, Cit and Phe, preferably Arg or Cit.

S in formulae (II) and (II′) represents a divalent group having 1 or more atoms selected from carbon, nitrogen, oxygen, and sulfur. S links amino acid Bxx₁ or if Bxx₁ is absent to Byy (through covalent attachment to the N-terminus of Bxx₁ or Bxx₂) to moiety V (described below).

According to one embodiment, S represents a divalent alkylene group, a divalent alkenylene group or a divalent polyalkylene oxide group. Preferably, S represents a divalent group having formula —(CH₂)_{q}-Azz₅-, or a divalent group having the formula —(OCH₂CH₂)_{q}-Azz₅-; wherein q is an integer of 1 to 50, preferably an integer of 2 to 10; and Azz₅ is either absent, or represents a solubilizing group such as a divalent group containing an ammonium group, a sulfate group or an amino acid as a substituent. Azz₅ may for instance be an amino acid with a polar side chain.

According to one preferred embodiment, S represents a divalent group having formula —Y-Azz₅-(CH₂)_{q}—, or a divalent group having formula —Y-Azz₅-(OCH₂CH₂)_{q}—; wherein Y represents a divalent moiety covalently attached to the N-terminus of Azz₅ and to moiety V; if Azz₅ is absent, Y represents a divalent moiety covalently attached to the alkyl or polyethylene oxide group and to moiety V; Y being a divalent moiety selected from maleimides, triazoles, hydrazones, carbonyl-containing groups, and derivatives thereof, preferably a divalent maleimide derivative or triazole moiety. The divalent group of formula —Y-Azz₅-(CH₂)_{q}— can be obtained as described below.

Each of o and p in formulae (II) and (II′) independently is an integer of 1 to 10, preferably an integer of 1 to 4.

V represents a moiety derived from a vector group capable of interacting with a target cell. The expression “capable of interacting with a target cell” as used herein indicates that the vector group can bind, complex with, or react with a moiety, e.g. a protein or receptor, of a target cell, thus causing internalization of the compound of formula (II) into the target cell. V will be described in more detail below.

Z represents a group covalently attached to the C-terminus of Byy (and in case of p>1 the Byy group, which is located at the C-terminus) selected from —OH; —N(H)(R), wherein R represents a hydroxyl group, a hydrogen atom, an alkyl group, a cycloalkyl group, or an aromatic group, preferably a hydroxyl group; and a labeling agent such as a coumarin derivative.

According to one embodiment, R represents an alkyl group having from 1 to 20 carbon atoms, preferably a methyl or an ethyl group, a cycloalkyl group having from 3 to 20 carbon atoms, preferably 5 to 8 carbon atoms or an aromatic group having from 6 to carbon atoms, preferably 6 or 10 carbon atoms.

The peptides Bxx(D)-Byy and Byy-Bxx(D) in respective formulae (II) and (II′) selectively act as substrate for the exopeptidase activity of Cat B. That is, Cat B cleaves the compound of formula (II) or (II′) at the N-terminus of (each) Bxx (formula (II)) or Byy (formula (II′)) residue, releasing peptide moiety V—S-Bxx₁-Bxx₂, one Bxx(D)-Byy-Z peptide moiety and (p-1) Bxx₁-Bxx₂ peptide moieties as well as ((o*p)-1) Bxx(D)-Byy-OH peptide moieties. According to some embodiment, Bxx(D)-Byy-OH and Bxx(D)-Byy-Z can be self-immolative moieties, which can undergo intramolecular aminolysis or hydrolysis resulting in the release of moiety D as a product. In some aspects, dipeptide Bxx(D)-Byy-OH/Byy-Bxx(D)-OH can exhibit pharmacological (e.g. cytotoxic) activity.

According to one further embodiment, the compound of the present invention is represented by the following general formulae (IIa):

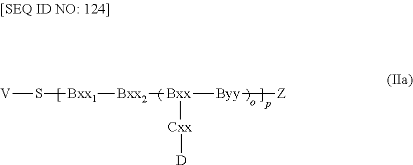

In formula (IIa), Bxx represents a carboxylic amino acid (i.e. having an COOH group on its side chain) such as Ama, Glu, Aaa, Apa or a trifunctional amino acid selected from Dap, Dab, Ser, Thr, Lys, Orn, homoLys, homoSer and homoThr; with the proviso that Bxx is not an amino acid in the (D) configuration. Preferably, Bxx represents a trifunctional amino acid selected from Ama, Glu, Aaa, Dap, Dab, Ser, Thr, Apa, Lys, Orn, homoLys, homoSer and homoThr.

Cxx represents a single covalent bond unless Bxx is Ama. If Bxx is Ama, Cxx represents (L)- or (D)-Pro, or an N-alkyl amino acid such as Sar, the N-terminus of Cxx binds to a carboxyl end of Ama and the C-terminus of Cxx binds via e.g. ester bond to drug moiety D (e.g. CPT).

According to one preferred embodiment, Cxx represents an amino acid selected from (L)- or (D)-Pro, Sarcosine (Sar), N-methyl Val and N-methyl Leu.

Byy represents an amino acid selected from Phe, Ala, Trp, Tyr, Phg, Val, His, Lys, Abu, Met, Cit, Orn, Ser, Thr, Leu, IIe, Arg, homo-Phe, beta-Phe and beta-homo-Phe with the proviso that if o*p>1, only the C-terminal Byy may represent an amino acid selected from homo-Phe, beta-Phe and beta-homo-Phe. Preferably, Byy represents Cit, Phe, homo-Phe, Ser, Trp, Tyr or Tyr(OR₁) wherein R₁ is —(CH₂CH₂O)ₙ₁—R₂, wherein R₂ is a hydrogen atom or a methyl group and n₁ is an integer of 2 to 24; more preferably Phe, Tyr or Tyr(OR₁); if o*p>1, Byy represents preferably Tyr or Tyr(OR₁). Without wishing to be bound by theory, it is believed that Byy provides the compound of formula (IIa) with the structural requirements for recognition and cleavage by Cat B.

The peptide Bxx(Cxx-D)-Byy in formulae (IIa) selectively acts as a substrate for the exopeptidase activity of Cat B, i.e. Cat B cleaves the compound of formula (IIa) at the N-terminus of (each) Bxx residue, releasing the peptide moiety V—S-Bxx₁-Bxx₂, one Bxx(Cxx-D)-Byy-Z peptide moiety and (p-1) Bxx₁-Bxx₂ peptide moieties as well as ((o*p)-1) Bxx(Cxx-D)-Byy-OH peptide moieties. Bxx(Cxx-D)-Byy-OH and Bxx(Cxx-D)-Byy-Z are self-immolative moieties, which can undergo intramolecular aminolysis (DKP formation) resulting in the release of moiety D as a product. In peptides Ama(Cxx-D₂)-Byy-OH and Ama(Cxx-D₂)-Byy-Z, the mechanism of DKP formation involves nucleophilic attack of the N-terminal nitrogen of Ama on the ester carbonyl of Cxx, thus releasing moiety D₂ (e.g. CPT).

D, Bxx₁, Bxx₂, S and V in formulae (IIa) are as defined above in respect of formulae (II) and (II′). In those instances where Bxx in formula (IIa) carries a hydrogen as D group, Bxx may also be any other amino acid, with the proviso that Bxx is not an amino acid in the (D) configuration.

In the present invention, it was surprisingly found that the presence of the sterically demanding moiety D on the side chain of residue Bxx (or Cxx if present) has no detrimental effect on the binding affinity of the compound of the present invention to Cat B, nor on the cleavage rate of the compound by the exopeptidase mechanism of Cat B. Without being bond to any theory, it is believed that the sterically demanding moiety D is directed towards the outside of the Cat B binding groove (known as the “hydrophobic pocket” of Cat B), thus leading to superior selectivity and cleavage rate via the exopeptidase mechanism.

In the compound of formula (II)/(II′), the moiety V (vector group) is covalently attached to one group S as shown above, i.e. the linker system is attached to the vector group via a single attachement point (e.g. via a cysteine-maleimide ligation). The attachment of more than one linker system at multiple sites of a moiety V is not meant to be emcompassed by the present disclosure. As a result, high drug loading (high DAR) can be achieved and, at the same time, the problems of overloading of the vector group and/or premature extracellular cleavage of the conjugate (e.g. unspecific cell killing) can be overcome.

In some aspects, the linker system provides a novel and highly tunable technology platform leading to at least one of the following items: (1) release of one molecule of a drug (payload) into a target cell, (2) release of multiple molecules (e.g. 2 to 20 or 4 to 10) of the same drug into a target cell (high DAR), (3) release of multiple molecules (e.g. 2 to 20 or 4 to 10) of different drugs (dual-payload or multi-payload) into a target cell (high DAR).

According to one embodiment, the compound of formula (II) is selected from the following compounds, wherein Z is preferably —OH: V—S-Phe-Lys-Lys(D)-Phe-Z (SEQ ID NO: 103), V—S-Phe-Cit-Lys(D)-Cit-Z (SEQ ID NO: 104), V—S-Phe-Cit-Lys(D)-Tyr-Z (SEQ ID NO: 105), V—S-Phe-Cit-Lys(D)-homoTyr-Z (SEQ ID NO: 106), V—S-Phe-Arg-Lys(D)-Arg-Lys(D)-Phe-Z (SEQ ID NO: 107), V—S-Phe-Arg-(Lys(D)-Cit)o-Z (SEQ ID NO: 108), V—S-Phe-Arg-(Lys(D)-Tyr(OR₁))ₒ—Z (SEQ ID NO: 109) wherein R₁ is —(CH₂CH₂O)ₙ₁— R₂, wherein R₂ is a hydrogen atom or a methyl group and n₁ is an integer of 2 to 24 e.g. 12, V—S-Phe-Arg-(Lys(D)-Cit)ₒ-Tyr(OR₁)-Tyr-Z (SEQ ID NO: 110); preferably from V—S-Phe-Lys-Lys(D)-Phe-Z (SEQ ID NO: 103), V—S-Phe-Cit-Lys(D)-Cit-Z (SEQ ID NO: 104), V—S-Phe-Cit-Lys(D)-homoTyr-Z (SEQ ID NO: 106) or V—S-Phe-Arg-Lys(D)-Arg-Lys(D)-Phe-Z (SEQ ID NO: 107).

According to one preferred embodiment, the compound of formula (II) is selected from the following compounds, wherein Z is preferably —OH: V—S-Phe-Lys-Lys(Mal-DM1)-Phe-Z (SEQ ID NO: 111), V—S-Phe-Lys-Lys(AF)-Phe-Z (SEQ ID NO: 112), V—S-Phe-Cit-Lys(Mal-DM1)-Cit-Z (SEQ ID NO: 113), V—S-Phe-Cit-Lys(Mal-DM1)-Tyr-Z (SEQ ID NO: 114), V—S-Phe-Cit-Lys(Mal-DM1)-homoTyr-Z (SEQ ID NO: 115), V—S-Phe-Arg-Lys(Mal-DM1)-Arg-Lys(AF)-Phe-Z (SEQ ID NO: 116), V—S-Phe-Arg-(Lys(Mal-DM1)-Cit)ₒ-Z (SEQ ID NO: 117), V—S-Phe-Arg-(Lys(Mal-DM1)-Tyr(OR₁))ₒ—Z (SEQ ID NO: 118) wherein R₁ is —(CH₂CH₂O)ₙ₁— R₂, wherein R₂ is a hydrogen atom or a methyl group and n₁ is an integer of 2 to 24 e.g. 12, V—S-Phe-Arg--(Lys(Mal-DM1)-Cit)ₒ-Tyr(OR₁)-Tyr-Z (SEQ ID NO: 119) and V—S-Phe-Arg-(Lys(AF)-Cit)ₒ-Z (SEQ ID NO: 120); preferably from V—S-Phe-Lys-Lys(Mal-DM1)-Phe-Z (SEQ ID NO: 111), V—S-Phe-Lys-Lys(AF)-Phe-Z (SEQ ID NO: 112), V—S-Phe-Cit-Lys(Mal-DM1)-homoTyr-Z (SEQ ID NO: 115) or V—S-Phe-Arg-Lys(Mal-DM1)-Arg-Lys(AF)-Phe-Z (SEQ ID NO: 116).

According to one embodiment, the compound of formula (II′) is selected from the following compounds, wherein Z is preferably —OH: V—S-Phe-Arg-Phe-Lys(D)-Ser-Lys(D)-Z (SEQ ID NO: 30), V—S-Phe-Arg-(Phe-Lys(D))ₒ—Z (SEQ ID NO: 31), V—S-Phe-Arg-(Ser-Lys(D))ₒ—Z (SEQ ID NO: 32), V—S-Phe-Arg-(Tyr(OR₁)-Lys(D))ₒ—Z (SEQ ID NO: 33), V—S-Phe-Arg-(Phe-Lys(D))ₒ-Phe-Tyr(OR₁)—Z (SEQ ID NO: 34); preferably V—S-Phe-Arg-Phe-Lys(D)-Ser-Lys(D)-Z (SEQ ID NO:30), V—S-Phe-Arg-(Phe-Lys(D))ₒ—Z (SEQ ID NO: 31) or V—S-Phe-Arg-(Ser-Lys(D))ₒ—Z (SEQ ID NO: 32); more preferably V—S-Phe-Arg-(Phe-Lys(D))ₒ—Z (SEQ ID NO: 31).

According to one preferred embodiment, the compound of formula (II′) is selected from V—S-Phe-Arg-Phe-Lys(Mal-DM1)-Ser-Lys(AF)—Z (SEQ ID NO: 121), V—S-Phe-Arg-(Phe-Lys(Mal-DM1))ₒ—Z (SEQ ID NO: 91), V—S-Phe-Arg-(Ser-Lys(Mal-DM1))ₒ—Z (SEQ ID NO: 90), V—S-Phe-Arg-(Tyr(OR₁)-Lys(Mal-DM1))ₒ—Z (SEQ ID NO: 89), V—S-Phe-Arg-(Phe-Lys(Mal-DM1))ₒ-Phe-Tyr(OR₁)—Z (SEQ ID NO: 88); preferably V—S-Phe-Arg-Phe-Lys(Mal-DM1)-Ser-Lys(AF)—Z (SEQ ID NO: 121), V—S-Phe-Arg-(Phe-Lys(Mal-DM1))ₒ—Z (SEQ ID NO: 91) or V—S-Phe-Arg-(Ser-Lys(Mal-DM1))ₒ—Z (SEQ ID NO: 90); more preferably V—S-Phe-Arg-(Phe-Lys(Mal-DM1))ₒ—Z (SEQ ID NO: 91).

According to one embodiment, the compound of formula (II) can be selected from:

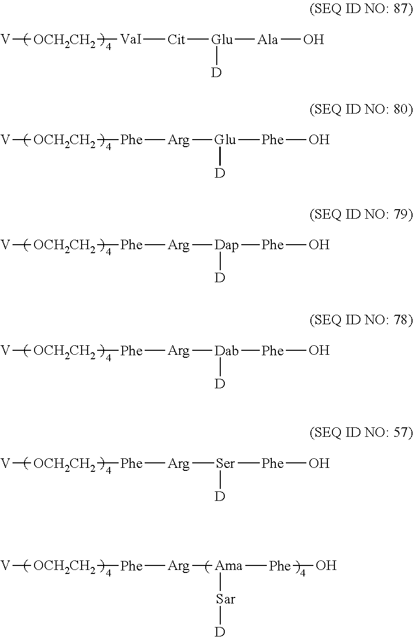

In one embodiment of the present invention, the compound of formula (II) is selected from:

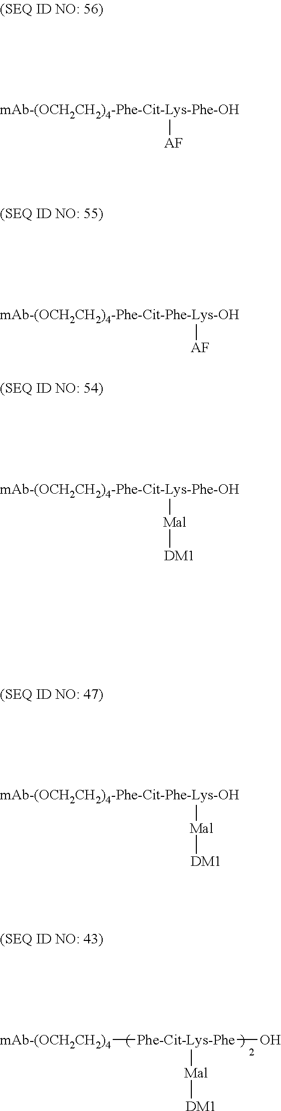

wherein in the above compounds DM1 represents a maytansinoid drug (i.e. mertansine) and mAb represents a monoclonal antibody vector capable of interacting with a target cell (described below).

### 5. Drugs

In the compound of the present invention, each moiety derived from a drug is independently selected from:
(i) Antineoplastic drugs;
(ii) Immunomodulatory drugs;
(iii) Anti-infectious disease drugs;

and radioisotopes and/or pharmaceutically acceptable salts, acids or derivatives thereof.

According to one embodiment, each moiety derived from a drug is independently derived from a drug having one or more groups selected from hydroxyl, carboxyl, thiol, or amino group.

The drug can be unmodified (in its natural form except for the replacement of a hydrogen atom by a covalent bond) or chemically modified in order to incorporate one or more functional groups (e.g. one or more groups selected from hydroxyl, carboxyl, amino and thiol groups) allowing covalent attachment(s) to an amino acid, e.g. amino acid Axx in formulae (I) and (I′), amino acid A′xx in formulae (Ia) and (Ib), amino acid Bxx in formulae (II), (II′) and (IIa), and/or Cxx in formulae (Ib) and (IIa). The drug can also be modified by covalent attachment to a divalent group, e.g. an amino acid, a peptide, a linker or spacer as described above etc.

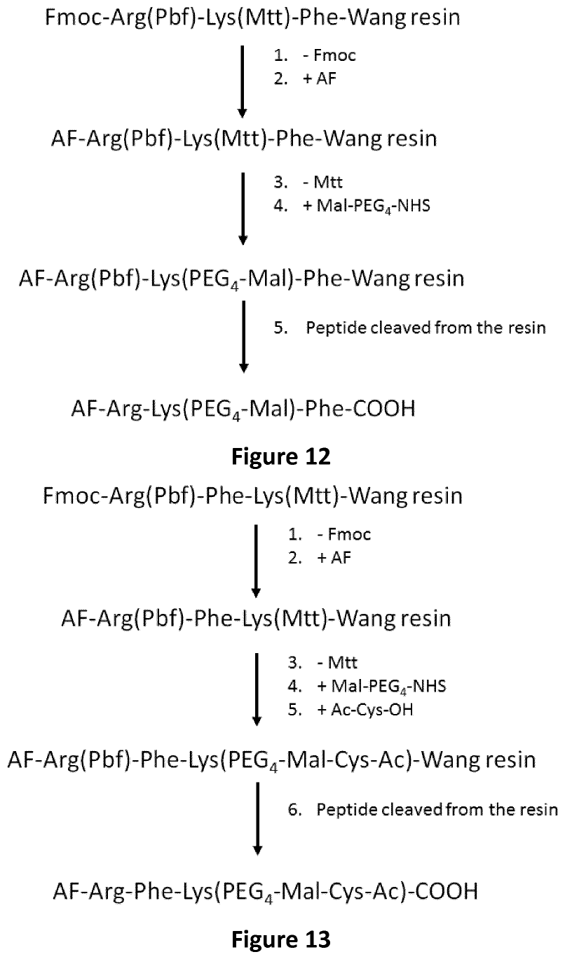

According to one embodiment, the drug can be modified by introduction of a divalent group, e.g. an amino acid or a peptide, which can increase the affinity of the conjugate for Cat B, in particular for the exopeptidase (carboxypeptidase) activity of Cat B. For instance, the drug can be modified by introducing an amino acid such as Phe, Lys, Cit or Arg, between the (native) drug and amino acid Axx of formula (I) or Ayy of formula (I′). An example of such modified drug is provided in FIG. 12, showing a maytansinoid drug containing amino acid Dyy such as Arg, Phe, Cit or Lys, between the drug and a peptide according to formula (I′) (i.e. drug and amino acid together forming moiety W according to formula (I′)). As shown in FIG. 12, Cat B-induced enzymatic cleavage at the N-terminus of Axx releases moiety W (i.e. drug derived from maytansine) in the target cell.

In some aspects of the present invention, each moiety derived from a drug independently represents a prodrug-group which is not pharmacologically active in the conjugated form (e.g. when found in the compound of formula (I), (I′), (II), (II′) or (IIa)), but which becomes pharmacologically active either once released from the conjugate or further activated intracellularly.

Accordingly, the drug to be used in the ligand-drug-conjugate of the present invention can be a native drug (e.g. a drug naturally containing one or more functional groups allowing covalent attachment to the conjugate), or can be a chemically modified drug, provided that the drug is pharmacologically active either once it is released from the conjugate or further activated intracellularly. In a preferred embodiment, the drug is a modified drug that is pharmacologically active in such a sense that it retains at least 20%, more preferably at least 50% of the pharmacological activity of the unmodified (native) drug.

Below are exemplary drugs that may be used in a ligand-drug-conjugate of the present invention.

(i) Antineoplastic agents include:

(a) Alkylatinq aqents such as nitrogen mustard analogues (e.g. cyclophosphamide chlorambucil, melphalan, chlormethine, ifosfamide, trofosfamide, prednimustine, bendamustine, chlornaphazine, estramustine, mechlorethamine, mechlorethamine oxide hydrochloride, mannomustine, mitolactol, novembichin, phenesterine, uracil mustard); alkyl sulphonates (e.g. busulfan, treosulfan, mannosulfan, improsulfan and piposulfan); ethylene imines (e.g. thiotepa, triaziquone, carboquone); nitrosoureas (e.g. carmustine, lomustine, semustine, streptozocin, chlorozotocin, fotemustine, nimustine, ranimustine); epoxides (e.g. etoglucid); other alkylating agents (e.g. mitobronitol, pipobroman, temozolomide, dacarbazine);

(b) Alkaloids such as vinca alkaloids (e.g. vincristine, vinblastine, vindesine, vinorelbine, navelbin, vinflunide, vintafolide); taxanes (e.g. paclitaxel, docetaxel, paclitaxel polyglumex, cabazitaxel) and their analogs, maytansinoids (e.g. DM1, DM2, DM3, DM4, maytansine and ansamitocins) and their analogs, cryptophycins (e.g. cryptophycin 1 and cryptophycin 8); epothilones, eleutherobin, discodermolide, bryostatins, dolostatins, auristatins (e.g. monomethyl auristatin E, monomethyl auristatin F), tubulysins, cephalostatins; pancratistatin; sarcodictyin; spongistatin; demecolcine; epipodophyllins (e.g. 9-aminocamptothecin, camptothecin, crisnatol, daunomycin, etoposide, etoposide phosphate, irinotecan and metabolites thereof such as SN-38, mitoxantrone, novantrone, retinoic acids (retinols), teniposide, topotecan, 9-nitrocamptothecin (RFS 2000)); mitomycins (e.g. mitomycin C);

(c) Anti-metabolites such as DHFR inhibitors (e.g. methotrexate, trimetrexate, denopterin, pteropterin, aminopterin (4-aminopteroic acid) or other folic acid analogues such as raltitrexed, pemetrexed, pralatrexate); IMP dehydrogenase inhibitors (e.g. mycophenolic acid, tiazofurin, ribavirin, EICAR); ribonucleotide reductase inhibitors (e.g. hydroxyurea, deferoxamine); pyrimidine analogs (e.g. cytarabine, fluorouracil, 5-fluorouracil and metabolites thereof, tegafur, carmofur, gemcitabine, capecitabine, azacitidine, decitabine, fluorouracil combinations, tegafur combinations, trifluridine combinations, cytosine arabinoside, ancitabine, floxuridine, doxifluridine), uracil analogs (e.g. 6-azauridine, deoxyuridine); cytosine analogs (e.g. enocitabine); purine analogs (e.g. azathioprine, fludarabine, mercaptopurine, thiamiprine, thioguanine, cladribine, clofarabine, nelarabine); folic acid replenisher such as folinic acid;

(d) Endocrine therapies used specifically in the treatment of neoplastic diseases, such as estrogens, progestogens, gonadotropin releasing hormone analogues, anti-estrogens, anti-androgens, aromatase inhibitors;

(e) Kinase inhibitors such as BIBW 2992 (anti-EGFR/Erb2), imatinib, gefitinib, pegaptanib, sorafenib, dasatinib, sunitinib, erlotinib, nilotinib, lapatinib, axitinib, pazopanib, vandetanib, afatinib, vemurafenib, crizotinib, regorafenib, masitinib, dabrafenib, trametinib, ibrutinib, ceritinib, lenvatinib, nintedanib, cediranib, palbocidib, osimertinib, alectinib, alectinib, rociletinib, cobimetinib, midostaurin, olmutinib, E7080 (anti-VEGFR2), mubritinib, ponatinib (AP24534), bafetinib (INNO-406), bosutinib (SKI-606), cabozantinib, vismodegib, iniparib, ruxolitinib, CYT387, tivozanib, ispinesib, temsirolimus, everolimus, ridaforolimus;

(f) Others such as duocarmycin (including synthetic analogues: adozelesin, carzelesin, bizelesin, KW-2189 and CBI-TMI); benzodiazepine dimers (dimers of pyrrolobenzodiazepine or tomaymycin, indolinobenzodiazepines, imidazobenzothiadiazepines, or oxazolidinobenzodiazepines); platinum containing compounds (e.g. carboplatin, cisplatin, oxaliplatin, satraplatin, polyplattilen); aziridines such as benzodopa, meturedopa, and uredopa; methylamelamines including altretamine, triethylenemelamine, trietylenephosphoramide, triethylenethiophosphaoramide and trimethylolomelamine; dynemicin, esperamicin, kedarcidin, maduropeptin, aclacinomysins, actinomycin, authramycin, azaserine, bleomycins, cactinomycin, carabicin, carminomycin, carzinophilin; chromomycins, dactinomycin, daunorubicin, detorubicin, 6-diazo-5-oxo-L-norleucine, doxorubicin, morpholino-doxorubicin, cyanomorpholino-doxorubicin, 2-pyrrolino-doxorubicin and deoxydoxorubicin, epirubicin, esorubicin, idarubicin, marcellomycin, nitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; polyketides (e.g. acetogenins); gemcitabine, epoxomicins (e.g. carfilzomib).

(ii) Immunomodulatory agents include immunostimulants, immunosuppressants, cyclosporine, cyclosporine A, aminocaproic acid, azathioprine, bromocriptine, chlorambucil, chloroquine, cyclophosphamide, corticosteroids (e.g. amcinonide, betamethasone, budesonide, hydrocortisone, flunisolide, fluticasone propionate, fluocortolone danazol, dexamethasone, prednisone, triamcinolone acetonide, beclometasone dipropionate), DHEA, hydroxychloroquine, meloxicam, methotrexate, mofetil, mycophenylate, sirolimus, tacrolimus, everolimus, fingolimod, ibrutinib.

(iii) Anti-infectious disease agents include antibacterial drugs, antimitotic drugs, antimycobacterial drugs and antiviral drugs. A non-limiting example of antibiotic used in an antibiotic-antibody drug conjugate is rifalogue, a rafamycin derivative.

The drugs used herein also include radioisotopes thereof. Examples of radioisotopes (radionuclides) are for instance ³H, ^{U}C, ¹⁴C, ¹⁸F, ³²P, ³⁵S, ⁶⁴CU, ⁶⁸Ga, ⁸⁶Y, ⁹⁹Tc, ¹¹¹In, ¹²³I, ¹²⁴I, ¹²⁵I, ¹³¹I, ¹⁷⁷Lu, ¹⁸⁶Re, ¹⁸⁸Re, ²¹¹At, ²¹²Bi, ²¹³Bi or ²²⁵Ac. Radioisotope labeled drugs can be used in targeted imaging experiments, or in targeted treatments (Wu et al Nat. Biotech. 2005, 23, 1137-1146).

The drugs used herein also include pharmaceutically acceptable salts, acids or derivatives thereof.

According to one preferred embodiment, each moiety derived from a drug is independently derived from a drug selected from duocarmycin, auristatin (an auristatin analog), maytansine, tubulysin, calicheamicin, camptothecin, SN-38, taxol, daunomycin, vinblastine, doxorubicin, methotrexate, pyrrolobenzodiazepine, or radioisotopes and/or pharmaceutically acceptable salts thereof; preferably derived from a drug selected from auristatin, maytansine, camptothecin, doxorubicin, pyrrolobenzodiazepine or radioisotopes and/or pharmaceutically acceptable salts thereof. In one embodiment embodiment, the drug is not an auristatin analog.

According to one embodiment, each moiety D₁ in formulae (Ia), (Ia′) and (Ib) is independently represented by the following formula (III):

W₁-Dxx-Dyy---- (III)

W₁ represents a moiety derived from a drug that differs from a native drug only by virtue of the covalent attachment to Dxx (as shown above). In one embodiment W₁ represents a moiety derived from duocarmycin, auristatin, maytansine, tubulysin, calicheamicin, camptothecin, SN-38, taxol, daunomycin, vinblastine, doxorubicin, methotrexate, pyrrolobenzodiazepine, or radioisotopes and/or pharmaceutically acceptable salts thereof; preferably a moiety derived from auristatin, maytansine, camptothecin, doxorubicin, pyrrolobenzodiazepine or radioisotopes and/or pharmaceutically acceptable salts thereof.

According to one embodiment, W₁ represents a moiety derived from auristatin, preferably a moiety derived from auristatin F (AF), auristatin E (AE), auristatin Cit (ACit), monomethyl auristatin F (MMAF), monomethyl auristatin Cit (MMACit) or monomethyl auristatin E (MMAE), more preferably a moiety derived from AF or MMAF, or represents a moiety derived from maytansine, such as mertansine (also known as DM1) or ravtansine (also known as DM4). In some instances, W₁ is not an auristatin analog. In other embodiments, W₁ is not auristatin Asp (AAsp), auristatin Glu (AGlu), auristatin PhosphoThr (AphThr) or auristatin Thr (AThr).

Dxx represents a single covalent bond or an amino acid having a hydrophobic side chain, preferably an amino acid selected from Phe, Val, Tyr and Ala. According to one embodiment, Dxx represents a combination of an amino acid having a hydrophobic side chain as specified above and a divalent moiety selected from maleimides, triazoles, hydrazones, carbonyl-containing groups, and derivatives thereof that is attached (by the N-terminus of the amino acid with hydrophobic side chain) to moiety W₁ via the divalent moiety selected from maleimides, triazoles, hydrazones, carbonyl-containing groups, and derivatives thereof. Preferably, Dxx is a moiety consisting of an amino acid having a hydrophobic side chain as specified above and a divalent maleimide or triazole derivative wherein attachment to moiety W₁ is via the divalent maleimide or triazole derivative.

Dyy represents a single covalent bond or an amino acid having a basic side chain, preferably an amino acid selected from Arg, Lys, Phe, Cit, Orn, Dap, and Dab, more preferably Arg or Cit.

The broken line indicates covalent attachment to the N-terminus of Axx in formula (I), the N-terminus of Ayy in formula (I′), the N-terminus of A′xx in formulae (Ia) and (Ib), or the N-terminus of A′yy in formula (Ia′).

According to one preferred embodiment, W₁ represents a moiety derived from auristatin, preferably AF, Dxx represents a single covalent bond, and Dyy represents an amino acid selected from Arg, Lys, Phe, Cit, Orn, Dap, and Dab, preferably Arg or Cit.

According to one further preferred embodiment, W₁ represents a moiety derived from maytansine, preferably DM1; Dyy is Arg, Lys or Cit, preferably Cit or Lys; Dxx is an amino acid having a hydrophobic side chain, e.g. Phe, that is attached to maytansine via a divalent maleimide derivative.

According to one further embodiment, each moiety D₂ and D in formulae (Ia), (Ia′), (Ib), (II), (II′) and (IIa) is independently represented by the following formula (IIIa):

W₂-Exx---- (IIIa)

W₂ represents a moiety derived from duocarmycin, auristatin, maytansine, tubulysin, calicheamicin, camptothecin, SN-38, taxol, daunomycin, vinblastine, doxorubicin, methotrexate, pyrrolobenzodiazepine, or radioisotopes and/or pharmaceutically acceptable salts thereof.

Exx represents a single covalent bond or a divalent moiety selected from maleimides, triazoles, hydrazones, carbonyl-containing groups, amino acids, dipeptide moieties and derivatives thereof, preferably a divalent maleimide or triazole derivative, more preferably a maleimide derivative.

The broken line indicates covalent attachment to the side chain of A′xx in formulae (Ia) and (Ia′), the side chain of A′xx or the C-terminus of Cxx if present in formula (Ib), the side chain of Bxx in formulae (II) and (II′), the side chain of Bxx or the C-terminus of Cxx if present in formula (IIa).

According to one preferred embodiment, W₂ represents a moiety derived from auristatin (e.g. AF) or maytansine (e.g. DM1). If W₂ is a moiety derived from auristatin (e.g. AF), the attachment can occur via the C-terminal carboxyl group of the drug and the ω-amino group of Bxx (formulae (II) and (II′)) or A′xx (formulae (Ia), (Ia′) and (Ib)). If W₂ is a moiety derived from maytansine (e.g. DM1) the attachment to the ω-amino group of Bxx or A′xx preferably occurs via a divalent maleimide derivative.

### 6. Vector Group

V in formulae (I), (I′), (Ia1), (Ia2), (Ia3), (II), (II′) and (IIa) represents a moiety derived from a vector group capable of interacting with a target cell. The expression “capable of interacting with a target cell”, as used herein, indicates that the vector group can bind to, complex with, or react with a moiety, e.g. an antigen or a receptor, on the surface of a target cell. Such an interaction with the target cell can be experimentally verified by methods known in the art, for instance by providing a compound of formula (I), which carries a label (such as a fluorescence marker), by contacting said compound with tissue containing target cells and by detecting the distribution of the fluorescence marker within the tissue (e.g. by fluorescence microscopy). An increase of fluorescence intensity at the target cells indicates an interaction with the target cell in accordance with the present invention. In some preferred embodiments, the vector group is also capable of causing or contributing to internalization of the targeted-drug-conjugate (i.e. compound of formula (I) or formula (II)) into the target cell.

According to one embodiment, V represents a moiety derived from a vector group selected from antibodies, antibody fragments, proteins, peptides, and non-peptidic molecules.

According to one preferred embodiment, V represents a moiety derived from an antibody or an antibody fragment such as a single chain antibody, a monoclonal antibody, a single chain monoclonal antibody, a monoclonal antibody fragment, a chimeric antibody, a chimeric antibody fragment, a domain antibody or fragment thereof, a cytokine, a hormone, a growth factor, a colony stimulating factor, a neurotransmitter or a nutrient-transport molecule.

According to another preferred embodiment, V represents a moiety derived from a peptide capable of interacting with a target of interest. Non-limiting examples of peptides include somatostatin or analogues thereof, such as octreotide, Angiopep-2, Gastrin-releasing peptide, transferrin-derived peptide, derivative of the Neuropeptide Y, RGD peptides, alpha-melanocyte stimulating hormone peptide analogs, vasoactive intestinal peptide, neurotensin and luteinizing hormone-releasing hormone (LHRH) analogs.

According to yet another preferred embodiment, V represents a moiety derived from a non-peptidic molecule such as folic acid, hyaluronic acid, a Neurotensin Receptor 1 (NRT1) antagonist such as SR 142948A derivatives and a ligand of the prostate specific membrane antigen (PSMA) such as PSMA-617 and PSMA-11.

According to one embodiment, the target cell is selected from tumor cells, virus infected cells, microorganism infected cells, parasite infected cells, cells involved in autoimmune diseases, activated cells, myeloid cells, lymphoid cells, melanocytes, and infectious agents including bacteria, viruses, mycobacteria, fungi.

According to one preferred embodiment, the target cell is any tumor cell from a solid or liquid tumor, including but not limited to lymphoma cells, myeloma cells, renal cancer cells, breast cancer cells, prostate cancer cells, ovarian cancer cells, colorectal cancer cells, gastric cancer cells, squamous cancer cells, small-cell lung cancer cells, testicular cancer cells, or any cells growing and dividing at an unregulated and quickened pace to cause cancers.

### 7. Pharmaceutical Compositions

The compounds of the present invention can be provided in the form of pharmaceutical compositions for human or animal usage in human and veterinary medicine. Such compositions typically comprise a therapeutically effective amount of LDC according to the present invention or a pharmaceutically acceptable salt thereof, and one or more components selected from a carrier, a diluent and other excipients.

Suitable carriers, diluents and other excipients for use in pharmaceutical compositions are well known in the art, and are for instance described in Remington's Pharmaceutical Sciences, Mack Publishing Co. (Gennaro AR, 1985). The carrier, diluent and/or other excipient can be selected with regard to the intended route of administration and pharmaceutical practice. The pharmaceutical compositions can comprise as the carrier, diluents and/or other excipients, or in addition to, any suitable binder(s), lubricant(s), suspending agent(s), coating agent(s), solubilizing agent(s).

The therapeutically effective amount can be determined by a physician on a routine basis. The specific dose level and frequency of dosage for any particular subject/patient can vary and depends on a variety of factors including the activity of the specific drug compound employed, the metabolic stability and length of action of that compound, the patient's age, body weight, general health, sex, diet, mode and time of administration, rate of excretion, drug combination, the severity of the particular condition, and the individual undergoing therapy. These factors are taken into account by the physician when determining the therapeutically affective dose.

### 8. Use of LDCs or Compositions Thereof in Methods of Preventing or Treating Diseases

The compounds of the present invention including the compound of formula (I)/(I′) or the compound of formula (II)/(II′) can be used to treat disease. The treatment can be a therapeutic and/or prophylactic treatment, with the aim being to prevent, reduce or stop an undesired physiological change or disorder. In some aspects, the treatment can prolong survival of a subject as compared to expected survival if not receiving the treatment.

The disease that is treated by the LDC can be any disease that benefits from the treatment, including chronic and acute disorders or diseases and also those pathological conditions which predispose to the disorder. In some aspects, the disease is a neoplastic disease such as cancer that can be treated via the targeted destruction of tumor cells. Non-limiting examples of cancers that may be treated include benign and malignant tumors, either solid or liquid; leukemia and lymphoid malignancies, as well as breast, ovarian, stomach, endometrial, salivary gland, lung, kidney, colon, thyroid, pancreatic, prostate or bladder cancer. The disease may be a neuronal, glial, astrocytal, hypothalamic or other glandular, macrophagal, epithelial, stromal and blastocoelic disease; or inflammatory, angiogenic or an immunologic disease. An exemplary disease is a solid, malignant tumor.

According to one embodiment, the compound of the present invention or composition thereof is used in a method of treating or preventing a cancer, an autoimmune disease and/or an infectious disease, for instance by administering a therapeutically effective amount of the compound of the present invention or composition thereof to a patient in need thereof.

The molecule can be administered to a subject (e.g. a patient) at one time or over a series of treatments. Depending on the type and severity of the disease, between about 0.1 μg/kg to 1 mg/kg of drug may be used as an initial candidate dosage for first administration in a first-in-human trial, e.g. by one or more separate administrations, or by continuous infusion. A typical daily dosage can range from about 0.1 mg/kg to 50 mg/kg or more, or from about 0.5 to about 25 mg/kg of patient weight.

When treating cancer, the therapeutically effect that is observed can be a reduction in the number of cancer cells; a reduction in tumor size; inhibition or retardation of cancer cell infiltration into peripheral organs; inhibition of tumor growth; and/or relief of one or more of the symptoms associated with cancer.

The routes for administration (delivery) include one or more of oral (e.g. tablet, capsule, ingestable solution), topical, mucosal (e.g. nasal spray, aerosol for inhalation), nasal, parenteral (e.g. an injectable form), gastrointestinal, intraspinal, intraperitoneal, intramuscular, intravenous, intrauterine, intraocular, intradermal, intracranial, intratracheal, intravaginal, intracerebroventricular, intracerebral, subcutaneous, ophthalmic (including intravitreal or intracameral), transdermal, rectal, buccal, vaginal, epidural, sublingual. According to a preferred embodiment, the compound of the present invention is administered by injection, such as parenterally, intravenously, subcutaneously, intramuscularly, transdermally.

According to one further embodiment, the compound of the present invention is used in a method of treating or preventing a cancer, an autoimmune disease and/or an infectious disease, and is administered concurrently with one or more other therapeutic agents such as chemotherapeutic agents, radiation therapy, immunotherapy agents, autoimmune disorder agents, anti-infectious agents, or one or more other compounds of formula (I)/(I′) and/or (II)/(II′) and/or (IIa). It is also possible to administer the other therapeutic agent before or after the compound of the present invention.

### 9. Use of Labelled LDCs for Diagnostic and/or Therapeutic Purposes

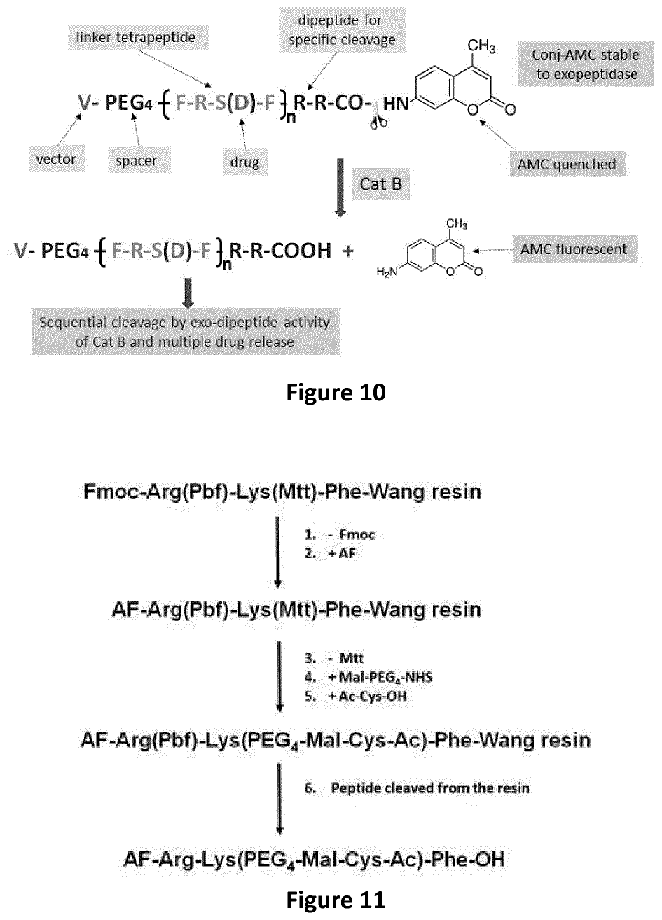

In the compound (LDC) of the present invention, Z can be a labelling agent such as a coumarin derivative or the like. Labelling agents include moieties derived from fluorescent or luminescent compounds, electron transfer agents, or other labelling agents known in the art. The compound of the present invention can be cleaved by the exopeptidase activity of Cat B at its C-terminus thus releasing the labelling agent, e.g. a fluorescent amino coumarin (AMC) derivative, in the target cell (FIG. 10).

The labelled LDCs of the present invention can be used for in vitro diagnostic purposes, e.g. for monitoring drug release in a target cell, for immuno-assays, or for immuno-histology, as well as for in vivo diagnostic and/or therapeutic applications. For instance, the labelled LDCs can be used as an aid in therapeutic applications such as (oncologic) surgery, e.g. as real time fluorescent probes for image-guided surgery.

Administration of the labelled compound according to the present invention for in vivo diagnostic and/or therapeutic applications (e.g. surgery) will be by analogous methods to unlabeled compounds. Such modes of administration are already described above, and are also found in the literature, so that they will be well-known to the skilled person.

### 10. Preparation of the Compounds of the Invention

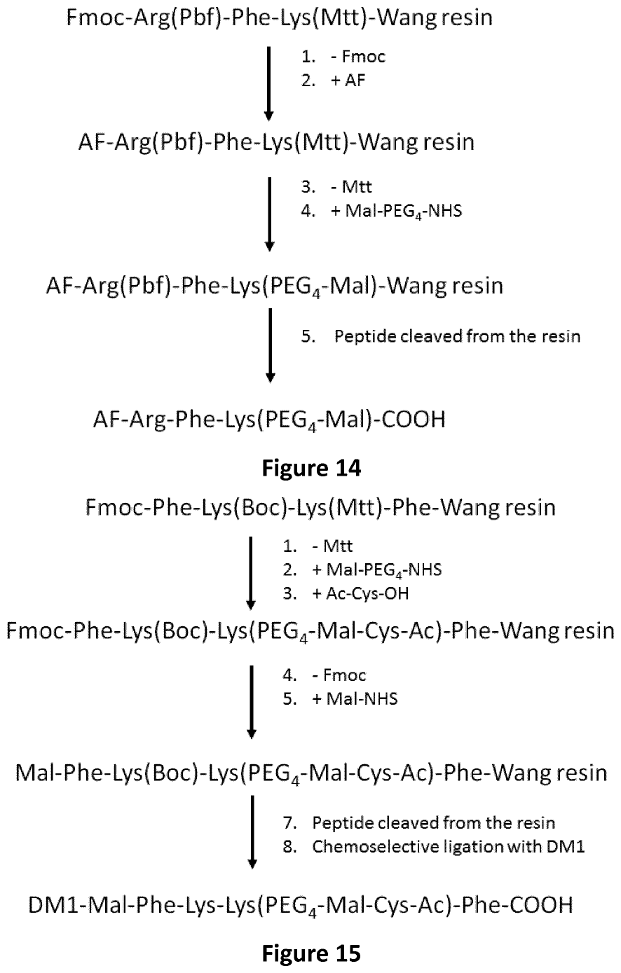

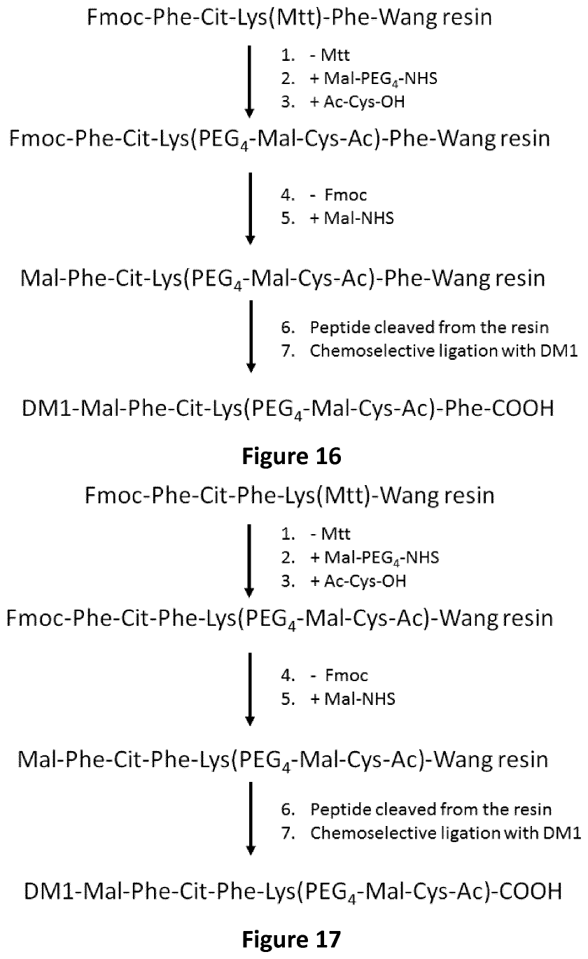

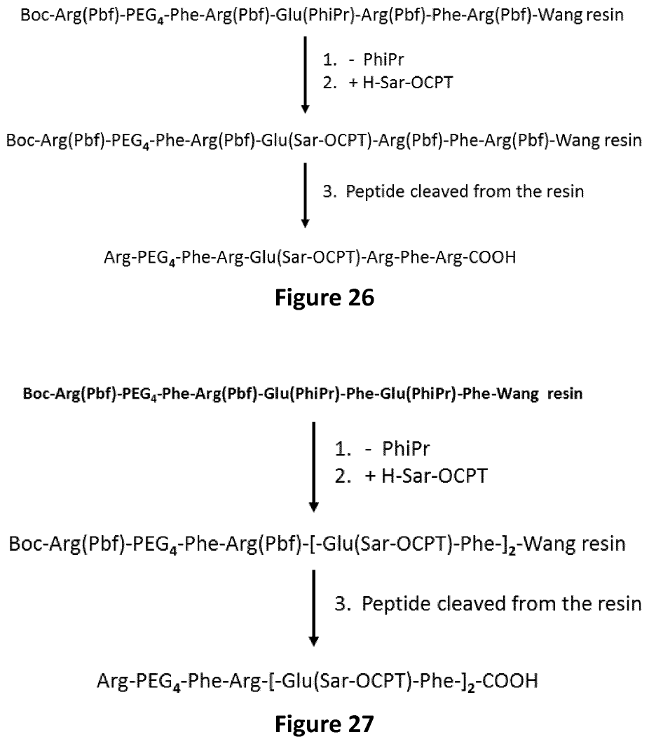

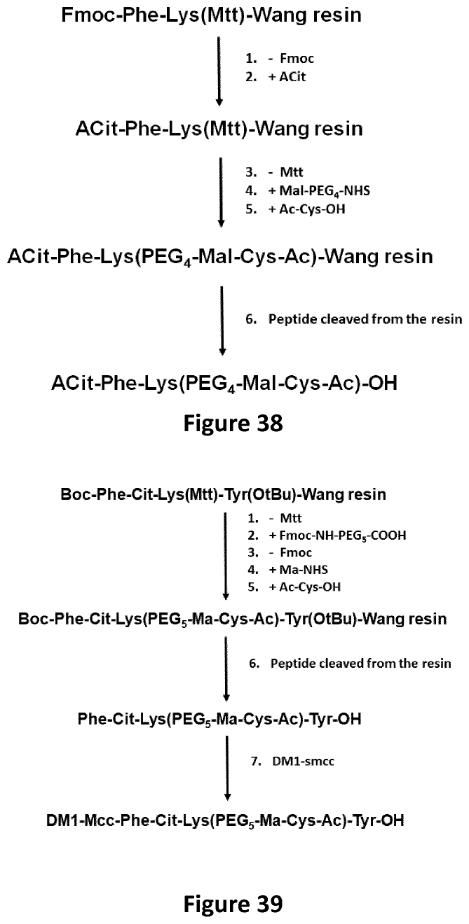

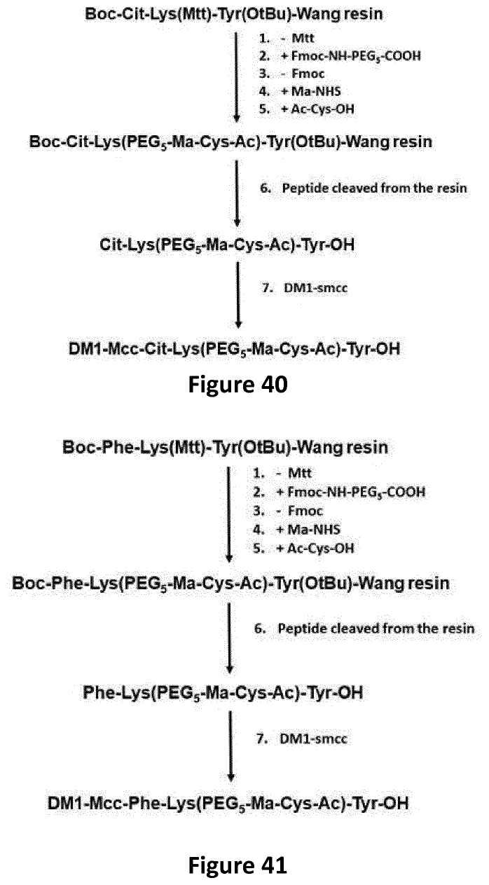

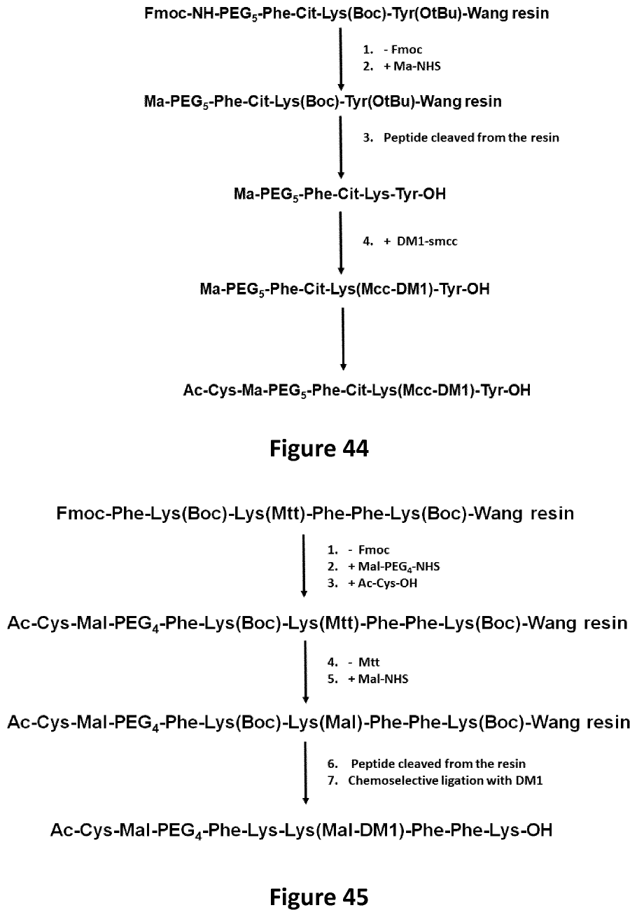

In the following, methods are provided for the preparation of linkers, drug-linkers and ligand-drug-conjugates. The compounds of the invention can be synthesized relying on standard Fmoc-based solid-phase peptide synthesis (SPPS), including on-resin peptide coupling and convergent strategies. The introduction of various maleimido-derivatives and subsequent chemoselective ligation to moieties derived from a vector group is also exemplified below. The general strategies and methodology which can be used for preparing the compounds of the present invention are well-known to the skilled person and illustrated in FIGS. 11-28 and 36-49.

### 11. Examples

### 11.1 List of Abbreviations Used in the Examples

Ac: Acetyl
AF: Auristatin Phe or Auristatin F
ACit: Auristatin Cit
Cit: Citrulline
CPT: Camptothecin
Dab: Diamino butyric acid
Dap: Diamino propionic acid
DCM: Dichloromethane
DIEA: diisopropylethylamine
DM1: N₂′-deacetyl-N₂′-(3-mercapto-1-oxopropyl)-maytansine (Mertansine)
DM1-smcc: N₂′-Deacetyl-N₂′-[3-[[1-[[4-[[(2,5-dioxo-1-pyrrolidinyl)oxy]carbonyl]cyclohexyl]methyl]-2,5-dioxo-3-pyrrolidinyl]thio]-1-oxopropyl]-maytansine (CAS: 1228105-51-8)
DMAP: Dimethylaminopyridine
DMF: Dimethylformamide
DMSO: Dimethylsulfoxyde
DPBS=Dulbecco's Phosphate Buffer Saline (reference D₈₅₃₇ from Sigma)
DTT: Dithiothreitol
eq: equivalent
HATU: 1-[Bis(dimethylamino)methylene]-1H-1,2,3-triazolo[4,5-b]pyridinium 3-oxid hexafluorophosphate
HBTU: 2-(1H-Benzotriazole-1-yl)-1,1,3,3-tetramethylaminium hexafluorophosphate
FA: Formic acid
Mal: 3-Maleimidopropionyl
Ma: 2-Maleimidoacetyl
Mal-NHS: Maleimido-N-hydroxy succinic acid
MC: Maleimidocaproyl
Mcc: 4-(N-Maleimidomethyl) cyclohexane-1-carboxyl
MES: 2-(N-morpholino)ethanesulfonic acid
MS: Mass spectroscopy
MMAF: Monomethyl Auristatin F
Mtt: Methyl trityl
Mw: Molecular weight
NHS: N-hydroxysuccinimid ester
PABC: Para-amino benzyloxycarbonyl
Pbf: 2,2,4,6,7-Pentamethyldihydrobenzofuran-5-sulfonyl
PEG₄: Tetraethylene glycol
PNP: p-Nitrophenyl
Sar: Sarcosine
SPPS: Solid-Phase Peptide Synthesis
SQD: Single Quadruple Detector
TFA: Trifluoroacetic acid
TIS: Triisopropyl silane
Trt: Trityl
TQ: Triple Quadrupole
UPLC: Ultra performance liquid chromatography
v/v: volume/volume

### 11.2 Starting Materials and Chemicals

The main starting materials and chemicals used in the following examples are listed below:
Resins and protected amino acids for solid-phase peptide synthesis from Bachem or Novabiochem (Switzerland) unless indicated otherwise;
Maleimidopropionic acid, 4-nitrophenyl chloroformate, TFA, TIS and DIEA from Sigma-Aldrich (Switzerland);
HBTU from Merck (Switzerland) and HATU from Combi-Blocks (Switzerland);
PEG derivatives (Fmoc-NH-PEG₄-COOH, Fmoc-NH-PEG₅-COOH and Mal-PEG₄-NHS) from Iris Biotech Gmbh (Germany);
AF (Auristatin F) from Levena Biopharma (USA);
ACit (Auristatin Cit) from Bachem (Switzerland);
DM1 (Mertansine) from Active Biochem (Germany);
DM1-smcc form eNovation Chemicals
CPT (Camptothecin) and Mal-NHS from Fluorochem (United Kingdom);
Ma-NHS (AMAS) from AstaTech (USA);
H-Sar-OCPT from Almac (United Kingdom);
Herceptin (Trastuzumab) from Roche Pharma (Schweiz) AG
Sephadex® PD 10 column from GE-Healthcare (reference 17-0851-01)
Amicon Ultra-4 Centrifugal Filter Unit with Ultracel-30 membrane
Dulbecco's PBS buffer from Sigma (reference D8537)
Recombinant human Cathepsin B in a precursor form from R&D Systems (Bio-Techne AG, product cat #. 953-CY-010);
Cys-MC-Val-Cit-PABC-MMAF from IBIOsource (USA, product cat #. S10001);
Monomethyl Auristatin F (MMAF) from MedKoo (USA, product cat #: 407222);
Human and CD-1 mouse, K2 EDTA pooled mixed gender, plasma from Seralab (now called BioIVT) (UK);
Procaine hydrochloride from Sigma-Aldrich (Switzerland, product cat #: 46608).
Sandwich ELISA (EDI™ Intact MMAF ADC ELISA Kit, #KTR-783) from Epitope Diagnostics Inc.

### 11.3 Methods

The following methods can be used to evaluate the compounds of the present invention.

11.3.1 Recombinant Human Cathepsin B-Induced Cleavage

Cat B-induced cleavage of the compounds of the present invention was evaluated according to the in vitro enzymatic cleavage assay using recombinant human Cathepsin B and UHPLC-MS/MS analysis as described below.

Reference compounds Cys-MC-Val-Cit-PABC-MMAF and MMAF were used as positive controls. The enzyme was reconstituted in 25 mM MES buffer adjusted at pH 5.0 with a 1M NaOH solution and then activated with a 20 nM solution of DDT at room temperature for at least 15 min.

The in vitro enzymatic assay was conducted at 37° C. with the test compounds at a concentration of 10 μM (2.5 μM when the test compound is an antibody-drug conjugate) in the presence of activated recombinant human Cathepsin B enzyme at 2 μg/mL in a 25 mM MES buffer pH 5.0. The enzymatic cleavage reaction was stopped for each defined time point by mixing an equal volume of acetonitrile+0.1% FA containing an internal standard (warfarine at 8 μM).

Analysis was conducted using a Waters Acquity UPLC System coupled to a Waters Xevo TQ triple quad mass spectrometer. UHPLC was conducted depending on the test compounds with a BEH C8 1.7 μm 100×2.1 mm or BEH C18 1.7 μm 50×2.1 mm or HSS T3 1.7 μm 50×2.1 mm columns heated at 45° C. or 50° C. and fitted with 2 μm insert filter pre-columns (available from Waters), and solvent systems A1 (H₂O+0.1% FA) and B1 (acetonitrile+0.1% FA) at a flow rate of 0.6 mL/min and a 10-95% gradient of B1 over 1.9 min.

MS/MS was performed using electrospray ionization (ESI) interface in positive mode and specific MRM transitions for each tested compound.

11.3.2 Human and Mouse Plasma Stability

Human and mouse plasma stability of Ligand-Drug-Conjugates of the present invention were evaluated according to the plasma stability assay using UHPLC-MS/MS analysis as described below. When the test compound is an antibody-drug conjugate, supplementary immunoassays were conducted.

Procaine was used as positive control for human and mouse plasma stability. The in vitro plasma stability assay was performed at 37° C. with a 1 pM test compound (LDC) concentration in plasma over 24 h. The enzymatic reaction was stopped for each defined time point by mixing 1 volume of plasma with 3 volumes of acetonitrile+0.1% FA containing an internal standard (warfarine at 0.65 μM). Each sample was then centrifuged at 16′000×g for 5 min at 4° C. The supernatants were transferred in injection vials.

Analyses were performed using a Waters Acquity UPLC System coupled to a Waters Xevo TQ triple quad mass spectrometer. UHPLC was conducted depending on the test compounds with a BEH C8 1.7 μm 100×2.1 mm or BEH C18 1.7 μm 50×2.1 mm or HSS T3 1.7 μm 50×2.1 mm columns heated at 45° C. or 50° C. and fitted with 2 μm insert filter pre-columns (available form Waters), and solvent systems A1 (H₂O+0.1% FA) and B1 (acetonitrile+0.1% FA) at a flow rate of 0.6 mL/min and a 10-95% gradient of B1 over 1.9 min.

MS/MS was performed using electrospray ionization (ESI) interface in positive mode and specific MRM transitions for each test compound.

Integrity of antibody-drug conjugate was controlled by immunoassay. For example, in the case of ADC1 (described in more detail below), the concentration of intact ADC was quantified using sandwich ELISA (EDI™ Intact MMAF ADC ELISA Kit, #KTR-783) according to the manufacturer's instruction. Briefly, aliquots were collected at different time points during the plasma stability experiment described above. All samples were diluted 1:800 prior to immunodetection. Trastuzumab and AF-Arg were included as negative controls (data not shown). Standards and QC samples (low, medium, high) spiked with known concentrations of ADC1 were used to quantify unknown samples and validate the run respectively.

11.3.3 Binding Affinity Assay

The binding affinity of antibody-drug conjugates according to the present invention was assessed as follows.

In the case of ADC1 (described in more detail below), SK-BR-3 (ErbB2-expressing) and MD-MB-231 (ErbB2-negative) cells were incubated with either trastuzumab or ADC1. For SK-BR-3 cells, the concentration of both compounds ranged from 3 μg/mL to 3×10⁻⁴ μg/mL ( 1/10 dilutions). For MDA-MB-231 cells, only the 3 μg/mL concentration was used for both compounds. Next, cells were incubated with a secondary goat anti-human antibody conjugated with Alexa 488 (BioLegend) and a live/dead stain prior analysis on a BD LSRII instrument. Error bars: SD (n=2). Raw data were analyzed in FlowJo (FlowJo).

In the case of ADC3 (described in more detail below), BT-474 (ErbB2-expressing) and MD-MB-231 (ErbB2-negative) cells were incubated with either ADC3 or trastuzumab. For BT-474 cells, the concentration of all compounds ranged from 3 μg/mL to 3×10⁻⁶ μg/mL ( 1/10 dilutions). For MDA-MB-231 cells, only the 3 μg/mL concentration was used for all compounds. Next, cells were incubated with a secondary rat anti-human IgG FC antibody conjugated with Alexa 488 (BioLegend) and a live/dead stain prior analysis on an Attune Nxt flow cytometer. Error bars: SD (n=2). Raw data were analyzed in FlowJo (FlowJo).

11.3.4 Cytotoxic Activity

Log phase cultures of cell lines SK-OV-3 (ErbB2-expressing), SK-BR-3 (ErbB2-expressing), BT-474 (ErbB2-expressing) and MDA-MB-231 (ErbB2 negative) were collected and cells plated at seeding densities ranging from 1,500 to 12,000 cells/well in 96-well microtiter plates according to pre-determined conditions. After incubating overnight (at 37° C., 5% CO₂ or 0% CO₂ for MDA-MB-231 cells) to allow cell adhesion and surface protein reconstitution, serial dilutions of test compounds were added (0.1% DMSO final concentration for AF, AF-Arg and DM1; 5% water of injection for trastuzumab; 5% PBS for ADC1 and ADC3) and cultures incubated further during 72, 96 or 120 hours.

Assessment of cellular growth was done using Alamar Blue (available from Thermo Fisher Scientific) dye reduction assay. Alamar Blue was added to cells to constitute 10% culture volume. Cells were incubated for 4 to 6 hours, and dye reduction was measured by fluorescence on an EnSpire plate reader (Perkin Elmer). Background corrected fluorescence measurements were transformed in % scale by considering vehicle value as 100% activity (relative measurements). Next, relative measurements were analyzed using GraphPad Prism software to derive relative IC₅₀. All experiments were done twice with 3 replicates per concentration. Error bars: SEM (n=3). Cytotoxicity of AF and AF-Arg on ErbB2-expressing SK-OV-3 and SK-BR-3 cells after 72 and 120 h of treatment. SK-OV-3 and SK-BR-3 cells were seeded the day before treatment in complete culture medium. After overnight resting, cells were treated with decreasing concentrations of test compounds in complete culture medium (AF: 10 μM-1 pM; AF-Arg: 10 μM-1 pM, log-dilution).

Cytotoxicity of ADC1 and derivatives (trastuzumab, AF-Arg and Compound 2) on ErbB2-expressing SK-OV-3 and SK-BR-3 cells and ErbB2-negative MDA-MB-231 cells after 96 h of treatment. SK-OV-3, SK-BR-3 and MDA-MB-231 cells were seeded the day before treatment in complete culture medium. After overnight resting, cells were treated with decreasing concentrations of test compounds in complete culture medium (Compound 2: 10 μM-1 pM; AF-Arg: 10 μM-1 pM; trastuzumab: 7.22 μM-0.72 pM; ADC1: 0.4 μM-0.04 pM, log-dilution).

Cytotoxicity of ADC3 and derivatives (trastuzumab and DM1) on ErbB2-expressing BT-474 cells and ErbB2-negative MDA-MB-231 cells after 96 h of treatment. BT-474 and MDA-MB-231 cells were seeded the day before treatment in complete culture medium. After overnight resting, cells were treated with decreasing concentrations of test compounds in complete culture medium (DM1: 10 μM-1 pM; ADC3: 1 μM-0.1 pM; trastuzumab: 7.215 μM-1 pM, log-dilution).

11.3.5 Drug Antibody Ratio

The Drug Antibody Ratio (DAR) was measured by RP-LC using an UPLC Waters Acquity system equipped with a binary delivery pump, an autosampler operating at 25° C., a column oven and a diode array detector (DAD) operating in the range 190-500 nm. To separate the different ADC chains (Heavy and light chains) a Thermo mAb pack RP column (4 μm 2.1×100 mm) (Thermo Fisher Scientific AG, Sunnyvale, Calif., USA) was used.

The samples were prepared by adding 5 μL of a solution 100 mM of Dithiothreitol (DDT) to 45 μL of ADC solution at 2.5 mg/mL in water to separate the light and heavy chains linked by the disulfide bridges. The mixture was then incubated for 1 hour at 30° C.

A gradient mode was applied as described in the following table (mobile phase A was constituted of Trifluoroacetate 0.1% (volume) in water and mobile phase B trifluoracetate 0.1% (volume) in acetonitrile).

| t (min) | Flow (mL/min) | % A | % B |
|---|---|---|---|
| 0.0 | 0.6 | 73 | 27 |
| 13.0 | 0.6 | 60 | 40 |
| 13.1 | 0.6 | 73 | 27 |
| 16.0 | 0.6 | 73 | 27 |

Column temperature was 90° C., injection volume was 5 μL and chromatograms were acquired at 280 nm. DAR was then calculated using AUC of each peak.

The quantification was performed by UV spectrometry using a BioTeck Synergy HT microplate reader (BioTeck Instrument, Sursee, Switzerland) and Microplates Greiner Bio-one (Huberlab, Aesch, Switzerland).

Before quantification of the solution, a comparison between the absorbance spectrum at 280 nm of ADC before and after purification (with and without free drug/payload respectively) was performed to evaluate if UV-absorbance interference exists between mAb (e.g. trastuzumab) and drug.

Because UV absorbance of the drug (e.g. DM1) of ADC interferes with mAb (e.g. trastuzumab), quantification have to be performed according to the following equation taking into account both UV response of mAb and drug:

$${C}^{mAb} = ( \frac{{A}_{280} \times {ɛ}_{252}^{DM 1} - {A}_{252} \times {ɛ}_{280}^{DM 1}}{{ɛ}_{280}^{mAb} \times {ɛ}_{252}^{DM 1} - {ɛ}_{252}^{mAb} \times {ɛ}_{280}^{DM 1}} ) / l$$

where:
Aₓ corresponds to the total absorbance at the wavelength x
Aₓ^{y} corresponds to the UV absorbance at the wavelength x for the specie y (mAb or drug)
εₓ^{y} corresponds to the molar refraction coefficient at the wavelength x for the specie y
I corresponds to the optical path
C_{mAb} corresponds to the concentration of ADC/mAb into the solution.

Determination of ε_{mAb} and ε_{drug}: two calibration curves were performed at 280 nm and 252 nm with 5 solutions of trastuzumab (respectively drug) of known concentrations. The ε_{mAb} (respectively ε_{drug}) was then calculated using Lambert-Beer equation: Abs=ε×I×C.

Sample preparation: the solution of ADC (in water) was first centrifuged for 5 min at 21′500 g. Then the solution was diluted with adequate volume of water to correspond to the range of concentration of the calibration curve. The diluted solution was then centrifuged for 5 min at 21′500 g. 200 μL of supernatant was then dispensed into a UV-microplate for UV analysis.

### Example 1: Preparation of Compounds of Formula (I) or (I′)

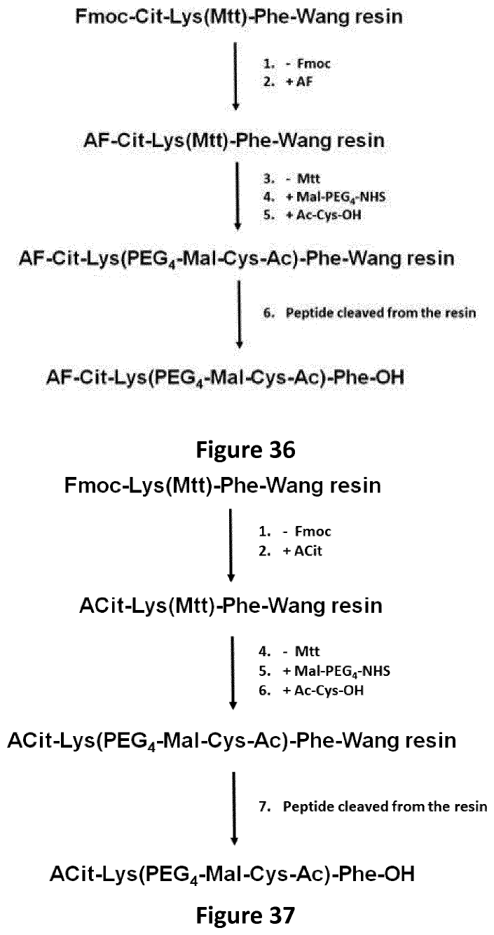

The compounds described herein were prepared by using standard Fmoc-based SPPS, including on-resin peptide coupling and convergent strategies as shown in FIGS. 11 to 17 and in FIGS. 36 to 41. The compounds prepared in Example 1 are shown in Table 1 below.

TABLE 1
Compounds of formula (I)/(I′)

| Compound | Structure |
|---|---|
| 1 | AF-Arg-Lys(PEG₄-Mal-Cys-Ac)-Phe-OH |
| 2 | AF-Arg-Lys(PEG₄-Mal)-Phe-OH |
| 3 | AF-Arg-Phe-Lys(PEG₄-Mal-Cys-Ac)-OH |
| 4 | AF-Arg-Phe-Lys(PEG₄-Mal)-OH |
| 5 | DM1-Mal-Phe-Lys-Lys(PEG₄-Mal-Cys- |
| | Ac)-Phe-OH (SEQ ID NO: 44) |
| 6 | DM1-Mal-Phe-Cit-Lys(PEG₄-Mal-Cys- |
| | Ac)-Phe-OH (SEQ ID NO: 45) |
| 7 | DM1-Mal-Phe-Cit-Phe-Lys(PEG₄-Mal- |
| | Cys-Ac)-OH (SEQ ID NO: 46) |
| 19 | AF-Cit-Lys(PEG₄-Mal-Cys-Ac)-Phe-OH |
| 20 | ACit-Lys(PEG₄-Mal-Cys-Ac)-Phe-OH |
| 21 | ACit-Phe-Lys(PEG₄-Mal-Cys-Ac)-OH |
| 22 | DM1-Mcc-Phe-Cit-Lys(PEG₅-Ma-Cys- |
| | Ac)-Tyr-OH (SEQ ID NO: 58) |
| 23 | DM1-Mcc-Cit-Lys(PEG₅-Ma-Cys-Ac)- |
| | Tyr-OH |
| 24 | DM1-Mcc-Phe-Lys(PEG₅-Ma-Cys-Ac)- |
| | Tyr-OH |

The peptides were prepared by standard Fmoc-based SPPS using an Activo P-11 Automated Peptide Synthesizer (available from Activotec), and a Fmoc-Xxx-Wang resin (Xxx: C-terminal amino acid; loading: 0.60 mmol/g; Bachem) as shown in FIGS. 11-17 and in FIGS. 36-41.

Coupling reactions for amide bond formation were performed over 30 min at room temperature using 3 eq of Fmoc-amino-acids, Fmoc-NH-PEG₄-COOH or Fmoc-NH-PEG₅-GOOH activated with HBTU (2.9 eq) in the presence of DIEA (7 eq). Fmoc deprotection was conducted with a solution of 20% piperidine in DMF. Selective removal of the Mtt side-chain protecting group (Lys) was performed using DCM/TFA/TIS (94/1/5, v/v/v).

For the synthesis of compounds 1 to 4 and 19, Auristatin F (AF) was coupled after Fmoc removal by fragment condensation (3 eq AF, 2.9 eq HBTU, 7 eq DIEA) during 30 min. For the synthesis of compounds 20 and 21, Auristatin Cit (ACit) was coupled after Fmoc removal at identical conditions (3 eq ACit, 2.9 eq HBTU, 7 eq DIEA).

For the synthesis of compounds 1 to 4 and 19 to 21, the derivative Mal-PEG₄-NHS was added on resin for 30 min (3 eq of Mal-PEG₄-NHS, 7 eq DIEA) after Mtt removal by DCM/TFA/TIS (94/1/5, v/v/v). Then, for compounds 1, 3, 19, 20 and 21, the maleimide residue on the PEG chain was reacted on resin with acetyl-cysteine (Ac-Cys-OH) via chemoselective ligation (3 eq of Ac-Cys-OH, DIEA, 7 eq) during 20 min. The peptides were cleaved from the resin under simultaneous side-chain deprotection by treatment with TFA/TIS/water (95/2.5/2.5, v/v/v) during 60 min. After concentration of the cleavage mixture, the crude peptides were precipitated with cold diethyl ether and centrifuged (FIG. 11-14 and FIG. 36-38).

For the synthesis of compounds 5 to 7, the derivative Mal-PEG₄-NHS was added on resin for 30 min (3 eq of Mal-PEG₄-NHS, 7 eq DIEA) after Mtt removal by DCM/TFA/TIS (94/1/5, v/v/v). Then, the maleimide residue on the PEG chain was reacted on resin with acetyl-cysteine (Ac-Cys-OH) via chemoselective ligation between maleimide and thiol (3 eq of Ac-Cys-OH, DIEA, 7 eq) during 20 min. The Mal-derivative was inserted by adding the moiety Mal-NHS to the N-terminus of Phe after Fmoc deprotection. The peptides were cleaved from the resin under simultaneous side-chain deprotection by treatment with TFA/TIS/water (95/2.5/2.5, v/v/v) during 60 min. After concentration of the cleavage mixture, the crude peptides were precipitated with cold diethyl ether and centrifuged. Then, Mertansine (DM1, 1.45 eq) was reacted with the terminal maleimide group via chemoselective ligation in PBS buffer at pH 7.4 and acetonitrile (ratio 2:1) (FIGS. 15-17).

For the synthesis of compounds 22 to 24, the derivative Ma-NHS was added on resin for 30 min (3 eq of Mal-NHS, 7 eq DIEA) after Fmoc removal. Then, the maleimide residue was reacted on resin with acetyl-cysteine (Ac-Cys-OH) via chemoselective ligation (3 eq of Ac-Cys-OH, DIEA, 7 eq) during 20 min. The peptides were cleaved from the resin under simultaneous side-chain deprotection by treatment with TFA/TIS/water (95/2.5/2.5, v/v/v) during 60 min. After concentration of the cleavage mixture, the crude peptides were precipitated with cold diethyl ether and centrifuged. After their purification, the derivative DM1-smcc (1.1 eq) was reacted to the N-terminus of the linker in solution in DMF and 4-methylmorpholine (6 eq) for 4 h (FIGS. 39-41).

The peptides were purified on a Waters Autopurification HPLC system coupled to SQD mass spectrometer with a XSelect Peptide CSH C18 OBD Prep column (130 Å, 5 μm, 19 mm×150 mm) using solvent system A (0.1% TFA in water) and B (0.1% TFA in acetonitrile) at a flow rate of 24 mL/min and a 20-60% gradient of B over 30 min.

The appropriate fractions were concentrated and yophilized. The purity was determined on a Waters Acquity UPLC System coupled to SOD mass spectrometer with a CSH 018 column (130 Å, 117 μm, 2.1 mm×50 mm) using solvent system A (0.1% FA in water) and B (0.1% FA in acetonitrile) at a flow rate of 0.6 mL/min and a 5-85% gradient of B over 5 min or SH Floro-phenyl column (130 Å, 197 μm, 2.1 mm×50 mm) using solvent system A (0.1% FA in water) and B (0.1% FA in acetonitrile) at a flow rate of 0.9 mLmin and a 5-95% gradient of B over 2.9 m.

MS-analysis was performed using electrospray ionization (ESI) interface in positive and negative mode. The results of the analysis of the compounds obtained in Example 1 are shown in Table 2 below.

TABLE 2
Analysis of compounds 1-7 and 19-24

| | | Purity | | | |
|---|---|---|---|---|---|
| Compound | Formula | (%) | Mw | [M + 2H⁺]⁺² | [M + 3H⁺]⁺³ |
| 1 | C₈₆H₁₃₉N₁₅O₂₃S | 94 | 1783.2 | 892.8 | 595.6 |
| 2 | C₇₉H₁₂₆N₁₄O₁₉ | 96 | 1575.9 | 789.0 | 526.7 |
| 3 | C₈₄H₁₃₅N₁₅O₂₂S | 90 | 1739.1 | 870.8 | 581.0 |
| 4 | C₇₉H₁₂₆N₁₄O₁₉ | 98 | 1575.9 | 789.3 | 526.7 |
| 5 | C₉₅H₁₃₂CIN₁₃O₂₉S₂ | 98 | 2019.7 | 1011.5 | 674.5 |
| 6 | C₉₅H₁₃₁CIN₁₄O₃₀S₂ | 97 | 2048.7 | 1025.6 | 683.8 |
| 7 | C₉₅H₁₃₁CIN₁₄O₃₀S₂ | 94 | 2048.7 | 1026.1 | 685.0 |
| 19 | C₈₄H₁₃₅N₁₅O₂₂S | 90 | 1739.2 | 871.9 | 581.6 |
| 20 | C₇₅H₁₂₅N₁₃O₂₂S | 96 | 1592.9 | 798.3 | 532.2 |
| 21 | C₇₅H₁₂₅N₁₃O₂₂S | 96 | 1592.9 | 798.4 | 532.1 |
| 22 | C₁₀₁H₁₄₁CIN₁₄O₃₂S₂ | 99 | 2162.9 | 1082.2 | 721.9 |
| 23 | C₉₂H₁₃₂CIN₁₃O₃₁S₂ | 89 | 2015.7 | 1008.7 | 673.0 |
| 24 | C₉₅H₁₃₀CIN₁₁O₃₀S₂ | 89 | 2005.7 | — | 669.0 |

### Example 2: Preparation of Compounds of Formula (II) or (III)

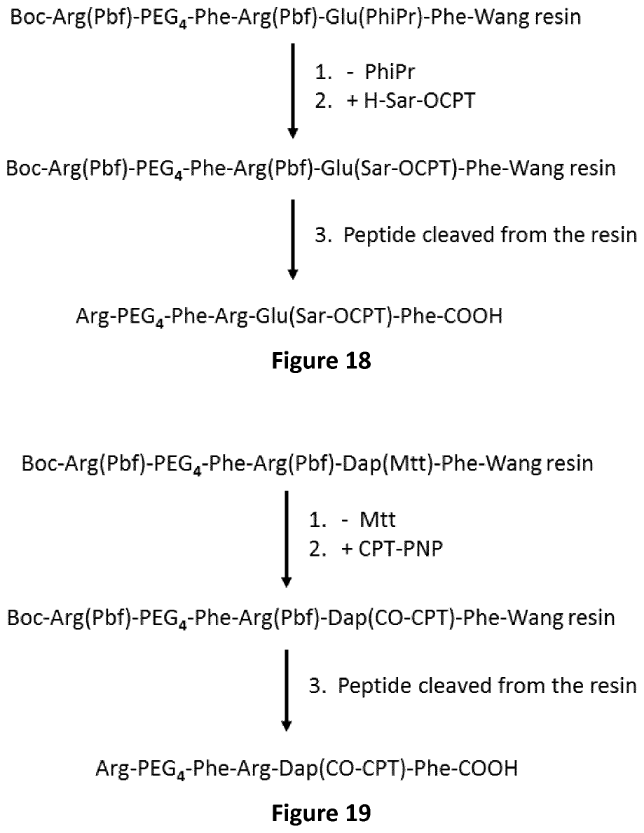

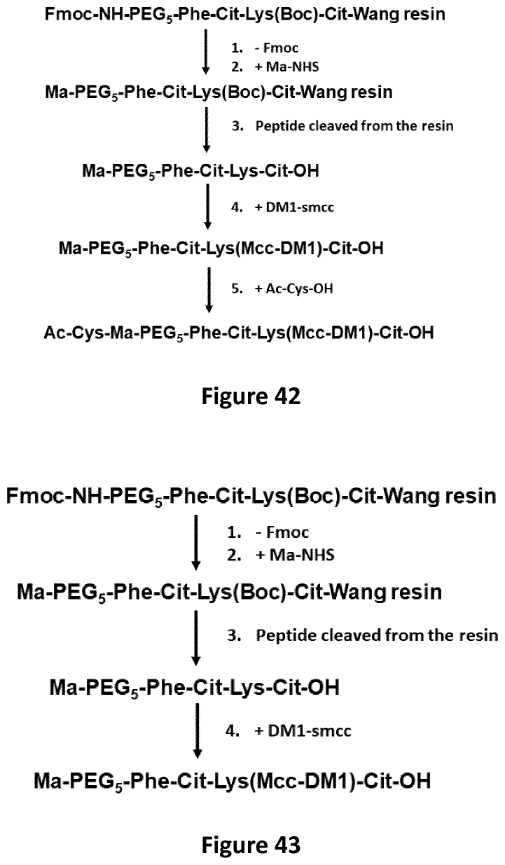

The compounds described herein were prepared using standard Fmoc-based SPPS, including on-resin peptide coupling and convergent strategies as shown in FIGS. 18-26 and in FIGS. 42-45. The compounds prepared in Example 2 are shown in Table 3 below.

TABLE 3
Compounds of formula (II)/(II′)

| Compound | Structure |
|---|---|
| 8 | Arg-PEG₄-Phe-Arg-Glu(Sar-OCPT)-Phe-OH |
| 9 | Arg-PEG₄-Phe-Arg-Dap(CO-CPT)-Phe-OH |
| | (SEQ ID NO: 48) |
| 10 | Arg-PEG₄-Phe-Arg-Dab(CO-CPT)-Phe-OH |
| | (SEQ ID NO: 49) |
| 11 | Arg-PEG₄-Phe-Arg-Ser(CO-CPT)-Phe-OH |
| | (SEQ ID NO: 50) |
| 12 | Ac-Cys-Mal-PEG₄-Phe-Lys-Lys(Mal-DM1)- |
| | Phe-OH (SEQ ID NO: 51) |
| 13 | Ac-Cys-Mal-PEG₄-Phe-Lys-Lys(AF)-Phe-OH |
| | (SEQ ID NO: 52) |
| 14 | Mal-PEG₄-Phe-Lys-Lys(AF)-Phe-OH |
| | (SEQ ID NO: 53) |
| 15 | Arg-PEG₄-Phe-Arg-Glu(Sar-OCPT)-Arg-OH |
| 16 | Arg-PEG₄-Phe-Arg-Glu(Sar-OCPT)-Arg- |
| | Phe-Arg-OH |
| 25 | Ac-Cys-Ma-PEG₅-Phe-Cit-Lys(Mcc-DM1)- |
| | Cit-OH (SEQ ID NO: 59) |
| 26 | Ma-PEG₅-Phe-Cit-Lys(Mcc-DM1)-Cit-OH |
| | (SEQ ID NO: 60) |
| 27 | Ac-Cys-Ma-PEG₅-Phe-Cit-Lys(Mcc-DM1)- |
| | Tyr-OH (SEQ ID NO: 61) |
| 28 | Ac-Cys-Mal-PEG₄-Phe-Lys-Lys(Mal-DM1)- |
| | Phe-Phe-Lys-OH (SEQ ID NO: 62) |

The peptides were prepared by standard Fmoc-based SPPS using an Activo P-11 Automated Peptide Synthesizer (available from Activotec), and a Fmoc-Xxx-Wang resin (Xxx: C-terminal amino acid; loading: 0.60 mmol/g; Bachem) as shown in FIGS. 18-26 and in FIGS. 42-45.

Coupling reactions for amide bond formation were performed over 30 min at room temperature using 3 eq of Fmoc-amino-acids, Fmoc-NH-PEG₄-COOH or Fmoc-NH-PEG₅—COOH activated with HBTU (2.9 eq) in the presence of DIEA (7 eq). Fmoc deprotection was conducted with a solution of 20% piperidine in DMF. Selective removal of the Mtt side-chain protecting group (Lys) was performed using DCM/TFA/TIS (94/1/5, v/v/v).

For the synthesis of compound 8, 15 and 16, glutamic acid was coupled as Fmoc-Glu(PhiPr)—OH and the N-terminal Arg residue was introduced as Boc-Arg(Pbf)-OH. The PhiPr side-chain protecting group was selectively removed in the presence of Boc/Pbf side-chain protecting groups by treatment with 1% (v) TFA in DCM. On-resin coupling of H-Sar-OCPT was conducted using 1.5 eq Sar-OCPT/1.4 eq HATU/4 eq DIEA in DMF for 90 min (FIGS. 18 and 25-26).

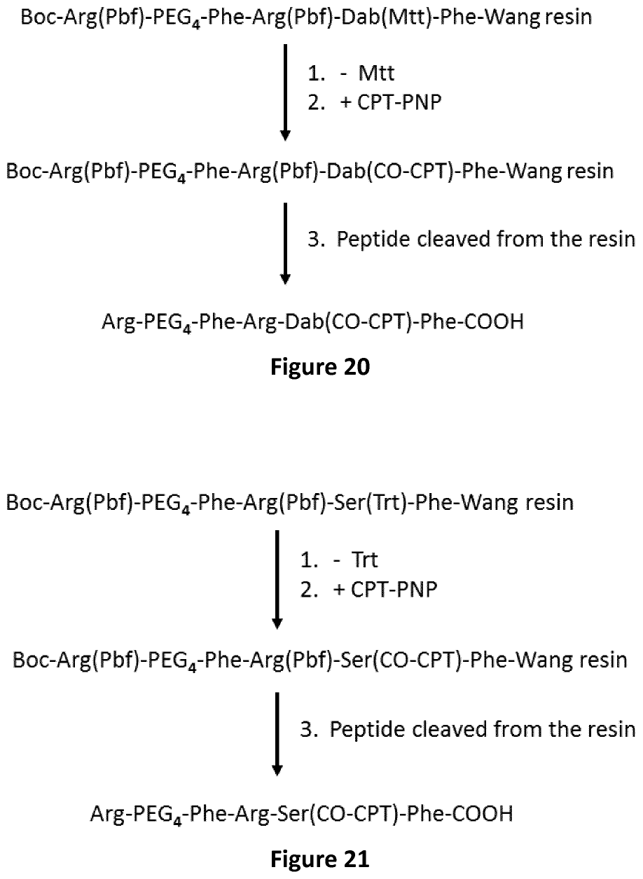

For the synthesis of compounds 9 and 10, Dap and Dab residues were introduced as Fmoc-Dap(Mtt)-OH and Fmoc-Dab(Mtt)-OH, respectively. The Mtt side-chain protecting group was selectively removed using 1% (v) TFA in DCM. Carbamate bond formation with CPT was conducted using 1.5 eq of CPT-PNP prepared as described (Pessah et al. Bioorg & Med Chem, 2004, 12, 1-8) and 4 eq of DIEA in DCM for 30 min (FIGS. 19-20).

For the synthesis of compound 11, Ser was introduced as Fmoc-Ser(Trt)-OH and the Trt protecting group was selectively removed using DCM/TFA/TIS (94/1/5, v/v/v). Carbonate bond formation with CPT was performed using 1.5 eq CPT-PNP and DMAP/DIEA (1 eq) in DCM during 12 hrs (FIG. 21).

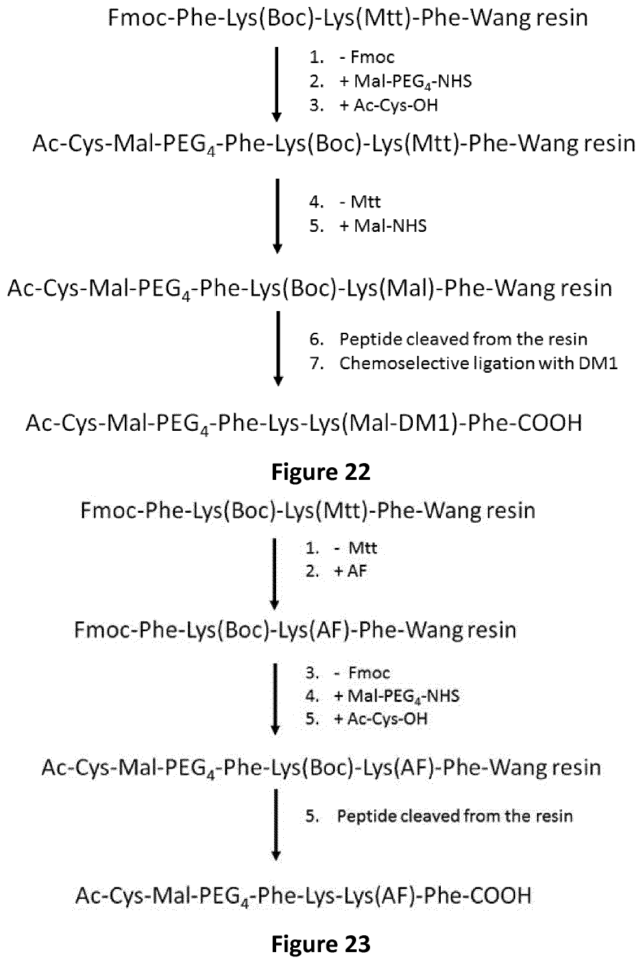

For the synthesis of compound 12 and 28, the derivative Mal-PEG₄-NHS was added on resin for 30 min (3 eq of Mal-PEG₄-NHS, 7 eq DIEA) after Fmoc deprotection conducted with a solution of 20% piperidine in DMF. Then, the maleimide residue on the PEG chain was reacted on resin with acetyl-cysteine (Ac-Cys-OH) via chemoselective ligation between maleimide and thiol (3 eq of Ac-Cys-OH, DIEA, 7 eq) during 20 min. Then, the Mal-derivative was inserted by adding the moiety Mal-NHS to the 8-amino-group of Lys after Mtt removal by DCM/TFA/TIS (94/1/5, v/v/v). The peptides were cleaved from the resin under simultaneous side-chain deprotection by treatment with TFA/TIS/water (95/2.5/2.5, v/v/v) during 60 min. After concentration of the cleavage mixture, the crude peptides were precipitated with cold diethyl ether and centrifuged. Then, Mertansine (DM1, 1.45 eq) was reacted with the terminal maleimide group via chemoselective ligation in PBS buffer pH 7.4 and acetonitrile (ratio 2:1) (FIGS. 22 and 45).

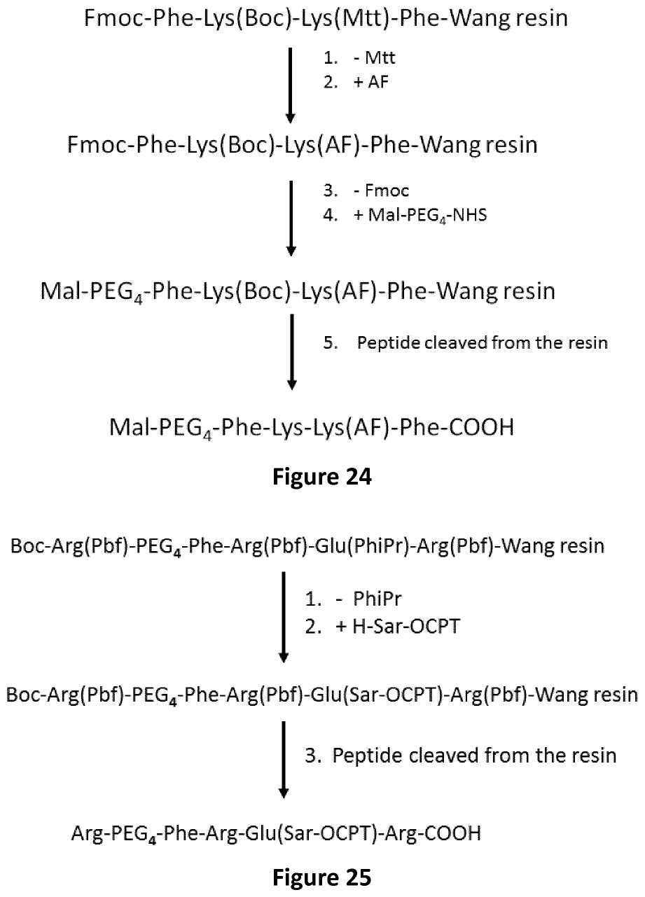

For the synthesis of compounds 13 and 14, AF was coupled by fragment condensation (3 eq AF, 2.9 eq HBTU, 7 eq DIEA) on resin to the N-terminus of the Lys residue after Mtt removal with DCM/TFA/TIS (94/1/5, v/v/v). Mal-PEG₄-NHS was added on resin for 30 min (3 eq of Mal-PEG₄-NHS, 7 eq DIEA) after Fmoc removal. For compound 13, the maleimide residue on the PEG chain was reacted on resin with acetyl-cysteine (Ac-Cys-OH) via chemoselective ligation between maleimide and thiol (3 eq of Ac-Cys-OH, DIEA, 7 eq) during 20 min (FIGS. 23-24).

For the synthesis of compounds 25 to 27, the derivative Ma-NHS was added on resin for 30 min (3 eq of Mal-NHS, 7 eq DIEA) after Fmoc deprotection conducted with a solution of 20% piperidine in DMF. Then, the peptides were cleaved from the resin under simultaneous side-chain deprotection by treatment with TFA/TIS/water (95/2.5/2.5, v/v/v) during 60 min. After concentration of the cleavage mixture, the crude peptides were precipitated with cold diethyl ether and centrifuged. After their purification, the derivative DM1-smcc (1.1 eq) was reacted to the N-terminus of the linker in solution in DMF and 4-methylmorpholine (6 eq) for 4 h. For compounds 25 and 27, the maleimide residue was reacted with acetyl-cysteine (Ac-Cys-OH) (20 eq) in acetonitrile and DPBS (ratio 1:1) for 6 h (FIGS. 42 to 44).

The peptides were purified and analyzed in the same manner and using the same equipment as described in Example 1 above. The results of the analysis of the compounds obtained in Example 2 are shown in Table 4 below.

TABLE 4
Analysis of compounds 8-16 and 25-28

| | | Purity | | | | |
|---|---|---|---|---|---|---|
| Compound | Formula | (%) | Mw | [M + H⁺]⁺ | [M + 2H⁺]⁺² | [M + 3H⁺]⁺³ |
| 8 | C₇₁H₉₅N₁₅O₁₈ | 94 | 1446.6 | 1447.9 | 724.2 | 483.7 |
| 9 | C₆₇H₈₉N₁₅O₁₇ | 96 | 1376.6 | — | 689.2 | 459.8 |
| 10 | C₆₈H₉₁N₁₅O₁₇ | 83 | 1390.5 | 1391.0 | 696.4 | 464.6 |
| 11 | C₆₇H₈₈N₁₄O₁₈ | 90 | 1377.5 | 1378.2 | 689.8 | 460.3 |
| 12 | C₉₅H₁₃₂CIN₁₃O₂₉S₂ | 94 | 2019.7 | — | 1010.4 | 674.3 |
| 13 | C₉₃H₁₄₄N₁₄O₂₃S | 98 | 1858.3 | 1859.0 | 931.0 | 620.7 |
| 14 | C₈₈H₁₃₅N₁₃O₂₀ | 95 | 1695.1 | — | 848.8 | 566.4 |
| 15 | C₆₈H₉₈N₁₈O₁₈ | 95 | 1455.6 | — | 729.0 | 486.5 |
| 16 | C₈₃H₁₁₈N₂₃O₂₀ | 96 | 1759.0 | — | 880.9 | 587.7 |
| 25 | C₉₈H₁₄₃CIN₁₆O₃₂S₂ | 94 | 2156.9 | — | 1080.5 | 720.1 |
| 26 | C₉₆H₁₃₄CIN₁₅O₂₉S | 97 | 1993.7 | 1994.6 | 988.4 | 665.7 |
| 27 | C₁₀₁H₁₄₁CIN₁₄O₃₂S₂ | 80 | 2162.9 | — | 1083.0 | 721.9 |
| 28 | C₁₁₀H₁₅₃CIN₁₆O₃₁S₂ | 94 | 2295.1 | — | 1148.6 | 765.9 |

### Example 3: Preparation of Compounds of Formula (II) for Multiple Drug Release

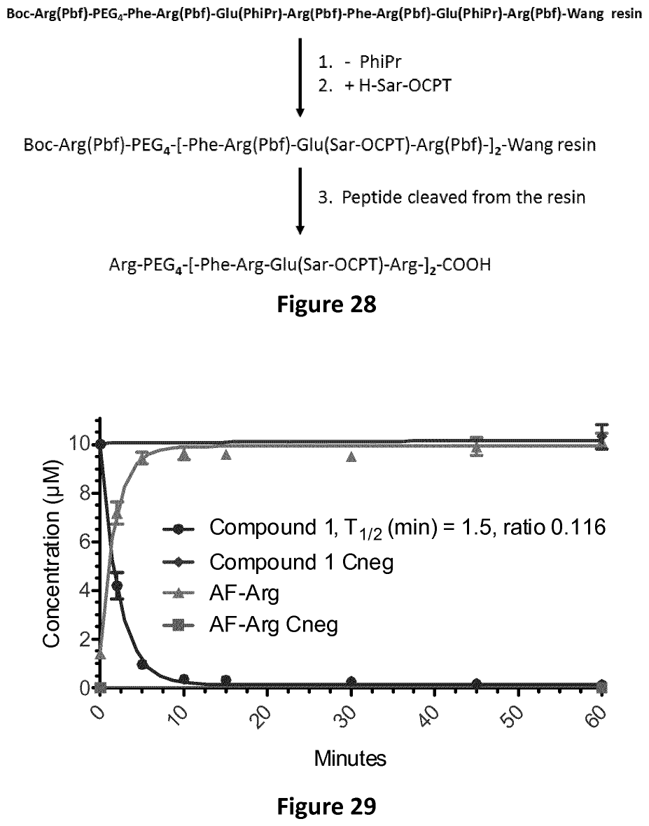

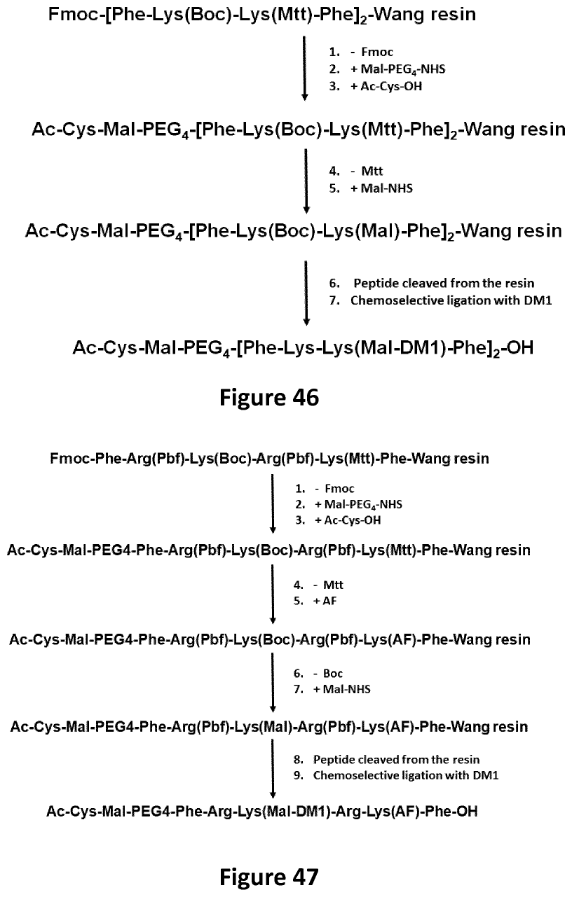

The compounds described herein were prepared using standard Fmoc-based SPPS, including on-resin peptide coupling and convergent strategies as shown in FIGS. 27 and 28 and in FIGS. 46 and 47. The compounds prepared in Example 3 are shown in Table 5 below.

TABLE 5
Compounds of formula (II) suitable
for multiple drug release

| Compound | Structure |
|---|---|
| 17 | Arg-PEG₄-Phe-Arg-[Glu(Sar-OCPT)- |
| | Phe]₂-OH |
| 18 | Arg-PEG₄-[Phe-Arg-Glu(Sar-OCPT)- |
| | Arg]₂-OH |
| 29 | Ac-Cys-Mal-PEG₄-[Phe-Lys-Lys(Mal- |
| | DM1)-Phe]₂-OH (SEQ ID NO: 63) |
| 30 | Ac-Cys-Mal-PEG₄-Phe-Arg-Lys(Mal- |
| | DM1)-Arg-Lys(AF)-Phe-OH |
| | (SEQ ID NO: 64) |

For the synthesis of compounds 17 and 18, glutamic acid was coupled as Fmoc-Glu(PhiPr)—OH and the N-terminal Arg residue was introduced as Boc-Arg(Pbf)-OH. The PhiPr side-chain protecting group was selectively removed in the presence of Boc/Pbf side-chain protecting groups by treatment with 1% (v) TFA in DCM. On resin coupling of H-Sar-OCPT was conducted using 1.5 eq Sar-OCPT (1.4 eq) HATU (4 eq) DIEA in DMF for 90 min.

The peptides were cleaved from the resin under simultaneous side-chain deprotection by treatment with TFA/TIS/water (95/2.5/2.5, v/v/v) during 60 min. After concentration of the cleavage mixture, the crude peptides were precipitated with cold diethyl ether and centrifuged (FIGS. 27-28).

For the synthesis of compound 29, the derivative Mal-PEG₄-NHS was added on resin for 30 min (3 eq of Mal-PEG₄-NHS, 7 eq DIEA) after Fmoc deprotection conducted with a solution of 20% piperidine in DMF. Then, the maleimide residue on the PEG chain was reacted on resin with acetyl-cysteine (Ac-Cys-OH) via chemoselective ligation between maleimide and thiol (3 eq of Ac-Cys-OH, DIEA, 7 eq) during 20 min. Then, the Mal-derivative was inserted by adding the moiety Mal-NHS to the 8-amino-group of Lys after Mtt removal by DCM/TFA/TIS (94/1/5, v/v/v). The peptide was cleaved from the resin under simultaneous side-chain deprotection by treatment with TFA/TIS/water (95/2.5/2.5, v/v/v) during 60 min. After concentration of the cleavage mixture, the crude peptide was precipitated with cold diethyl ether and centrifuged. Then, mertansine (DM1, 2.9 eq) was reacted with the terminal maleimide group via chemoselective ligation in PBS buffer pH 7.4 and acetonitrile (ratio 2:1) (FIG. 46).

For the synthesis of compound 30, the derivative Mal-PEG₄-NHS was added on resin for 30 min (3 eq of Mal-PEG₄-NHS, 7 eq DIEA) after Fmoc deprotection conducted with a solution of 20% piperidine in DMF. Then, the maleimide residue on the PEG chain was reacted on resin with acetyl-cysteine (Ac-Cys-OH) via chemoselective ligation between maleimide and thiol (3 eq of Ac-Cys-OH, DIEA, 7 eq) during 20 min. AF was then coupled by fragment condensation (3 eq AF, 2.9 eq HBTU, 7 eq DIEA) on resin to the Lys residue after Mtt removal with DCM/TFA/TIS (94/1/5, v/v/v). The Mal-derivative was inserted by adding the moiety Mal-NHS to the side chain of Lys after Boc removal by DCM/TMSOTf/TEA (97/1/2, v/v/v). The peptide was cleaved from the resin by treatment with TFA/TIS/water (95/2.5/2.5, v/v/v) during 60 min. After concentration of the cleavage mixture, the crude peptide was precipitated with cold diethyl ether and centrifuged. Then, mertansine (DM1, 1.45 eq) was reacted with the N-terminal maleimide group via chemoselective ligation in PBS buffer at pH 7.4 and acetonitrile (ratio 2:1) (FIG. 47).

The peptides were purified and analyzed in the same manner using the same equipment as described in Example 1 above. The results of the analysis of the compounds obtained in Example 3 are shown in Table 6 below.

TABLE 6
Analysis of compounds 17-18 and 29-30

| | | Purity | | | |
|---|---|---|---|---|---|
| Compound | Formula | (%) | Mw | [M + 2H⁺]⁺² | [M + 3H⁺]⁺³ |
| 17 | C₁₁₇H₁₅₇N₃₁O₂₈ | 91 | 2445.7 | — | 816.7 |
| 18 | C₁₀₈H₁₃₀N₂₀O₂₆ | 96 | 2124.3 | 1064.0 | 709.7 |
| 29 | C₁₆₇H₂₂₇Cl₂N₂₃O₄₆S₂ | 89 | 3459.8 | 1731.6 | 1154.1 |
| 30 | C₁₄₇H₂₂₂CIN₂₇O₃₇S₂ | 90 | 3059.1 | 1530.6 | 1021.6 |

### Example 4: Preparation of Compounds of Formula (I) for Multiple Drug Release

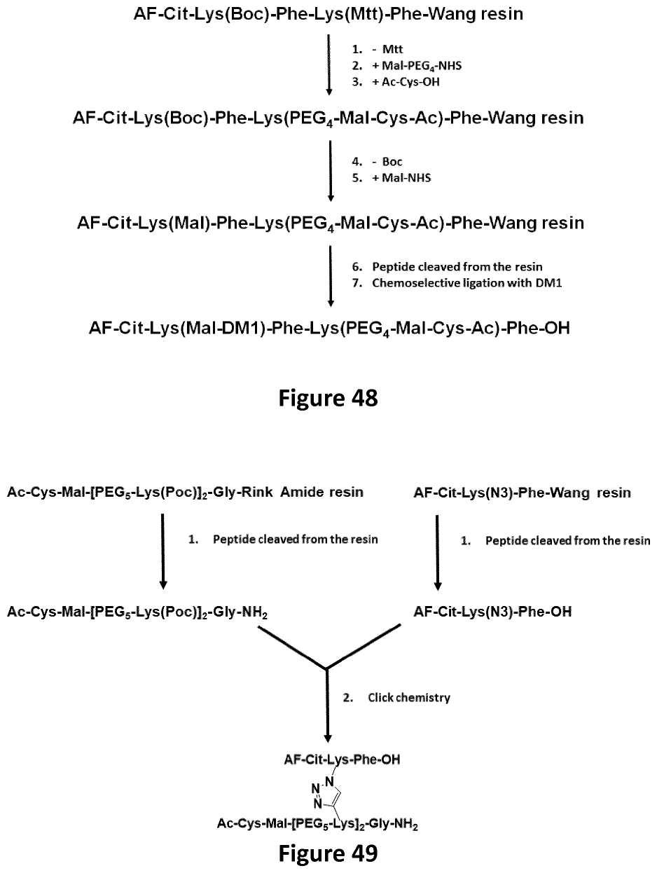

The compounds described herein were prepared using standard Fmoc-based SPPS, including on-resin peptide coupling and convergent strategies as shown in FIGS. 48 and 49. The compounds prepared in Example 4 are shown in Table 7 below.

TABLE 7
Compounds of formula (Ia)/(Ia₁)
suitable for multiple drug release;

| Compound | Structure |
|---|---|
| 31 | AF-Cit-Lys(Mal-DM1)-Phe-Lys |
| | (PEG₄-Mal-Cys-Ac)-Phe-OH |
| | (SEQ ID NO: 65) |
| 32 | Ac-Cys-Mal-[PEG₅-Lys(AF-Cit- |
| | Lys(Y)-Phe-OH)]₂-Gly-NH₂ |

Y = covalent ligation between moiety T and
drug-linker unit, e.g. by click-chemistry
(formation of triazole moiety)

For the synthesis of compounds 31, Auristatin F (AF) was coupled after Fmoc removal by fragment condensation (3 eq AF, 2.9 eq HBTU, 7 eq DIEA). Then, the derivative Mal-PEG₄-NHS was added on resin for 30 min (3 eq of Mal-PEG₄-NHS, 7 eq DIEA) after Mtt removal by DCM/TFA/TIS (94/1/5, v/v/v). Then, the maleimide residue on the PEG chain was reacted on resin with acetyl-cysteine (Ac-Cys-OH) via chemoselective ligation between maleimide and thiol (3 eq of Ac-Cys-OH, DIEA, 7 eq) during 20 min. The Mal-derivative was inserted by adding the moiety Mal-NHS to the side chain of Lys after Boc removal by DCM/TMSOTf/TEA (97/1/2, v/v/v).

The peptide was cleaved from the resin by treatment with TFA/TIS/water (95/2.5/2.5, v/v/v) during 60 min. After concentration of the cleavage mixture, the crude peptide was precipitated with cold diethyl ether and centrifuged. Then, Mertansine (DM1, 1.45 eq) was reacted with the N-terminal maleimide group via chemoselective ligation in PBS buffer at pH 7.4 and acetonitrile (ratio 2:1) (FIG. 48).

For the synthesis of compound 32, peptides Ac-Cys-Mal-[PEG₅-Lys(Poc)]₂-Gly-NH₂ (moiety T) and AF-Cit-Lys(N3)-Phe-OH (drug-linker) were prepared according to protocols described in Example 1 and 2. Derivatives Fmoc-Lys(Poc)-OH and Fmoc-Lys(N₃)—OH were used as alkyne and azide components for click chemistry. To this end, Ac-Cys-Mal-[PEG₅-Lys(Poc)]2-Gly-NH₂ (1 eq) was coupled in solution to AF-Cit-Lys(N₃)-Phe-OH (1 eq) following standard click-chemistry (FIG. 49).

The peptides were purified and analyzed as described in Example 1 above. The results of the analysis of the compounds obtained in Example 4 are shown in Table 8 below.

TABLE 8
Analysis of compounds 31-32

| | | Purity | | | |
|---|---|---|---|---|---|
| Compound | Formula | (%) | Mw | [M + 2H⁺]⁺² | [M + 3H⁺]⁺³ |
| 31 | C₁₆₇H₂₂₇Cl₂N₂₃O₄₆S₂ | 89 | 3459.8 | 1731.6 | 1154.1 |
| 32 | C₁₈₂H₂₉₂N₃₆O₄₉S | 85 | 3800.5 | 1901.8 | 1268.2 |

### Example 5: Cat B-Induced Cleavage Study Using Compounds 1 to 7, 19, 22 and 23 (Formula I/I′)

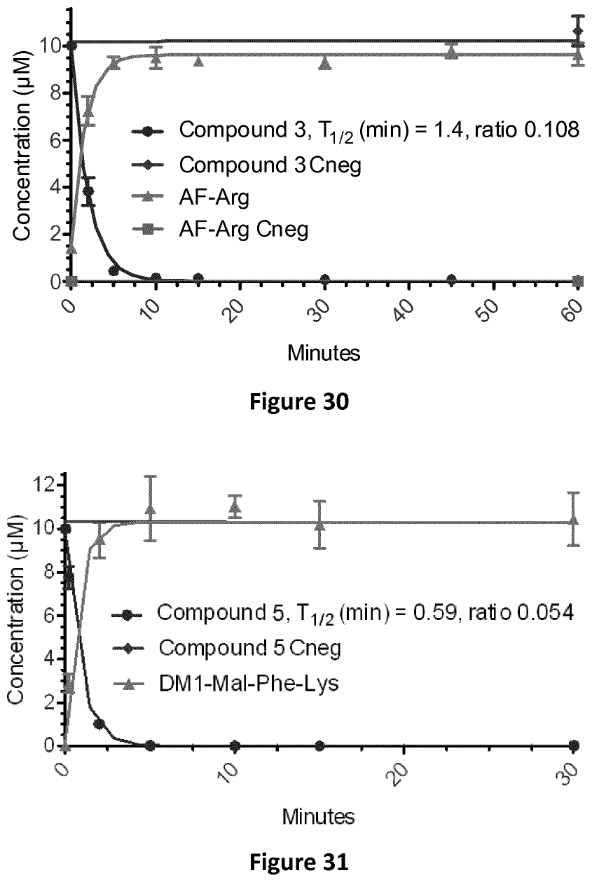

The propensity of compounds 1-7 and 19-23 (formula (I)/(I′)) to be cleaved by Cathepsin B—was evaluated using the in vitro enzymatic cleavage assay described above. The results are given in Table 9 below and shown in FIGS. 29-31.

TABLE 9
Cat B-induced cleavage study of compounds of formula (I)/(I′) (Reference
compound: Cys-MC-Val-Cit-PABC-MMAF)

| | | | Ratio |
|---|---|---|---|
| | T_{1/2} Compound | T_{1/2} Reference | (T_{1/2} Compound/ |
| Compound | (min) | (min) | T_{1/2} Reference) |
| 1 | 1.5 | 12.9 | 0.12 |
| 3 | 1.4 | 12.9 | 0.11 |
| 5 | 0.6 | 10.9 | 0.05 |
| 6 | 0.3 | 13.1 | 0.02 |
| 7 | 1.2 | 13.1 | 0.09 |
| 19 | 1.1 | 12.6 | 0.09 |
| 22 | 0.06 | 11.4 | 0.005 |
| 23 | 0.0004 | 11.4 | 0.00004 |

From these results, it is apparent that exo-Cat B cleavage and drug release (AF-Arg, AF-Cit, ACit, DM1-Mal-Phe-Lys, DM1-Mal-Phe-Cit, DM1-Mcc-Phe-Cit) in compounds of formula (I)/(I′) occurred simultaneously and were very fast. For instance, Cat B-induced drug release from compound 5 occurred 20 times faster as compared to the reference PABC compound Cys-MC-Val-Cit-PABC-MMAF. The fast cleavage kinetics achieved by compounds 1-7, 19 and 22-23 demonstrates that the compounds of the present invention exhibit high selectivity and binding affinity for the exopeptidase activity of Cathepsin B. Furthermore, it was surprisingly found that the presence of an Ac-Cys-PEG₄ moiety on the side-chain of the Lys residue (corresponding to residue Axx in formula (I or I′)) had no detrimental effect on the binding affinity of the compounds for Cat B. These results also indicate that, by contrast, cleavage by the endopeptidase based mechanism of Cat B as realized in the PABC linker systems (e.g. reference compound) occurs at significantly slower rates. As particular striking examples, compounds 22 and 23 are spontaneously cleaved by exo-Cat B (T ½<1 min), demonstrating the highly favorable binding properties of substrates based on formula (I); it is believed that the favorable interaction between the C-terminal Tyr and occluding loop of Cat B strongly contributes to the fast cleavage rate observed in these compounds.

### Example 6: Cat B-Induced Cleavage Study Using Compounds 8-13 and 27-28 (Formula II)

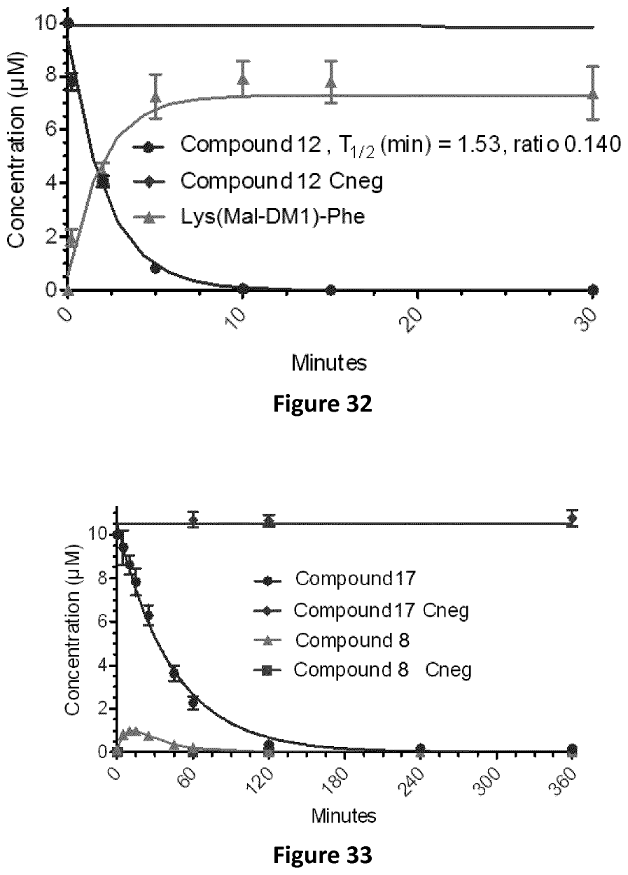

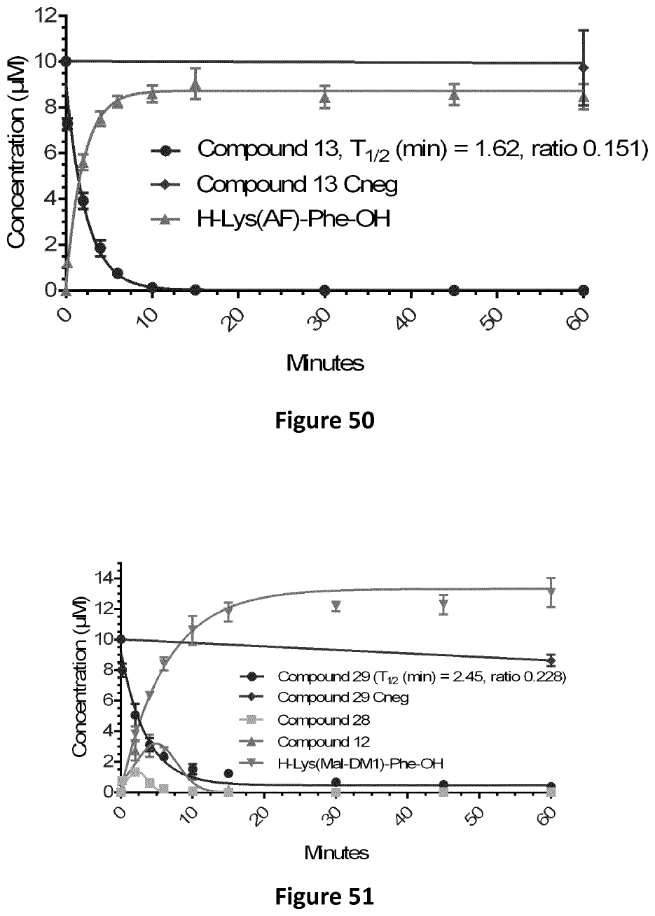

The propensity of compounds 8-13 and 27-28 (formula (II)) to be cleaved by Cathepsin B was evaluated using the in vitro enzymatic cleavage assay described above. The results are given in Table 10 below and shown in FIGS. 32 and 50.

TABLE 10
Cat B-induced cleavage study of compounds
of formula (II)/(II′) (Reference
compound: Cys-MC-Val-Cit-PABC-MMAF)

| | | | Ratio |
|---|---|---|---|
| | T_{1/2} Compound | T_{1/2} Reference | (T_{1/2} Compound/ |
| Compound | (min) | (min) | T_{1/2} Reference) |
| 8 | 2.2 | 11.6 | 0.19 |
| 9 | 4.5 | 7.2 | 0.63 |
| 10 | 0.9 | 10.2 | 0.1 |
| 11 | 2.2 | 11.1 | 0.2 |
| 12 | 1.5 | 10.9 | 0.14 |
| 13 | 1.6 | 10.7 | 0.15 |
| 27 | 0.06 | 11.4 | 0.005 |
| 28 | 0.3 | 10.7 | 0.03 |

These results demonstrate that Cat B-induced cleavage from compounds 8-13 carrying the vector attachment site at the N-terminus of the linker system was very fast.

Cleavage rates up to 10 times faster compared to the reference PABC linker-system were observed, thus indicating that compounds 8-13 were cleaved by the exopeptidase mechanism of Cat B. Surprisingly, the presence of sterically demanding drug moieties such as CPT, DM1 or AF at the side-chain of residue Bxx in formula (II) had no detrimental effect on the observed cleavage rates, indicating that the sterically demanding moieties were directed outside the binding groove of Cat B. In compound 8, drug release (CPT) occurs via acid or enzyme (esterase) catalyzed hydrolysis, whereas compounds 9 and 11 may undergo intramolecular aminolysis (cyclic urea or carbamate formation) for CPT release. Most notably, in compounds 12 and 13, the pharmacologically active moiety—i.e. H-Lys(Mal-DM1)-Phe-OH or H-Lys(AF)-Phe-OH — was released simultaneously by Cat B-induced cleavage.

Compound 27 demonstrates the importance of the C-terminal residue upon the cleavage rate. As observed above, Tyr (in compound 27) is prone for favorable interactions (presumably by H-bonding) leading to very fast cleavage via the exopeptidase activity of Cat B (ca 2300 fold faster as compared to the PABC reference).

### Example 7: Cat B-Induced Cleavage Study Using Multimeric Compounds (Releasing Multiple Drugs) as Per Formula (II)

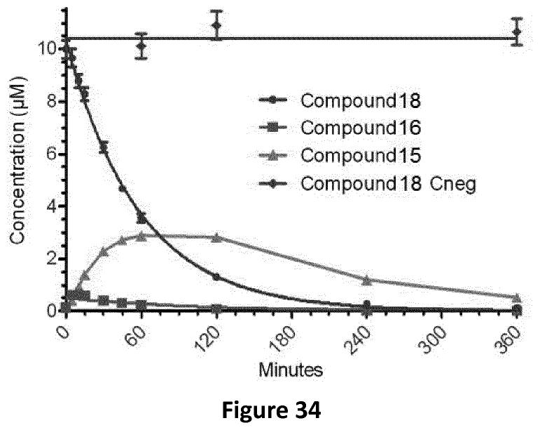

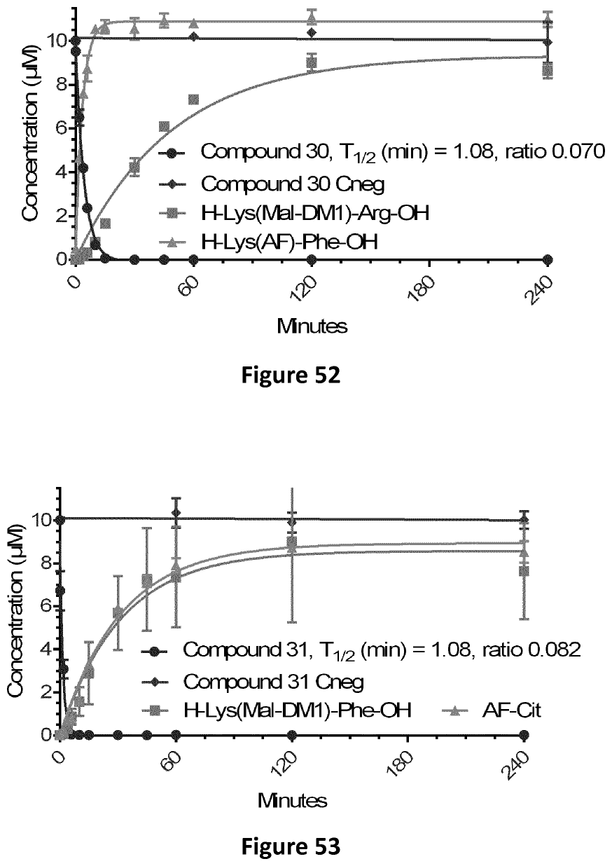

The propensity of the multimeric compounds 17-18 and 29-30 (formula (II)) to be cleaved by Cathepsin B was evaluated using the in vitro enzymatic cleavage assay described above. The results are shown in FIGS. 33-34 and in FIGS. 51-52.

As shown in FIG. 33, Cat B-induced cleavage of compound 17 rapidly released C-terminal dipeptide-drug unit Glu(Sar-OCPT)-Phe-OH and compound 8 as an intermediate, indicating that cleavage occurred according to exopeptidase mechanism of Cat B. In turn, compound 8 was rapidly cleaved to release the C-terminal dipeptide-drug unit H-Glu(Sar-OCPT)-Phe-OH. Each dipeptide-drug unit H-Glu(Sar-OCPT)-Phe-OH can in turn undergo acid- or enzyme-catalyzed hydrolysis to release native CPT.

As shown in FIG. 34, Cat B-induced cleavage of compound 18 rapidly released the C-terminal dipeptide-drug unit H-Glu(Sar-OCPT)-Arg-OH and compound 16 as a first intermediate, which is in turn rapidly cleaved via exo-Cat B mechanism to release compound 15 as a second intermediate. Cat B-induced cleavage of compound 15 releases a second C-terminal dipeptide-drug unit H-Glu(Sar-OCPT)-Arg-OH. Each dipeptide-drug unit H-Glu(Sar-OCPT)-Phe-OH can in turn undergo acid- or enzyme catalized hydrolysis to release native CPT. Owing to the identification of the expected intermediate compounds (HPLC and MS/MS), the selective cleavage according to the exopeptidase mechanism of Cat B could be established.

As shown in FIG. 51, Cat B-induced cleavage of compound 29 rapidly (ca 5-fold compared to reference PABC-system) released the C-terminal dipeptide-drug unit H-Lys(Mal-DM1)-Phe-OH and compound 28 as a first intermediate, which is in turn rapidly cleaved via exo-Cat B mechanism to release compound 12 as a second intermediate. Cat B-induced cleavage of compound 12 releases the second dipeptide-drug unit H-Lys(Mal-DM)-Phe-OH. Again, the identification of the expected intermediate compounds allows establishing the selective and fast cleavage according to the exopeptidase mechanism of Cat B.

As shown in FIG. 52, Cat B-induced cleavage of compound 30 rapidly released the C-terminal dipeptide-drug unit H-Lys(AF)-Phe-OH. After this very fast cleavage step (more than 10-fold compared to reference PABC-system), the intermediate drug-linker containing a C-terminal Arg (residue Byy) was cleaved moderately fast (T ½<30 min) to release the second (different) dipeptide-drug H-Lys(Mal-DM1)-Arg-OH. The sequential cleavage of the dipeptide-drugs clearly demonstrates the selective cleavage by the exopeptidase activity of Cat B.

The results for compounds 29 and 30 are given in Table 11.

TABLE 11
Cat B-induced cleavage study of compounds 29 and
30 releasing multiple drugs as per formula (II)
(Reference compound: Cys-MC-Val-Cit-PABC-MMAF)

| | | | Ratio |
|---|---|---|---|
| | T_{1/2} Compound | T_{1/2} Reference | (T_{1/2} Compound/ |
| Compound | (min) | (min) | T_{1/2} Reference) |
| 29 | 2.4 | 10.7 | 0.23 |
| 30 | 1.1 | 15.4 | 0.07 |

### Example 8: Cat B-Induced Cleavage Study Using Multimeric Compounds (Formula (I) and (I′))

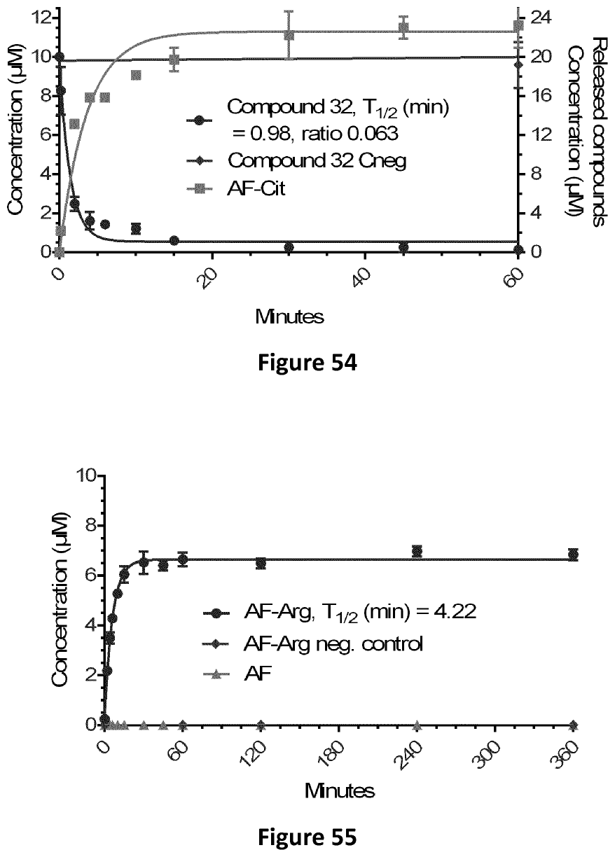

The propensity of the multimeric compounds 31-32 (formula (Ia and Ia1)) to be cleaved by Cat B was evaluated using the in vitro enzymatic cleavage assay described above. The results are given in Table 12 and are shown in FIGS. 53-54.

TABLE 12
Cat B-induced cleavage study of compounds releasing
multiple drugs as per formula (Ia) and (Ia₁) (Reference
compound: Cys-MC-Val-Cit-PABC-MMAF)

| | | | Ratio |
|---|---|---|---|
| | T_{1/2} Compound | T_{1/2} Reference | (T_{1/2} Compound/ |
| Compound | (min) | (min) | T_{1/2} Reference) |
| 31 | 1.08 | 12.7 | 0.08 |
| 32 | 0.98 | 15.4 | 0.06 |

As shown in FIG. 53, Cat B-induced cleavage of compound 31 rapidly released the C-terminal dipeptide unit, i.e. the vector containing H-Lys(PEG₄-Mal-Cys-Ac)-Phe-OH. After this very fast cleavage step (more than 10-fold compared to reference PABC-system), the intermediate dual drug-linker was cleaved to simultaneously release the different drugs AF-Cit and H-Lys(Mal-DM1)-Phe-OH, again proving the mechanism of exo-Cat B cleavage.

As shown in FIG. 54, Cat B-induced cleavage of compound 32 rapidly released drug AF-Cit (cleavage rate ca 20-fold compared to reference PABC-system). Most notably, the release of the 2 drug moieties AF-Cit occurred nearly spontaneously, confirming that the linker system is suited for increasing the DAR-values in ADCs. The data confirm the dual and synergistic function of formula (I) and (Ia1), i.e. enabling rapid drug release due to the linker of formula (I) and enhancing water solubility due to the solubilizing effect of the moiety of formula (Ia1).

### Example 9-Cytotoxic Activity of AF-Arg and AF

The in vitro cytotoxic activities of native AF and AF-Arg, i.e. a chemically modified drug in accordance with formula (III) wherein W₁ represents AF, Dxx represents a single covalent bond, and Dyy represents Arg, were evaluated in two ErbB2-expressing cell lines, namely SK-BR-3 and SK-OV-3 cells. The cytotoxic activity test was conducted according to the method described under item 11.3.4 above.

The results of the cytotoxic activity test at incubation times of 72 h and 120 h are given in respective Tables 13 and 14 below.

TABLE 13
Cytotoxicity study of AF and AF-Arg in ErbB2-expressing
SK-OV-3 and SK-BR-3 cells after 72 h

| Relative IC₅₀ (nM) at 72 h (day 3) | |
|---|---|
| SK-OV-3 | SK-BR-3 |

| Drug | run 1 | run 2 | run 1 | run 2 |
|---|---|---|---|---|
| AF | 82.79 | 153.8 | 37.43 | 53.66 |
| AF-Arg | 179.6 | 239.5 | ~98.02 | ~110.7 |

TABLE 14
Cytotoxicity study of AF and AF-Arg in ErbB2-expressing
SK-OV-3 and SK-BR-3 cells after 120 h

| Relative IC₅₀ (nM) at 120 h (day 5) | |
|---|---|
| SK-OV-3 | SK-BR-3 |

| Drug | run 1 | run 2 | run 1 | run 2 |
|---|---|---|---|---|
| AF | ~100.2 | 145.2 | 18.82 | 21.75 |
| AF-Arg | 146.7 | 204.7 | 20.9 | 24.92 |

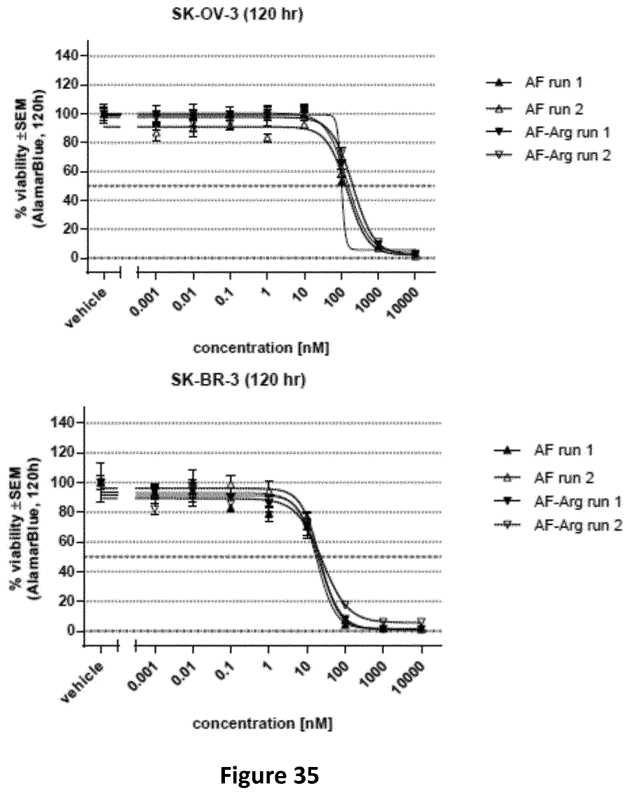

These results indicate that the chemically modified drug (AF-Arg)—retains cytotoxic activity, e.g. greater than 85% of the native drug (AF) cytotoxic activity in SK-BR-3 cells and about 70% in SK-OV-3 cells at 120 h (FIG. 35). Moreover, these results also indicate that the introduction of a divalent group, i.e. Arg (amino acid Dyy in formula (III)), between the drug and the linker system of the present invention does not detrimentally affect the pharmacological activity of the (modified) drug moiety released in the target cell. In particular, if the drug is internalized into the target cell via vectorization, the cytotoxicity of the drug (AF-Arg) is no longer attenuated by the reduced cell permeability of the modified drug due to the increase in polarity, i.e. the charged side chain of Arg.

### Example 10—Preparation of Antibody-Drug Conjugates

For the preparation of ADC1, a solution of commercial trastuzumab (10.0 mg, 0.066 μmol) in water (0.48 mL) and DPBS at pH 7.4 (0.52 mL) at room temperature (RT), was partially reduced by addition of a solution of tris(2-carboxyethyl)phosphine hydrochloride (0.058 mg, 0.24 μmol) in PBS pH 7.4 buffer (50 μL). After 60 min stirring, a solution of compound 2 (AF-Arg-Lys(PEG₄-Mal)-Phe-OH) (1.04 mg, 0.66 μmol) in DMSO (50 μL) was added. The reaction was stirred for 1 h at room temperature and dissolved with more PBS pH 7.4 buffer (1.92 mL). The solution was then loaded on the top of a Sephadex® PD-10 column (GE Healthcare) equilibrated with PBS pH 7.4 buffer. The first 2.5 mL of eluent generated upon loading was discarded. The column was further eluted with PBS pH 7.4 buffer (3.5 mL) and all eluents were collected. All suspended material were removed by centrifugation and the supernatant was concentrated in an Amicon® Centrifugal Filters Unit to a volume of 0.3 mL and dissolved in PBS pH 7.4 (7 mL).

For the preparation of ADC3, to a solution of commercial trastuzumab (50 mg) in water (2.38 mL) and DPBS at pH 7.4 (1.87 mL) at RT, was added a solution of tris(2-carboxyethyl)phosphine hydrochloride (0.38 mg, 1.33 μmol) in DPBS (450 μL). The reaction was stirred for 75 min. A solution of compound 26 (Ma-PEG₅-Phe-Cit-Lys(Mcc-DM1)-Cit-OH) (6.65 mg, 3.33 μmol) in DMSO (300 μL) was added to the reaction, which was stirred at RT for 60 min. 100 mL of commercial DPBS was adjusted to pH 8 with an aqueous solution of sodium hydroxide (1 mol/L). Two PD 10 columns were then pre-washed with the solution of DPBS (25 mL each) at pH 8. The reaction mixture was applied on the top of 2 columns (2.5 mL on each column). The eluents generated during the loading phase were discarded. The two columns were then eluted with pH 8 DPBS buffer (3.5 mL each). The collected eluents (2×3.5 mL) were combined and stirred for 15 h at room temperature to stabilize the thiomaleimide by ring opening. All suspended material were removed by centrifugation at 4000 rpm (10 min). The solution was split (2×3.5 mL) and transferred into 2 Amicon® Centrifugal Filters. The two solutions were concentrated by centrifugation at 4000 rpm for 2 h to reach a final volume of 0.5 mL in each cells. The two solutions were then combined. The membranes of the 2 filters were washed Dulbecco's PBS buffer (4 mL). The rinsing solution was added to the concentrated ADC to obtain the final ADC solution (V=5.0 mL).

The respective DAR values of ADCs 1 and 3 presented in Table 15 were determined in accordance with the method described in item 11.3.5 above.

TABLE 15
DAR values of ADC1 and ADC3

| Compound | Drug Linker | Formula | DAR | Concentration |
|---|---|---|---|---|
| ADC1 | Compound 2 | I | 4.2 | 1.17 mg/mL |
| ADC3 | Compound 26 | II | 4.4 | 10.4 mg/mL |

### Example 11: Cat B-Induced Cleavage Study Using ADC1

Cleavage of ADC1 by Cat B occurred fast as shown by the fast release of AF-Arg (T ½<5 min), confirming the mechanism by exo-Cat B activity in constructs of formula (I) (FIG. 55) The results show that the attachment of a mAb to the side chain of Lys does not lead to a decrease of the observed cleavage rate as compared to the model moiety V (-Cys-Ac).

ADC3 was similarly cleaved by exo-Cat B (data not shown).

### Example 12: Plasma Stability of ADCs

The analyses by UHPLC-MS/MS show that no free drug (AF nor AF-Arg for ADC1 or DM1 derivative for ADC3) was detected.

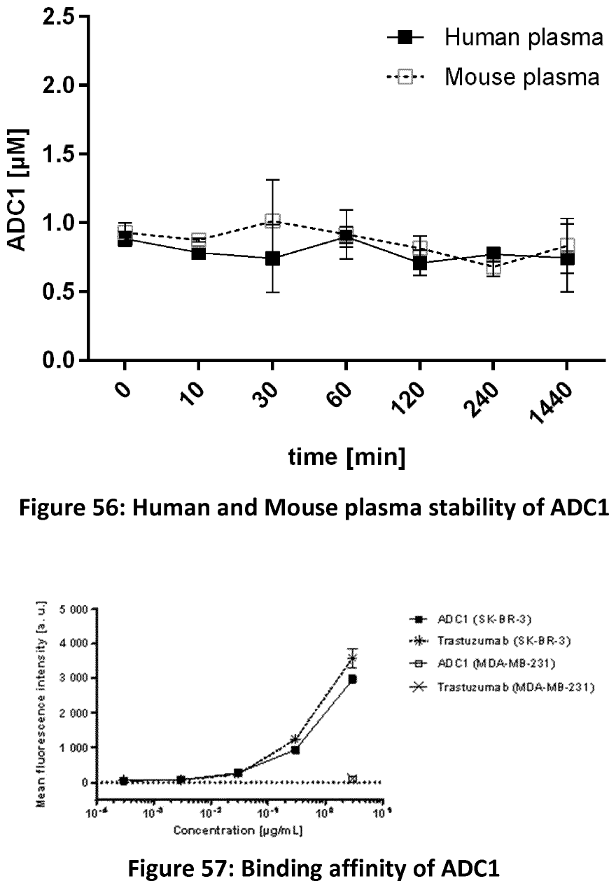

The graph (FIG. 56) shows the calculated mean ADC concentrations in human and mouse plasma samples. Error bars: SD (n=2). The results show that ADC1 was stable in mouse and human plasma over 24 h.

### Example 13: Binding Assays of ADCs

ADC1: Binding assay of ADC1 and trastuzumab on SK-BR-3 (ErbB2-expressing) and MDA-MB-231 (ErbB2 negative) cells showed that ADC1 has the same affinity and specificity for ErbB2 expressing cells than trastuzumab (FIG. 57).

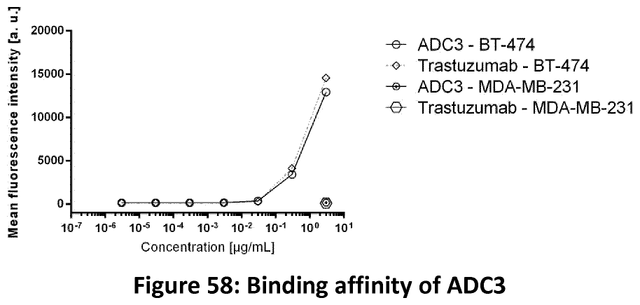

ADC3: Binding assay of ADC3 and trastuzumab on BT-474 (ErbB2-expressing) and MDA-MB-231 (ErbB2 negative) cells showed that ADC3 has the same affinity and specificity for ErbB2 expressing cells than trastuzumab (FIG. 58).

### Example 14: Cytotoxic Activity of ADCs

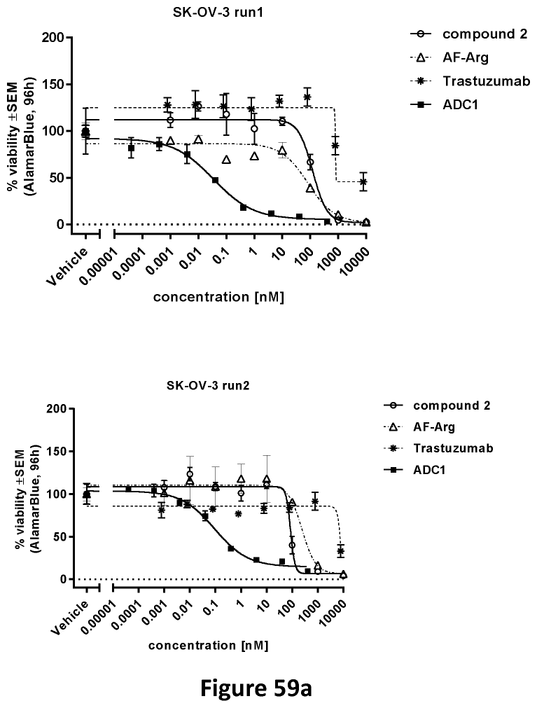

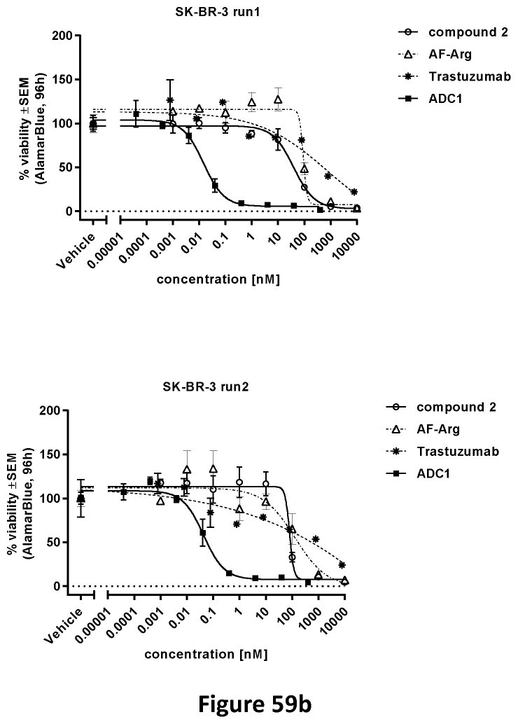

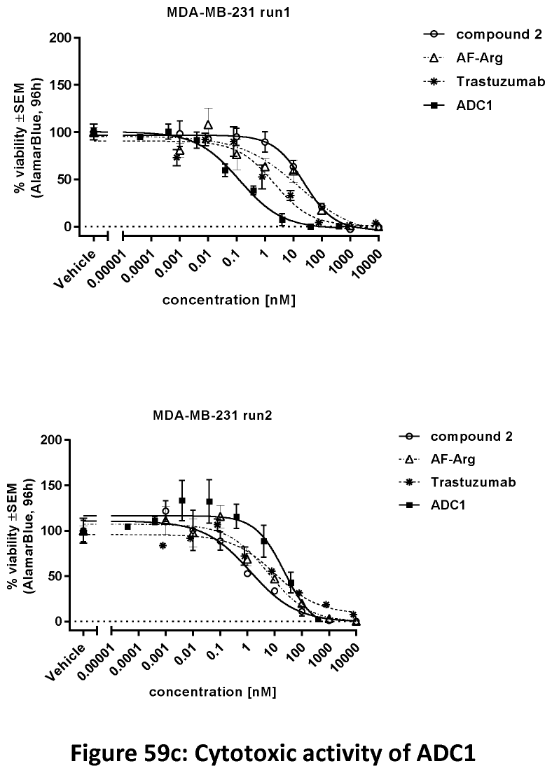

Cytotoxicity assay of ADC1 and derivatives (trastuzumab, compound 2 or AF-Arg) on ErbB2-expressing SK-OV-3 and SK-BR-3 cells and ErbB2-negative MDA-MB-231 cells was conducted according to the method described under item 11.3.4 above. This assay demonstrated the increased cytotoxic activity of ADC1 compared to the monoclonal antibody (trastuzumab), compound 2 or AF-Arg. FIG. 59(a)-(c) show the dose-response curves of two independent runs with the relative IC₅₀ values as determined with the Alamar Blue assay after 96 h of incubation. The corresponding results of the cytotoxic activity tests are given in Table 16 below.

TABLE 16
Cytotoxicity study of ADC1, trastuzumab, AF-Arg and compound 2 in ErbB2-expressing
SK-OV-3 and SK-BR-3 cells and in ErbB2 negative cells after 96h

| | Relative IC₅₀ (nM) at 96 h (day 4) | | | | | |
|---|---|---|---|---|---|---|
| | SK-OV-3 | | SK-BR-3 | | MDA-MB-231 | |
| Drug | run 1 | run 2 | run 1 | run 2 | run 1 | run 2 |
| Compound 2 | 122.7 | ~86.74 | 39.35 | ~80.51 | 23.7 | 1.288 |
| AF-Arg | 82.57 | 243.5 | ~92.13 | 128.3 | 15.23 | 5.844 |
| trastuzumab | ~793.4 | ~16122 | 759.9 | 15481127 | 2.144 | 10.89 |
| ADC1 | 0.03438 | 0.09391 | 0.01476 | 0.04327 | 0.1279 | 23.46 |

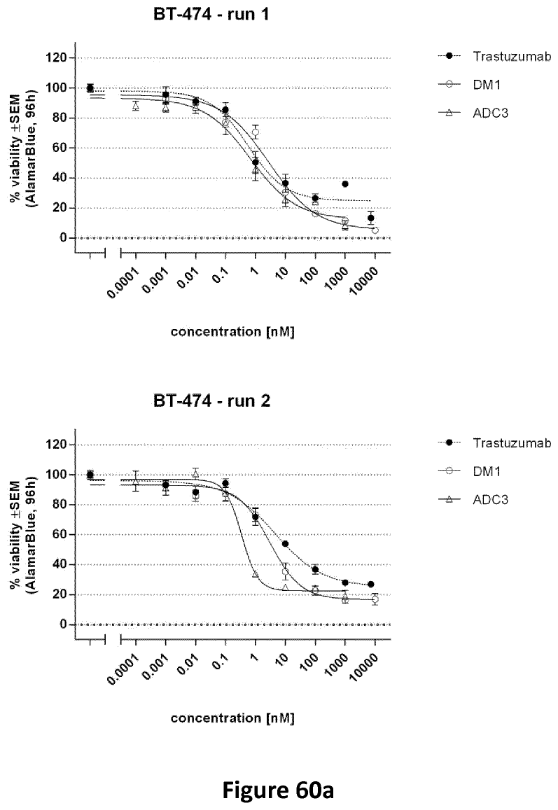

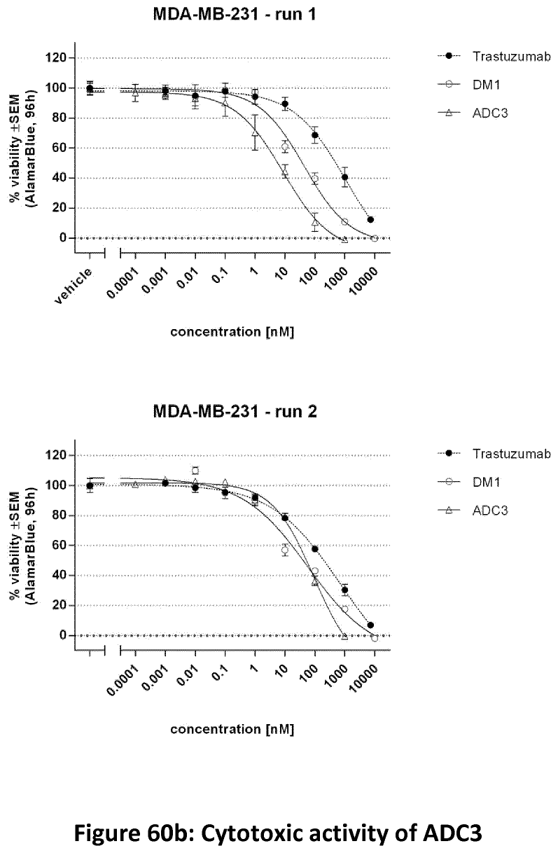

Cytotoxicity assay of ADC3 and derivatives (trastuzumab and DM1) on ErbB2-expressing BT-474 cells and ErbB2-negative MDA-MB-231 cells was conducted according to the method described under item 11.3.4 above. This assay confirmed the increased cytotoxic activity of AD3 compared to trastuzumab and DM1. FIG. 60(a)-(b) shows the dose-response curves of two independent runs with the relative 150 values as determined with the Alamar Blue assay after 96 h of incubation. The corresponding results of the cytotoxic activity tests are given in Table 17 below.

TABLE 17
Cytotoxicity study of ADC3, trastuzumab and DM1 in ErbB2-expressing
BT-474 cells and in ErbB2 negative cells after 96 h

| Relative IC₅₀ (nM) at 96 h (day 4) | |
|---|---|
| BT-474 | MDA-MB-231 |

| Drug | run 1 | run 2 | run 1 | run 2 |
|---|---|---|---|---|
| DM1 | 2.802 | 3.002 | 44.21 | 57.02 |
| Trastuzumab | 0.5711 | 4.558 | 1080 | 686 |
| ADC3 | 0.683 | 0.3456 | 8.689 | 91.79 |

1. A compound represented by the general formula (I) or (I′): 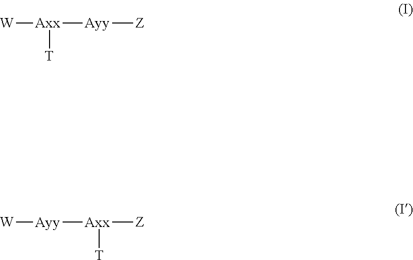
wherein, in formulae (I) and (I′),
W represents a moiety represented by the following formula (III):
W₁-Dxx-Dyy---- (III)
wherein
W₁ represents a moiety derived from a drug that differs from a native drug only by virtue of the covalent attachment to Dxx as shown in formula (III), if the drug is an auristatin analog, the auristatin analog is auristatin Phe (AF), auristatin Cit (ACit), auristatin Arg (AArg), auristatin Lys (ALys), auristatin Orn (AOrn), auristatin Dab (ADab) or auristatin Dap (ADap);
Dxx represents a single covalent bond or an amino acid having a hydrophobic side chain, wherein the single covalent bond or amino acid having a hydrophobic side chain is optionally attached to moiety W₁ via a divalent moiety selected from maleimides, triazoles, hydrazones, carbonyl-containing groups, and derivatives thereof;
Dyy represents a single covalent bond, Phe or an amino acid having a basic side chain,
with the proviso that if Dxx is an amino acid having a hydrophobic side chain, Dyy is Phe or an amino acid having a basic side chain, and if Dxx is a single covalent bond, Dyy is a single covalent bond, Phe or an amino acid with a basic side chain;
and the broken line indicates covalent attachment to the N-terminus of Axx in formula (I) or the N-terminus of Ayy in formula (I′);
Axx represents a trifunctional amino acid; with the proviso that Axx in formula (I) is not an amino acid in the (D) configuration;
Ayy represents an amino acid selected from Phe, Ala, Trp, Tyr, Phenylglycine (Phg), Met, Val, His, Lys, Arg, Cit, 2-amino-butyric acid (Abu), Orn, Ser, Thr, Leu and Ile; or Ayy in formula (I) represents an amino acid selected from homo-tyrosine (homo-Tyr), homo-phenylalanine (homo-Phe), beta-phenylalanine (beta-Phe) and beta-homo-phenylalanine (beta-homo-Phe), Tyr(OR₁) and homo-Tyr(OR₁) wherein R₁ is —(CH₂CH₂O)ₙ₁—R₂, wherein R₂ is a hydrogen atom or a methyl group and n1 is an integer of 2 to 24; with the proviso that Ayy in formula (I′) is not an amino acid in the (D) configuration;
T is a moiety being represented by the following formula (Ia₁): 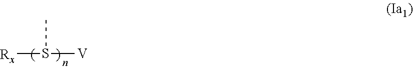
wherein, in formula (Ia₁),
S represents a group containing one or more atoms selected from carbon, nitrogen, oxygen, and sulfur;
V represents a moiety derived from a vector group capable of interacting with a target cell;
n is an integer of 1 to 10;
Rₓ is an atom or group which is optionally present to saturate a free valency of S, if present;
and the broken line indicates covalent attachment to the side chain of Axx; if n is more than 1, each broken line represents a covalent bond to an individual, separate group of formula (I) or formula (I′), wherein multiple groups of formula (I) or formula (I′) can be the same or different; if n is more than 1, each S can be the same or different;
Z represents a group covalently bonded to the C-terminus of Ayy or Axx selected from —OH; —N(H)(R), wherein R represents a hydrogen atom, an alkyl group, a cycloalkyl group or an aromatic group; and a labeling agent.

2. The compound of claim 1, 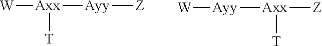
W₁-Dxx-Dyy----
wherein
W₁ is not an auristatin analog.

3. A compound represented by the general formula (I) or (I′): 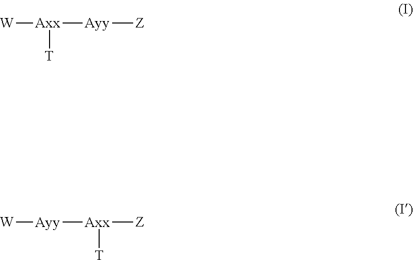
wherein, in formulae (I) and (I′),
W represents a peptide moiety represented by formula (Ia), (Ia′) or (Ib): 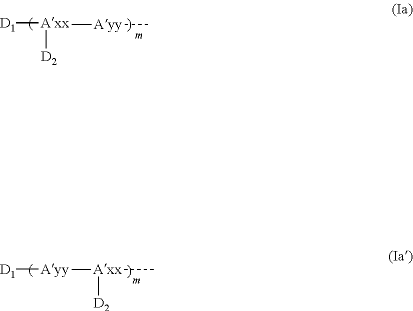
wherein, in formulae (Ia) and (Ia′),
A′yy represents an amino acid selected from Phe, Ala, Trp, Tyr, Phg, Met, Val, His, Lys, Arg, Cit, Abu, Orn, with the proviso that A′yy in formula (Ia′) is not an amino acid in the (D) configuration;
D₁ represents a moiety derived from a drug;
m is an integer of 1 to 10;
if m=1, then A′xx represents a trifunctional amino acid with the proviso that A′xx in formula (Ia) is not an amino acid in the (D) configuration, D₂ represents a moiety derived from a drug, optionally a moiety derived from the same drug as D₁;
if m is more than 1, then each D₂ is independently selected from a hydrogen atom and moieties derived from a drug, wherein multiple moieties D₂ can be the same or different with the proviso that at least one D₂ is not a hydrogen atom, if D₂ is a hydrogen atom then A′xx represents an amino acid with the proviso that A′xx in formula (Ia) is not an amino acid in the (D) configuration, if D₂ is a moiety derived from a drug, then A′xx represents a trifunctional amino with the proviso that A′xx in formula (Ia) is not an amino acid in the (D) configuration;
and the broken line indicates covalent attachment to the N-terminus of Axx or Ayy; 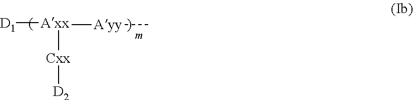
wherein, in formula (Ib),
A′yy represents an amino acid selected from Phe, Ala, Trp, Tyr, Phg, Met, Val, His, Lys, Arg, Cit, Abu, Orn;
D₁ represents a moiety derived from a drug;
m is an integer of 1 to 10;
if m=1, then A′xx represents a trifunctional amino acid selected from Glu, α-amino adipic acid (Aaa), Dap, Dab, Ser, Thr, homo-serine (homo-Ser), homo-threonine (homo-Thr) and amino-malonic acid (Ama) with the proviso that A′xx is not an amino acid in the (D) configuration; D₂ represents a moiety derived from a drug, optionally a moiety derived from the same drug as D₁, Cxx represents a single covalent bond unless A′xx is Ama, if A′xx is Ama, Cxx represents (L)- or (D)-Pro, or an N-methyl amino acid, the N-terminus of Cxx binds to a carboxyl end of Ama and the C-terminus of Cxx binds to a moiety D₂;
if m is more than 1, then each D₂ is independently selected from a hydrogen atom and moieties derived from a drug, wherein multiple moieties D₂ can be the same or different with the proviso that at least one D₂ is not a hydrogen atom, if D₂ is a hydrogen atom then A′xx represents an amino acid with the proviso that A′xx is not in the (D) configuration and Cxx represents a single covalent bond, if D₂ is a moiety derived from a drug then A′xx represents an amino acid selected from Glu, Aaa, Dap, Dab, Ser, Thr, homo-Ser, Homo-Thr and Ama with the proviso that A′xx is not an amino acid in the (D) configuration, Cxx represents a single covalent bond unless A′xx is Ama, if A′xx is Ama, Cxx represents (L)- or (D)-Pro, or an N-methyl amino acid such-as-Sam wherein the N-terminus of Cxx binds to a carboxyl end of Ama and the C-terminus of Cxx binds to moiety D₂;
and the broken line indicates covalent attachment to the N-terminus of Axx or Ayy;
Axx represents a trifunctional amino acid; with the proviso that Axx in formula (I) is not an amino acid in the (D) configuration;
Ayy represents an amino acid selected from Phe, Ala, Trp, Tyr, Phg, Met, Val, His, Lys, Arg, Cit, Abu, Orn, Ser, Thr, Leu and Ile; or Ayy in formula (I) represents an amino acid selected from homo-Tyr, homo-Phe, beta-Phe and beta-homo-Phe, Tyr(OR₁) and homo-Tyr(OR₁) wherein R₁ is —(CH₂CH₂O)ₙ₁—R₂, wherein R₂ is a hydrogen atom or a methyl group and n₁ is an integer of 2 to 24; with the proviso that Ayy in formula (I′) is not an amino acid in the (D) configuration;
T is a moiety represented by the following formula (Ia₁): 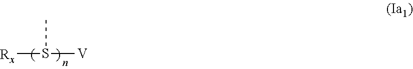
wherein, in formula (Ia₁),
S represents a group containing one or more atoms selected from carbon, nitrogen, oxygen, and sulfur;
V represents a moiety derived from a vector group capable of interacting with a target cell;
n is an integer of 1 to 10;
Rₓ is an atom or group which is optionally present to saturate a free valency of S, if present;
and the broken line indicates covalent attachment to the side chain of Axx; if n is more than 1, each broken line represents a covalent bond to an individual, separate group of formula (I) or formula (I′), wherein multiple groups of formula (I) or formula (I′) can be the same or different; if n is more than 1, each S can be the same or different;
Z represents a group covalently bonded to the C-terminus of Ayy or Axx selected from —OH; —N(H)(R), wherein R represents a hydrogen atom, an alkyl group, a cycloalkyl group or an aromatic group; and a labeling agent.

4. The compound of claim 1, wherein at least one of Axx and Ayy is defined as follows:
Axx represents an amino acid selected from Glu, 2-amino-pimelic acid (Apa), Aaa, Dap, Dab, Lys, Orn, Ser, Ama, and homo-lysine (homo-Lys);
Ayy in formula (I) represents an amino acid selected from Phe, homo-Phe, Ala, Trp, Phg, Leu, Val, Tyr, homo-Tyr, Tyr(ORI) and homo-Tyr(ORI) wherein R₁ is —(CH₂CH₂O)ₙ₁—R₂, wherein R₂ is a hydrogen atom or a methyl group and n1 is an integer of 2 to 24;
Ayy in formula (I′) represents an amino acid selected from Phe, homo-Phe, Ala, Trp, Phg, Leu, Val, Tyr and Ser.

5. The compound of claim 3, wherein at least one of A′xx, A′yy and m is defined as follows:
A′xx in formulae (Ia) and (Ia′) represents an amino acid selected from Dap, Dap, Lys, Orn and homo-Lys;
A′yy in formulae (Ia), (Ia′) and (Ib) represents an amino acid selected from Phe, Ala, Trp, Phg and Tyr; and
m is an integer of 1 to 4.

6. A compound represented by one of the following general formulae (II), (II′) and (IIa): 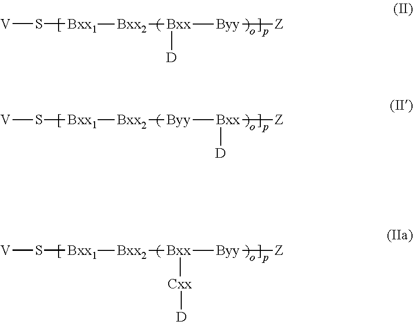
wherein,
D represents a moiety derived from a drug; if o*p>1 one or more D's may be hydrogen or a solubilizing group, with the proviso that at least one D represents a moiety derived from a drug;
Bxx in formulae (II) and (II′) represents a trifunctional amino acid; with the proviso that Bxx in formula (II) is not an amino acid in the (D) configuration;
Bxx in formula (IIa) represents a carboxylic amino acid or a trifunctional amino acid selected from Dap, Dab, Ser, Thr, Lys, Orn, homoLys, homoSer and homoThr; with the proviso that Bxx is not an amino acid in the (D) configuration; Cxx represents a single covalent bond unless Bxx is Ama; if Bxx is Ama, Cxx represents (L)- or (D)-Pro, or an N-methyl amino acid, the N-terminus of Cxx binds to a carboxyl end of Ama and the C-terminus of Cxx binds to moiety D;
in those instances where Bxx in formulae (II), (II′) and (IIa) carries a hydrogen as D group, Bxx is any other amino acid, with the proviso that Bxx in formulae (II) and (IIa) is not an amino acid in the (D) configuration;
Byy represents an amino acid selected from Phe, homo-Phe, Ala, Trp, Tyr, Phg, Val, His, Lys, Abu, Met, Cit, Orn, Ser, Thr, Leu, Ile, Arg and Tyr(OR₁) wherein R₁ is —(CH₂CH₂O)ₙ₁— R₂, wherein R₂ is a hydrogen atom or a methyl group and n1 is an integer of 2 to 24; or Byy in formulae (II) and (IIa) represents an amino acid selected from homo-Tyr, homo-Tyr(ORI), homo-Phe, beta-Phe and beta-homo-Phe; with the proviso that Byy in formula (II′) is not an amino acid in the (D) configuration and with the proviso that if o*p>1, only the C-terminal Byy in formulae (II) and (IIa) may represent an amino acid selected from beta-Phe and beta-homo-Phe;
Bxx₁ represents a single covalent bond or an amino acid having a hydrophobic or basic side chain;
Bxx₂ represents an amino acid having a hydrophobic or basic side chain;
S represents a group containing one or more atoms selected from carbon, nitrogen, oxygen, and sulfur;
V represents a moiety derived from a vector group capable of interacting with a target cell;
Z is covalently bonded to the C-terminus of Byy in formulae (II) and (IIa) and the C-terminus of Bxx in formula (II′), and represents a group selected from —OH; —N(H)(R), wherein R represents a hydrogen atom, an alkyl group, a cycloalkyl group or an aromatic group; and a labelling agent; and
o and p each independently is an integer of 1 to 10, if p is more than 1, Bxx₁ is not an amino acid in the (D) configuration; if p is more than 1 or if o is more than 1, or if both p and o are more than 1, then each D is independently selected from moieties derived from a drug.

7. The compound of claim 6, wherein at least one of Bxx₁, Bxx₂, Bxx, Byy, o and p is defined as follows:
Bxx₁ represents a single covalent bond or an amino acid selected from Phe, homo-Phe, Phg, Val, Ser, Tyr, Ala, Leu, Ile;
Bxx₂ represents an amino acid selected from Arg, Lys, Cit, Val, Leu, Ser, Ala, Gly, His, Gln, Phg and Phe;
Bxx in formulae (II) and (II′) represents an amino acid selected from Dap, Dab, Lys, Orn, Ser, Glu, Ama, Thr, Tyr, Aaa, homo-Ser and homo-Thr;
Byy represents Cit, Phe, homo-Phe, Ser, Trp, Tyr or Tyr(OR₁) wherein R₁ is —(CH₂CH₂O)ₙ₁—R₂, wherein R₂ is a hydrogen atom or a methyl group and n1 is an integer of 2 to 24, and if o*p>1, Byy represents Tyr or Tyr(OR₁); and
o and p each independently is an integer of 1 to 4.

8. The compound of claim 1, wherein in formulae (Ia₁), (II), (II′) and (IIa),
S represents a divalent group selected from a divalent alkylene group, a divalent alkenylene group, a divalent alkynylene group, and a divalent polyalkylene oxide.

9. The compound of claim 1, wherein in formulae (Ia₁), (II), (II′) and (IIa),
S represents a divalent group having formula —(CH₂)_{q}-Azz₅-Y—, or a divalent group having formula —(OCH₂CH₂)_{q}-Azz₅-Y—;
wherein Y represents a divalent moiety covalently bonded to the C-terminus of Azz₅ and to moiety V; if Azz₅ is absent, Y is covalently bonded to the alkyl group or polyethylene oxide group and to moiety V; Y is derived from a compound selected from maleimides, triazoles, especially 1,2,3-triazole, hydrazones, carbonyl-containing groups, and derivatives thereof; q is an integer of 1 to 50; and Azz₅ is absent, or represents a solubilizing group selected from an amino acid and a divalent group containing an ammonium group, a sulfate group, a sulfonate group or a pyrophosphate diester group.

10. The compound of claim 1, wherein the compound of formula (I) and formula (Ia) is selected from W₁-Arg-Lys(T)-Phe-Z, W₁-Arg-Lys(T)-homoPhe-Z, W₁-Cit-Lys(T)-Phe-Z, W₁-Cit-Lys(T)-Tyr-Z, W₁—Cit-Lys(T)-homoTyr-Z, W₁-Lys(T)-Phe-Z, W₁-Lys(T)-Tyr-Z, W₁-Lys(T)-homoTyr-Z, W₁-Mal-Phe-Cit-Lys(T)-Phe-Z, W₁-Mal-Phe-Cit-Lys(T)-Tyr-Z, W₁-Mal-Phe-Cit-Lys(T)-homoTyr-Z, W₁-Mal-Phe-Lys-Lys(T)-Phe-Z, W₁-Mal-homoPhe-Arg-Lys(T)-Phe-Z, W₁-Mal-homoPhe-Cit-Lys(T)-Tyr-Z, W₁-Mal-homoPhe-Cit-Lys(T)-Tyr(OR₁)—Z wherein R₁ is —(CH₂CH₂O)ₙ₁—R₂, wherein R₂ is a hydrogen atom or a methyl group and n1 is an integer of 2 to 24, W₁-Mal-Cit-Lys(T)-Tyr-Z, W₁-Mal-Cit-Lys(T)-homoTyr-Z, W₁-Mal-Arg-Lys(T)-homoTyr-Z, W₁-Cit-(Lys(D₂)-Phe)ₘ-Lys(T)-Phe-Z, W₁-Cit-(Lys(D₂)-Phe)ₘ-Lys(T)-homoTyr-Z, W₁-Cit-(Lys(D₂)-Phe)ₘ-Lys(T)-Tyr(OR₁)—Z wherein R₁ is —(CH₂CH₂O)ₙ₁—R₂, wherein R₂ is a hydrogen atom or a methyl group and n1 is an integer of 2 to 24, W₁-(Lys(D₂)-Phe)ₘ-Lys(T)-Phe-Z, W₁-Phe-(Phe-Lys(D₂))ₘ-Lys(T)-Tyr-Z, W₁-(Phe-Lys(D₂))ₘ-Lys(T)-Tyr-Z, W₁-Phe-(Phe-Lys(D₂))ₘ-Lys(T)-homoTyr-Z and W₁-Arg-(Phe-Lys(D₂))ₘ-Lys(T)-Tyr(OR₁)—Z;
and the compound of formula (I′) is selected from W₁-Arg-Phe-Lys(T)-Z, W₁-Arg-Ser-Lys(T)-Z, W₁-Cit-Phe-Lys(T)-Z, W₁-Cit-Ser-Lys(T)-Z, W₁-Cit-homoPhe-Lys(T)-Z, W₁-Phe-Lys(T)-Z, W₁-Ser-Lys(T)-Z, W₁-Mal-Phe-Cit-Phe-Lys(T)-Z, W₁-Mal-homoPhe-Cit-Phe-Lys(T)-Z, W₁-Mal-Phe-Arg-Phe-Lys(T)-Z, W₁-Mal-Cit-Phe-Lys(T)-Z, W₁-Mal-Phe-Ser-Lys(T)-Z, W₁-Mal-Ala-Phe-Lys(T)-Z, W₁-Mal-Cit-Ser-Lys(T)-Z and W₁-Mal-Arg-homoPhe-Lys(T)-Z.

11. The compound of claim 6 which is selected from from V—S-Phe-Arg-Phe-Lys(D)-Ser-Lys(D)-Z, V—S-Phe-Arg-(Phe-Lys(D))ₒ—Z, V—S-Phe-Arg-(Ser-Lys(D))ₒ—Z, V—S-Phe-Arg-(Tyr(OR₁)-Lys(D))ₒ—Z, and V—S-Phe-Arg-(Phe-Lys(D))ₒ-Phe-Tyr(OR₁)—Z;
wherein Z is —OH.

12. The compound of claim 1, wherein each moiety derived from a drug is independently selected from:
(i) antineoplastic agents;
(ii) immunomodulatory agents;
(iii) anti-infectious disease agents;
and radioisotopes, pharmaceutically acceptable salts thereof and combinations thereof.

13. The compound of claim 1, wherein each moiety derived from a drug is independently derived from amanitin, duocarmycin, auristatin, maytansine, tubulysin, calicheamicin, camptothecin, SN-38, taxol, daunomycin, vinblastine, doxorubicin, methotrexate, pyrrolobenzodiazepine, or radioisotopes, pharmaceutically acceptable salts thereof, or combinations thereof.

14. The compound of claim 3, wherein each moiety D₁ is independently represented by the following formula (III):
W₁-Dxx-Dyy---- (III)
wherein:
W₁ represents a moiety derived from amanitin, duocarmycin, auristatin, maytansine, tubulysin, calicheamicin, camptothecin, SN-38, taxol, daunomycin, vinblastine, doxorubicin, methotrexate, pyrrolobenzodiazepine, or radioisotopes, pharmaceutically acceptable salts thereof or combinations thereof;
Dxx represents a single covalent bond or an amino acid having a hydrophobic side chain, wherein the single covalent bond or amino acid having a hydrophobic side chain is optionally attached to moiety W₁ via a divalent moiety selected from maleimides, triazoles, hydrazones, carbonyl-containing groups, and derivatives thereof;
Dyy represents a single covalent bond, Phe or an amino acid having a basic side chain;
with the proviso that if Dxx is an amino acid having a hydrophobic side chain, Dyy is Phe or an amino acid having a basic side chain, and if Dxx is a single covalent bond, Dyy is a single covalent bond, Phe or an amino acid with a basic side chain;
and the broken line indicates covalent attachment to the N-terminus of Axx in formula (I), the N-terminus of Ayy in formula (I′), the N-terminus of A′xx in formulae (Ia) and (Ib), or the N-terminus A′yy in formula (Ia′).

15. The compound of claim 3, wherein each moiety D₂ and D is independently represented by the following formula (IIIa):
W₂-Exx---- (IIIa)
wherein
W₂ represents a moiety derived from amanitin, duocarmycin, auristatin, maytansine, tubulysin, calicheamicin, camptothecin, SN-38, taxol, daunomycin, vinblastine, doxorubicin, methotrexate, pyrrolobenzodiazepine, or radioisotopes, pharmaceutically acceptable salts thereof or combinations thereof;
Exx represents a single covalent bond or a divalent moiety selected from maleimides, triazoles, hydrazones, carbonyl-containing groups, amino acids, dipeptide moieties and derivatives thereof;
and the broken line indicates covalent attachment to the side chain of A′xx in formulae (Ia) and (Ia′), the side chain of A′xx or C-terminus of Cxx if present in formula (Ib), the side chain of Bxx in formulae (II) and (II′), the side chain of Bxx or C-terminus of Cxx if present in formula (IIa).

16. The compound of claim 1, wherein V represents a moiety derived from a vector group selected from antibodies, antibody fragments, proteins, peptides and non-peptidic molecules.

17. The compound of claim 1, wherein
V represents a moiety derived from a vector group capable of interacting with a target cell, wherein the target cell is selected from tumor cells, virus infected cells, microorganism infected cells, parasite infected cells, cells involved in autoimmune diseases, activated cells, myeloid cells, lymphoid cells, melanocytes and infectious agents.

18. A composition comprising a therapeutically effective amount of the compound of claim 1, or a pharmaceutically acceptable salt thereof, and one or more components selected from a carrier, a diluent and other excipients.

19-20. (canceled)

21. A method for treating or preventing a cancer, an autoimmune disease, an infectious disease, or combinations thereof, comprising administering a therapeutically effective amount of the compound of claim 1 to a patient in need thereof.

22. The method of claim 21, wherein the compound is administered concurrently with, before or after one or more other therapeutic agents or therapies or other compounds of formula (I) or (I′).


SEQUENCE LISTING (125 sequences)

SEQ ID NO: 1 - protein, 22 aa, Artificial Sequence
  Synthetic Sequence
  VARIANT (1)..(1): Xaa is an amino acid having a hydrophobic side chain or is absent
  VARIANT (2)..(2): Xaa is an amino acid having a hydrophobic or basic side chain
  VARIANT (3)..(3): Xaa is Phe, a trifunctional amino acid such as an amino-dicarboxylic acid or a diamino-carboxylic acid, preferably selected from Glu, Asp, Aaa, Lys, Dap, Dab, Ser, Thr, homo-Ser and homo-Thr
  VARIANT (4)..(4): Xaa is Phe, Ala, Trp, Tyr, Phg, Val, His, Lys, Abu, Met, Cit, Orn, Ser, Thr, Leu, Ile, Arg, homo-Tyr, homo-Phe, beta-Phe, or beta-homo-Phe
  VARIANT (5)..(5): Xaa is Phe, a trifunctional amino acid such as an amino-dicarboxylic acid or a diamino-carboxylic acid, preferably selected from Glu, Asp, Aaa, Lys, Dap, Dab, Ser, Thr, homo-Ser and homo-Thr or absent
  VARIANT (6)..(6): Xaa is absent if Xaa at position 5 is absent, or Xaa is Phe, Ala, Trp, Tyr, Phg, Val, His, Lys, Abu, Met, Cit, Orn, Ser, Thr, Leu, Ile, Arg, homo-Tyr, homo-Phe, beta-Phe, or beta-homo-Phe
  VARIANT (7)..(7): Xaa is Phe, a trifunctional amino acid such as an amino-dicarboxylic acid or a diamino-carboxylic acid, preferably selected from Glu, Asp, Aaa, Lys, Dap, Dab, Ser, Thr, homo-Ser and homo-Thr or absent
  VARIANT (8)..(8): Xaa is absent if Xaa at position 7 is absent, or Xaa is Phe, Ala, Trp, Tyr, Phg, Val, His, Lys, Abu, Met, Cit, Orn, Ser, Thr, Leu, Ile, Arg, homo-Tyr, homo-Phe, beta-Phe, or beta-homo-Phe
  VARIANT (9)..(9): Xaa is Phe, a trifunctional amino acid such as an amino-dicarboxylic acid or a diamino-carboxylic acid, preferably selected from Glu, Asp, Aaa, Lys, Dap, Dab, Ser, Thr, homo-Ser and homo-Thr, or absent
  VARIANT (10)..(10): Xaa is absent if Xaa at position 9 is absent, or Xaa is Phe, Ala, Trp, Tyr, Phg, Val, His, Lys, Abu, Met, Cit, Orn, Ser, Thr, Leu, Ile, Arg, homo-Tyr, homo-Phe, beta-Phe, or beta-homo-Phe
  VARIANT (11)..(11): Xaa is Phe, a trifunctional amino acid such as an amino-dicarboxylic acid or a diamino-carboxylic acid, preferably selected from Glu, Asp, Aaa, Lys, Dap, Dab, Ser, Thr, homo-Ser and homo-Thr, or absent
  VARIANT (12)..(12): Xaa is absent if Xaa at position 11 is absent, or Xaa is Phe, Ala, Trp, Tyr, Phg, Val, His, Lys, Abu, Met, Cit, Orn, Ser, Thr, Leu, Ile, Arg, homo-Tyr, homo-Phe, beta-Phe, or beta-homo-Phe
  VARIANT (13)..(13): Xaa is Phe, a trifunctional amino acid such as an amino-dicarboxylic acid or a diamino-carboxylic acid, preferably selected from Glu, Asp, Aaa, Lys, Dap, Dab, Ser, Thr, homo-Ser and homo-Thr, or absent
  VARIANT (14)..(14): Xaa is absent if Xaa at position 13 is absent, or Xaa is Phe, Ala, Trp, Tyr, Phg, Val, His, Lys, Abu, Met, Cit, Orn, Ser, Thr, Leu, Ile, Arg, homo-Tyr, homo-Phe, beta-Phe, or beta-homo-Phe
  VARIANT (15)..(15): Xaa is Phe, a trifunctional amino acid such as an amino-dicarboxylic acid or a diamino-carboxylic acid, preferably selected from Glu, Asp, Aaa, Lys, Dap, Dab, Ser, Thr, homo-Ser and homo-Thr, or absent
  VARIANT (16)..(16): Xaa is absent if Xaa at position 15 is absent, or Xaa is Phe, Ala, Trp, Tyr, Phg, Val, His, Lys, Abu, Met, Cit, Orn, Ser, Thr, Leu, Ile, Arg, homo-Tyr, homo-Phe, beta-Phe, or beta-homo-Phe
  VARIANT (17)..(17): Xaa is Phe, a trifunctional amino acid such as an amino-dicarboxylic acid or a diamino-carboxylic acid, preferably selected from Glu, Asp, Aaa, Lys, Dap, Dab, Ser, Thr, homo-Ser and homo-Thr, or absent
  VARIANT (18)..(18): Xaa is absent if Xaa at position 17 is absent, or Xaa is Phe, Ala, Trp, Tyr, Phg, Val, His, Lys, Abu, Met, Cit, Orn, Ser, Thr, Leu, Ile, Arg, homo-Tyr, homo-Phe, beta-Phe, or beta-homo-Phe
  VARIANT (19)..(19): Xaa is Phe, a trifunctional amino acid such as an amino-dicarboxylic acid or a diamino-carboxylic acid, preferably selected from Glu, Asp, Aaa, Lys, Dap, Dab, Ser, Thr, homo-Ser and homo-Thr, or absent
  VARIANT (20)..(20): Xaa is absent if Xaa at position 19 is absent, or Xaa is Phe, Ala, Trp, Tyr, Phg, Val, His, Lys, Abu, Met, Cit, Orn, Ser, Thr, Leu, Ile, Arg, homo-Tyr, homo-Phe, beta-Phe, or beta-homo-Phe
  VARIANT (21)..(21): Xaa is Phe, a trifunctional amino acid such as an amino-dicarboxylic acid or a diamino-carboxylic acid, preferably selected from Glu, Asp, Aaa, Lys, Dap, Dab, Ser, Thr, homo-Ser and homo-Thr, or absent
  VARIANT (22)..(22): Xaa is absent if Xaa at position 21 is absent, or Xaa is Phe, Ala, Trp, Tyr, Phg, Val, His, Lys, Abu, Met, Cit, Orn, Ser, Thr, Leu, Ile, Arg, homo-Tyr, homo-Phe, beta-Phe, or beta-homo-Phe
XXXXXXXXXXXXXXXXXXXXXX

SEQ ID NO: 2 - protein, 22 aa, Artificial Sequence
  Synthetic Sequence
  VARIANT (1)..(1): Xaa is Xaa is an amino acid having a hydrophobic side chain or is absent
  VARIANT (2)..(2): Xaa is an amino acid having a hydrophobic or basic side chain
  VARIANT (3)..(3): Xaa is Phe, Ala, Trp, Tyr, Phg, Val, His, Lys, Abu, Met, Cit, Orn, Ser, Thr, Leu, Ile, or Arg
  VARIANT (4)..(4): Xaa is Phe, a trifunctional amino acid such as an amino-dicarboxylic acid or a diamino-carboxylic acid, preferably selected from Glu, Asp, Aaa, Lys, Dap, Dab, Ser, Thr, homo-Ser and homo-Thr or absent
  VARIANT (5)..(5): Xaa is Phe, Ala, Trp, Tyr, Phg, Val, His, Lys, Abu, Met, Cit, Orn, Ser, Thr, Leu, Ile, Arg, or absent
  VARIANT (6)..(6): Xaa is absent if Xaa at position 5 is absent, or Xaa is Phe, a trifunctional amino acid such as an amino-dicarboxylic acid or a diamino-carboxylic acid, preferably selected from Glu, Asp, Aaa, Lys, Dap, Dab, Ser, Thr, homo-Ser and homo-Thr
  VARIANT (7)..(7): Xaa is Phe, Ala, Trp, Tyr, Phg, Val, His, Lys, Abu, Met, Cit, Orn, Ser, Thr, Leu, Ile, Arg, or absent
  VARIANT (8)..(8): Xaa is absent if Xaa at position 7 is absent, or Xaa is Phe, a trifunctional amino acid such as an amino-dicarboxylic acid or a diamino-carboxylic acid, preferably selected from Glu, Asp, Aaa, Lys, Dap, Dab, Ser, Thr, homo-Ser and homo-Thr
  VARIANT (9)..(9): Xaa is Phe, Ala, Trp, Tyr, Phg, Val, His, Lys, Abu, Met, Cit, Orn, Ser, Thr, Leu, Ile, Arg, or absent
  VARIANT (10)..(10): Xaa is absent if Xaa at position 9 is absent, or Xaa is Phe, a trifunctional amino acid such as an amino-dicarboxylic acid or a diamino-carboxylic acid, preferably selected from Glu, Asp, Aaa, Lys, Dap, Dab, Ser, Thr, homo-Ser and homo-Thr
  VARIANT (11)..(11): Xaa is Phe, Ala, Trp, Tyr, Phg, Val, His, Lys, Abu, Met, Cit, Orn, Ser, Thr, Leu, Ile, Arg, or absent
  VARIANT (12)..(12): Xaa is absent if Xaa at position 11 is absent, or Xaa is Phe, a trifunctional amino acid such as an amino-dicarboxylic acid or a diamino-carboxylic acid, preferably selected from Glu, Asp, Aaa, Lys, Dap, Dab, Ser, Thr, homo-Ser and homo-Thr
  VARIANT (13)..(13): Xaa is Phe, Ala, Trp, Tyr, Phg, Val, His, Lys, Abu, Met, Cit, Orn, Ser, Thr, Leu, Ile, Arg, or absent
  VARIANT (14)..(14): Xaa is absent if Xaa at position 13 is absent, or Xaa is Phe, a trifunctional amino acid such as an amino-dicarboxylic acid or a diamino-carboxylic acid, preferably selected from Glu, Asp, Aaa, Lys, Dap, Dab, Ser, Thr, homo-Ser and homo-Thr
  VARIANT (15)..(15): Xaa is Phe, Ala, Trp, Tyr, Phg, Val, His, Lys, Abu, Met, Cit, Orn, Ser, Thr, Leu, Ile, Arg, or absent
  VARIANT (16)..(16): Xaa is absent if Xaa at position 15 is absent, or Xaa is Phe, a trifunctional amino acid such as an amino-dicarboxylic acid or a diamino-carboxylic acid, preferably selected from Glu, Asp, Aaa, Lys, Dap, Dab, Ser, Thr, homo-Ser and homo-Thr
  VARIANT (17)..(17): Xaa is Phe, Ala, Trp, Tyr, Phg, Val, His, Lys, Abu, Met, Cit, Orn, Ser, Thr, Leu, Ile, Arg, or absent
  VARIANT (18)..(18): Xaa is absent if Xaa at position 15 is absent, or Xaa is Phe, a trifunctional amino acid such as an amino-dicarboxylic acid or a diamino-carboxylic acid, preferably selected from Glu, Asp, Aaa, Lys, Dap, Dab, Ser, Thr, homo-Ser and homo-Thr
  VARIANT (19)..(19): Xaa is Phe, Ala, Trp, Tyr, Phg, Val, His, Lys, Abu, Met, Cit, Orn, Ser, Thr, Leu, Ile, Arg, or absent
  VARIANT (20)..(20): Xaa is absent if Xaa at position 19 is absent, or Xaa is Phe, a trifunctional amino acid such as an amino-dicarboxylic acid or a diamino-carboxylic acid, preferably selected from Glu, Asp, Aaa, Lys, Dap, Dab, Ser, Thr, homo-Ser and homo-Thr
  VARIANT (21)..(21): Xaa is Phe, Ala, Trp, Tyr, Phg, Val, His, Lys, Abu, Met, Cit, Orn, Ser, Thr, Leu, Ile, Arg, or absent
  VARIANT (22)..(22): Xaa is absent if Xaa at position 21 is absent, or Xaa is Phe, a trifunctional amino acid such as an amino-dicarboxylic acid or a diamino-carboxylic acid, preferably selected from Glu, Asp, Aaa, Lys, Dap, Dab, Ser, Thr, homo-Ser and homo-Thr
XXXXXXXXXXXXXXXXXXXXXX

SEQ ID NO: 3 - protein, 22 aa, Artificial Sequence
  Synthetic Sequence
  VARIANT (1)..(1): Xaa is an amino acid having a hydrophobic side chain or is absent
  VARIANT (2)..(2): Xaa is an amino acid having a hydrophobic or basic side chain
  VARIANT (3)..(3): Xaa is an amino acid having a carboxylic acid group on its side chain, such as Ama, Glu, Aaa, Apa, or a trifunctional amino acid selected from Dap, Dab, Ser, Thr, Lys, Orn, homoLys, homoSer and homoThr
  VARIANT (4)..(4): Xaa is Phe, Ala, Trp, Tyr, Phg, Val, His, Lys, Abu, Met, Cit, Orn, Ser, Thr, Leu, Ile, Arg, homo-Tyr, homo-Phe, beta-Phe, or beta-homo-Phe
  VARIANT (5)..(5): Xaa is an amino acid having a carboxylic acid group on its side chain, such as Ama, Glu, Aaa, Apa, or a trifunctional amino acid selected from Dap, Dab, Ser, Thr, Lys, Orn, homoLys, homoSer and homoThr, or absent
  VARIANT (6)..(6): Xaa is absent if Xaa at position 5 is absent, or Xaa is Phe, Ala, Trp, Tyr, Phg, Val, His, Lys, Abu, Met, Cit, Orn, Ser, Thr, Leu, Ile, Arg, homo-Tyr, homo-Phe, beta-Phe, or beta-homo-Phe
  VARIANT (7)..(7): Xaa is an amino acid having a carboxylic acid group on its side chain, such as Ama, Glu, Aaa, Apa, or a trifunctional amino acid selected from Dap, Dab, Ser, Thr, Lys, Orn, homoLys, homoSer and homoThr, or absent
  VARIANT (8)..(8): Xaa is absent if Xaa at position 7 is absent, or Xaa is Phe, Ala, Trp, Tyr, Phg, Val, His, Lys, Abu, Met, Cit, Orn, Ser, Thr, Leu, Ile, Arg, homo-Tyr, homo-Phe, beta-Phe, or beta-homo-Phe
  VARIANT (9)..(9): Xaa is an amino acid having a carboxylic acid group on its side chain, such as Ama, Glu, Aaa, Apa, or a trifunctional amino acid selected from Dap, Dab, Ser, Thr, Lys, Orn, homoLys, homoSer and homoThr, or absent
  VARIANT (10)..(10): Xaa is absent if Xaa at position 9 is absent, or Xaa is Phe, Ala, Trp, Tyr, Phg, Val, His, Lys, Abu, Met, Cit, Orn, Ser, Thr, Leu, Ile, Arg, homo-Tyr, homo-Phe, beta-Phe, or beta-homo-Phe
  VARIANT (11)..(11): Xaa is an amino acid having a carboxylic acid group on its side chain, such as Ama, Glu, Aaa, Apa, or a trifunctional amino acid selected from Dap, Dab, Ser, Thr, Lys, Orn, homoLys, homoSer and homoThr, or absent
  VARIANT (12)..(12): Xaa is absent if Xaa at position 11 is absent, or Xaa is Phe, Ala, Trp, Tyr, Phg, Val, His, Lys, Abu, Met, Cit, Orn, Ser, Thr, Leu, Ile, Arg, homo-Tyr, homo-Phe, beta-Phe, or beta-homo-Phe
  VARIANT (13)..(13): Xaa is an amino acid having a carboxylic acid group on its side chain, such as Ama, Glu, Aaa, Apa, or a trifunctional amino acid selected from Dap, Dab, Ser, Thr, Lys, Orn, homoLys, homoSer and homoThr, or absent
  VARIANT (14)..(14): Xaa is absent if Xaa at position 13 is absent, or Xaa is Phe, Ala, Trp, Tyr, Phg, Val, His, Lys, Abu, Met, Cit, Orn, Ser, Thr, Leu, Ile, Arg, homo-Tyr, homo-Phe, beta-Phe, or beta-homo-Phe
  VARIANT (15)..(15): Xaa is an amino acid having a carboxylic acid group on its side chain, such as Ama, Glu, Aaa, Apa, or a trifunctional amino acid selected from Dap, Dab, Ser, Thr, Lys, Orn, homoLys, homoSer and homoThr, or absent
  VARIANT (16)..(16): Xaa is absent if Xaa at position 15 is absent, or Xaa is Phe, Ala, Trp, Tyr, Phg, Val, His, Lys, Abu, Met, Cit, Orn, Ser, Thr, Leu, Ile, Arg, homo-Tyr, homo-Phe, beta-Phe, or beta-homo-Phe
  VARIANT (17)..(17): Xaa is an amino acid having a carboxylic acid group on its side chain, such as Ama, Glu, Aaa, Apa, or a trifunctional amino acid selected from Dap, Dab, Ser, Thr, Lys, Orn, homoLys, homoSer and homoThr, or absent
  VARIANT (18)..(18): Xaa is absent if Xaa at position 17 is absent, or Xaa is Phe, Ala, Trp, Tyr, Phg, Val, His, Lys, Abu, Met, Cit, Orn, Ser, Thr, Leu, Ile, Arg, homo-Tyr, homo-Phe, beta-Phe, or beta-homo-Phe
  VARIANT (19)..(19): Xaa is an amino acid having a carboxylic acid group on its side chain, such as Ama, Glu, Aaa, Apa, or a trifunctional amino acid selected from Dap, Dab, Ser, Thr, Lys, Orn, homoLys, homoSer and homoThr, or absent
  VARIANT (20)..(20): Xaa is absent if Xaa at position 19 is absent, or Xaa is Phe, Ala, Trp, Tyr, Phg, Val, His, Lys, Abu, Met, Cit, Orn, Ser, Thr, Leu, Ile, Arg, homo-Tyr, homo-Phe, beta-Phe, or beta-homo-Phe
  VARIANT (21)..(21): Xaa is an amino acid having a carboxylic acid group on its side chain, such as Ama, Glu, Aaa, Apa, or a trifunctional amino acid selected from Dap, Dab, Ser, Thr, Lys, Orn, homoLys, homoSer and homoThr, or absent
  VARIANT (22)..(22): Xaa is absent if Xaa at position 21 is absent, or Xaa is Phe, Ala, Trp, Tyr, Phg, Val, His, Lys, Abu, Met, Cit, Orn, Ser, Thr, Leu, Ile, Arg, homo-Tyr, homo-Phe, beta-Phe, or beta-homo-Phe
XXXXXXXXXXXXXXXXXXXXXX

SEQ ID NO: 4 - protein, 4 aa, Artificial Sequence
  Synthetic Sequence
  VARIANT (2)..(2): Xaa is Cit
FXKF

SEQ ID NO: 5 - protein, 4 aa, Artificial Sequence
  Synthetic Sequence
  VARIANT (2)..(2): Xaa is Cit
FXKY

SEQ ID NO: 6 - protein, 4 aa, Artificial Sequence
  Synthetic Sequence
  VARIANT (2)..(2): Xaa is Cit
  VARIANT (4)..(4): Xaa is homoTyr
FXKX

SEQ ID NO: 7 - protein, 4 aa, Artificial Sequence
  Synthetic Sequence
FKKF

SEQ ID NO: 8 - protein, 4 aa, Artificial Sequence
  Synthetic Sequence
  VARIANT (1)..(1): Xaa is homoPhe
XRKF

SEQ ID NO: 9 - protein, 4 aa, Artificial Sequence
  Synthetic Sequence
  VARIANT (1)..(1): Xaa is homoPhe
  VARIANT (2)..(2): Xaa is Cit
XXKY

SEQ ID NO: 10 - protein, 4 aa, Artificial Sequence
  Synthetic Sequence
  VARIANT (1)..(1): Xaa is homoPhe
  VARIANT (2)..(2): Xaa is Cit
XXKY

SEQ ID NO: 11 - protein, 9 aa, Artificial Sequence
  Synthetic Sequence
  VARIANT (2)..(2): Xaa is Cit
  VARIANT (6)..(6): Xaa is Cit
KXKFKXKFE

SEQ ID NO: 12 - protein, 20 aa, Artificial Sequence
  Synthetic Sequence
  VARIANT (1)..(1): Xaa is a trifunctional amino acid such as an amino-dicarboxylic acid or a diamino-carboxylic acid
  VARIANT (2)..(2): Xaa is Phe, Ala, Trp, Tyr, Phg, Met, Val, His, Lys, Arg, Cit, Abu, or Orn
  VARIANT (3)..(3): Xaa is a trifunctional amino acid such as an amino-dicarboxylic acid or a diamino-carboxylic acid, or absent
  VARIANT (4)..(4): Xaa is absent if Xaa at position 3 is absent, or Xaa is Phe, Ala, Trp, Tyr, Phg, Met, Val, His, Lys, Arg, Cit, Abu, or Orn
  VARIANT (5)..(5): Xaa is a trifunctional amino acid such as an amino-dicarboxylic acid or a diamino-carboxylic acid, or absent
  VARIANT (6)..(6): Xaa is absent if Xaa at position 5 is absent, or Xaa is Phe, Ala, Trp, Tyr, Phg, Met, Val, His, Lys, Arg, Cit, Abu, or Orn
  VARIANT (7)..(7): Xaa is a trifunctional amino acid such as an amino-dicarboxylic acid or a diamino-carboxylic acid, or absent
  VARIANT (8)..(8): Xaa is absent if Xaa at position 7 is absent, or Xaa is Phe, Ala, Trp, Tyr, Phg, Met, Val, His, Lys, Arg, Cit, Abu, or Orn
  VARIANT (9)..(9): Xaa is a trifunctional amino acid such as an amino-dicarboxylic acid or a diamino-carboxylic acid, or absent
  VARIANT (10)..(10): Xaa is absent if Xaa at position 9 is absent, or Xaa is Phe, Ala, Trp, Tyr, Phg, Met, Val, His, Lys, Arg, Cit, Abu, or Orn
  VARIANT (11)..(11): Xaa is a trifunctional amino acid such as an amino-dicarboxylic acid or a diamino-carboxylic acid, or absent
  VARIANT (12)..(12): Xaa is absent if Xaa at position 11 is absent, or Xaa is Phe, Ala, Trp, Tyr, Phg, Met, Val, His, Lys, Arg, Cit, Abu, or Orn
  VARIANT (13)..(13): Xaa is a trifunctional amino acid such as an amino-dicarboxylic acid or a diamino-carboxylic acid, or absent
  VARIANT (14)..(14): Xaa is absent if Xaa at position 13 is absent, or Xaa is Phe, Ala, Trp, Tyr, Phg, Met, Val, His, Lys, Arg, Cit, Abu, or Orn
  VARIANT (15)..(15): Xaa is a trifunctional amino acid such as an amino-dicarboxylic acid or a diamino-carboxylic acid, or absent
  VARIANT (16)..(16): Xaa is absent if Xaa at position 15 is absent, or Xaa is Phe, Ala, Trp, Tyr, Phg, Met, Val, His, Lys, Arg, Cit, Abu, or Orn
  VARIANT (17)..(17): Xaa is a trifunctional amino acid such as an amino-dicarboxylic acid or a diamino-carboxylic acid, or absent
  VARIANT (18)..(18): Xaa is absent if Xaa at position 17 is absent, or Xaa is Phe, Ala, Trp, Tyr, Phg, Met, Val, His, Lys, Arg, Cit, Abu, or Orn
  VARIANT (19)..(19): Xaa is a trifunctional amino acid such as an amino-dicarboxylic acid or a diamino-carboxylic acid, or absent
  VARIANT (20)..(20): Xaa is absent if Xaa at position 19 is absent, or Xaa is Phe, Ala, Trp, Tyr, Phg, Met, Val, His, Lys, Arg, Cit, Abu, or Orn
XXXXXXXXXXXXXXXXXXXX

SEQ ID NO: 13 - protein, 20 aa, Artificial Sequence
  Synthetic Sequence
  VARIANT (1)..(1): Xaa is Phe, Ala, Trp, Tyr, Phg, Met, Val, His, Lys, Arg, Cit, Abu, or Orn
  VARIANT (2)..(2): Xaa is a trifunctional amino acid such as an amino dicarboxylic acid or a diamino carboxylic acid
  VARIANT (3)..(3): Xaa is Phe, Ala, Trp, Tyr, Phg, Met, Val, His, Lys, Arg, Cit, Abu, Orn, or absent
  VARIANT (4)..(4): Xaa is absent if Xaa at position 3 is absent, or Xaa is a trifunctional amino acid such as an amino dicarboxylic acid or a diamino carboxylic acid
  VARIANT (5)..(5): Xaa is Phe, Ala, Trp, Tyr, Phg, Met, Val, His, Lys, Arg, Cit, Abu, Orn, or absent
  VARIANT (6)..(6): Xaa is absent if Xaa at position 5 is absent, or Xaa is a trifunctional amino acid such as an amino dicarboxylic acid or a diamino carboxylic acid
  VARIANT (7)..(7): Xaa is Phe, Ala, Trp, Tyr, Phg, Met, Val, His, Lys, Arg, Cit, Abu, Orn, or absent
  VARIANT (8)..(8): Xaa is absent if Xaa at position 7 is absent, or Xaa is a trifunctional amino acid such as an amino dicarboxylic acid or a diamino carboxylic acid
  VARIANT (9)..(9): Xaa is Phe, Ala, Trp, Tyr, Phg, Met, Val, His, Lys, Arg, Cit, Abu, Orn, or absent
  VARIANT (10)..(10): Xaa is absent if Xaa at position 9 is absent, or Xaa is a trifunctional amino acid such as an amino dicarboxylic acid or a diamino carboxylic acid
  VARIANT (11)..(11): Xaa is Phe, Ala, Trp, Tyr, Phg, Met, Val, His, Lys, Arg, Cit, Abu, Orn, or absent
  VARIANT (12)..(12): Xaa is absent if Xaa at position 11 is absent, or Xaa is a trifunctional amino acid such as an amino dicarboxylic acid or a diamino carboxylic acid
  VARIANT (13)..(13): Xaa is Phe, Ala, Trp, Tyr, Phg, Met, Val, His, Lys, Arg, Cit, Abu, Orn, or absent
  VARIANT (14)..(14): Xaa is absent if Xaa at position 13 is absent, or Xaa is a trifunctional amino acid such as an amino dicarboxylic acid or a diamino carboxylic acid
  VARIANT (15)..(15): Xaa is Phe, Ala, Trp, Tyr, Phg, Met, Val, His, Lys, Arg, Cit, Abu, Orn, or absent
  VARIANT (16)..(16): Xaa is absent if Xaa at position 15 is absent, or Xaa is a trifunctional amino acid such as an amino dicarboxylic acid or a diamino carboxylic acid
  VARIANT (17)..(17): Xaa is Phe, Ala, Trp, Tyr, Phg, Met, Val, His, Lys, Arg, Cit, Abu, Orn, or absent
  VARIANT (18)..(18): Xaa is absent if Xaa at position 17 is absent, or Xaa is a trifunctional amino acid such as an amino dicarboxylic acid or a diamino carboxylic acid
  VARIANT (19)..(19): Xaa is Phe, Ala, Trp, Tyr, Phg, Met, Val, His, Lys, Arg, Cit, Abu, Orn, or absent
  VARIANT (20)..(20): Xaa is absent if Xaa at position 19 is absent, or Xaa is a trifunctional amino acid such as an amino dicarboxylic acid or a diamino carboxylic acid
XXXXXXXXXXXXXXXXXXXX

SEQ ID NO: 14 - protein, 23 aa, Artificial Sequence
  Synthetic Sequence
  VARIANT (1)..(1): Xaa is Cit
  VARIANT (4)..(4): Xaa is Lys or absent
  VARIANT (5)..(5): Xaa is absent if Xaa at position 4 is absent, or Xaa is Phe
  VARIANT (6)..(6): Xaa is Lys or absent
  VARIANT (7)..(7): Xaa is absent if Xaa at position 6 is absent, or Xaa is Phe
  VARIANT (8)..(8): Xaa is Lys or absent
  VARIANT (9)..(9): Xaa is absent if Xaa at position 8 is absent, or Xaa is Phe
  VARIANT (10)..(10): Xaa is Lys or absent
  VARIANT (11)..(11): Xaa is absent if Xaa at position 10 is absent, or Xaa is Phe
  VARIANT (12)..(12): Xaa is Lys or absent
  VARIANT (13)..(13): Xaa is absent if Xaa at position 12 is absent, or Xaa is Phe
  VARIANT (14)..(14): Xaa is Lys or absent
  VARIANT (15)..(15): Xaa is absent if Xaa at position 14 is absent, or Xaa is Phe
  VARIANT (16)..(16): Xaa is Lys or absent
  VARIANT (17)..(17): Xaa is absent if Xaa at position 16 is absent, or Xaa is Phe
  VARIANT (18)..(18): Xaa is Lys or absent
  VARIANT (19)..(19): Xaa is absent if Xaa at position 18 is absent, or Xaa is Phe
  VARIANT (20)..(20): Xaa is Lys or absent
  VARIANT (21)..(21): Xaa is absent if Xaa at position 20 is absent, or Xaa is Phe
XKFXXXXXXXXXXXXXXXXXXKF

SEQ ID NO: 15 - protein, 23 aa, Artificial Sequence
  Synthetic Sequence
  VARIANT (1)..(1): Xaa is Cit
  VARIANT (4)..(4): Xaa is Lys or absent
  VARIANT (5)..(5): Xaa is absent if Xaa at position 4 is absent, or Xaa is Phe
  VARIANT (6)..(6): Xaa is Lys or absent
  VARIANT (7)..(7): Xaa is absent if Xaa at position 6 is absent, or Xaa is Phe
  VARIANT (8)..(8): Xaa is Lys or absent
  VARIANT (9)..(9): Xaa is absent if Xaa at position 8 is absent, or Xaa is Phe
  VARIANT (10)..(10): Xaa is Lys or absent
  VARIANT (11)..(11): Xaa is absent if Xaa at position 10 is absent, or Xaa is Phe
  VARIANT (12)..(12): Xaa is Lys or absent
  VARIANT (13)..(13): Xaa is absent if Xaa at position 12 is absent, or Xaa is Phe
  VARIANT (14)..(14): Xaa is Lys or absent
  VARIANT (15)..(15): Xaa is absent if Xaa at position 14 is absent, or Xaa is Phe
  VARIANT (16)..(16): Xaa is Lys or absent
  VARIANT (17)..(17): Xaa is absent if Xaa at position 16 is absent, or Xaa is Phe
  VARIANT (18)..(18): Xaa is Lys or absent
  VARIANT (19)..(19): Xaa is absent if Xaa at position 18 is absent, or Xaa is Phe
  VARIANT (20)..(20): Xaa is Lys or absent
  VARIANT (21)..(21): Xaa is absent if Xaa at position 20 is absent, or Xaa is Phe
  VARIANT (23)..(23): Xaa is homoTyr
XKFXXXXXXXXXXXXXXXXXXKX

SEQ ID NO: 16 - protein, 23 aa, Artificial Sequence
  Synthetic Sequence
  VARIANT (1)..(1): Xaa is Cit
  VARIANT (4)..(4): Xaa is Lys or absent
  VARIANT (5)..(5): Xaa is absent if Xaa at position 4 is absent, or Xaa is Phe
  VARIANT (6)..(6): Xaa is Lys or absent
  VARIANT (7)..(7): Xaa is absent if Xaa at position 6 is absent, or Xaa is Phe
  VARIANT (8)..(8): Xaa is Lys or absent
  VARIANT (9)..(9): Xaa is absent if Xaa at position 8 is absent, or Xaa is Phe
  VARIANT (10)..(10): Xaa is Lys or absent
  VARIANT (11)..(11): Xaa is absent if Xaa at position 10 is absent, or Xaa is Phe
  VARIANT (12)..(12): Xaa is Lys or absent
  VARIANT (13)..(13): Xaa is absent if Xaa at position 12 is absent, or Xaa is Phe
  VARIANT (14)..(14): Xaa is Lys or absent
  VARIANT (15)..(15): Xaa is absent if Xaa at position 14 is absent, or Xaa is Phe
  VARIANT (16)..(16): Xaa is Lys or absent
  VARIANT (17)..(17): Xaa is absent if Xaa at position 16 is absent, or Xaa is Phe
  VARIANT (18)..(18): Xaa is Lys or absent
  VARIANT (19)..(19): Xaa is absent if Xaa at position 18 is absent, or Xaa is Phe
  VARIANT (20)..(20): Xaa is Lys or absent
  VARIANT (21)..(21): Xaa is absent if Xaa at position 20 is absent, or Xaa is Phe
XKFXXXXXXXXXXXXXXXXXXKY

SEQ ID NO: 17 - protein, 22 aa, Artificial Sequence
  Synthetic Sequence
  VARIANT (3)..(3): Xaa is Lys or absent
  VARIANT (4)..(4): Xaa is absent if Xaa at position 3 is absent, or Xaa is Phe
  VARIANT (5)..(5): Xaa is Lys or absent
  VARIANT (6)..(6): Xaa is absent if Xaa at position 5 is absent, or Xaa is Phe
  VARIANT (7)..(7): Xaa is Lys or absent
  VARIANT (8)..(8): Xaa is absent if Xaa at position 7 is absent, or Xaa is Phe
  VARIANT (9)..(9): Xaa is Lys or absent
  VARIANT (10)..(10): Xaa is absent if Xaa at position 9 is absent, or Xaa is Phe
  VARIANT (11)..(11): Xaa is Lys or absent
  VARIANT (12)..(12): Xaa is absent if Xaa at position 11 is absent, or Xaa is Phe
  VARIANT (13)..(13): Xaa is Lys or absent
  VARIANT (14)..(14): Xaa is absent if Xaa at position 13 is absent, or Xaa is Phe
  VARIANT (15)..(15): Xaa is Lys or absent
  VARIANT (16)..(16): Xaa is absent if Xaa at position 15 is absent, or Xaa is Phe
  VARIANT (17)..(17): Xaa is Lys or absent
  VARIANT (18)..(18): Xaa is absent if Xaa at position 17 is absent, or Xaa is Phe
  VARIANT (19)..(19): Xaa is Lys or absent
  VARIANT (20)..(20): Xaa is absent if Xaa at position 19 is absent, or Xaa is Phe
KFXXXXXXXXXXXXXXXXXXKF

SEQ ID NO: 18 - protein, 23 aa, Artificial Sequence
  Synthetic Sequence
  VARIANT (4)..(4): Xaa is Phe or absent
  VARIANT (5)..(5): Xaa is absent if Xaa at position 4 is absent, or Xaa is Lys
  VARIANT (6)..(6): Xaa is Phe or absent
  VARIANT (7)..(7): Xaa is absent if Xaa at position 6 is absent, or Xaa is Lys
  VARIANT (8)..(8): Xaa is Phe or absent
  VARIANT (9)..(9): Xaa is absent if Xaa at position 8 is absent, or Xaa is Lys
  VARIANT (10)..(10): Xaa is Phe or absent
  VARIANT (11)..(11): Xaa is absent if Xaa at position 10 is absent, or Xaa is Lys
  VARIANT (12)..(12): Xaa is Phe or absent
  VARIANT (13)..(13): Xaa is absent if Xaa at position 12 is absent, or Xaa is Lys
  VARIANT (14)..(14): Xaa is Phe or absent
  VARIANT (15)..(15): Xaa is absent if Xaa at position 14 is absent, or Xaa is Lys
  VARIANT (16)..(16): Xaa is Phe or absent
  VARIANT (17)..(17): Xaa is absent if Xaa at position 16 is absent, or Xaa is Lys
  VARIANT (18)..(18): Xaa is Phe or absent
  VARIANT (19)..(19): Xaa is absent if Xaa at position 18 is absent, or Xaa is Lys
  VARIANT (20)..(20): Xaa is Phe or absent
  VARIANT (21)..(21): Xaa is absent if Xaa at position 20 is absent, or Xaa is Lys
FFKXXXXXXXXXXXXXXXXXXKY

SEQ ID NO: 19 - protein, 22 aa, Artificial Sequence
  Synthetic Sequence
  VARIANT (3)..(3): Xaa is Phe or absent
  VARIANT (4)..(4): Xaa is absent if Xaa at position 3 is absent, or Xaa is Lys
  VARIANT (5)..(5): Xaa is Phe or absent
  VARIANT (6)..(6): Xaa is absent if Xaa at position 5 is absent, or Xaa is Lys
  VARIANT (7)..(7): Xaa is Phe or absent
  VARIANT (8)..(8): Xaa is absent if Xaa at position 7 is absent, or Xaa is Lys
  VARIANT (9)..(9): Xaa is Phe or absent
  VARIANT (10)..(10): Xaa is absent if Xaa at position 9 is absent, or Xaa is Lys
  VARIANT (11)..(11): Xaa is Phe or absent
  VARIANT (12)..(12): Xaa is absent if Xaa at position 11 is absent, or Xaa is Lys
  VARIANT (13)..(13): Xaa is Phe or absent
  VARIANT (14)..(14): Xaa is absent if Xaa at position 13 is absent, or Xaa is Lys
  VARIANT (15)..(15): Xaa is Phe or absent
  VARIANT (16)..(16): Xaa is absent if Xaa at position 15 is absent, or Xaa is Lys
  VARIANT (17)..(17): Xaa is Phe or absent
  VARIANT (18)..(18): Xaa is absent if Xaa at position 17 is absent, or Xaa is Lys
  VARIANT (19)..(19): Xaa is Phe or absent
  VARIANT (20)..(20): Xaa is absent if Xaa at position 19 is absent, or Xaa is Lys
FKXXXXXXXXXXXXXXXXXXKY

SEQ ID NO: 20 - protein, 23 aa, Artificial Sequence
  Synthetic Sequence
  VARIANT (4)..(4): Xaa is Phe or absent
  VARIANT (5)..(5): Xaa is absent if Xaa at position 4 is absent, or Xaa is Lys
  VARIANT (6)..(6): Xaa is Phe or absent
  VARIANT (7)..(7): Xaa is absent if Xaa at position 6 is absent, or Xaa is Lys
  VARIANT (8)..(8): Xaa is Phe or absent
  VARIANT (9)..(9): Xaa is absent if Xaa at position 8 is absent, or Xaa is Lys
  VARIANT (10)..(10): Xaa is Phe or absent
  VARIANT (11)..(11): Xaa is absent if Xaa at position 10 is absent, or Xaa is Lys
  VARIANT (12)..(12): Xaa is Phe or absent
  VARIANT (13)..(13): Xaa is absent if Xaa at position 12 is absent, or Xaa is Lys
  VARIANT (14)..(14): Xaa is Phe or absent
  VARIANT (15)..(15): Xaa is absent if Xaa at position 14 is absent, or Xaa is Lys
  VARIANT (16)..(16): Xaa is Phe or absent
  VARIANT (17)..(17): Xaa is absent if Xaa at position 16 is absent, or Xaa is Lys
  VARIANT (18)..(18): Xaa is Phe or absent
  VARIANT (19)..(19): Xaa is absent if Xaa at position 18 is absent, or Xaa is Lys
  VARIANT (20)..(20): Xaa is Phe or absent
  VARIANT (21)..(21): Xaa is absent if Xaa at position 20 is absent, or Xaa is Lys
  VARIANT (23)..(23): Xaa is homoTyr
FFKXXXXXXXXXXXXXXXXXXKX

SEQ ID NO: 21 - protein, 23 aa, Artificial Sequence
  Synthetic Sequence
  VARIANT (4)..(4): Xaa is Phe or absent
  VARIANT (5)..(5): Xaa absent if Xaa at position 4 is absent, or Xaa is Lys
  VARIANT (6)..(6): Xaa is Phe or absent
  VARIANT (7)..(7): Xaa absent if Xaa at position 6 is absent, or Xaa is Lys
  VARIANT (8)..(8): Xaa is Phe or absent
  VARIANT (9)..(9): Xaa absent if Xaa at position 8 is absent, or Xaa is Lys
  VARIANT (10)..(10): Xaa is Phe or absent
  VARIANT (11)..(11): Xaa absent if Xaa at position 10 is absent, or Xaa is Lys
  VARIANT (12)..(12): Xaa is Phe or absent
  VARIANT (13)..(13): Xaa absent if Xaa at position 12 is absent, or Xaa is Lys
  VARIANT (14)..(14): Xaa is Phe or absent
  VARIANT (15)..(15): Xaa absent if Xaa at position 14 is absent, or Xaa is Lys
  VARIANT (16)..(16): Xaa is Phe or absent
  VARIANT (17)..(17): Xaa absent if Xaa at position 16 is absent, or Xaa is Lys
  VARIANT (18)..(18): Xaa is Phe or absent
  VARIANT (19)..(19): Xaa absent if Xaa at position 18 is absent, or Xaa is Lys
  VARIANT (20)..(20): Xaa is Phe or absent
  VARIANT (21)..(21): Xaa absent if Xaa at position 20 is absent, or Xaa is Lys
RFKXXXXXXXXXXXXXXXXXXKY

SEQ ID NO: 22 - protein, 4 aa, Artificial Sequence
  Synthetic Sequence
  VARIANT (2)..(2): Xaa is Cit
FXFK

SEQ ID NO: 23 - protein, 4 aa, Artificial Sequence
  Synthetic Sequence
  VARIANT (1)..(1): Xaa is homoPhe
  VARIANT (2)..(2): Xaa is Cit
XXFK

SEQ ID NO: 24 - protein, 4 aa, Artificial Sequence
  Synthetic Sequence
FRFK

SEQ ID NO: 25 - protein, 5 aa, Artificial Sequence
  Synthetic Sequence
RKFKF

SEQ ID NO: 26 - protein, 5 aa, Artificial Sequence
  Synthetic Sequence
  VARIANT (1)..(1): Xaa is Cit
XKFKF

SEQ ID NO: 27 - protein, 5 aa, Artificial Sequence
  Synthetic Sequence
RKFKF

SEQ ID NO: 28 - protein, 4 aa, Artificial Sequence
  Synthetic Sequence
  VARIANT (1)..(1): Xaa is APhe
XRSK

SEQ ID NO: 29 - protein, 4 aa, Artificial Sequence
  Synthetic Sequence
  VARIANT (1)..(1): Xaa is APhe
XRFK

SEQ ID NO: 30 - protein, 6 aa, Artificial Sequence
  Synthetic Sequence
FRFKSK

SEQ ID NO: 31 - protein, 22 aa, Artificial Sequence
  Synthetic Sequence
  VARIANT (5)..(5): Xaa is Phe or absent
  VARIANT (6)..(6): Xaa is absent if Xaa at position 5 is absent, or Xaa is Lys
  VARIANT (7)..(7): Xaa is Phe or absent
  VARIANT (8)..(8): Xaa is absent if Xaa at position 7 is absent, or Xaa is Lys
  VARIANT (9)..(9): Xaa is Phe or absent
  VARIANT (10)..(10): Xaa is absent if Xaa at position 9 is absent, or Xaa is Lys
  VARIANT (11)..(11): Xaa is Phe or absent
  VARIANT (12)..(12): Xaa is absent if Xaa at position 11 is absent, or Xaa is Lys
  VARIANT (13)..(13): Xaa is Phe or absent
  VARIANT (14)..(14): Xaa is absent if Xaa at position 13 is absent, or Xaa is Lys
  VARIANT (15)..(15): Xaa is Phe or absent
  VARIANT (16)..(16): Xaa is absent if Xaa at position 15 is absent, or Xaa is Lys
  VARIANT (17)..(17): Xaa is Phe or absent
  VARIANT (18)..(18): Xaa is absent if Xaa at position 17 is absent, or Xaa is Lys
  VARIANT (19)..(19): Xaa is Phe or absent
  VARIANT (20)..(20): Xaa is absent if Xaa at position 19 is absent, or Xaa is Lys
  VARIANT (21)..(21): Xaa is Phe or absent
  VARIANT (22)..(22): Xaa is absent if Xaa at position 21 is absent, or Xaa is Lys
FRFKXXXXXXXXXXXXXXXXXX

SEQ ID NO: 32 - protein, 22 aa, Artificial Sequence
  Synthetic Sequence
  VARIANT (5)..(5): Xaa is Ser or absent
  VARIANT (6)..(6): Xaa is absent if Xaa at position 5 is absent, or Xaa is Lys
  VARIANT (7)..(7): Xaa is Ser or absent
  VARIANT (8)..(8): Xaa is absent if Xaa at position 7 is absent, or Xaa is Lys
  VARIANT (9)..(9): Xaa is Ser or absent
  VARIANT (10)..(10): Xaa is absent if Xaa at position 9 is absent, or Xaa is Lys
  VARIANT (11)..(11): Xaa is Ser or absent
  VARIANT (12)..(12): Xaa is absent if Xaa at position 11 is absent, or Xaa is Lys
  VARIANT (13)..(13): Xaa is Ser or absent
  VARIANT (14)..(14): Xaa is absent if Xaa at position 13 is absent, or Xaa is Lys
  VARIANT (15)..(15): Xaa is Ser or absent
  VARIANT (16)..(16): Xaa is absent if Xaa at position 15 is absent, or Xaa is Lys
  VARIANT (17)..(17): Xaa is Ser or absent
  VARIANT (18)..(18): Xaa is absent if Xaa at position 17 is absent, or Xaa is Lys
  VARIANT (19)..(19): Xaa is Ser or absent
  VARIANT (20)..(20): Xaa is absent if Xaa at position 19 is absent, or Xaa is Lys
  VARIANT (21)..(21): Xaa is Ser or absent
  VARIANT (22)..(22): Xaa is absent if Xaa at position 21 is absent, or Xaa is Lys
FRSKXXXXXXXXXXXXXXXXXX

SEQ ID NO: 33 - protein, 22 aa, Artificial Sequence
  Synthetic Sequence
  VARIANT (5)..(5): Xaa is Tyr or absent
  VARIANT (6)..(6): Xaa is absent if Xaa at position 5 is absent, or Xaa is Lys
  VARIANT (7)..(7): Xaa is Tyr or absent
  VARIANT (8)..(8): Xaa is absent if Xaa at position 7 is absent, or Xaa is Lys
  VARIANT (9)..(9): Xaa is Tyr or absent
  VARIANT (10)..(10): Xaa is absent if Xaa at position 9 is absent, or Xaa is Lys
  VARIANT (11)..(11): Xaa is Tyr or absent
  VARIANT (12)..(12): Xaa is absent if Xaa at position 11 is absent, or Xaa is Lys
  VARIANT (13)..(13): Xaa is Tyr or absent
  VARIANT (14)..(14): Xaa is absent if Xaa at position 13 is absent, or Xaa is Lys
  VARIANT (15)..(15): Xaa is Tyr or absent
  VARIANT (16)..(16): Xaa is absent if Xaa at position 15 is absent, or Xaa is Lys
  VARIANT (17)..(17): Xaa is Tyr or absent
  VARIANT (18)..(18): Xaa is absent if Xaa at position 17 is absent, or Xaa is Lys
  VARIANT (19)..(19): Xaa is Tyr or absent
  VARIANT (20)..(20): Xaa is absent if Xaa at position 19 is absent, or Xaa is Lys
  VARIANT (21)..(21): Xaa is Tyr or absent
  VARIANT (22)..(22): Xaa is absent if Xaa at position 21 is absent, or Xaa is Lys
FRYKXXXXXXXXXXXXXXXXXX

SEQ ID NO: 34 - protein, 24 aa, Artificial Sequence
  Synthetic Sequence
  VARIANT (5)..(5): Xaa is Phe or absent
  VARIANT (6)..(6): Xaa is absent if Xaa at position 5 is absent, or Xaa is Lys
  VARIANT (7)..(7): Xaa is Phe or absent
  VARIANT (8)..(8): Xaa is absent if Xaa at position 7 is absent, or Xaa is Lys
  VARIANT (9)..(9): Xaa is Phe or absent
  VARIANT (10)..(10): Xaa is absent if Xaa at position 9 is absent, or Xaa is Lys
  VARIANT (11)..(11): Xaa is Phe or absent
  VARIANT (12)..(12): Xaa is absent if Xaa at position 11 is absent, or Xaa is Lys
  VARIANT (13)..(13): Xaa is Phe or absent
  VARIANT (14)..(14): Xaa is absent if Xaa at position 13 is absent, or Xaa is Lys
  VARIANT (15)..(15): Xaa is Phe or absent
  VARIANT (16)..(16): Xaa is absent if Xaa at position 15 is absent, or Xaa is Lys
  VARIANT (17)..(17): Xaa is Phe or absent
  VARIANT (18)..(18): Xaa is absent if Xaa at position 17 is absent, or Xaa is Lys
  VARIANT (19)..(19): Xaa is Phe or absent
  VARIANT (20)..(20): Xaa is absent if Xaa at position 19 is absent, or Xaa is Lys
  VARIANT (21)..(21): Xaa is Phe or absent
  VARIANT (22)..(22): Xaa is absent if Xaa at position 21 is absent, or Xaa is Lys
FRFKXXXXXXXXXXXXXXXXXXFY

SEQ ID NO: 35 - protein, 5 aa, Artificial Sequence
  Synthetic Sequence
  VARIANT (1)..(1): Xaa is Sar
  VARIANT (2)..(2): Xaa is Ama
XXAKF

SEQ ID NO: 36 - protein, 5 aa, Artificial Sequence
  Synthetic Sequence
  VARIANT (1)..(1): Xaa is Sar
  VARIANT (2)..(2): Xaa is Ama
XXAAK

SEQ ID NO: 37 - protein, 11 aa, Artificial Sequence
  Synthetic Sequence
  VARIANT (1)..(1): Xaa is Sar
  VARIANT (2)..(2): Xaa is Ama
  VARIANT (4)..(4): Xaa is Ama
  VARIANT (6)..(6): Xaa is Ama
  VARIANT (8)..(8): Xaa is Ama
XXAXAXAXAAK

SEQ ID NO: 38 - protein, 4 aa, Artificial Sequence
  Synthetic Sequence
  VARIANT (2)..(2): Xaa is Cit
VXEA

SEQ ID NO: 39 - protein, 4 aa, Artificial Sequence
  Synthetic Sequence
FREF

SEQ ID NO: 40 - protein, 4 aa, Artificial Sequence
  Synthetic Sequence
  VARIANT (3)..(3): Xaa is Dap
FRXF

SEQ ID NO: 41 - protein, 4 aa, Artificial Sequence
  Synthetic Sequence
  VARIANT (3)..(3): Xaa is Dab
FRXF

SEQ ID NO: 42 - protein, 4 aa, Artificial Sequence
  Synthetic Sequence
FRSF

SEQ ID NO: 43 - protein, 8 aa, Artificial Sequence
  Synthetic Sequence
  VARIANT (2)..(2): Xaa is Cit
  VARIANT (6)..(6): Xaa is Cit
FXKFFXKF

SEQ ID NO: 44 - protein, 4 aa, Artificial Sequence
  Synthetic Sequence
FKKF

SEQ ID NO: 45 - protein, 4 aa, Artificial Sequence
  Synthetic Sequence
  VARIANT (2)..(2): Xaa is Cit
FXKF

SEQ ID NO: 46 - protein, 4 aa, Artificial Sequence
  Synthetic Sequence
  VARIANT (2)..(2): Xaa is Cit
FXFK

SEQ ID NO: 47 - protein, 4 aa, Artificial Sequence
  Synthetic Sequence
  VARIANT (2)..(2): Xaa is Cit
FXFK

SEQ ID NO: 48 - protein, 4 aa, Artificial Sequence
  Synthetic Sequence
  VARIANT (3)..(3): Xaa is Dap
FRXF

SEQ ID NO: 49 - protein, 4 aa, Artificial Sequence
  Synthetic Sequence
  VARIANT (3)..(3): Xaa is Dab
FRXF

SEQ ID NO: 50 - protein, 4 aa, Artificial Sequence
  Synthetic Sequence
FRSF

SEQ ID NO: 51 - protein, 4 aa, Artificial Sequence
  Synthetic Sequence
FKKF

SEQ ID NO: 52 - protein, 4 aa, Artificial Sequence
  Synthetic Sequence
FKKF

SEQ ID NO: 53 - protein, 4 aa, Artificial Sequence
  Synthetic Sequence
FKKF

SEQ ID NO: 54 - protein, 4 aa, Artificial Sequence
  Synthetic Sequence
  VARIANT (2)..(2): Xaa is Cit
FXKF

SEQ ID NO: 55 - protein, 4 aa, Artificial Sequence
  Synthetic Sequence
  VARIANT (2)..(2): Xaa is Cit
FXFK

SEQ ID NO: 56 - protein, 4 aa, Artificial Sequence
  Synthetic Sequence
  VARIANT (2)..(2): Xaa is Cit
FXKF

SEQ ID NO: 57 - protein, 4 aa, Artificial Sequence
  Synthetic Sequence
FRSF

SEQ ID NO: 58 - protein, 4 aa, Artificial Sequence
  Synthetic Sequence
  VARIANT (2)..(2): Xaa is Cit
FXKY

SEQ ID NO: 59 - protein, 4 aa, Artificial Sequence
  Synthetic Sequence
  VARIANT (2)..(2): Xaa is Cit
  VARIANT (4)..(4): Xaa is Cit
FXKX

SEQ ID NO: 60 - protein, 4 aa, Artificial Sequence
  Synthetic Sequence
  VARIANT (2)..(2): Xaa is Cit
  VARIANT (4)..(4): Xaa is Cit
FXKX

SEQ ID NO: 61 - protein, 4 aa, Artificial Sequence
  Synthetic Sequence
  VARIANT (2)..(2): Xaa is Cit
FXKY

SEQ ID NO: 62 - protein, 6 aa, Artificial Sequence
  Synthetic Sequence
FKKFFK

SEQ ID NO: 63 - protein, 8 aa, Artificial Sequence
  Synthetic Sequence
FKKFFKKF

SEQ ID NO: 64 - protein, 6 aa, Artificial Sequence
  Synthetic Sequence
FRKRKF

SEQ ID NO: 65 - protein, 5 aa, Artificial Sequence
  Synthetic Sequence
  VARIANT (1)..(1): Xaa is Cit
XKFKF

SEQ ID NO: 66 - protein, 4 aa, Artificial Sequence
  Synthetic Sequence
  VARIANT (1)..(1): Xaa is APhe
XRKF

SEQ ID NO: 67 - protein, 4 aa, Artificial Sequence
  Synthetic Sequence
  VARIANT (1)..(1): Xaa is APhe
  VARIANT (4)..(4): Xaa is homoPhe
XRKX

SEQ ID NO: 68 - protein, 4 aa, Artificial Sequence
  Synthetic Sequence
  VARIANT (1)..(1): Xaa is APhe
  VARIANT (2)..(2): Xaa is Cit
XXKF

SEQ ID NO: 69 - protein, 4 aa, Artificial Sequence
  Synthetic Sequence
  VARIANT (1)..(1): Xaa is APhe
  VARIANT (2)..(2): Xaa is Cit
XXKY

SEQ ID NO: 70 - protein, 4 aa, Artificial Sequence
  Synthetic Sequence
  VARIANT (1)..(1): Xaa is APhe
  VARIANT (2)..(2): Xaa is Cit
  VARIANT (4)..(4): Xaa is homoTyr
XXKX

SEQ ID NO: 71 - protein, 4 aa, Artificial Sequence
  Synthetic Sequence
  VARIANT (2)..(2): Xaa is Cit
FXKF

SEQ ID NO: 72 - protein, 4 aa, Artificial Sequence
  Synthetic Sequence
  VARIANT (2)..(2): Xaa is Cit
FXKY

SEQ ID NO: 73 - protein, 4 aa, Artificial Sequence
  Synthetic Sequence
  VARIANT (2)..(2): Xaa is Cit
  VARIANT (4)..(4): Xaa is homoTyr
FXKX

SEQ ID NO: 74 - protein, 4 aa, Artificial Sequence
  Synthetic Sequence
FKKF

SEQ ID NO: 75 - protein, 4 aa, Artificial Sequence
  Synthetic Sequence
  VARIANT (1)..(1): Xaa is homoPhe
XRKF

SEQ ID NO: 76 - protein, 4 aa, Artificial Sequence
  Synthetic Sequence
  VARIANT (1)..(1): Xaa is homoPhe
  VARIANT (2)..(2): Xaa is Cit
XXKY

SEQ ID NO: 77 - protein, 4 aa, Artificial Sequence
  Synthetic Sequence
  VARIANT (1)..(1): Xaa is homoPhe
  VARIANT (2)..(2): Xaa is Cit
XXKY

SEQ ID NO: 78 - protein, 4 aa, Artificial Sequence
  Synthetic Sequence
  VARIANT (3)..(3): Xaa is Dab
FRXF

SEQ ID NO: 79 - protein, 4 aa, Artificial Sequence
  Synthetic Sequence
  VARIANT (3)..(3): Xaa is Dap
FRXF

SEQ ID NO: 80 - protein, 4 aa, Artificial Sequence
  Synthetic Sequence
FREF

SEQ ID NO: 81 - protein, 4 aa, Artificial Sequence
  Synthetic Sequence
  VARIANT (1)..(1): Xaa is APhe
  VARIANT (2)..(2): Xaa is Cit
XXFK

SEQ ID NO: 82 - protein, 4 aa, Artificial Sequence
  Synthetic Sequence
  VARIANT (1)..(1): Xaa is APhe
  VARIANT (2)..(2): Xaa is Cit
XXSK

SEQ ID NO: 83 - protein, 4 aa, Artificial Sequence
  Synthetic Sequence
  VARIANT (1)..(1): Xaa is APhe
  VARIANT (2)..(2): Xaa is Cit
  VARIANT (3)..(3): Xaa is homoPhe
XXXK

SEQ ID NO: 84 - protein, 4 aa, Artificial Sequence
  Synthetic Sequence
  VARIANT (2)..(2): Xaa is Cit
FXFK

SEQ ID NO: 85 - protein, 4 aa, Artificial Sequence
  Synthetic Sequence
  VARIANT (1)..(1): Xaa is homoPhe
  VARIANT (2)..(2): Xaa is Cit
XXFK

SEQ ID NO: 86 - protein, 4 aa, Artificial Sequence
  Synthetic Sequence
FRFK

SEQ ID NO: 87 - protein, 4 aa, Artificial Sequence
  Synthetic Sequence
  VARIANT (2)..(2): Xaa is Cit
VXEA

SEQ ID NO: 88 - protein, 24 aa, Artificial Sequence
  Synthetic Sequence
  VARIANT (5)..(5): Xaa is Phe or absent
  VARIANT (6)..(6): Xaa is absent if Xaa at position 5 is absent, or Xaa is Lys
  VARIANT (7)..(7): Xaa is Phe or absent
  VARIANT (8)..(8): Xaa is absent if Xaa at position 7 is absent, or Xaa is Lys
  VARIANT (9)..(9): Xaa is Phe or absent
  VARIANT (10)..(10): Xaa is absent if Xaa at position 9 is absent, or Xaa is Lys
  VARIANT (11)..(11): Xaa is Phe or absent
  VARIANT (12)..(12): Xaa is absent if Xaa at position 11 is absent, or Xaa is Lys
  VARIANT (13)..(13): Xaa is Phe or absent
  VARIANT (14)..(14): Xaa is absent if Xaa at position 13 is absent, or Xaa is Lys
  VARIANT (15)..(15): Xaa is Phe or absent
  VARIANT (16)..(16): Xaa is absent if Xaa at position 15 is absent, or Xaa is Lys
  VARIANT (17)..(17): Xaa is Phe or absent
  VARIANT (18)..(18): Xaa is absent if Xaa at position 17 is absent, or Xaa is Lys
  VARIANT (19)..(19): Xaa is Phe or absent
  VARIANT (20)..(20): Xaa is absent if Xaa at position 19 is absent, or Xaa is Lys
  VARIANT (21)..(21): Xaa is Phe or absent
  VARIANT (22)..(22): Xaa is absent if Xaa at position 21 is absent, or Xaa is Lys
FRFKXXXXXXXXXXXXXXXXXXFY

SEQ ID NO: 89 - protein, 22 aa, Artificial Sequence
  Synthetic Sequence
  VARIANT (5)..(5): Xaa is Tyr or absent
  VARIANT (6)..(6): Xaa is absent if Xaa at position 5 is absent, or Xaa is Lys
  VARIANT (7)..(7): Xaa is Tyr or absent
  VARIANT (8)..(8): Xaa is absent if Xaa at position 7 is absent, or Xaa is Lys
  VARIANT (9)..(9): Xaa is Tyr or absent
  VARIANT (10)..(10): Xaa is absent if Xaa at position 9 is absent, or Xaa is Lys
  VARIANT (11)..(11): Xaa is Tyr or absent
  VARIANT (12)..(12): Xaa is absent if Xaa at position 11 is absent, or Xaa is Lys
  VARIANT (13)..(13): Xaa is Tyr or absent
  VARIANT (14)..(14): Xaa is absent if Xaa at position 13 is absent, or Xaa is Lys
  VARIANT (15)..(15): Xaa is Tyr or absent
  VARIANT (16)..(16): Xaa is absent if Xaa at position 15 is absent, or Xaa is Lys
  VARIANT (17)..(17): Xaa is Tyr or absent
  VARIANT (18)..(18): Xaa is absent if Xaa at position 17 is absent, or Xaa is Lys
  VARIANT (19)..(19): Xaa is Tyr or absent
  VARIANT (20)..(20): Xaa is absent if Xaa at position 19 is absent, or Xaa is Lys
  VARIANT (21)..(21): Xaa is Tyr or absent
  VARIANT (22)..(22): Xaa is absent if Xaa at position 21 is absent, or Xaa is Lys
FRYKXXXXXXXXXXXXXXXXXX

SEQ ID NO: 90 - protein, 22 aa, Artificial Sequence
  Synthetic Sequence
  VARIANT (5)..(5): Xaa is Ser or absent
  VARIANT (6)..(6): Xaa is absent if Xaa at position 5 is absent, or Xaa is Lys
  VARIANT (7)..(7): Xaa is Ser or absent
  VARIANT (8)..(8): Xaa is absent if Xaa at position 7 is absent, or Xaa is Lys
  VARIANT (9)..(9): Xaa is Ser or absent
  VARIANT (10)..(10): Xaa is absent if Xaa at position 9 is absent, or Xaa is Lys
  VARIANT (11)..(11): Xaa is Ser or absent
  VARIANT (12)..(12): Xaa is absent if Xaa at position 11 is absent, or Xaa is Lys
  VARIANT (13)..(13): Xaa is Ser or absent
  VARIANT (14)..(14): Xaa is absent if Xaa at position 13 is absent, or Xaa is Lys
  VARIANT (15)..(15): Xaa is Ser or absent
  VARIANT (16)..(16): Xaa is absent if Xaa at position 15 is absent, or Xaa is Lys
  VARIANT (17)..(17): Xaa is Ser or absent
  VARIANT (18)..(18): Xaa is absent if Xaa at position 17 is absent, or Xaa is Lys
  VARIANT (19)..(19): Xaa is Ser or absent
  VARIANT (20)..(20): Xaa is absent if Xaa at position 19 is absent, or Xaa is Lys
  VARIANT (21)..(21): Xaa is Ser or absent
  VARIANT (22)..(22): Xaa is absent if Xaa at position 21 is absent, or Xaa is Lys
FRSKXXXXXXXXXXXXXXXXXX

SEQ ID NO: 91 - protein, 22 aa, Artificial Sequence
  Synthetic Sequence
  VARIANT (5)..(5): Xaa is Phe or absent
  VARIANT (6)..(6): Xaa is absent if Xaa at position 5 is absent, or Xaa is Lys
  VARIANT (7)..(7): Xaa is Phe or absent
  VARIANT (8)..(8): Xaa is absent if Xaa at position 7 is absent, or Xaa is Lys
  VARIANT (9)..(9): Xaa is Phe or absent
  VARIANT (10)..(10): Xaa is absent if Xaa at position 9 is absent, or Xaa is Lys
  VARIANT (11)..(11): Xaa is Phe or absent
  VARIANT (12)..(12): Xaa is absent if Xaa at position 11 is absent, or Xaa is Lys
  VARIANT (13)..(13): Xaa is Phe or absent
  VARIANT (14)..(14): Xaa is absent if Xaa at position 13 is absent, or Xaa is Lys
  VARIANT (15)..(15): Xaa is Phe or absent
  VARIANT (16)..(16): Xaa is absent if Xaa at position 15 is absent, or Xaa is Lys
  VARIANT (17)..(17): Xaa is Phe or absent
  VARIANT (18)..(18): Xaa is absent if Xaa at position 17 is absent, or Xaa is Lys
  VARIANT (19)..(19): Xaa is Phe or absent
  VARIANT (20)..(20): Xaa is absent if Xaa at position 19 is absent, or Xaa is Lys
  VARIANT (21)..(21): Xaa is Phe or absent
  VARIANT (22)..(22): Xaa is absent if Xaa at position 21 is absent, or Xaa is Lys
FRFKXXXXXXXXXXXXXXXXXX

SEQ ID NO: 92 - protein, 5 aa, Artificial Sequence
  Synthetic Sequence
  VARIANT (1)..(1): Xaa is Cit
XKFKF

SEQ ID NO: 93 - protein, 5 aa, Artificial Sequence
  Synthetic Sequence
RKFFK

SEQ ID NO: 94 - protein, 5 aa, Artificial Sequence
  Synthetic Sequence
  VARIANT (1)..(1): Xaa is Cit
XKFFK

SEQ ID NO: 95 - protein, 5 aa, Artificial Sequence
  Synthetic Sequence
RKFFK

SEQ ID NO: 96 - protein, 5 aa, Artificial Sequence
  Synthetic Sequence
  VARIANT (1)..(1): Xaa is Cit
XKFFK

SEQ ID NO: 97 - protein, 11 aa, Artificial Sequence
  Synthetic Sequence
RKFKFKFKFKF

SEQ ID NO: 98 - protein, 11 aa, Artificial Sequence
  Synthetic Sequence
  VARIANT (1)..(1): Xaa is Cit
XKFKFKFKFKF

SEQ ID NO: 99 - protein, 11 aa, Artificial Sequence
  Synthetic Sequence
RKFKFKFKFFK

SEQ ID NO: 100 - protein, 11 aa, Artificial Sequence
  Synthetic Sequence
  VARIANT (1)..(1): Xaa is Cit
XKFKFKFKFFK

SEQ ID NO: 101 - protein, 4 aa, Artificial Sequence
  Synthetic Sequence
  VARIANT (2)..(2): Xaa is Cit
FXKF

SEQ ID NO: 102 - protein, 4 aa, Artificial Sequence
  Synthetic Sequence
  VARIANT (2)..(2): Xaa is Cit
FXFK

SEQ ID NO: 103 - protein, 4 aa, Artificial Sequence
  Synthetic Sequence
FKKF

SEQ ID NO: 104 - protein, 4 aa, Artificial Sequence
  Synthetic Sequence
  VARIANT (2)..(2): Xaa is Cit
  VARIANT (4)..(4): Xaa is Cit
FXKX

SEQ ID NO: 105 - protein, 4 aa, Artificial Sequence
  Synthetic Sequence
  VARIANT (2)..(2): Xaa is Cit
FXKY

SEQ ID NO: 106 - protein, 4 aa, Artificial Sequence
  Synthetic Sequence
  VARIANT (2)..(2): Xaa is Cit
  VARIANT (4)..(4): Xaa is homoTyr
FXKX

SEQ ID NO: 107 - protein, 6 aa, Artificial Sequence
  Synthetic Sequence
FRKRKF

SEQ ID NO: 108 - protein, 22 aa, Artificial Sequence
  Synthetic Sequence
  VARIANT (4)..(4): Xaa is Cit
  VARIANT (5)..(5): Xaa is Lys or absent
  VARIANT (6)..(6): Xaa is absent if Xaa at position 5 is absent, or Xaa is Cit
  VARIANT (7)..(7): Xaa is Lys or absent
  VARIANT (8)..(8): Xaa is absent if Xaa at position 7 is absent, or Xaa is Cit
  VARIANT (9)..(9): Xaa is Lys or absent
  VARIANT (10)..(10): Xaa is absent if Xaa at position 9 is absent, or Xaa is Cit
  VARIANT (11)..(11): Xaa is Lys or absent
  VARIANT (12)..(12): Xaa is absent if Xaa at position 11 is absent, or Xaa is Cit
  VARIANT (13)..(13): Xaa is Lys or absent
  VARIANT (14)..(14): Xaa is absent if Xaa at position 13 is absent, or Xaa is Cit
  VARIANT (15)..(15): Xaa is Lys or absent
  VARIANT (16)..(16): Xaa is absent if Xaa at position 15 is absent, or Xaa is Cit
  VARIANT (17)..(17): Xaa is Lys or absent
  VARIANT (18)..(18): Xaa is absent if Xaa at position 17 is absent, or Xaa is Cit
  VARIANT (19)..(19): Xaa is Lys or absent
  VARIANT (20)..(20): Xaa is absent if Xaa at position 19 is absent, or Xaa is Cit
  VARIANT (21)..(21): Xaa is Lys or absent
  VARIANT (22)..(22): Xaa is absent if Xaa at position 21 is absent, or Xaa is Cit
FRKXXXXXXXXXXXXXXXXXXX

SEQ ID NO: 109 - protein, 22 aa, Artificial Sequence
  Synthetic Sequence
  VARIANT (5)..(5): Xaa is Lys or absent
  VARIANT (6)..(6): Xaa is absent if Xaa at position 5 is absent, or Xaa is Tyr
  VARIANT (7)..(7): Xaa is Lys or absent
  VARIANT (8)..(8): Xaa is absent if Xaa at position 7 is absent, or Xaa is Tyr
  VARIANT (9)..(9): Xaa is Lys or absent
  VARIANT (10)..(10): Xaa is absent if Xaa at position 9 is absent, or Xaa is Tyr
  VARIANT (11)..(11): Xaa is Lys or absent
  VARIANT (12)..(12): Xaa is absent if Xaa at position 11 is absent, or Xaa is Tyr
  VARIANT (13)..(13): Xaa is Lys or absent
  VARIANT (14)..(14): Xaa is absent if Xaa at position 13 is absent, or Xaa is Tyr
  VARIANT (15)..(15): Xaa is Lys or absent
  VARIANT (16)..(16): Xaa is absent if Xaa at position 15 is absent, or Xaa is Tyr
  VARIANT (17)..(17): Xaa is Lys or absent
  VARIANT (18)..(18): Xaa is absent if Xaa at position 17 is absent, or Xaa is Tyr
  VARIANT (19)..(19): Xaa is Lys or absent
  VARIANT (20)..(20): Xaa is absent if Xaa at position 19 is absent, or Xaa is Tyr
  VARIANT (21)..(21): Xaa is Lys or absent
  VARIANT (22)..(22): Xaa is absent if Xaa at position 21 is absent, or Xaa is Tyr
FRKYXXXXXXXXXXXXXXXXXX

SEQ ID NO: 110 - protein, 24 aa, Artificial Sequence
  Synthetic Sequence
  VARIANT (4)..(4): Xaa is Cit
  VARIANT (5)..(5): Xaa is Lys or absent
  VARIANT (6)..(6): Xaa is absent if Xaa at position 5 is absent, or Xaa is Cit
  VARIANT (7)..(7): Xaa is Lys or absent
  VARIANT (8)..(8): Xaa is absent if Xaa at position 7 is absent, or Xaa is Cit
  VARIANT (9)..(9): Xaa is Lys or absent
  VARIANT (10)..(10): Xaa is absent if Xaa at position 9 is absent, or Xaa is Cit
  VARIANT (11)..(11): Xaa is Lys or absent
  VARIANT (12)..(12): Xaa is absent if Xaa at position 11 is absent, or Xaa is Cit
  VARIANT (13)..(13): Xaa is Lys or absent
  VARIANT (14)..(14): Xaa is absent if Xaa at position 13 is absent, or Xaa is Cit
  VARIANT (15)..(15): Xaa is Lys or absent
  VARIANT (16)..(16): Xaa is absent if Xaa at position 15 is absent, or Xaa is Cit
  VARIANT (17)..(17): Xaa is Lys or absent
  VARIANT (18)..(18): Xaa is absent if Xaa at position 17 is absent, or Xaa is Cit
  VARIANT (19)..(19): Xaa is Lys or absent
  VARIANT (20)..(20): Xaa is absent if Xaa at position 19 is absent, or Xaa is Cit
  VARIANT (21)..(21): Xaa is Lys or absent
  VARIANT (22)..(22): Xaa is absent if Xaa at position 21 is absent, or Xaa is Cit
FRKXXXXXXXXXXXXXXXXXXXYY

SEQ ID NO: 111 - protein, 4 aa, Artificial Sequence
  Synthetic Sequence
FKKF

SEQ ID NO: 112 - protein, 4 aa, Artificial Sequence
  Synthetic Sequence
FKKF

SEQ ID NO: 113 - protein, 4 aa, Artificial Sequence
  Synthetic Sequence
  VARIANT (2)..(2): Xaa is Cit
  VARIANT (4)..(4): Xaa is Cit
FXKX

SEQ ID NO: 114 - protein, 4 aa, Artificial Sequence
  Synthetic Sequence
  VARIANT (2)..(2): Xaa is Cit
FXKY

SEQ ID NO: 115 - protein, 4 aa, Artificial Sequence
  Synthetic Sequence
  VARIANT (2)..(2): Xaa is Cit
  VARIANT (4)..(4): Xaa is homoTyr
FXKX

SEQ ID NO: 116 - protein, 6 aa, Artificial Sequence
  Synthetic Sequence
FRKRKF

SEQ ID NO: 117 - protein, 22 aa, Artificial Sequence
  Synthetic Sequence
  VARIANT (4)..(4): Xaa is Cit
  VARIANT (5)..(5): Xaa is Lys or absent
  VARIANT (6)..(6): Xaa is absent if Xaa at position 5 is absent, or Xaa is Cit
  VARIANT (7)..(7): Xaa is Lys or absent
  VARIANT (8)..(8): Xaa is absent if Xaa at position 7 is absent, or Xaa is Cit
  VARIANT (9)..(9): Xaa is Lys or absent
  VARIANT (10)..(10): Xaa is absent if Xaa at position 9 is absent, or Xaa is Cit
  VARIANT (11)..(11): Xaa is Lys or absent
  VARIANT (12)..(12): Xaa is absent if Xaa at position 11 is absent, or Xaa is Cit
  VARIANT (13)..(13): Xaa is Lys or absent
  VARIANT (14)..(14): Xaa is absent if Xaa at position 13 is absent, or Xaa is Cit
  VARIANT (15)..(15): Xaa is Lys or absent
  VARIANT (16)..(16): Xaa is absent if Xaa at position 15 is absent, or Xaa is Cit
  VARIANT (17)..(17): Xaa is Lys or absent
  VARIANT (18)..(18): Xaa is absent if Xaa at position 17 is absent, or Xaa is Cit
  VARIANT (19)..(19): Xaa is Lys or absent
  VARIANT (20)..(20): Xaa is absent if Xaa at position 19 is absent, or Xaa is Cit
  VARIANT (21)..(21): Xaa is Lys or absent
  VARIANT (22)..(22): Xaa is absent if Xaa at position 21 is absent, or Xaa is Cit
FRKXXXXXXXXXXXXXXXXXXX

SEQ ID NO: 118 - protein, 22 aa, Artificial Sequence
  Synthetic Sequence
  VARIANT (5)..(5): Xaa is Lys or absent
  VARIANT (6)..(6): Xaa is absent if Xaa at position 5 is absent, or Xaa is Tyr
  VARIANT (7)..(7): Xaa is Lys or absent
  VARIANT (8)..(8): Xaa is absent if Xaa at position 7 is absent, or Xaa is Tyr
  VARIANT (9)..(9): Xaa is Lys or absent
  VARIANT (10)..(10): Xaa is absent if Xaa at position 9 is absent, or Xaa is Tyr
  VARIANT (11)..(11): Xaa is Lys or absent
  VARIANT (12)..(12): Xaa is absent if Xaa at position 11 is absent, or Xaa is Tyr
  VARIANT (13)..(13): Xaa is Lys or absent
  VARIANT (14)..(14): Xaa is absent if Xaa at position 13 is absent, or Xaa is Tyr
  VARIANT (15)..(15): Xaa is Lys or absent
  VARIANT (16)..(16): Xaa is absent if Xaa at position 15 is absent, or Xaa is Tyr
  VARIANT (17)..(17): Xaa is Lys or absent
  VARIANT (18)..(18): Xaa is absent if Xaa at position 17 is absent, or Xaa is Tyr
  VARIANT (19)..(19): Xaa is Lys or absent
  VARIANT (20)..(20): Xaa is absent if Xaa at position 19 is absent, or Xaa is Tyr
  VARIANT (21)..(21): Xaa is Lys or absent
  VARIANT (22)..(22): Xaa is absent if Xaa at position 21 is absent, or Xaa is Tyr
FRKYXXXXXXXXXXXXXXXXXX

SEQ ID NO: 119 - protein, 24 aa, Artificial Sequence
  Synthetic Sequence
  VARIANT (4)..(4): Xaa is Cit
  VARIANT (5)..(5): Xaa is Lys or absent
  VARIANT (6)..(6): Xaa is absent if Xaa at position 5 is absent, or Xaa is Cit
  VARIANT (7)..(7): Xaa is Lys or absent
  VARIANT (8)..(8): Xaa is absent if Xaa at position 7 is absent, or Xaa is Cit
  VARIANT (9)..(9): Xaa is Lys or absent
  VARIANT (10)..(10): Xaa is absent if Xaa at position 9 is absent, or Xaa is Cit
  VARIANT (11)..(11): Xaa is Lys or absent
  VARIANT (12)..(12): Xaa is absent if Xaa at position 11 is absent, or Xaa is Cit
  VARIANT (13)..(13): Xaa is Lys or absent
  VARIANT (14)..(14): Xaa is absent if Xaa at position 13 is absent, or Xaa is Cit
  VARIANT (15)..(15): Xaa is Lys or absent
  VARIANT (16)..(16): Xaa is absent if Xaa at position 15 is absent, or Xaa is Cit
  VARIANT (17)..(17): Xaa is Lys or absent
  VARIANT (18)..(18): Xaa is absent if Xaa at position 17 is absent, or Xaa is Cit
  VARIANT (19)..(19): Xaa is Lys or absent
  VARIANT (20)..(20): Xaa is absent if Xaa at position 19 is absent, or Xaa is Cit
  VARIANT (21)..(21): Xaa is Lys or absent
  VARIANT (22)..(22): Xaa is absent if Xaa at position 21 is absent, or Xaa is Cit
FRKXXXXXXXXXXXXXXXXXXXYY

SEQ ID NO: 120 - protein, 22 aa, Artificial Sequence
  Synthetic Sequence
  VARIANT (4)..(4): Xaa is Cit
  VARIANT (5)..(5): Xaa is Lys or absent
  VARIANT (6)..(6): Xaa is absent if Xaa at position 5 is absent, or Xaa is Cit
  VARIANT (7)..(7): Xaa is Lys or absent
  VARIANT (8)..(8): Xaa is absent if Xaa at position 7 is absent, or Xaa is Cit
  VARIANT (9)..(9): Xaa is Lys or absent
  VARIANT (10)..(10): Xaa is absent if Xaa at position 9 is absent, or Xaa is Cit
  VARIANT (11)..(11): Xaa is Lys or absent
  VARIANT (12)..(12): Xaa is absent if Xaa at position 11 is absent, or Xaa is Cit
  VARIANT (13)..(13): Xaa is Lys or absent
  VARIANT (14)..(14): Xaa is absent if Xaa at position 13 is absent, or Xaa is Cit
  VARIANT (15)..(15): Xaa is Lys or absent
  VARIANT (16)..(16): Xaa is absent if Xaa at position 15 is absent, or Xaa is Cit
  VARIANT (17)..(17): Xaa is Lys or absent
  VARIANT (18)..(18): Xaa is absent if Xaa at position 17 is absent, or Xaa is Cit
  VARIANT (19)..(19): Xaa is Lys or absent
  VARIANT (20)..(20): Xaa is absent if Xaa at position 19 is absent, or Xaa is Cit
  VARIANT (21)..(21): Xaa is Lys or absent
  VARIANT (22)..(22): Xaa is absent if Xaa at position 21 is absent, or Xaa is Cit
FRKXXXXXXXXXXXXXXXXXXX

SEQ ID NO: 121 - protein, 6 aa, Artificial Sequence
  Synthetic Sequence
FRFKSK

SEQ ID NO: 122 - protein, 22 aa, Artificial Sequence
  Synthetic Sequence
  VARIANT (1)..(1): Xaa is an amino acid having a hydrophobic or basic side chain, such as Phe, Tyr, Val, Ala, Ile, Leu, Ser, His, Met, Phg, Cha, Aib, Tle, Nle, Nva, or homo-Phe, or absent
  VARIANT (2)..(2): Xaa is an amino acid having a hydrophobic or basic side chain, such as Arg, Lys, Cit, Val, Leu, Ser, Ala, Gly, His, Gln, Phg and Phe
  VARIANT (3)..(3): Xaa is a trifunctional amino acid such as an amino-dicarboxylic acid or a diamino-carboxylic acid, for example Aaa, Dap, Dab, Ama, Glu, Apa, Lys, Orn, Ser, homoSer, homoThr, and homoLys
  VARIANT (4)..(4): Xaa is Phe, Ala, Trp, Tyr, Phg, Val, His, Lys, Abu, Met, Cit, Orn, Ser, Thr, Leu, Ile, Arg, Tyr, homo-Tyr, homo-Phe, beta-Phe,or beta-homo-Phe
  VARIANT (5)..(5): Xaa is a trifunctional amino acid such as an amino-dicarboxylic acid or a diamino-carboxylic acid, for example Aaa, Dap, Dab, Ama, Glu, Apa, Lys, Orn, Ser, homoSer, homoThr, and homoLys, or absent
  VARIANT (6)..(6): Xaa is absent if Xaa at position 5 is absent, or Xaa is Phe, Ala, Trp, Tyr, Phg, Val, His, Lys, Abu, Met, Cit, Orn, Ser, Thr, Leu, Ile, Arg, Tyr, homo-Tyr, homo-Phe, beta-Phe, or beta-homo-Phe
  VARIANT (7)..(7): Xaa is a trifunctional amino acid such as an amino-dicarboxylic acid or a diamino-carboxylic acid, for example Aaa, Dap, Dab, Ama, Glu, Apa, Lys, Orn, Ser, homoSer, homoThr, and homoLys, or absent
  VARIANT (8)..(8): Xaa is absent if Xaa at position 7 is absent, or Xaa is Phe, Ala, Trp, Tyr, Phg, Val, His, Lys, Abu, Met, Cit, Orn, Ser, Thr, Leu, Ile, Arg, Tyr, homo-Tyr, homo-Phe, beta-Phe, or beta-homo-Phe
  VARIANT (9)..(9): Xaa is a trifunctional amino acid such as an amino-dicarboxylic acid or a diamino-carboxylic acid, for example Aaa, Dap, Dab, Ama, Glu, Apa, Lys, Orn, Ser, homoSer, homoThr, and homoLys, or absent
  VARIANT (10)..(10): Xaa is absent if Xaa at position 9 is absent, or Xaa is Phe, Ala, Trp, Tyr, Phg, Val, His, Lys, Abu, Met, Cit, Orn, Ser, Thr, Leu, Ile, Arg, Tyr, homo-Tyr, homo-Phe, beta-Phe, or beta-homo-Phe
  VARIANT (11)..(11): Xaa is a trifunctional amino acid such as an amino-dicarboxylic acid or a diamino-carboxylic acid, for example Aaa, Dap, Dab, Ama, Glu, Apa, Lys, Orn, Ser, homoSer, homoThr, and homoLys, or absent
  VARIANT (12)..(12): Xaa is absent if Xaa at position 11 is absent, or Xaa is Phe, Ala, Trp, Tyr, Phg, Val, His, Lys, Abu, Met, Cit, Orn, Ser, Thr, Leu, Ile, Arg, Tyr, homo-Tyr, homo-Phe, beta-Phe, or beta-homo-Phe
  VARIANT (13)..(13): Xaa is a trifunctional amino acid such as an amino-dicarboxylic acid or a diamino-carboxylic acid, for example Aaa, Dap, Dab, Ama, Glu, Apa, Lys, Orn, Ser, homoSer, homoThr, and homoLys, or absent
  VARIANT (14)..(14): Xaa is absent if Xaa at position 13 is absent, or Xaa is Phe, Ala, Trp, Tyr, Phg, Val, His, Lys, Abu, Met, Cit, Orn, Ser, Thr, Leu, Ile, Arg, Tyr, homo-Tyr, homo-Phe, beta-Phe, or beta-homo-Phe
  VARIANT (15)..(15): Xaa is a trifunctional amino acid such as an amino-dicarboxylic acid or a diamino-carboxylic acid, for example Aaa, Dap, Dab, Ama, Glu, Apa, Lys, Orn, Ser, homoSer, homoThr, and homoLys, or absent
  VARIANT (16)..(16): Xaa is absent if Xaa at position 15 is absent, or Xaa is Phe, Ala, Trp, Tyr, Phg, Val, His, Lys, Abu, Met, Cit, Orn, Ser, Thr, Leu, Ile, Arg, Tyr, homo-Tyr, homo-Phe, beta-Phe, or beta-homo-Phe
  VARIANT (17)..(17): Xaa is a trifunctional amino acid such as an amino-dicarboxylic acid or a diamino-carboxylic acid, for example Aaa, Dap, Dab, Ama, Glu, Apa, Lys, Orn, Ser, homoSer, homoThr, and homoLys, or absent
  VARIANT (18)..(18): Xaa is absent if Xaa at position 17 is absent, or Xaa is Phe, Ala, Trp, Tyr, Phg, Val, His, Lys, Abu, Met, Cit, Orn, Ser, Thr, Leu, Ile, Arg, Tyr, homo-Tyr, homo-Phe, beta-Phe, or beta-homo-Phe
  VARIANT (19)..(19): Xaa is a trifunctional amino acid such as an amino-dicarboxylic acid or a diamino-carboxylic acid, for example Aaa, Dap, Dab, Ama, Glu, Apa, Lys, Orn, Ser, homoSer, homoThr, and homoLys, or absent
  VARIANT (20)..(20): Xaa is absent if Xaa at position 19 is absent, or Xaa is Phe, Ala, Trp, Tyr, Phg, Val, His, Lys, Abu, Met, Cit, Orn, Ser, Thr, Leu, Ile, Arg, Tyr, homo-Tyr, homo-Phe, beta-Phe, or beta-homo-Phe
  VARIANT (21)..(21): Xaa is a trifunctional amino acid such as an amino-dicarboxylic acid or a diamino-carboxylic acid, for example Aaa, Dap, Dab, Ama, Glu, Apa, Lys, Orn, Ser, homoSer, homoThr, and homoLys, or absent
  VARIANT (22)..(22): Xaa is absent if Xaa at position 21 is absent, or Xaa is Phe, Ala, Trp, Tyr, Phg, Val, His, Lys, Abu, Met, Cit, Orn, Ser, Thr, Leu, Ile, Arg, Tyr, homo-Tyr, homo-Phe, beta-Phe, or beta-homo-Phe
XXXXXXXXXXXXXXXXXXXXXX

SEQ ID NO: 123 - protein, 22 aa, Artificial Sequence
  Synthetic Sequence
  VARIANT (1)..(1): Xaa is an amino acid having a hydrophobic or basic side chain, such as Phe, Tyr, Val, Ala, Ile, Leu, Ser, His, Met, Phg, Cha, Aib, Tle, Nle, Nva, or homo-Phe, or absent
  VARIANT (2)..(2): Xaa is an amino acid having a hydrophobic or basic side chain, such as Arg, Lys, Cit, Val, Leu, Ser, Ala, Gly, His, Gln, Phg and Phe
  VARIANT (3)..(3): Xaa is Phe, Ala, Trp, Tyr, Phg, Val, His, Lys, Abu, Met, Cit, Orn, Ser, Thr, Leu, Ile, Arg, or Tyr
  VARIANT (4)..(4): Xaa is a trifunctional amino acid such as an amino-dicarboxylic acid or a diamino-carboxylic acid, for example Aaa, Dap, Dab, Ama, Glu, Apa, Lys, Orn, Ser, homoSer, homoThr, and homoLys
  VARIANT (5)..(5): Xaa is Phe, Ala, Trp, Tyr, Phg, Val, His, Lys, Abu, Met, Cit, Orn, Ser, Thr, Leu, Ile, Arg, or Tyr, or absent
  VARIANT (6)..(6): Xaa is absent if Xaa at position 5 is absent, or Xaa is a trifunctional amino acid such as an amino-dicarboxylic acid or a diamino-carboxylic acid, for example Aaa, Dap, Dab, Ama, Glu, Apa, Lys, Orn, Ser, homoSer, homoThr, and homoLys
  VARIANT (7)..(7): Xaa is Phe, Ala, Trp, Tyr, Phg, Val, His, Lys, Abu, Met, Cit, Orn, Ser, Thr, Leu, Ile, Arg, or Tyr, or absent
  VARIANT (8)..(8): Xaa is absent if Xaa at position 7 is absent, or Xaa is a trifunctional amino acid such as an amino-dicarboxylic acid or a diamino-carboxylic acid, for example Aaa, Dap, Dab, Ama, Glu, Apa, Lys, Orn, Ser, homoSer, homoThr, and homoLys
  VARIANT (9)..(9): Xaa is Phe, Ala, Trp, Tyr, Phg, Val, His, Lys, Abu, Met, Cit, Orn, Ser, Thr, Leu, Ile, Arg, or Tyr, or absent
  VARIANT (10)..(10): Xaa is absent if Xaa at position 9 is absent, or Xaa is a trifunctional amino acid such as an amino-dicarboxylic acid or a diamino-carboxylic acid, for example Aaa, Dap, Dab, Ama, Glu, Apa, Lys, Orn, Ser, homoSer, homoThr, and homoLys
  VARIANT (11)..(11): Xaa is Phe, Ala, Trp, Tyr, Phg, Val, His, Lys, Abu, Met, Cit, Orn, Ser, Thr, Leu, Ile, Arg, or Tyr, or absent
  VARIANT (12)..(12): Xaa is absent if Xaa at position 11 is absent, or Xaa is a trifunctional amino acid such as an amino-dicarboxylic acid or a diamino-carboxylic acid, for example Aaa, Dap, Dab, Ama, Glu, Apa, Lys, Orn, Ser, homoSer, homoThr, and homoLys
  VARIANT (13)..(13): Xaa is Phe, Ala, Trp, Tyr, Phg, Val, His, Lys, Abu, Met, Cit, Orn, Ser, Thr, Leu, Ile, Arg, or Tyr, or absent
  VARIANT (14)..(14): Xaa is absent if Xaa at position 13 is absent, or Xaa is a trifunctional amino acid such as an amino-dicarboxylic acid or a diamino-carboxylic acid, for example Aaa, Dap, Dab, Ama, Glu, Apa, Lys, Orn, Ser, homoSer, homoThr, and homoLys
  VARIANT (15)..(15): Xaa is Phe, Ala, Trp, Tyr, Phg, Val, His, Lys, Abu, Met, Cit, Orn, Ser, Thr, Leu, Ile, Arg, or Tyr, or absent
  VARIANT (16)..(16): Xaa is absent if Xaa at position 15 is absent, or Xaa is a trifunctional amino acid such as an amino-dicarboxylic acid or a diamino-carboxylic acid, for example Aaa, Dap, Dab, Ama, Glu, Apa, Lys, Orn, Ser, homoSer, homoThr, and homoLys
  VARIANT (17)..(17): Xaa is Phe, Ala, Trp, Tyr, Phg, Val, His, Lys, Abu, Met, Cit, Orn, Ser, Thr, Leu, Ile, Arg, or Tyr, or absent
  VARIANT (18)..(18): Xaa is absent if Xaa at position 17 is absent, or Xaa is a trifunctional amino acid such as an amino-dicarboxylic acid or a diamino-carboxylic acid, for example Aaa, Dap, Dab, Ama, Glu, Apa, Lys, Orn, Ser, homoSer, homoThr, and homoLys
  VARIANT (19)..(19): Xaa is Phe, Ala, Trp, Tyr, Phg, Val, His, Lys, Abu, Met, Cit, Orn, Ser, Thr, Leu, Ile, Arg, or Tyr, or absent
  VARIANT (20)..(20): Xaa is absent if Xaa at position 19 is absent, or Xaa is a trifunctional amino acid such as an amino-dicarboxylic acid or a diamino-carboxylic acid, for example Aaa, Dap, Dab, Ama, Glu, Apa, Lys, Orn, Ser, homoSer, homoThr, and homoLys
  VARIANT (21)..(21): Xaa is Phe, Ala, Trp, Tyr, Phg, Val, His, Lys, Abu, Met, Cit, Orn, Ser, Thr, Leu, Ile, Arg, or Tyr, or absent
  VARIANT (22)..(22): Xaa is absent if Xaa at position 21 is absent, or Xaa is a trifunctional amino acid such as an amino-dicarboxylic acid or a diamino-carboxylic acid, for example Aaa, Dap, Dab, Ama, Glu, Apa, Lys, Orn, Ser, homoSer, homoThr, and homoLys
XXXXXXXXXXXXXXXXXXXXXX

SEQ ID NO: 124 - protein, 22 aa, Artificial Sequence
  Synthethic Sequence
  VARIANT (1)..(1): Xaa is an amino acid having a hydrophobic or basic side chain, such as Phe, Tyr, Val, Ala, Ile, Leu, Ser, His, Met, Phg, Cha, Aib, Tle, Nle, Nva, or homo-Phe, or absent
  VARIANT (2)..(2): Xaa is an amino acid having a hydrophobic or basic side chain, such as Arg, Lys, Cit, Val, Leu, Ser, Ala, Gly, His, Gln, Phg and Phe
  VARIANT (3)..(3): Xaa is a carboxylic amino acid such as Ama, Glu, Aaa, Apa or a trifunctional amino acid selected from Dap, Dab, Ser, Thr, Lys, Orn, homoLys, homoSer and homoThr
  VARIANT (4)..(4): Xaa is Phe, Ala, Trp, Tyr, Phg, Val, His, Lys, Abu, Met, Cit, Orn, Ser, Thr, Leu, Ile, Arg, homo-Phe, beta-Phe, or beta-homo-Phe
  VARIANT (5)..(5): Xaa is a carboxylic amino acid such as Ama, Glu, Aaa, Apa or a trifunctional amino acid selected from Dap, Dab, Ser, Thr, Lys, Orn, homoLys, homoSer and homoThr, or absent
  VARIANT (6)..(6): Xaa is absent if Xaa at postition 5 is absent, or Xaa is Phe, Ala, Trp, Tyr, Phg, Val, His, Lys, Abu, Met, Cit, Orn, Ser, Thr, Leu, Ile, Arg, homo-Phe, beta-Phe, or beta-homo-Phe
  VARIANT (7)..(7): Xaa is a carboxylic amino acid such as Ama, Glu, Aaa, Apa or a trifunctional amino acid selected from Dap, Dab, Ser, Thr, Lys, Orn, homoLys, homoSer and homoThr, or absent
  VARIANT (8)..(8): Xaa is absent if Xaa at postition 7 is absent, or Xaa is Phe, Ala, Trp, Tyr, Phg, Val, His, Lys, Abu, Met, Cit, Orn, Ser, Thr, Leu, Ile, Arg, homo-Phe, beta-Phe, or beta-homo-Phe
  VARIANT (9)..(9): Xaa is a carboxylic amino acid such as Ama, Glu, Aaa, Apa or a trifunctional amino acid selected from Dap, Dab, Ser, Thr, Lys, Orn, homoLys, homoSer and homoThr, or absent
  VARIANT (10)..(10): Xaa is absent if Xaa at postition 9 is absent, or Xaa is Phe, Ala, Trp, Tyr, Phg, Val, His, Lys, Abu, Met, Cit, Orn, Ser, Thr, Leu, Ile, Arg, homo-Phe, beta-Phe, or beta-homo-Phe
  VARIANT (11)..(11): Xaa is a carboxylic amino acid such as Ama, Glu, Aaa, Apa or a trifunctional amino acid selected from Dap, Dab, Ser, Thr, Lys, Orn, homoLys, homoSer and homoThr, or absent
  VARIANT (12)..(12): Xaa is absent if Xaa at postition 11 is absent, or Xaa is Phe, Ala, Trp, Tyr, Phg, Val, His, Lys, Abu, Met, Cit, Orn, Ser, Thr, Leu, Ile, Arg, homo-Phe, beta-Phe, or beta-homo-Phe
  VARIANT (13)..(13): Xaa is a carboxylic amino acid such as Ama, Glu, Aaa, Apa or a trifunctional amino acid selected from Dap, Dab, Ser, Thr, Lys, Orn, homoLys, homoSer and homoThr, or absent
  VARIANT (14)..(14): Xaa is absent if Xaa at postition 13 is absent, or Xaa is Phe, Ala, Trp, Tyr, Phg, Val, His, Lys, Abu, Met, Cit, Orn, Ser, Thr, Leu, Ile, Arg, homo-Phe, beta-Phe, or beta-homo-Phe
  VARIANT (15)..(15): Xaa is a carboxylic amino acid such as Ama, Glu, Aaa, Apa or a trifunctional amino acid selected from Dap, Dab, Ser, Thr, Lys, Orn, homoLys, homoSer and homoThr, or absent
  VARIANT (16)..(16): Xaa is absent if Xaa at postition 15 is absent, or Xaa is Phe, Ala, Trp, Tyr, Phg, Val, His, Lys, Abu, Met, Cit, Orn, Ser, Thr, Leu, Ile, Arg, homo-Phe, beta-Phe, or beta-homo-Phe
  VARIANT (17)..(17): Xaa is a carboxylic amino acid such as Ama, Glu, Aaa, Apa or a trifunctional amino acid selected from Dap, Dab, Ser, Thr, Lys, Orn, homoLys, homoSer and homoThr, or absent
  VARIANT (18)..(18): Xaa is absent if Xaa at postition 17 is absent, or Xaa is Phe, Ala, Trp, Tyr, Phg, Val, His, Lys, Abu, Met, Cit, Orn, Ser, Thr, Leu, Ile, Arg, homo-Phe, beta-Phe, or beta-homo-Phe
  VARIANT (19)..(19): Xaa is a carboxylic amino acid such as Ama, Glu, Aaa, Apa or a trifunctional amino acid selected from Dap, Dab, Ser, Thr, Lys, Orn, homoLys, homoSer and homoThr, or absent
  VARIANT (20)..(20): Xaa is absent if Xaa at postition 19 is absent, or Xaa is Phe, Ala, Trp, Tyr, Phg, Val, His, Lys, Abu, Met, Cit, Orn, Ser, Thr, Leu, Ile, Arg, homo-Phe, beta-Phe, or beta-homo-Phe
  VARIANT (21)..(21): Xaa is a carboxylic amino acid such as Ama, Glu, Aaa, Apa or a trifunctional amino acid selected from Dap, Dab, Ser, Thr, Lys, Orn, homoLys, homoSer and homoThr, or absent
  VARIANT (22)..(22): Xaa is absent if Xaa at postition 21 is absent, or Xaa is Phe, Ala, Trp, Tyr, Phg, Val, His, Lys, Abu, Met, Cit, Orn, Ser, Thr, Leu, Ile, Arg, homo-Phe, beta-Phe, or beta-homo-Phe
XXXXXXXXXXXXXXXXXXXXXX

SEQ ID NO: 125 - protein, 20 aa, Artificial Sequence
  Synthetic Sequence
  VARIANT (1)..(1): Xaa is Glu, Aaa, Dap, Dab, Ser, Thr, homoSer, homoThr, or Ama
  VARIANT (2)..(2): Xaa is Phe, Ala, Trp, Tyr, Phg, Met, Val, His, Lys, Arg, Cit, Abu, or Orn
  VARIANT (3)..(3): Xaa is Glu, Aaa, Dap, Dab, Ser, Thr, homoSer, homoThr, Ama, or absent
  VARIANT (4)..(4): Xaa is absent if Xaa at position 3 is absent, or Xaa is Phe, Ala, Trp, Tyr, Phg, Met, Val, His, Lys, Arg, Cit, Abu, or Orn
  VARIANT (5)..(5): Xaa is Glu, Aaa, Dap, Dab, Ser, Thr, homoSer, homoThr, Ama, or absent
  VARIANT (6)..(6): Xaa is absent if Xaa at position 5 is absent, or Xaa is Phe, Ala, Trp, Tyr, Phg, Met, Val, His, Lys, Arg, Cit, Abu, or Orn
  VARIANT (7)..(7): Xaa is Glu, Aaa, Dap, Dab, Ser, Thr, homoSer, homoThr, Ama, or absent
  VARIANT (8)..(8): Xaa is absent if Xaa at position 7 is absent, or Xaa is Phe, Ala, Trp, Tyr, Phg, Met, Val, His, Lys, Arg, Cit, Abu, or Orn
  VARIANT (9)..(9): Xaa is Glu, Aaa, Dap, Dab, Ser, Thr, homoSer, homoThr, Ama, or absent
  VARIANT (10)..(10): Xaa is absent if Xaa at position 9 is absent, or Xaa is Phe, Ala, Trp, Tyr, Phg, Met, Val, His, Lys, Arg, Cit, Abu, or Orn
  VARIANT (11)..(11): Xaa is Glu, Aaa, Dap, Dab, Ser, Thr, homoSer, homoThr, Ama, or absent
  VARIANT (12)..(12): Xaa is absent if Xaa at position 11 is absent, or Xaa is Phe, Ala, Trp, Tyr, Phg, Met, Val, His, Lys, Arg, Cit, Abu, or Orn
  VARIANT (13)..(13): Xaa is Glu, Aaa, Dap, Dab, Ser, Thr, homoSer, homoThr, Ama, or absent
  VARIANT (14)..(14): Xaa is absent if Xaa at position 13 is absent, or Xaa is Phe, Ala, Trp, Tyr, Phg, Met, Val, His, Lys, Arg, Cit, Abu, or Orn
  VARIANT (15)..(15): Xaa is Glu, Aaa, Dap, Dab, Ser, Thr, homoSer, homoThr, Ama, or absent
  VARIANT (16)..(16): Xaa is absent if Xaa at position 15 is absent, or Xaa is Phe, Ala, Trp, Tyr, Phg, Met, Val, His, Lys, Arg, Cit, Abu, or Orn
  VARIANT (17)..(17): Xaa is Glu, Aaa, Dap, Dab, Ser, Thr, homoSer, homoThr, Ama, or absent
  VARIANT (18)..(18): Xaa is absent if Xaa at position 17 is absent, or Xaa is Phe, Ala, Trp, Tyr, Phg, Met, Val, His, Lys, Arg, Cit, Abu, or Orn
  VARIANT (19)..(19): Xaa is Glu, Aaa, Dap, Dab, Ser, Thr, homoSer, homoThr, Ama, or absent
  VARIANT (20)..(20): Xaa is absent if Xaa at position 19 is absent, or Xaa is Phe, Ala, Trp, Tyr, Phg, Met, Val, His, Lys, Arg, Cit, Abu, or Orn
XXXXXXXXXXXXXXXXXXXX